SEQUENCE LISTING (61 sequences)

SEQ ID NO: 1 - DNA, 4634 bp, synthetic construct
  misc_feature 1..4634: note = vector genome
  repeat_region 1..130: note = 5'ITR
  misc_feature 130..168: note = AAV 2 seq upstream of 3'ITR
  regulatory 213..678: note = promoter - hSynapsin; regulatory_class = promoter
  misc_feature 3596..4183: note = WRPE
  regulatory 4201..4439: note = polyA_signal - SV40 polyA; regulatory_class = polyA_signal_sequence
  misc_feature 4467..4505: note = AAV2 seq upstream of 3'ITR
  repeat_region 4505..4634: note = 3'ITR
  source 1..4634: mol_type = other DNA
  CDS 699..3581: note = hCDKL5_1; protein_id = 2; translation = MKIPNIGNVMNKFEILGVVGEGAYGVVLKCRHKETHEIVAIKKF KDSEENEEVKETTLRELKMLRTLKQENIVELKEAFRRRGKLYLVFEYVEKNMLELLEE MPNGVPPEKVKSYIYQLIKAIHWCHKNDIVHRDIKPENLLISHNDVLKLCDFGFARNL SEGNNANYTEYVATRWYRSPELLLGAPYGKSVDMWSVGCILGELSDGQPLFPGESEID QLFTIQKVLGPLPSEQMKLFYSNPRFHGLRFPAVNHPQSLERRYLGILNSVLLDLMKN LLKLDPADRYLTEQCLNHPTFQTQRLLDRSPSRSAKRKPYHVESSTLSNRNQAGKSTA LQSHHRSNSKDIQNLSVGLPRADEGLPANESFLNGNLAGASLSPLHTKTYQASSQPGS TSKDLTNNNIPHLLSPKEAKSKTEFDFNIDPKPSEGPGTKYLKSNSRSQQNRHSFMES SQSKAGTLQPNEKQSRHSYIDTIPQSSRSPSYRTKAKSHGALSDSKSVSNLSEARAQI AEPSTSRYFPSSCLDLNSPTSPTPTRHSDTRTLLSPSGRNNRNEGTLDSRRTTTRHSK TMEELKLPEHMDSSHSHSLSAPHESFSYGLGYTSPFSSQQRPHRHSMYVTRDKVRAKG LDGSLSIGQGMAARANSLQLLSPQPGEQLPPEMTVARSSVKETSREGTSSFHTRQKSE GGVYHDPHSDDGTAPKENRHLYNDPVPRRVGSFYRVPSPRPDNSFHENNVSTRVSSLP SESSSGTNHSKRQPAFDPWKSPENISHSEQLKEKEKQGFFRSMKKKKKKSQTVPNSDS PDLLTLQKSIHSASTPSSRPKEWRPEKISDLQTQSQPLKSLRKLLHLSSASNHPASSD PRFQPLTAQQTKNSFSEIRIHPLSQASGGSSNIRQEPAPKGRPALQLPGQMDPGWHVS SVTRSATEGPSYSEQLGAKSGPNGHPYNRTNRSRMPNLNDLKETAL
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgagagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - protein, 960 aa, synthetic construct
  REGION 1..960: note = Synthetic Construct
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 3 - DNA, 4750 bp, synthetic construct
  misc_feature 1..4750: note = Vector Genome
  repeat_region 1..130: note = 5'ITR
  misc_feature 130..168: note = AAV2 seq usptream of 3'ITR
  regulatory 213..678: note = promoter - hSynapsin; regulatory_class = promoter
  misc_feature 3590..3705: note = miR183
  misc_feature 3712..4299: note = WRPE
  regulatory 4317..4555: note = polyA_signal - SV40 polyA; regulatory_class = polyA_signal_sequence
  misc_feature 4583..4621: note = AAV2 seq upstream of 3'ITR
  repeat_region 4621..4750: note = 3'ITR
  source 1..4750: mol_type = other DNA
  CDS 699..3581: note = hCDKL5-1; protein_id = 4; translation = MKIPNIGNVMNKFEILGVVGEGAYGVVLKCRHKETHEIVAIKKF KDSEENEEVKETTLRELKMLRTLKQENIVELKEAFRRRGKLYLVFEYVEKNMLELLEE MPNGVPPEKVKSYIYQLIKAIHWCHKNDIVHRDIKPENLLISHNDVLKLCDFGFARNL SEGNNANYTEYVATRWYRSPELLLGAPYGKSVDMWSVGCILGELSDGQPLFPGESEID QLFTIQKVLGPLPSEQMKLFYSNPRFHGLRFPAVNHPQSLERRYLGILNSVLLDLMKN LLKLDPADRYLTEQCLNHPTFQTQRLLDRSPSRSAKRKPYHVESSTLSNRNQAGKSTA LQSHHRSNSKDIQNLSVGLPRADEGLPANESFLNGNLAGASLSPLHTKTYQASSQPGS TSKDLTNNNIPHLLSPKEAKSKTEFDFNIDPKPSEGPGTKYLKSNSRSQQNRHSFMES SQSKAGTLQPNEKQSRHSYIDTIPQSSRSPSYRTKAKSHGALSDSKSVSNLSEARAQI AEPSTSRYFPSSCLDLNSPTSPTPTRHSDTRTLLSPSGRNNRNEGTLDSRRTTTRHSK TMEELKLPEHMDSSHSHSLSAPHESFSYGLGYTSPFSSQQRPHRHSMYVTRDKVRAKG LDGSLSIGQGMAARANSLQLLSPQPGEQLPPEMTVARSSVKETSREGTSSFHTRQKSE GGVYHDPHSDDGTAPKENRHLYNDPVPRRVGSFYRVPSPRPDNSFHENNVSTRVSSLP SESSSGTNHSKRQPAFDPWKSPENISHSEQLKEKEKQGFFRSMKKKKKKSQTVPNSDS PDLLTLQKSIHSASTPSSRPKEWRPEKISDLQTQSQPLKSLRKLLHLSSASNHPASSD PRFQPLTAQQTKNSFSEIRIHPLSQASGGSSNIRQEPAPKGRPALQLPGQMDPGWHVS SVTRSATEGPSYSEQLGAKSGPNGHPYNRTNRSRMPNLNDLKETAL
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgagagcttataccggtagtgaattctaccagtgccataggatagtgaattctaccagtgccatacacgtgagtgaattctaccagtgccatagcatgcagtgaattctaccagtgccataggtacccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - protein, 960 aa, synthetic construct; identical to SEQ ID NO: 2
  REGION 1..960: note = Synthetic Construct

SEQ ID NO: 5 - DNA, 4757 bp, synthetic construct
  misc_feature 1..4757: note = Vector Genome
  repeat_region 1..130: note = 5'ITR
  misc_feature 130..168: note = AAV2 seq usptream of 3'ITR
  regulatory 213..678: note = promoter - hSynapsin; regulatory_class = promoter
  misc_feature 690..695: note = Kozak
  misc_feature 3719..4306: note = WRPE
  regulatory 4324..4562: note = polyA_signal - SV40 polyA; regulatory_class = polyA_signal_sequence
  misc_feature 4590..4628: note = AAV2 seq usptream of 3'ITR
  repeat_region 4628..4757: note = 3'ITR
  source 1..4757: mol_type = other DNA
  misc_feature 696..3704: note = nucleic acid sequence encoding hCDKL5-2GS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagatccccaatatcggcaacgtgatgaataagttcgagatcctgggagtggtgggagagggagcatacggcgtggtgctgaagtgcagacacaaggagacacacgagatcgtggccatcaagaagtttaaggatagcgaggagaatgaggaggtgaaggagacaaccctgcgcgagctgaagatgctgcggacactgaagcaggagaacatcgtggagctgaaggaggccttccggagaaggggcaagctgtacctggtgtttgagtacgtggagaagaacatgctggagctgctggaggagatgcctaatggcgtgccccctgagaaggtgaagagctacatctatcagctgatcaaggccatccactggtgccacaagaacgacatcgtgcaccgcgatatcaagccagagaacctgctgatcagccacaatgacgtgctgaagctgtgcgatttcggctttgcccggaacctgtccgagggcaacaacgccaattacacagagtacgtggcaaccaggtggtaccggtcccctgagctgctgctgggagcaccatacggcaagtctgtggacatgtggagcgtgggctgcatcctgggagagctgtccgacggacagccactgttccctggcgagtctgagatcgatcagctgtttaccatccagaaggtgctgggccccctgccttccgagcagatgaagctgttctactctaacccaaggttccacggcctgaggtttccagccgtgaatcacccccagtctctggagcgccggtatctgggcatcctgaacagcgtgctgctggatctgatgaagaatctgctgaagctggaccctgccgatcgctacctgaccgagcagtgtctgaatcacccaacatttcagacccagagactgctggacaggagcccctcccgctctgccaagaggaagccttatcacgtggagagctccaccctgagcaatcgcaaccaggccggcaagtccacagccctgcagtctcaccaccggagcaactccaaggacatccagaatctgagcgtgggcctgccaagggcagacgagggcctgcctgccaacgagtccttcctgaatggaaacctggcaggagcctctctgagcccactgcacacaaagacctaccaggcctctagccagcctggctccacatctaaggacctgaccaacaataacatccctcacctgctgagcccaaaggaggccaagtccaagaccgagttcgactttaatatcgatccaaagcccagcgagggcccaggcacaaagtatctgaagagcaactccagatctcagcagaataggcactctttcatggagtcctctcagagcaaggccggcaccctgcagcccaacgagaagcagagcaggcactcctacatcgataccatcccacagagctcccgcagcccctcctatcggacaaaggccaagagccacggcgccctgtctgacagcaagtccgtgtctaatctgtccgaggcaagagcacagatcgcagagccaagcacctccaggtactttccctcttcctgtctggacctgaactctccaacaagccctacaccaaccagacacagcgatacaaggaccctgctgtctcccagcggcagaaataacaggaacgagggcaccctggatagcagaaggaccacaaccagacactccaagacaatggaggagctgaagctgcccgagcacatggactcctctcactcccactctctgagcgcccctcacgagtccttctcttacggcctgggctataccagcccttttagctcccagcagcgcccacacaggcactccatgtacgtgacaagagacaaggtgagggccaagggcctggacggcagcctgtccatcggacagggaatggcagcaagggcaaatagcctgcagctgctgtccccacagcctggagagcagctgccaccagagatgaccgtggccagatctagcgtgaaggagacaagcagggagggcacctcctctttccacacaagacagaagagcgagggcggcgtgtatcacgacccacactccgacgacggaaccgcacccaaggagaacaggcacctgtacaacgatccagtgccccgccgggtgggctctttctatcgggtgcctagcccacggcccgacaactcctttcacgagaataacgtgtctaccagagtgagctccctgcctagcgagtctagctccggcacaaaccactccaagaggcagccagcctttgatccctggaagtctcctgagaatatctctcacagcgagcagctgaaggagaaggagaagcagggcttctttcgcagcatgaagaagaagaagaagaagagccagacaaccgacagcaccaacggcgagaatcctagcatcaagaagtccctgttcccactgtttaactccaagaatcacctgaagcactctagctccctgaagaagctgcccgtggtgacacctccaatggtgcccaattccgactctcctgatctgctgaccctgcagaagagcatccacagcgcctccacaccatctagcagacccaaggagtggaggccagagaagatctccgatctgcagacccagtcccagcccctgaagtctctgcggaagctgctgcacctgtcctctgccagcaaccaccctgcaagctccgaccctaggttccagccactgacagcccagcagaccaagaactcttttagcgagatcagaatccacccactgtcccaggcctctggaggctctagcaacatccgccaggagcctgccccaaagggccggccagccctgcagctgccaggacagatggaccccggctggcacgtgtcctctgtgacaagatccgccaccgagggaccatcctactctgagcagctgggagcaaagtctggaccaaacggacacccttataacaggaccaatagatccaggatgcctaatctgaacgacctgaaggagacagccctgtgatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - protein, 1001 aa, synthetic construct
  REGION 1..1001: note = Synthetic Construct; note = hCDKL5-2GS
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTTDSTNGENPSIKKSLFPLFNSKNHLKHSSSLKKLPVVTPPMVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 7 - DNA, 4583 bp, synthetic construct
  misc_feature 1..4583: note = Vector Genome
  repeat_region 1..130: note = 5'ITR
  misc_feature 130..168: note = AAV2 seq upstream of 3'ITR
  regulatory 213..678: note = promoter - hSynapsin; regulatory_class = promoter
  misc_feature 690..695: note = Kozak
  misc_feature 3545..4132: note = WRPE
  regulatory 4150..4388: note = polyA_signal - SV40 polyA; regulatory_class = polyA_signal_sequence
  misc_feature 4416..4454: note = AAV2 seq upstream of 3'ITR
  repeat_region 4454..4583: note = 3'ITR
  source 1..4583: mol_type = other DNA
  misc_feature 696..3530: note = nucleic acid sequence encoding hCDKL5-3GS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagatccccaatatcggcaacgtgatgaataagttcgagatcctgggagtggtgggagagggagcatacggagtggtgctgaagtgccggcacaaggagacacacgagatcgtggccatcaagaagtttaaggatagcgaggagaatgaggaggtgaaggagacaaccctgagggagctgaagatgctgcgcacactgaagcaggagaacatcgtggagctgaaggaggccttccggagaaggggcaagctgtacctggtgtttgagtacgtggagaagaacatgctggagctgctggaggagatgcctaatggcgtgccccctgagaaggtgaagagctacatctatcagctgatcaaggccatccactggtgccacaagaacgacatcgtgcacagggatatcaagccagagaacctgctgatcagccacaatgacgtgctgaagctgtgcgatttcggctttgcccgcaacctgtccgagggcaacaacgccaattacacagagtacgtggcaaccaggtggtaccggtccccagagctgctgctgggagcaccatacggcaagagcgtggacatgtggtccgtgggctgcatcctgggcgagctgtctgacggccagcctctgttcccaggcgagagcgagatcgatcagctgtttaccatccagaaggtgctgggccctctgccaagcgagcagatgaagctgttctactccaaccctaggttccacggcctgcgctttcccgccgtgaatcaccctcagagcctggagcgccggtatctgggcatcctgaactccgtgctgctggatctgatgaagaatctgctgaagctggaccccgccgataggtacctgaccgagcagtgtctgaaccaccccacattccagacccagagactgctggacaggtccccttctcgcaatcaggccggcaagtctacagccctgcagagccaccacaggagcaactccaaggacatccagaatctgagcgtgggcctgcctagggcagacgagggcctgccagcaaacgagtcctttctgaatggaaacctggcaggagcctctctgagcccactgcacacaaagacctaccaggccagctcccagccaggctccacatctaaggatctgaccaacaataacatcccacacctgctgtctcccaaggaggccaagagcaagaccgagttcgactttaatatcgatcccaagcctagcgagggcccaggcacaaagtatctgaagagcaactcccggagccagcagaatagacactccttcatggagtctagccagtctaaggccggcaccctgcagcccaacgagaagcagagccgccactcctacatcgataccatcccccagtcctctaggagcccttcctatcgcacaaaggccaagagccacggcgccctgtctgacagcaagtccgtgtctaatctgtccgaggcaagggcacagatcgcagagcccagcacctccagatactttcctagctcctgtctggacctgaactctccaacaagcccaacacccacccggcacagcgatacaagaaccctgctgtctcccagcggcagaaataacaggaacgagggcaccctggattctagaaggaccacaacccggcacagcaagacaatggaggagctgaagctgcccgagcacatggactctagccactcccactctctgagcgcccctcacgagtccttctcttacggcctgggctatacctccccattttcctctcaacagcgcccccaccggcactctatgtacgtgacacgggacaaggtgagagccaagggcctggacggcagcctgtccatcggacagggaatggcagcaagagcaaactccctgcagctgctgtctcctcagccaggagagcagctgccaccagagatgaccgtggcacggagctccgtgaaggagacaagcagagagggcacctctagcttccacacacggcagaagtccgagggcggcgtgtatcacgaccctcactctgacgacggcaccgccccaaaggagaacagacacctgtacaacgatccagtgcctaggagggtgggctccttctatcgggtgccatctccccgccctgacaatagctttcacgagaataacgtgtccacccgggtgtcctctctgccctctgagagctcctctggcacaaaccactctaagagacagcccgcctttgacccttggaagagcccagagaatatctctcacagcgagcagctgaaggagaaggagaagcagggcttctttaggagcatgaagaagaagaagaagaagagccagaccgtgcctaactccgactctcccgatctgctgaccctgcagaagagcatccacagcgcctccacaccaagctcccggcctaaggagtggcggcccgagaagatctccgatctgcagacccagagccagccactgaagtccctgcgcaagctgctgcacctgtctagcgccagcaaccaccctgcctcctctgacccaaggttccagcccctgacagcccagcagaccaagaactcttttagcgagatccgcatccacccactgtcccaagcctctggaggcagctccaacatcaggcaggagccagcaccaaagggccgccccgccctgcagctgcctggacagatggaccccggatggcacgtgtctagcgtgacacggtccgccaccgagggaccatcctactctgagcagctgggagcaaagagcggacctaacggacacccatataacagaaccaatagatccaggatgcctaatctgaacgacctgaaggagacagccctgtgatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - protein, 943 aa, synthetic construct
  REGION 1..943: note = Synthetic Construct; note = hCDKL5-3GS
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 9 - DNA, 4706 bp, synthetic construct
  misc_feature 1..4706: note = Vector Genome
  repeat_region 1..130: note = 5'ITR
  misc_feature 130..168: note = AAV2 seq upstream of 3'ITR
  regulatory 213..678: note = promoter - hSynapsin; regulatory_class = promoter
  misc_feature 690..695: note = Kozak
  misc_feature 3668..4255: note = WRPE
  regulatory 4273..4511: note = polyA_signal - SV40 PolyA; regulatory_class = polyA_signal_sequence
  misc_feature 4539..4577: note = AAV2 seq upstream of 3'ITR
  repeat_region 4577..4706: note = 3'ITR
  source 1..4706: mol_type = other DNA
  misc_feature 696..3653: note = nucleic acid sequence encoding hCDKL5-4GS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatgaagatccccaatatcggcaacgtgatgaataagttcgagatcctgggagtggtgggagagggagcatacggagtggtgctgaagtgccggcacaaggagacacacgagatcgtggccatcaagaagtttaaggatagcgaggagaatgaggaggtgaaggagacaaccctgagggagctgaagatgctgcgcacactgaagcaggagaacatcgtggagctgaaggaggccttccggagaaggggcaagctgtacctggtgtttgagtacgtggagaagaacatgctggagctgctggaggagatgccaaatggcgtgccccctgagaaggtgaagtcctacatctatcagctgatcaaggccatccactggtgccacaagaacgacatcgtgcacagggatatcaagcccgagaacctgctgatctcccacaatgacgtgctgaagctgtgcgatttcggctttgcccgcaacctgagcgagggcaacaacgccaattacacagagtacgtggcaaccaggtggtaccggagccctgagctgctgctgggagcaccatacggcaagtctgtggacatgtggagcgtgggctgcatcctgggagagctgtccgacggacagccactgttccctggcgagtctgagatcgatcagctgtttaccatccagaaggtgctgggccccctgccttccgagcagatgaagctgttctactctaaccctaggttccacggcctgcgctttccagccgtgaatcacccccagtctctggagcgccggtatctgggcatcctgaacagcgtgctgctggatctgatgaagaatctgctgaagctggaccccgccgataggtacctgaccgagcagtgtctgaaccaccctacattccagacccagagactgctggacaggtccccatctcgcaatcaggccggcaagtccacagccctgcagtctcaccacaggagcaactccaaggacatccagaatctgtccgtgggcctgccaagggcagacgagggcctgcctgccaacgagagctttctgaatggaaacctggcaggagcctctctgagcccactgcacacaaagacctaccaggccagctcccagcctggctccacatctaaggacctgaccaacaataacatccctcacctgctgagcccaaaggaggccaagtccaagaccgagttcgactttaatatcgatccaaagccatccgagggaccaggaacaaagtatctgaagagcaactcccggagccagcagaatagacactctttcatggagtctagccagagcaaggccggcaccctgcagcctaacgagaagcagagccgccactcctacatcgataccatcccacagtcctctaggagcccctcctatcgcacaaaggccaagtcccacggcgccctgtctgacagcaagtccgtgtctaatctgagcgaggcaagggcacagatcgcagagccaagcacctccagatactttcccagctcctgtctggacctgaactctcccacaagccctacaccaacccggcacagcgatacaagaaccctgctgtctcctagcggcagaaataacaggaacgagggcaccctggatagcagaaggaccacaaccaggcactccaagacaatggaggagctgaagctgcctgagcacatggactctagccactcccactctctgagcgccccacacgagtccttctcttacggcctgggctataccagccctttttcctctcaacaacgcccacaccggcactccatgtacgtgacacgggacaaggtgagagccaagggcctggacggcagcctgtccatcggacagggaatggcagcaagggcaaatagcctgcagctgctgtccccacagcctggagagcagctgccaccagagatgaccgtggcacggagctccgtgaaggagacaagcagagagggcacctctagcttccacacacggcagaagagcgagggcggcgtgtatcacgacccacactccgacgacggaaccgcacctaaggagaacagacacctgtacaacgatccagtgccccgccgggtgggctctttctatcgggtgcctagcccacgccccgacaactcctttcacgagaataacgtgtctacccgggtgtcctctctgcccagcgagagctcctctggcacaaaccactccaagagacagccagcctttgatccctggaagtctcctgagaatatctctcacagcgagcagctgaaggagaaggagaagcagggcttctttcggtccatgaagaagaagaagaagaagagccagacaaccgacagcaccaacggcgagaatccaagcatcaagaagtccctgttccccctgtttaacagcaagaatcacctgaagcacagctcctctctgaagaagctgcccgtggtgacacctccaatggtgcccaattccgactctcctgatctgctgaccctgcagaagtccatccacagcgcctccacaccaagctcccggcccaaggagtggcggcccgagaagatcagcgatctgcagacccagtcccagcccctgaagtctctgagaaagctgctgcacctgtctagcgcctccaaccacccagcctcctctgaccctcggttccagccactgacagcccagcagaccaagaactcttttagcgagatccgcatccacccactgtcccaagcctctggaggcagctccaacatcaggcaggagcctgccccaaagggccgcccagccctgcagctgccaggacagatggaccccggctggcacgtgtctagcgtgacacggagcgccaccgagggaccttcctactctgagcagctgggagcaaagtctggaccaaacggacacccttataacagaaccaatagaagcaggatgccaaatctgaacgacctgaaggagacagccctgtgatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgatcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - protein, 984 aa, synthetic construct
  REGION 1..984: note = Synthetic Construct; note = hCDKL5-4GS
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTTDSTNGENPSIKKSLFPLFNSKNHLKHSSSLKKLPVVTPPMVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPGQMDPGWHVSSVTRSATEGPSYSEQLGAKSGPNGHPYNRTNRSRMPNLNDLKETAL

SEQ ID NO: 11 - DNA, 22 bp, synthetic construct
  misc_feature 1..22: note = targeting site
  source 1..22: mol_type = other DNA
agtgaattctaccagtgccata

SEQ ID NO: 12 - DNA, 23 bp, synthetic construct
  misc_feature 1..23: note = targeting site
  source 1..23: mol_type = other DNA
agcaaaaatgtgctagtgccaaa

SEQ ID NO: 13 - DNA, 24 bp, synthetic construct
  misc_feature 1..24: note = targeting sequence
  source 1..24: mol_type = other DNA
agtgtgagttctaccattgccaaa

SEQ ID NO: 14 - DNA, 23 bp, synthetic construct
  misc_feature 1..23: note = targeting sequence
  source 1..23: mol_type = other DNA
agggattcctgggaaaactggac

SEQ ID NO: 15 - DNA, 16 bp, synthetic construct
  misc_feature 1..16: note = spacer
  source 1..16: mol_type = other DNA
atgacttaaaccaggt

SEQ ID NO: 16 - DNA, 3430 bp, synthetic construct
  misc_feature 1..3430: note = transcript variant
  source 1..3430: mol_type = other DNA
gggggagttggaggaggaagaccctacaggaagttgagctgtactctccaacaggcgagcacatggtcgtctcctctctcccttcagggagtcatttaatacttcatgattagaacaaatatgtgaaagttcccaccaaccagtgagaatttcttccttcagacggttttggatcttactgcacagctttctgagaagttcttttggtgccatgttttgtggcttgcatcaaaagaggagtttgtcttcatgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtgacttctgcaagcctgcggctggtcccaatgccctgaatcacctctctcatggaagaaccaattaacaccaatgaatcaaccattaaaaa

SEQ ID NO: 17 - DNA, 14572 bp, synthetic construct
  misc_feature 1..14572: note = transcript variant
  source 1..14572: mol_type = other DNA
agttgtctctgccgctggggaaggtaaagcggcgacggcgtcctcaggagctgtggggtcccctgctagaagtgggggactcggcgggggagtcatttaatacttcatgattagaacaaatatgtgaaagttcccaccaaccagtgagaatttcttccttcagacggttttggatcttactgcacagctttctgagaagttcttttggtgccatgttttgtggcttgcatcaaaagaggagtttgtcttcatgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttgtaatttgtgctggtaggggggaggggtggacagacaagccagtggggaggggtgggaaagggtgggctgggcttggggcatgggccgggccaagtggtgagccaatattttcagctttatgaaggtgtgtgcaatattgcatgtgttggggccgttgagctcctcgcggccacaaatgctagtcagggatcttagagccacaggagttccttagggatcgccactccccacaggtcttgtgtgagaatagatagagtgtgccattgaggaagaagaaattcttgccagtttctcccctttacattccagctttagtgcaatctctgttgaacttgggaatgccaggatgtgtcctggcactgcaagggataggaaagtcgtgttgaccgatgcccttactacatatttttttctgcctagagtaaatgtggggtttattgtaatccaagagtatccctttgaagggttagcagatgaactgtatgtcttaaattgttttctaaacttccatatttgataggatttgtaacatttatttataattaatattgtactgttaaaatcatatcttttatgttataatgtaaagttattttgagctgtttgaaacattgtgaagcagtgggacagctacagtagtttctataaaaactaaacagtaacatttatatattgcatcaggagtattttattctatatataatatatatatatggtaaatatctgttttataaatatattcatttaaagaaagtatcgttatgacactatctgccatatttttatacatttgtgatatgaagtgagtattatacttctactaaagggagttgaattgtggctacacgtgactgtaacagtatgaaccttgctcaactttgaggccccagcagtagcctctagacatgatccttggtagcagacgagatgcagcatctctttccaaacctgcaggggaagaaagtcagataggccagtgagaattccagcaggcctggtttccgtcctcacacacttggctcctgtttgtgtccagggacgttacttgcacacaggggagaatagattgaggcgttactcgtgggtctttgtagagagaatgtgctcttccacttagcattagtgtagagcgtagaccaaagatttgccagtgaacattccttgttgtgagatcataagaggtacctgtctgcagatcttgcaaaccatctctatgctcctgaatattgtccactgtcccggagactcttggctgatggggtgtgagatatatgtgtggcatttctatttctgaatgccttttttgctattgctctctccaataccatatttaaaaggctcctggagcaattccagatagataaaaaattactctatccatttttttcttcctggggctgtccacttatgacaagattttcatgcacacctgttacgcacacaacccccatcagaacaggacctatcttcccctcatctgatctctttcagctcccaagttactcccaagtatcatcaaaccctttggccatcaagtgttatcctcccatgcctctgggtcatccttgagggaagtcacctcagcaccatcatctatcagtagtagtagatttttggaattagtctctgagcaataatacttttaaaggtccctcccacccctcaaagagataacttctcagtatttattacttgtcctcaaattctgagtgtcagcccctgcatcaagtcagctatgatcaaaatgtgctgttactgttaaccctcccccgagattttgagaatcatagctctgatgattatgaagattataaagactaaggtcctagtttccctgaagcttaaattgtgcacatattgcatttttatataaatactataatgtgtattgattatatagataggtcattttaaggtgctttttatttaattttaatgagtgtaataggcactaaaatgaaactcgactgggtactattactgaaaaaatttgactcaaaccaatagttttgcatgctctttagttctttttctgtcttacttctttcaccagttcagtactgtttgggtagctctgaattctggcttttccaggcagctgcttgatgatacaaatgaggtggaccatcagcagttttgcctggacactaatgactctaaaagggtgtgtatttaactcttctttttcatactagatctctaccttgtattccccccttgcaaaccagaaactacactttttttctctggaaagacttctcactgtgctatgcgcttaggaactgcacggtcccgttgccatgctgtggcattcgaggctcgtcacctaggggctggtttcccatgttgtcatccttgctaacactgggatctcagatgtttgcagaacatttctattcatgacggttcttcaaagaagggctagaatatttgccttctacctagagcaaagtaaacctaattgttgaagagtcaatacgtactttttgtactttcccagatttgagccagaaaatacctacagatatctacaaatgttttcaaccagtgttttcgaggtagctctttaatcctcttctcagcttttgtctgttctgacttttcatccaataagctgtaagtgaactacaatctgcatttccagcatgcaattctcttcctgctcacggagggggatgcaaaagttttctctcacacctaatgggccctctcttagcttttaactgttgttttcccctgttgatgaaaaattactgacatcacaaagattgccatccatggtagaaaggacttgatttaaagtcagaaaaatttgccttttctttaggaaggggaattcatgacatccatgtcctattatcggccgtaacttcctaaagaagaaaaagggtgaatgaaagcttatgttacttttatgaaagtatcactgatctcctttggaaaagaaactctgcttcactgtttttctacctttatttctctgccatcctcgtttttcctctctgaagttctaaataccctttaactgaaccttgttggtcttgctctaaaatttagcctctttctagagcccaagaccaaaagggagtgcttcatgctgaggggctcaagtgagctgactctacaactgtggttctcaactaggggcagatttacccccaagggtacatttggcaatgtctggggacatttggttgtcatagctgggggggaggtgatggtatgctactggcatcaagtggttagaggccacagatgctactaaacaccctgccatgcacaggacagccccccaaacaaacagtggtccagccccaagcactactagtgctgaggttgagaaaccctgccctacaccatcccaagcccaccagtctgcagagcttggctctggccttgttctgtccaggctacagtactgcaggtgtgatttcttcaacatcacattctcttaagagtcttcaaccactatgcctgaatccttggaatcctattgttcgtttccattgtcttgggcctgctaaaaggtgcatactttggaaggagaccagagttactttataggtgttggagaacttaaattctaggttagcatccttagaaaatctttcatagagccatgaggcttatatttagaagcaagcaacagcctggcagattggcatgatttttaccaactgctcaaaggcatccgtggctttcagctgtgtctcccgaaagaaaatgtctttgttttttattcaagtcatttaatagctctttacaaatattagcatatggcagaccaattagaagcaaaaaagtccgtattggtggttgtgatgtggctgttatagaagtatttgtgctgtatgctttggttaaatctgttttaaaacatgctttaagattcaccttacgcagttgtactttaattctaagctcagagctatgctgtcagtctctagtcacgccatgtattataaagtatgttgtggttgtagagttagccagtttagcatgttcctaatctgagaattagtggactatactaggaaatgctatcccattttccctttccagcccctcttagttaagatcatagtatcctcacttcttaaactagtctttagagtaaataaggaaaaaagccatttattttcttctagccatgtattattgagaatgactcatagtacaactttttttcaaaggccagaggattactttgcaagtgtgtagaaagatctgtctatttctgttcacctgggacctggtggtgactgaactctttcagcaagggcaatggctcaatgtgattcggtgctggagctcagctgggactcagtaaatctgcacgttctctgctggtcagaacatgtttcctggagagcattgcttttctatctagaagttcgttacctcctgcatgatagtgggatattgagatgtgggttctaagatccacagactcatacttttgtgaactttggaatgtggtagggtacactgaaccaagtcagaaaatagtgaaatatttccttgcctccttcagttcatcactaagaaagtgtgtaggcagagaaaacttacttatggtttgaagaaccacaattgtttttgaaaggggacatagaaaagacagtacaaaggttgtaagtgtcctgtcgctaattcccagggccagaatctcacaagaagatcctctcaaagacttaaaagcctggcttacaaaaataaccagaaaccaaacttgtaagcagttacaatttcttccttttgcttcttgaccagctgagatacagaatatcttgaggtctggttcagctgatgaaaaattctgtctcagaaacgatgagtaagaaaccatacaagaaaaagaagtccctcatctatctgcatacacgcaatgtgggagaagtggggtggctaggaaactctgggcctgctgcccccttcgattctttttttttttctttgtgaattgaatgtacgatacaaatggtaggccttcatgtgagccagttactacatgaatcttcatttcccacagtggtttgttcattcatcagcgttaggcttggtcctggctccacctttctcctctccgggcactgaccccacctttccgtgtatttactgtaggctattaaatatgatcatgacccgccttgcattttcattcatcacctgtttatgcccaaatttaaaggaagtttgtctcattttgccagaaaaaaattgtaatagtcggcacgctggatttgtagggccagcaaaattgcggcagtgaaactagtttcacttctaaagcccttcatttcccacaaggttaagctctcgaaaccccatttgatccttggttcctatttcgatcctcctttggaatctgaaaatcggtctccatgttgtatgcagattagaagttgccttgtttgttactcttccaacacagggtatcagggagaaagaggccttatctgttcctccatcccccctgttttgacagactgctaagaattcctcaggacttcctttggttggggattttactttcccaaaagtctgatctgatttctttcaggggtagacaagcttgtcctagtgctctgcttcaggtcttatcagaagaaacccaggaatagaaaaggtagatgccttgacttttgtccctgttgtggggactaaagtgttttttgccagaattgtcaaaagctccggttcaaactctgtagagtttcatggaaaaacaaaacaaaacaaaaaagatgcttatcgtcccggagaatgtgtaagtaagttctccagcacggcttaggttttccaaaatggaaagcaaagcttgcagacaacctgttttctctagagacacacaatgattctggccctcagtctgtttccctccccttgtttactctttggtcaccgatcgagtcaggctagaggggtaatacggtgattaaaagaagtaaattctcactgtaaacttggattgattaacagaaacaattaaaccaaacaaaatcattgccccaaatttctatctccaagaattgctttgtgccattttgaattcaggtagttattctgtatatacttagctaactattcagagcttcttcaatctctagtaagcttgaaggtggtgtctgggaagacacagggcagggtgacatttagactctgttcattcttaaaaatagggataagccagaatgatgtgacatctagtatgtggtagatgaagaatcagtggaaattcttaattgcacagactttataatccattatataaagtgtaattccatactttttactgtggcaagggtgtgatgtgattgttaatctttttaatttttgaatccaaactgaatttaaagtgttgaatacttaaatcctcacaagtgaattatgaaccatttgtaaagtgtttctataactctttgtatcatgtgttggtacatcttatcaagttttttctggcatgtaattccattctaaacttatgtaaaatttctattgttgctgtaaatattacacaaatatctattccgtgataaccttcattatcagcatgaaatggttaatttttactgagcacaatgtatttttgaatggatcttcccaaatgtctttaataaaatgcagattttgcactgactgatgtgactgccaggccgtcccaatcttgagcactgtggtccttcgtgtttgaactgcgaacagctccattttagttccttcttgggaagaaagaatgcattcgaattcaaatgagggatgatgggcctttgaggttgtaatcagtttgagacagaaattaatgtaaaactaggcaaatcctgtaacttaatgatgttgatctttgttttcctatttataaagcttttttccccttacggcattttgaaaactttggtttgttttatacttgagtgttgtcacctttgcacgtcgctagacactcacgtggtagtttgaggaggatgggaagagaggccaatcttgtgctaagtgctatggagaaagcaaagatgcacagcttaataagggttttttgattctagaggagaggtgggcaagcttttcctgtaaagggccagatggtaaatattgggggctctgtggcccatcagttccctgtcaacattactcagctctgcctttgtattgcagaggctgcatggataatatgtaaatcagtaagtgtggctgtggtcccgtaaaactttacttatagaaacaggccatagtttactaacccgttttaggccctcagtttcatcatttagggtaacaccaaccgagtacatgttcaatcaggagggaaagaacttagcttttgtgataacttatagatgaaatattccccagttttctttctctttctaaacaaagggtaggagctgaaagtcagtgctctccattcttaaaagtcctctgtcaaccacctcttcactctatctccttaagtgacatggaaacttgtggcctgtgctttcaactataatcttaccaagactgaatgaaagtatttggtgcttatactttttaatgttcatagcacttactatggccaggcacaatgcccaagtgttttgctttattattattattattattattattattattattattattattattttcacaacaacccagcgaaacaggtactattattatcccctttttgtaaatgaggaaacagatttccatacttgacattaacctgcccaaggtcacccagtttggaagtggcagggccaggatttgaagccgaatatgttcattgtaaagcccatatacccttaaaagtcactgcacgtccacgtatggtaatgtggtcacaatcagtattgtttctctttagtaattcagatttattcaagtaactccaaggttcaaattttcaaaaacagtttccaacacaggttatattaaaaactttgcctgaagtttactctaggtgcagaaataaagacaacttagtcaagtgaaggaaagggtaacagacttagaaatggctatctacactgatggaataagtatactaattagagcaggtaacaagtccccacgatgtataacatattcttctctaaaagctcacaacaatttatgtgccaccttccatcccctttcccagcagatggtaattgatgcaatttgagaatagaacacttcattgaaacaagtcactggaggtcctctggggatgcagcttaaaagcccaggatcaggccaggtgtggtggctcacgcctgtaatcccagcactgtgggagactgagacgggcagatcacctgaagtcaggagttcgagaccagcctggccaatatagagaaaccccgtctgtactaaaaatacaaaaattaggtctgggcggtggctcacgcccgtaatcctagcactttgggaggccaaggtgggtggatcacgaggtcaggagttcgagaccagcctggccaacatggtgaaaccccgtctctactaaaaatacaaaaattgggtgtggtggtacatgcctgtaatcccagctgcttaggaggctgaggcaggagaatcacttgaacccaaaaggcagaggttgcagtgagccgagatcacgccactgcactccagcctggcaacagagcgagactctgtctcaaaaaaaaaaaaaaaaaaatacaaaaattagccgggaatggtggtgaatgtctataatcccagccactcgggaggctgaggcaggagaatcacttgaacccaaaaggcagaggttgcagtgagccaagatcgcaccactgcactcccacctggacgacagagcgagacgccatctcaaaaataaataaataaataaatccctaggatatagagtccgcttgcctgggtggagtcttggctccatctcctacatctcatgtgacttgaagcaaggcacttcactatgcctcagtttccttatctttaaatggaggtaatcacagtacctacctcagtgttgttgtgaagatttaatgagataataacctgcagagcacttgagagtctcaagaaatatgagctcttataacattattgctattgtaaaacattgctattacttctgtcccaatttggcagtttcaggaaattgagaaggaatgtcactgaatctttaatgccagttacacagagctcagtaggtgtccgcacagccctttagatgcacgtagcctatttgatcttcgtatcagccctgtaaggttgggcttaagaatgacccagcatggaagtactcttgggaccacaggggaagcaacagtccttgtaagctgtttgtcagtgggccttagcaaccaagccccaagtggacagaaagatggggttctgtgagcatccattccaagggtcactttaggggtgcaatcttctgggctctgagaatggattcacagcagcatctctatctggaaatacctttgctttgtccaagtaacctcctcccacacagagtacccgaatcagctcagattttttttccttaatattggcaacacttaatgctacttctttgttcgtaacatttcatttaattatttaaaaatctgcaaatgttgtgtatattttaccgcaatttttaaacaaactccagtgtagaccgtgctctagcatttctaaaaataaaagtaaatatgttaaaataaggtaagagtgagtttttcctacactatgtataatttttaattgaaagtttcccccacaaaagaaatgatacttggaagcatagtttgtgtgtttgacagcaagattctattaggcttcagatttccctcaataaaagttttagtttttatacattctgtcatgaattgaatgaagtaaataatatgctgttgtaagcaaattatctgcagataaatgtcttatgaatcgtatatcttactgtttattaaattctgaagtattgagcagtttccaatgtgaaatcccagctggcttctttgcaatgatggacaagttagttataattcatatggaaattcaaggaacccagaatagccaaaacaatcccgaagagcaaagttggaggactcacatgccccaatttcaaaacttactacaaagctacaataatgaagaaagtgaggtaatagaatttggagtccagaaataaaccctctcacatttatggtcaattgttttttgacaagggtgccaagatcattcaatgggggaaagaatagtcttttcagcaaatagtggtgggacaactggatatccacatgcaaaagaatgacgttggactcctatttcacaccatatacaaaaattaactcaaaatggaacaaagatctaactgtaagaggtagaactataaaactcatagaagaaaacataggggtaaatcttcatgaccttggatttggcagtggtttctcagatatgacaccaaaggcacaagcaaagagaaaaataaattggacttcatcaaatatttaaaattttgtgcttaagaggactctatcgagagggttaaaaagatccacagaatgggagaaaatatttgcaaatcatatccaataagggaattagaatatacaaagaattcttaaactcagtaagttgtaagccgatttaaaatgggcagaggatctgaaaaacctttatccaaagatgtacaaatggccagtaaacacatgaaaagatgctcaacatcattagtcactagggaaatgcaaatcaaaaccacagttgagatgcaacttcacacattatgggtatagtcaaagatggacagtaacaagtgttgctgaggatgtggagaaagtggaagcctcctacattgctggtggaaatgtaaaatgatgcagccacttgggataatagttcctccaaagattaaacaagagtgactgtgtgtcccagcagttttactcctaagtatatacccaagataaatgaaaacacacatccgcacaaaaacttatacacaaatgttcatagcagcattattcataatagctaaagagtggaaacccaaatatccatcaatggatgaatggataaataacaatgtgctcttgacatgatactgaacaatgatgttcagcaataaaaaggagtgacaagtgacatagtgatacacatgttataacgtggatgaactttaaaacactatgctaagtgaaagaagccagtcacgaaataccacatactatatgatttcatttatgtagaacgtccagaaggccaatctacagagacagtcgattcgtggttgcagagggcttggggtggccgatgggttggggagtgttggctgatgggtgcaggatttctctttggggtgataaactttctaaaattgatctagtgataatggttgcacaactctgaatataccaaaagtcatcaaattgtacactttaaatggatacaaattgtatgtgaacttaatctcaataaagccgtgacaaaagtttccaacatgaa

SEQ ID NO: 18 - DNA, 3434 bp, synthetic construct
  misc_feature 1..3434: note = transcript variant
  source 1..3434: mol_type = other DNA
attagttgtctctgccgctggggaaggtaaagcggcgacggcgtcctcaggagctgtggggtcccctgctagaagtgggggactcggcgggggagtcatttaatacttcatgattagaacaaatatgtgaaagttcccaccaaccagtgagaatttcttccttcagacggttttggatcttactgcacagctttctgagaagttcttttggtgccatgttttgtggcttgcatcaaaagaggagtttgtcttcatgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaagtgacttctgcaagcctgcggctggtcccaatgccctgaatcacctctctcatggaagaaccaattaacaccaatgaatcaaccattaaaaa

SEQ ID NO: 19 - protein, 1030 aa, synthetic construct
  REGION 1..1030: note = amino acid sequence
MKIPNIGNVMNKFEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLNHPTFQTQRLLDRSPSRSAKRKPYHVESSTLSNRNQAGKSTALQSHHRSNSKDIQNLSVGLPRADEGLPANESFLNGNLAGASLSPLHTKTYQASSQPGSTSKDLTNNNIPHLLSPKEAKSKTEFDFNIDPKPSEGPGTKYLKSNSRSQQNRHSFMESSQSKAGTLQPNEKQSRHSYIDTIPQSSRSPSYRTKAKSHGALSDSKSVSNLSEARAQIAEPSTSRYFPSSCLDLNSPTSPTPTRHSDTRTLLSPSGRNNRNEGTLDSRRTTTRHSKTMEELKLPEHMDSSHSHSLSAPHESFSYGLGYTSPFSSQQRPHRHSMYVTRDKVRAKGLDGSLSIGQGMAARANSLQLLSPQPGEQLPPEMTVARSSVKETSREGTSSFHTRQKSEGGVYHDPHSDDGTAPKENRHLYNDPVPRRVGSFYRVPSPRPDNSFHENNVSTRVSSLPSESSSGTNHSKRQPAFDPWKSPENISHSEQLKEKEKQGFFRSMKKKKKKSQTVPNSDSPDLLTLQKSIHSASTPSSRPKEWRPEKISDLQTQSQPLKSLRKLLHLSSASNHPASSDPRFQPLTAQQTKNSFSEIRIHPLSQASGGSSNIRQEPAPKGRPALQLPDGGCDGRRQRHHSGPQDRRFMLRTTEQQGEYFCCGDPKKPHTPCVPNRALHRPISSPAPYPVLQVRGTSMCPTLQVRGTDAFSCPTQQSGFSFFVRHVMREALIHRAQVNQAALLTYHENAALTGK

SEQ ID NO: 20 - protein, 960 aa, synthetic construct; identical to SEQ ID NO: 2
  REGION 1..960: note = amino acid sequence

SEQ ID NO: 21 - protein, 1030 aa, synthetic construct; identical to SEQ ID NO: 19
  REGION 1..1030: note = amino acid sequence

SEQ ID NO: 22 - DNA, 2883 bp, synthetic construct
  misc_feature 1..2883: note = nucleic acid sequence
  source 1..2883: mol_type = other DNA
atgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctga

SEQ ID NO: 23 - DNA, 466 bp, synthetic construct
  misc_feature 1..466: note = promoter
  source 1..466: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcg

SEQ ID NO: 24 - DNA, 3009 bp, synthetic construct
  misc_feature 1..3009: note = nucleic acid sequence
  source 1..3009: mol_type = other DNA
atgaagatccccaatatcggcaacgtgatgaataagttcgagatcctgggagtggtgggagagggagcatacggcgtggtgctgaagtgcagacacaaggagacacacgagatcgtggccatcaagaagtttaaggatagcgaggagaatgaggaggtgaaggagacaaccctgcgcgagctgaagatgctgcggacactgaagcaggagaacatcgtggagctgaaggaggccttccggagaaggggcaagctgtacctggtgtttgagtacgtggagaagaacatgctggagctgctggaggagatgcctaatggcgtgccccctgagaaggtgaagagctacatctatcagctgatcaaggccatccactggtgccacaagaacgacatcgtgcaccgcgatatcaagccagagaacctgctgatcagccacaatgacgtgctgaagctgtgcgatttcggctttgcccggaacctgtccgagggcaacaacgccaattacacagagtacgtggcaaccaggtggtaccggtcccctgagctgctgctgggagcaccatacggcaagtctgtggacatgtggagcgtgggctgcatcctgggagagctgtccgacggacagccactgttccctggcgagtctgagatcgatcagctgtttaccatccagaaggtgctgggccccctgccttccgagcagatgaagctgttctactctaacccaaggttccacggcctgaggtttccagccgtgaatcacccccagtctctggagcgccggtatctgggcatcctgaacagcgtgctgctggatctgatgaagaatctgctgaagctggaccctgccgatcgctacctgaccgagcagtgtctgaatcacccaacatttcagacccagagactgctggacaggagcccctcccgctctgccaagaggaagccttatcacgtggagagctccaccctgagcaatcgcaaccaggccggcaagtccacagccctgcagtctcaccaccggagcaactccaaggacatccagaatctgagcgtgggcctgccaagggcagacgagggcctgcctgccaacgagtccttcctgaatggaaacctggcaggagcctctctgagcccactgcacacaaagacctaccaggcctctagccagcctggctccacatctaaggacctgaccaacaataacatccctcacctgctgagcccaaaggaggccaagtccaagaccgagttcgactttaatatcgatccaaagcccagcgagggcccaggcacaaagtatctgaagagcaactccagatctcagcagaataggcactctttcatggagtcctctcagagcaaggccggcaccctgcagcccaacgagaagcagagcaggcactcctacatcgataccatcccacagagctcccgcagcccctcctatcggacaaaggccaagagccacggcgccctgtctgacagcaagtccgtgtctaatctgtccgaggcaagagcacagatcgcagagccaagcacctccaggtactttccctcttcctgtctggacctgaactctccaacaagccctacaccaaccagacacagcgatacaaggaccctgctgtctcccagcggcagaaataacaggaacgagggcaccctggatagcagaaggaccacaaccagacactccaagacaatggaggagctgaagctgcccgagcacatggactcctctcactcccactctctgagcgcccctcacgagtccttctcttacggcctgggctataccagcccttttagctcccagcagcgcccacacaggcactccatgtacgtgacaagagacaaggtgagggccaagggcctggacggcagcctgtccatcggacagggaatggcagcaagggcaaatagcctgcagctgctgtccccacagcctggagagcagctgccaccagagatgaccgtggccagatctagcgtgaaggagacaagcagggagggcacctcctctttccacacaagacagaagagcgagggcggcgtgtatcacgacccacactccgacgacggaaccgcacccaaggagaacaggcacctgtacaacgatccagtgccccgccgggtgggctctttctatcgggtgcctagcccacggcccgacaactcctttcacgagaataacgtgtctaccagagtgagctccctgcctagcgagtctagctccggcacaaaccactccaagaggcagccagcctttgatccctggaagtctcctgagaatatctctcacagcgagcagctgaaggagaaggagaagcagggcttctttcgcagcatgaagaagaagaagaagaagagccagacaaccgacagcaccaacggcgagaatcctagcatcaagaagtccctgttcccactgtttaactccaagaatcacctgaagcactctagctccctgaagaagctgcccgtggtgacacctccaatggtgcccaattccgactctcctgatctgctgaccctgcagaagagcatccacagcgcctccacaccatctagcagacccaaggagtggaggccagagaagatctccgatctgcagacccagtcccagcccctgaagtctctgcggaagctgctgcacctgtcctctgccagcaaccaccctgcaagctccgaccctaggttccagccactgacagcccagcagaccaagaactcttttagcgagatcagaatccacccactgtcccaggcctctggaggctctagcaacatccgccaggagcctgccccaaagggccggccagccctgcagctgccaggacagatggaccccggctggcacgtgtcctctgtgacaagatccgccaccgagggaccatcctactctgagcagctgggagcaaagtctggaccaaacggacacccttataacaggaccaatagatccaggatgcctaatctgaacgacctgaaggagacagccctgtgatag

SEQ ID NO: 25 - DNA, 2835 bp, synthetic construct
  misc_feature 1..2835: note = nucleic acid sequence
  source 1..2835: mol_type = other DNA
atgaagatccccaatatcggcaacgtgatgaataagttcgagatcctgggagtggtgggagagggagcatacggagtggtgctgaagtgccggcacaaggagacacacgagatcgtggccatcaagaagtttaaggatagcgaggagaatgaggaggtgaaggagacaaccctgagggagctgaagatgctgcgcacactgaagcaggagaacatcgtggagctgaaggaggccttccggagaaggggcaagctgtacctggtgtttgagtacgtggagaagaacatgctggagctgctggaggagatgcctaatggcgtgccccctgagaaggtgaagagctacatctatcagctgatcaaggccatccactggtgccacaagaacgacatcgtgcacagggatatcaagccagagaacctgctgatcagccacaatgacgtgctgaagctgtgcgatttcggctttgcccgcaacctgtccgagggcaacaacgccaattacacagagtacgtggcaaccaggtggtaccggtccccagagctgctgctgggagcaccatacggcaagagcgtggacatgtggtccgtgggctgcatcctgggcgagctgtctgacggccagcctctgttcccaggcgagagcgagatcgatcagctgtttaccatccagaaggtgctgggccctctgccaagcgagcagatgaagctgttctactccaaccctaggttccacggcctgcgctttcccgccgtgaatcaccctcagagcctggagcgccggtatctgggcatcctgaactccgtgctgctggatctgatgaagaatctgctgaagctggaccccgccgataggtacctgaccgagcagtgtctgaaccaccccacattccagacccagagactgctggacaggtccccttctcgcaatcaggccggcaagtctacagccctgcagagccaccacaggagcaactccaaggacatccagaatctgagcgtgggcctgcctagggcagacgagggcctgccagcaaacgagtcctttctgaatggaaacctggcaggagcctctctgagcccactgcacacaaagacctaccaggccagctcccagccaggctccacatctaaggatctgaccaacaataacatcccacacctgctgtctcccaaggaggccaagagcaagaccgagttcgactttaatatcgatcccaagcctagcgagggcccaggcacaaagtatctgaagagcaactcccggagccagcagaatagacactccttcatggagtctagccagtctaaggccggcaccctgcagcccaacgagaagcagagccgccactcctacatcgataccatcccccagtcctctaggagcccttcctatcgcacaaaggccaagagccacggcgccctgtctgacagcaagtccgtgtctaatctgtccgaggcaagggcacagatcgcagagcccagcacctccagatactttcctagctcctgtctggacctgaactctccaacaagcccaacacccacccggcacagcgatacaagaaccctgctgtctcccagcggcagaaataacaggaacgagggcaccctggattctagaaggaccacaacccggcacagcaagacaatggaggagctgaagctgcccgagcacatggactctagccactcccactctctgagcgcccctcacgagtccttctcttacggcctgggctatacctccccattttcctctcaacagcgcccccaccggcactctatgtacgtgacacgggacaaggtgagagccaagggcctggacggcagcctgtccatcggacagggaatggcagcaagagcaaactccctgcagctgctgtctcctcagccaggagagcagctgccaccagagatgaccgtggcacggagctccgtgaaggagacaagcagagagggcacctctagcttccacacacggcagaagtccgagggcggcgtgtatcacgaccctcactctgacgacggcaccgccccaaaggagaacagacacctgtacaacgatccagtgcctaggagggtgggctccttctatcgggtgccatctccccgccctgacaatagctttcacgagaataacgtgtccacccgggtgtcctctctgccctctgagagctcctctggcacaaaccactctaagagacagcccgcctttgacccttggaagagcccagagaatatctctcacagcgagcagctgaaggagaaggagaagcagggcttctttaggagcatgaagaagaagaagaagaagagccagaccgtgcctaactccgactctcccgatctgctgaccctgcagaagagcatccacagcgcctccacaccaagctcccggcctaaggagtggcggcccgagaagatctccgatctgcagacccagagccagccactgaagtccctgcgcaagctgctgcacctgtctagcgccagcaaccaccctgcctcctctgacccaaggttccagcccctgacagcccagcagaccaagaactcttttagcgagatccgcatccacccactgtcccaagcctctggaggcagctccaacatcaggcaggagccagcaccaaagggccgccccgccctgcagctgcctggacagatggaccccggatggcacgtgtctagcgtgacacggtccgccaccgagggaccatcctactctgagcagctgggagcaaagagcggacctaacggacacccatataacagaaccaatagatccaggatgcctaatctgaacgacctgaaggagacagccctgtgatag

SEQ ID NO: 26 - DNA, 2958 bp, synthetic construct
  misc_feature 1..2958: note = nucleic acid sequence
  source 1..2958: mol_type = other DNA
atgaagatccccaatatcggcaacgtgatgaataagttcgagatcctgggagtggtgggagagggagcatacggagtggtgctgaagtgccggcacaaggagacacacgagatcgtggccatcaagaagtttaaggatagcgaggagaatgaggaggtgaaggagacaaccctgagggagctgaagatgctgcgcacactgaagcaggagaacatcgtggagctgaaggaggccttccggagaaggggcaagctgtacctggtgtttgagtacgtggagaagaacatgctggagctgctggaggagatgccaaatggcgtgccccctgagaaggtgaagtcctacatctatcagctgatcaaggccatccactggtgccacaagaacgacatcgtgcacagggatatcaagcccgagaacctgctgatctcccacaatgacgtgctgaagctgtgcgatttcggctttgcccgcaacctgagcgagggcaacaacgccaattacacagagtacgtggcaaccaggtggtaccggagccctgagctgctgctgggagcaccatacggcaagtctgtggacatgtggagcgtgggctgcatcctgggagagctgtccgacggacagccactgttccctggcgagtctgagatcgatcagctgtttaccatccagaaggtgctgggccccctgccttccgagcagatgaagctgttctactctaaccctaggttccacggcctgcgctttccagccgtgaatcacccccagtctctggagcgccggtatctgggcatcctgaacagcgtgctgctggatctgatgaagaatctgctgaagctggaccccgccgataggtacctgaccgagcagtgtctgaaccaccctacattccagacccagagactgctggacaggtccccatctcgcaatcaggccggcaagtccacagccctgcagtctcaccacaggagcaactccaaggacatccagaatctgtccgtgggcctgccaagggcagacgagggcctgcctgccaacgagagctttctgaatggaaacctggcaggagcctctctgagcccactgcacacaaagacctaccaggccagctcccagcctggctccacatctaaggacctgaccaacaataacatccctcacctgctgagcccaaaggaggccaagtccaagaccgagttcgactttaatatcgatccaaagccatccgagggaccaggaacaaagtatctgaagagcaactcccggagccagcagaatagacactctttcatggagtctagccagagcaaggccggcaccctgcagcctaacgagaagcagagccgccactcctacatcgataccatcccacagtcctctaggagcccctcctatcgcacaaaggccaagtcccacggcgccctgtctgacagcaagtccgtgtctaatctgagcgaggcaagggcacagatcgcagagccaagcacctccagatactttcccagctcctgtctggacctgaactctcccacaagccctacaccaacccggcacagcgatacaagaaccctgctgtctcctagcggcagaaataacaggaacgagggcaccctggatagcagaaggaccacaaccaggcactccaagacaatggaggagctgaagctgcctgagcacatggactctagccactcccactctctgagcgccccacacgagtccttctcttacggcctgggctataccagccctttttcctctcaacaacgcccacaccggcactccatgtacgtgacacgggacaaggtgagagccaagggcctggacggcagcctgtccatcggacagggaatggcagcaagggcaaatagcctgcagctgctgtccccacagcctggagagcagctgccaccagagatgaccgtggcacggagctccgtgaaggagacaagcagagagggcacctctagcttccacacacggcagaagagcgagggcggcgtgtatcacgacccacactccgacgacggaaccgcacctaaggagaacagacacctgtacaacgatccagtgccccgccgggtgggctctttctatcgggtgcctagcccacgccccgacaactcctttcacgagaataacgtgtctacccgggtgtcctctctgcccagcgagagctcctctggcacaaaccactccaagagacagccagcctttgatccctggaagtctcctgagaatatctctcacagcgagcagctgaaggagaaggagaagcagggcttctttcggtccatgaagaagaagaagaagaagagccagacaaccgacagcaccaacggcgagaatccaagcatcaagaagtccctgttccccctgtttaacagcaagaatcacctgaagcacagctcctctctgaagaagctgcccgtggtgacacctccaatggtgcccaattccgactctcctgatctgctgaccctgcagaagtccatccacagcgcctccacaccaagctcccggcccaaggagtggcggcccgagaagatcagcgatctgcagacccagtcccagcccctgaagtctctgagaaagctgctgcacctgtctagcgcctccaaccacccagcctcctctgaccctcggttccagccactgacagcccagcagaccaagaactcttttagcgagatccgcatccacccactgtcccaagcctctggaggcagctccaacatcaggcaggagcctgccccaaagggccgcccagccctgcagctgccaggacagatggaccccggctggcacgtgtctagcgtgacacggagcgccaccgagggaccttcctactctgagcagctgggagcaaagtctggaccaaacggacacccttataacagaaccaatagaagcaggatgccaaatctgaacgacctgaaggagacagccctgtgatag

SEQ ID NO: 27 - DNA, 588 bp, synthetic construct
  misc_feature 1..588: note = nucleic acid sequence
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 28 - DNA, 239 bp, synthetic construct
  misc_feature 1..239: note = nucleic acid sequence
  source 1..239: mol_type = other DNA
ggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatc

SEQ ID NO: 29 - DNA, 4806 bp, synthetic construct
  misc_feature 1..4806: note = Vector Genome
  repeat_region 1..130: note = 5' ITR
  misc_feature 130..168: note = AAV2 seq upstream of 3'ITR
  regulatory 226..1454: note = promoter - UbC promoter; regulatory_class = promoter
  misc_feature 308: note = Insertion (C)
  misc_feature 1009: note = Insertion (G)
  misc_feature 4353: note = Deletion (G)
  misc_feature 4370..4391: note = miRNA183
  misc_feature 4396..4417: note = miRNA183
  misc_feature 4424..4445: note = miRNA183
  misc_feature 4452..4473: note = miRNA183
  regulatory 4482..4608: note = polyA_signal - Rabbit Beta Globin polyA; regulatory_class = polyA_signal_sequence
  misc_feature 4639..4677: note = AAV2 seq upstream of 3'ITR
  repeat_region 4677..4806: note = 3'ITR
  source 1..4806: mol_type = other DNA
  CDS 1470..4352: note = hCDKL5_1; protein_id = 30; translation = MKIPNIGNVMNKFEILGVVGEGAYGVVLKCRHKETHEIVAIKKF KDSEENEEVKETTLRELKMLRTLKQENIVELKEAFRRRGKLYLVFEYVEKNMLELLEE MPNGVPPEKVKSYIYQLIKAIHWCHKNDIVHRDIKPENLLISHNDVLKLCDFGFARNL SEGNNANYTEYVATRWYRSPELLLGAPYGKSVDMWSVGCILGELSDGQPLFPGESEID QLFTIQKVLGPLPSEQMKLFYSNPRFHGLRFPAVNHPQSLERRYLGILNSVLLDLMKN LLKLDPADRYLTEQCLNHPTFQTQRLLDRSPSRSAKRKPYHVESSTLSNRNQAGKSTA LQSHHRSNSKDIQNLSVGLPRADEGLPANESFLNGNLAGASLSPLHTKTYQASSQPGS TSKDLTNNNIPHLLSPKEAKSKTEFDFNIDPKPSEGPGTKYLKSNSRSQQNRHSFMES SQSKAGTLQPNEKQSRHSYIDTIPQSSRSPSYRTKAKSHGALSDSKSVSNLSEARAQI AEPSTSRYFPSSCLDLNSPTSPTPTRHSDTRTLLSPSGRNNRNEGTLDSRRTTTRHSK TMEELKLPEHMDSSHSHSLSAPHESFSYGLGYTSPFSSQQRPHRHSMYVTRDKVRAKG LDGSLSIGQGMAARANSLQLLSPQPGEQLPPEMTVARSSVKETSREGTSSFHTRQKSE GGVYHDPHSDDGTAPKENRHLYNDPVPRRVGSFYRVPSPRPDNSFHENNVSTRVSSLP SESSSGTNHSKRQPAFDPWKSPENISHSEQLKEKEKQGFFRSMKKKKKKSQTVPNSDS PDLLTLQKSIHSASTPSSRPKEWRPEKISDLQTQSQPLKSLRKLLHLSSASNHPASSD PRFQPLTAQQTKNSFSEIRIHPLSQASGGSSNIRQEPAPKGRPALQLPGQMDPGWHVS SVTRSATEGPSYSEQLGAKSGPNGHPYNRTNRSRMPNLNDLKETAL
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccggtagtgaattctaccagtgccataggatagtgaattctaccagtgccatacacgtgagtgaattctaccagtgccatagcatgcagtgaattctaccagtgccatagcggccgcgatctttttccctctgccaaaaattatggggacatcatgaagccccttgagcatctgacttctggctaataaaggaaatttattttcattgcaatagtgtgttggaattttttgtgtctctcactcgatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - protein, 960 aa, synthetic construct; identical to SEQ ID NO: 2
  REGION 1..960: note = Synthetic Construct

SEQ ID NO: 31 - DNA, 4389 bp, synthetic construct
  misc_feature 1..4389: note = Vector Genome
  repeat_region 1..130: note = 5' ITR
  misc_feature 130..168: note = AAV2 seq upstream of 3' ITR
  regulatory 224..1021: note = promoter - CBh; regulatory_class = promoter
  misc_feature 801: note = A deletion
  misc_feature 3936: note = Deletion (G)
  misc_feature 3953..3974: note = miRNA183
  misc_feature 3979..4000: note = miRNA183
  misc_feature 4007..4028: note = miRNA183
  misc_feature 4035..4056: note = miRNA183
  regulatory 4065..4191: note = polyA_signal - Rabbit Beta Globin PolyA; regulatory_class = polyA_signal_sequence
  misc_feature 4222..4260: note = AAV2 seq upstream of 3' ITR
  repeat_region 4260..4389: note = 3' ITR
  source 1..4389: mol_type = other DNA
  CDS 1053..3935: note = hCDKL5-1; protein_id = 32; translation = MKIPNIGNVMNKFEILGVVGEGAYGVVLKCRHKETHEIVAIKKF KDSEENEEVKETTLRELKMLRTLKQENIVELKEAFRRRGKLYLVFEYVEKNMLELLEE MPNGVPPEKVKSYIYQLIKAIHWCHKNDIVHRDIKPENLLISHNDVLKLCDFGFARNL SEGNNANYTEYVATRWYRSPELLLGAPYGKSVDMWSVGCILGELSDGQPLFPGESEID QLFTIQKVLGPLPSEQMKLFYSNPRFHGLRFPAVNHPQSLERRYLGILNSVLLDLMKN LLKLDPADRYLTEQCLNHPTFQTQRLLDRSPSRSAKRKPYHVESSTLSNRNQAGKSTA LQSHHRSNSKDIQNLSVGLPRADEGLPANESFLNGNLAGASLSPLHTKTYQASSQPGS TSKDLTNNNIPHLLSPKEAKSKTEFDFNIDPKPSEGPGTKYLKSNSRSQQNRHSFMES SQSKAGTLQPNEKQSRHSYIDTIPQSSRSPSYRTKAKSHGALSDSKSVSNLSEARAQI AEPSTSRYFPSSCLDLNSPTSPTPTRHSDTRTLLSPSGRNNRNEGTLDSRRTTTRHSK TMEELKLPEHMDSSHSHSLSAPHESFSYGLGYTSPFSSQQRPHRHSMYVTRDKVRAKG LDGSLSIGQGMAARANSLQLLSPQPGEQLPPEMTVARSSVKETSREGTSSFHTRQKSE GGVYHDPHSDDGTAPKENRHLYNDPVPRRVGSFYRVPSPRPDNSFHENNVSTRVSSLP SESSSGTNHSKRQPAFDPWKSPENISHSEQLKEKEKQGFFRSMKKKKKKSQTVPNSDS PDLLTLQKSIHSASTPSSRPKEWRPEKISDLQTQSQPLKSLRKLLHLSSASNHPASSD PRFQPLTAQQTKNSFSEIRIHPLSQASGGSSNIRQEPAPKGRPALQLPGQMDPGWHVS SVTRSATEGPSYSEQLGAKSGPNGHPYNRTNRSRMPNLNDLKETAL
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcgcccttaagggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcttaagctgcagaccggaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccggtagtgaattctaccagtgccataggatagtgaattctaccagtgccatacacgtgagtgaattctaccagtgccatagcatgcagtgaattctaccagtgccatagcggccgcgatctttttccctctgccaaaaattatggggacatcatgaagccccttgagcatctgacttctggctaataaaggaaatttattttcattgcaatagtgtgttggaattttttgtgtctctcactcgatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - protein, 960 aa, synthetic construct; identical to SEQ ID NO: 2
  REGION 1..960: note = Synthetic Construct

SEQ ID NO: 33 - DNA, 2211 bp, adeno-associated virus rh91
  source 1..2211: mol_type = other DNA
  CDS 1..2211: note = AAVrh.91; protein_id = 34; translation = MAADGYLPDWLEDNLSEGIREWWALKPGAPKPKANQQKQDDGRG LVLPGYKYLGPFNGLDKGEPVNAADAAALEHDKAYDQQLKAGDNPYLRYNHADAEFQE RLQEDTSFGGNLGRAVFQAKKRVLEPFGLVEEAAKTAPGKKRPVEQSPQEPDSSSGIG KSGQQPAKKRLNFGQTGDSESVPDPQPLGEPPETPAAVGPTTMASGGGAPMADNNEGA DGVGNASGNWHCDSTWLGDRVITTSTRTWALPTYNNHLYKQISSASTGASNDNHYFGY STPWGYFDFNRFHCHFSPRDWQRLINNNWGFRPKRLNFKLFNIQVKEVTTNDGVTTIA NNLTSTVQVFSDSEYQLPYVLGSAHQGCLPPFPADVFMIPQYGYLTLNNGSQAVGRSS FYCLEYFPSQMLRTGNNFTFSYTFEDVPFHSSYAHSQSLDRLMNPLIDQYLYYLNRTQ NQSGSAQNKDLLFSRGSPAGMSVQPKNWLPGPCYRQQRVSKTKTDNNNSNFTWTGASK YNLNGRESIINPGTAMASHKDDEDKFFPMSGVMIFGKENAGASNTALDNVMITDEEEI KATNPVATERFGTVAVNLQSSNTDPATGDVHVMGALPGMVWQDRDVYLQGPIWAKIPH TDGHFHPSPLMGGFGLKHPPPQILIKNTPVPANPPAEFSATKFASFITQYSTGQVSVE IEWELQKENSKRWNPEVQYTSNYAKSANVDFTVDNNGLYTEPRPIGTRYLTRPL
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 34 - protein, 736 aa, adeno-associated virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 35 - DNA, 2211 bp, adeno-associated virus rh91
  source 1..2211: mol_type = other DNA
  CDS 1..2211: note = AAVrh.91; protein_id = 36; translation = MAADGYLPDWLEDNLSEGIREWWALKPGAPKPKANQQKQDDGRG LVLPGYKYLGPFNGLDKGEPVNAADAAALEHDKAYDQQLKAGDNPYLRYNHADAEFQE RLQEDTSFGGNLGRAVFQAKKRVLEPFGLVEEAAKTAPGKKRPVEQSPQEPDSSSGIG KSGQQPAKKRLNFGQTGDSESVPDPQPLGEPPETPAAVGPTTMASGGGAPMADNNEGA DGVGNASGNWHCDSTWLGDRVITTSTRTWALPTYNNHLYKQISSASTGASNDNHYFGY STPWGYFDFNRFHCHFSPRDWQRLINNNWGFRPKRLNFKLFNIQVKEVTTNDGVTTIA NNLTSTVQVFSDSEYQLPYVLGSAHQGCLPPFPADVFMIPQYGYLTLNNGSQAVGRSS FYCLEYFPSQMLRTGNNFTFSYTFEDVPFHSSYAHSQSLDRLMNPLIDQYLYYLNRTQ NQSGSAQNKDLLFSRGSPAGMSVQPKNWLPGPCYRQQRVSKTKTDNNNSNFTWTGASK YNLNGRESIINPGTAMASHKDDEDKFFPMSGVMIFGKENAGASNTALDNVMITDEEEI KATNPVATERFGTVAVNLQSSNTDPATGDVHVMGALPGMVWQDRDVYLQGPIWAKIPH TDGHFHPSPLMGGFGLKHPPPQILIKNTPVPANPPAEFSATKFASFITQYSTGQVSVE IEWELQKENSKRWNPEVQYTSNYAKSANVDFTVDNNGLYTEPRPIGTRYLTRPL
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 36 - protein, 736 aa, adeno-associated virus rh91; identical to SEQ ID NO: 34

SEQ ID NO: 37 - DNA, 2883 bp, synthetic construct
  misc_feature 1..2883: note = engineered nucleic acid sequence CDKL5co-A
  source 1..2883: mol_type = other DNA
atgaagatccccaacatcggcaacgtgatgaacaagttcgagatcctgggcgtggtgggcgagggcgcctacggcgtggtgctgaagtgcaggcacaaggagacccacgagatcgtggccatcaagaagttcaaggacagcgaggagaacgaggaggtgaaggagaccaccctgagggagctgaagatgctgaggaccctgaagcaggagaacatcgtggagctgaaggaggccttcaggaggaggggcaagctgtacctggtgttcgagtacgtggagaagaacatgctggagctgctggaggagatgcccaacggcgtgccccccgagaaggtgaagagctacatctaccagctgatcaaggccatccactggtgccacaagaacgacatcgtgcacagggacatcaagcccgagaacctgctgatcagccacaacgacgtgctgaagctgtgcgacttcggcttcgccaggaacctgagcgagggcaacaacgccaactacaccgagtacgtggccaccaggtggtacaggagccccgagctgctgctgggcgccccctacggcaagagcgtggacatgtggagcgtgggctgcatcctgggcgagctgagcgacggccagcccctgttccccggcgagagcgagatcgaccagctgttcaccatccagaaggtgctgggccccctgcccagcgagcagatgaagctgttctacagcaaccccaggttccacggcctgaggttccccgccgtgaaccacccccagagcctggagaggaggtacttgggcatcctgaacagcgtgctgctggacctgatgaagaacctgctgaagctggaccccgccgacaggtacttgaccgagcagtgcctgaaccaccccaccttccagacccagaggctgctggacaggagccccagcaggagcgccaagaggaagccctaccacgtggagagcagcaccctgagcaacaggaaccaggccggcaagagcaccgccctgcagagccaccacaggagcaacagcaaggacatccagaacctgagcgtgggcctgcccagggccgacgagggcctgcccgccaacgagagcttcctgaacggcaacctggccggcgccagcctgagccccctgcacaccaagacctaccaggccagcagccagcccggcagcaccagcaaggacctgaccaacaacaacatcccccacctgctgagccccaaggaggccaagagcaagaccgagttcgacttcaacatcgaccccaagcccagcgagggccccggcaccaagtacctgaagagcaacagcaggagccagcagaacaggcacagcttcatggagagcagccagagcaaggccggcaccctgcagcccaacgagaagcagagcaggcacagctacatcgacaccatcccccagagcagcaggagccccagctacaggaccaaggccaagagccacggcgccctgagcgacagcaagagcgtgagcaacctgagcgaggccagggcccagatcgccgagcccagcaccagcaggtacttccccagcagctgcctggacctgaacagccccaccagccccacccccaccaggcacagcgacaccaggaccctgctgagccccagcggcaggaacaacaggaacgagggcaccctggacagcaggaggaccaccaccaggcacagcaagaccatggaggagctgaagctgcccgagcacatggacagcagccacagccacagcctgagcgccccccacgagagcttcagctacggcctgggctacaccagccccttcagcagccagcagaggccccacaggcacagcatgtacgtgaccagggacaaggtgagggccaagggcctggacggcagcctgagcatcggccagggcatggccgccagggccaacagcctgcagctgctgagcccccagcccggcgagcagctgccccccgagatgaccgtggccaggagcagcgtgaaggagaccagcagggagggcaccagcagcttccacaccaggcagaagagcgagggcggcgtgtaccacgacccccacagcgacgacggcaccgcccccaaggagaacaggcacctgtacaacgaccccgtgcccaggagggtgggcagcttctacagggtgcccagccccaggcccgacaacagcttccacgagaacaacgtgagcaccagggtgagcagcctgcccagcgagagcagcagcggcaccaaccacagcaagaggcagcccgccttcgacccctggaagagccccgagaacatcagccacagcgagcagctgaaggagaaggagaagcagggcttcttcaggagcatgaagaagaagaagaagaagagccagaccgtgcccaacagcgacagccccgacctgctgaccctgcagaagagcatccacagcgccagcacccccagcagcaggcccaaggagtggaggcccgagaagatcagcgacctgcagacccagagccagcccctgaagagcctgaggaagctgctgcacctgagcagcgccagcaaccaccccgccagcagcgaccccaggttccagcccctgaccgcccagcagaccaagaacagcttcagcgagatcaggatccaccccctgagccaggccagcggcggcagcagcaacatcaggcaggagcccgcccccaagggcaggcccgccctgcagctgcccggccagatggaccccggctggcacgtgagcagcgtgaccaggagcgccaccgagggccccagctacagcgagcagctgggcgccaagagcggccccaacggccacccctacaacaggaccaacaggagcaggatgcccaacctgaacgacctgaaggagaccgccctgtga

SEQ ID NO: 38 - DNA, 2883 bp, synthetic construct
  misc_feature 1..2883: note = engineered nucleic acid sequence CDKL5co-B
  source 1..2883: mol_type = other DNA
atgaaaattcccaatatcggtaacgtgatgaataaattcgaaatacttggagtggtaggtgagggcgcctatggtgttgtacttaaatgtagacacaaagaaacacacgaaatcgtggcgatcaagaaatttaaggattccgaggagaatgaggaagtcaaagagaccacactacgcgagttgaaaatgttacgaactctaaaacaggaaaacattgtagagttaaaggaggcttttcgccgtcgcggtaaactatacctggtctttgagtatgttgagaaaaacatgctagaactgctcgaggaaatgcctaatggcgtcccgccagaaaaagtcaaatcgtatatttaccagcttataaaggccatccattggtgtcataaaaacgatatcgttcatcgcgacattaagccagaaaatctattgatatcccacaacgacgtgctaaaactttgcgacttcggcttcgctaggaacctatccgaagggaataacgcgaattataccgaatatgttgcgacacgatggtaccgtagtcctgaacttctattaggggcgccctacggtaaatctgtagatatgtggtcagtcgggtgtatattaggagaactttcagacggtcaacctctgttcccaggcgagtccgagatcgatcagctttttacaatacagaaggtattaggtcccctcccaagtgagcaaatgaagctattctattcaaatccccggtttcatggccttagatttccggcggtgaaccaccctcagagtctggagaggcgttatctcgggatacttaattcagtcttactcgatctgatgaagaatttgctaaaactggaccctgctgatcggtaccttacagagcaatgccttaatcacccgacttttcaaactcaacgactgttggaccggagtccgtcacgctcagctaaaaggaaaccataccacgtagaatcatcgacattatcgaatcgaaaccaagcgggtaagagtaccgctttgcaaagccatcatcgatccaatagtaaagacattcagaatctgtcagtgggattgcctagagctgacgaaggattaccggcgaacgagagttttctgaacggcaacctcgcaggcgcatctctgtctccgcttcatacgaaaacttaccaagcgtcatcacagccaggcagtacgagtaaagacctgactaacaataatattccacatctgttatcaccgaaggaagctaagagcaagaccgagttcgacttcaacatagaccccaaaccctccgagggtcctggcaccaagtatctcaaatcaaactctagatctcagcagaacaggcacagcttcatggagtcctcacaaagtaaagctgggacactgcagcccaacgaaaagcaaagccgacattcttatattgacacgatccctcagagttcacgcagccctagctatcggacaaaagctaagtcccacggtgccctttctgattcgaagagtgttagtaacctttccgaagctagagcccaaattgcggagcctagtacatcgagatacttcccctccagctgcctggaccttaactctccaaccagccccactccaacgcgccactcagataccaggacactgctctcaccatccggaagaaataatagaaatgaaggaacgcttgactctcggcgcactacgacaaggcacagcaagacgatggaagaacttaagctacctgagcacatggacagttcgcattcccactcactctcggcgccacatgagtcgttttcctatggactgggttacacaagtccctttagctctcagcagcgtcctcaccgccactctatgtacgttacaagggataaagttcgcgctaaggggttggatgggagtctttccattggccaaggcatggccgcgcgggcaaattctctacaacttctttcgccacagccaggtgagcaactcccaccagaaatgaccgtcgccagaagcagtgttaaagaaacttcgagggaggggacatcatcgtttcatacccgccagaaatctgagggcggtgtatatcatgatcctcattccgacgacggaacggcccccaaagagaaccgacacctatacaatgaccctgtcccaaggagagtgggatctttctatcgagttccttcaccaagacctgataacagtttccatgagaacaacgtttccacgagggtgtcaagtctaccttcggaatcatcttccggaaccaatcactcaaaacgccagccagctttcgatccatggaaatcaccggaaaacatcagtcatagcgagcaattgaaagagaaagaaaagcaaggttttttcagaagtatgaagaagaagaaaaagaagtcacagaccgtacctaattcagatagtccagacttgttaacgctccagaaatcaattcatagcgcatcgactccatcgtcccgaccaaaggaatggcgccctgagaagatctccgatctgcagacccaatcccaacccctgaagtctctcaggaaattactccacctctcgtctgctagtaaccaccccgcatcgtctgaccctcgcttccagccgctgacagcccagcaaacaaagaacagtttctcagaaatccggattcatcctttaagccaagctagcggcggtagttctaacataagacaagagcccgctccaaagggcagacccgctctgcagttgcctgggcagatggatcccgggtggcacgtctcatcagtaacccgaagtgccaccgagggcccgtcatattcagagcagctcggagcgaaaagtggtcctaacgggcacccatataatcgaacaaaccgttcaaggatgccaaatctcaacgatctaaaggaaacggccctttaa

SEQ ID NO: 39 - DNA, 2883 bp, synthetic construct
  misc_feature 1..2883: note = engineered nucleic acid sequence CDKL5co-C
  source 1..2883: mol_type = other DNA
atgaagatccccaatatcggaaacgtgatgaacaaattcgagattctcggagtggtgggcgagggcgcctatggagtggttttaaaatgcagacacaaggagacccacgagatcgtcgctatcaaaaagttcaaagactccgaggaaaacgaagaggtcaaggagaccaccttacgtgagctcaaaatgctcagaacactgaagcaagagaatatcgtcgagctcaaggaggccttcagaaggaggggcaaactgtatttagtgttcgagtacgtggagaagaacatgctggagctgctggaggaaatgcccaacggcgtcccccccgaaaaggtgaaatcctacatttaccaactgatcaaggccattcactggtgccacaaaaatgatatcgtccatcgtgacatcaagcccgagaatctgctgatcagccataacgacgtgctgaaactgtgcgacttcggattcgccagaaatttaagcgagggcaacaacgccaattacaccgagtacgtcgccacaaggtggtataggagccccgagctgctgctcggagccccttatggcaagagcgtggacatgtggagcgtgggctgcattctgggagaactgtccgacggccagcctctgttccccggcgagtccgagattgaccagctgttcacaatccagaaagtcctcggccctctgcccagcgaacaaatgaagctcttctacagcaaccctcgtttccacggtttaaggttccccgccgtgaatcacccccagagcctcgagagaaggtatctgggcattctgaacagcgtgctgctcgatttaatgaagaatttattaaagctggaccccgctgataggtacctcacagagcagtgtttaaatcaccccaccttccagacccagagactgctcgatcgtagccctagcagatccgccaaaaggaagccttaccacgtcgagagctccactttaagcaatcgtaaccaagctggaaaatccaccgctttacagtcccaccataggtccaacagcaaagacattcagaatttaagcgtgggactgcctagggctgacgagggcctccccgccaacgaatcctttttaaatggcaatctcgccggagccagcctcagccctttacatacaaaaacctaccaagctagctcccagcccggtagcacctccaaggacctcaccaacaacaacatcccccatttactgtcccccaaggaggccaagagcaagaccgagtttgactttaatatcgaccctaagcccagcgagggacccggcacaaagtatctgaagtccaattctcgtagccagcagaatcgtcacagcttcatggagagctcccaaagcaaggccggcacactgcagcccaatgagaagcaatctcgtcattcctacatcgacaccatcccccagagctctcgtagcccctcctataggacaaaggccaagtcccacggcgctctgtccgattccaagtccgtctccaatttatccgaggctcgtgcccagatcgccgaacctagcacctctcgttatttcccctcctcttgtctggacctcaattcccccacaagccctacacctactcgtcacagcgacaccagaactttactgagccccagcggaaggaataatcgtaacgagggcactttagactctcgtagaaccaccacaaggcacagcaagaccatggaggagctgaagctgcccgaacatatggactcctcccattcccattctttatccgccccccacgagagcttcagctatggtttaggctatacatcccccttcagctcccagcagagaccccacagacactccatgtacgtgactcgtgataaggtgagggccaaaggactggatggaagcctctccatcggccaaggcatggccgctcgtgccaatagcctccagttattatccccccaacccggagaacagctgcctcccgaaatgacagtggccagaagcagcgtcaaggaaacatctcgtgagggaacctccagctttcacacaaggcagaagagcgagggcggagtctatcatgacccccactccgatgacggcacagcccccaaagagaatcgtcatctctacaacgaccccgtgcctcgtagagtcggctccttctacagagtccccagccccagacccgacaacagctttcacgagaacaacgtgagcactcgtgtctcctctttacctagcgagagctcctccggaaccaaccacagcaaaaggcagcccgccttcgacccttggaagtcccccgaaaacatcagccacagcgagcagctgaaggagaaagagaagcaaggtttcttcagaagcatgaaaaagaagaagaaaaaatcccaaaccgtgcccaactccgactcccccgatttactgaccctccagaagtccatccactccgcttccacccctagctccagacccaaggagtggaggcccgagaagatttccgatttacagacacagagccagcctctcaagtctttaagaaagctgctgcatttatccagcgccagcaatcaccccgcttccagcgaccctaggttccagcctttaaccgcccagcagaccaagaatagcttttccgagatccgtattcatcctctgagccaagctagcggaggcagctccaacatcagacaagaacccgctcctaagggcagacccgctttacaactccccggtcagatggatcccggttggcacgtgagcagcgtcacaaggagcgccacagagggccccagctatagcgaacagctgggcgccaaaagcggacccaacggccatccctacaatagaaccaatcgttccagaatgcccaatctcaacgacctcaaggaaacagctttatga

SEQ ID NO: 40 - DNA, 2883 bp, synthetic construct
  misc_feature 1..2883: note = engineered nucleic acid sequence CDKL5co-D
  source 1..2883: mol_type = other DNA
atgaagatccccaacatcggcaacgtgatgaacaagttcgagatcctgggcgtcgtcggcgaaggcgcttatggcgttgtgctgaagtgcagacacaaagaaacccacgagatcgtggccatcaagaagttcaaggacagcgaagagaacgaggaagtgaaagagacaaccctgcgcgagctgaagatgctgcggaccctgaagcaagagaacatcgtggaactgaaagaggccttccggcggagaggcaagctgtacctggtgttcgagtacgtggaaaagaacatgctcgagctgctggaagagatgcccaacggcgtgccacctgagaaagtgaagtcctacatctaccagctgatcaaggccatccactggtgccacaagaacgacatcgtgcaccgggacatcaagcccgagaacctgctgatctcccacaacgacgtgctgaagctgtgcgacttcggcttcgccagaaatctgagcgagggcaacaacgccaactacaccgaatacgtggccaccagatggtacagaagccctgagctgctgctgggagccccttatggcaagtccgtggatatgtggagcgtgggctgtatcctgggagagctgtctgatggccagcctctgtttcctggcgagtccgagatcgaccagctgttcaccatccagaaggtgctgggccctctgcctagcgagcagatgaagctgttctacagcaaccccagattccacggcctgagattccccgccgtgaatcaccctcagagcctggaaagaagatacctgggcatcctgaacagcgtgctgctggacctgatgaagaatctgctgaaactggaccccgccgacagatacctgaccgagcagtgcctgaaccatcctacctttcagacccagagactgctggatcggagccctagcagaagcgccaagagaaagccctaccacgtggaaagcagcaccctgagcaacagaaaccaggccggcaagtctacagccctgcagagccaccacagaagcaacagcaaggacatccagaacctgtccgtgggcctgcctagagccgatgaaggactgcctgccaacgagagcttcctgaacggaaatctggctggcgcctctctgagccctctgcacacaaagacataccaggccagcagccagcctggcagcacaagcaaggatctgaccaacaacaacatccctcatctgctgagccccaaagaggccaagagcaagaccgagttcgacttcaacatcgaccccaagcctagcgaaggccctggcaccaagtacctgaagtccaacagcagaagccagcagaaccggcactccttcatggaaagctcccagagcaaggccggcacactgcagcctaatgagaagcagagcagacacagctacatcgacacaatcccacagagcagcaggtccccaagctacaggaccaaggccaaatctcacggcgccctgagcgatagcaagagcgtgtccaatctgtccgaggctagagcccagatcgccgagcctagcaccagcagatacttccctagctcctgcctggatctgaacagcccaacaagccccacacctaccaggcacagcgacaccagaacactgctgtccccatccggcagaaacaaccggaatgagggcaccctggacagcagacggaccaccacaagacacagcaagaccatggaagaactgaagctgcccgagcacatggactccagccactctcattctctgagcgcccctcacgagtccttctcttacggcctgggctacacaagccccttcagctctcagcagaggccccacagacactctatgtacgtgacccgggacaaagtgcgcgccaaaggcctggatggctctctgtctatcggccaaggcatggccgccagagccaattctctgcagctgctgtctcctcagcctggggaacaactgcctcctgagatgaccgtggccagatccagcgtgaaagaaaccagcagagagggcacctccagcttccacaccagacagaaaagcgaaggcggagtgtaccacgatcctcacagcgacgacggcacagcccctaaagagaacagacacctgtacaacgaccccgtgcctagaagagtgggctccttctacagagtgccttctcctagacctgacaactccttccacgagaacaatgtgtccaccagagtgtccagcctgccttccgaaagcagctccggcaccaaccactccaagagacagcctgctttcgacccctggaagtcccctgagaacatcagccactccgagcagctgaaagagaaagagaagcagggctttttccggtccatgaagaagaagaaaaagaagtcccagacggtccccaacagcgactccccagatctgctgaccctgcagaagtccatccactccgccagcacacctagcagcagacccaaagagtggcggccagagaagatcagcgatctgcagacccagtctcagcctctgaagtccctgaggaagctgctgcatctgagcagcgcctctaatcaccctgccagcagcgaccctagatttcagccactgacagcccagcagaccaagaactccttcagcgagatccggattcacccactgtctcaggccagcggcggcagcagcaatatcagacaagagcccgctcctaagggcagacctgctctgcaactgcctggccagatggatccaggatggcacgtgtcctctgtgaccagatccgccacagagggacctagctacagcgaacagctcggcgccaagtctggccctaatggacacccctacaaccggaccaaccggtccagaatgcccaacctgaacgacctcaaagaaacagccctgtga

SEQ ID NO: 41 - DNA, 2883 bp, synthetic construct
  misc_feature 1..2883: note = engineered nucleic acid sequence CDKL5coA1
  source 1..2883: mol_type = other DNA
atgaagatccccaacatcggcaacgtgatgaacaagttcgagatcctgggcgtcgtcggcgaaggcgcttatggcgttgtgctgaagtgcagacacaaagaaacccacgagatcgtggccatcaagaagttcaaggacagcgaagagaacgaggaagtgaaagagacaaccctgcgcgagctgaagatgctgcggaccctgaagcaagagaacatcgtggaactgaaagaggccttccggcggagaggcaagctgtacctggtgttcgagtacgtggaaaagaacatgctcgagctgctggaagagatgcccaacggcgtgccacctgagaaagtgaagtcctacatctaccagctgatcaaggccatccactggtgccacaagaacgacatcgtgcaccgggacatcaagcccgagaacctgctgatctcccacaacgacgtgctgaagctgtgcgacttcggcttcgccagaaatctgagcgagggcaacaacgccaactacaccgaatacgtggccaccagatggtacagaagccctgagctgctgctgggagccccttacggcaagtccgtggatatgtggagcgtgggctgtatcctgggagagctgtctgacggccagcctctgtttcctggcgagtccgagatcgaccagctgttcaccatccagaaggtgctgggccctctgcctagcgagcagatgaagctgttctacagcaaccccagattccacggcctgagattccccgccgtgaatcaccctcagagcctggaaagaagatacctgggcatcctgaacagcgtgctgctggacctgatgaagaatctgctgaaactggaccccgccgacagatacctgaccgagcagtgcctgaaccatcctacctttcaaacccagagactgctggatcggagccctagcagaagcgccaagagaaagccctaccacgtggaaagcagcaccctgagcaacagaaaccaggccggcaagtctacagccctgcagagccaccacagaagcaacagcaaggacatccagaacctgtccgtgggcctgcctagagccgacgaaggactgcctgccaacgagagcttcctgaacggaaatctggctggcgcctctctgagccctctgcacacaaagacataccaggccagcagccagcctggcagcacaagcaaggatctgaccaacaacaacatccctcatctgctgagccccaaagaggccaagagcaagaccgagttcgacttcaacatcgaccccaagcctagcgaaggccctggcaccaagtacctgaagtccaacagcagaagccagcagaaccggcactccttcatggaaagctcccagagcaaggccggcacactgcagcctaacgagaagcagagcagacacagctacatcgacacaatcccacagagcagcaggtccccaagctacaggaccaaggccaaatctcacggcgccctgagcgatagcaagagcgtgtccaatctgtccgaggctagagcccagatcgccgagcctagcaccagcagatacttccctagctcctgcctggatctgaacagcccaacaagccccacacctaccaggcacagcgacaccagaacactgctgtccccatccggcagaaacaaccggaacgagggcaccctggacagcagacggaccaccacaagacacagcaagaccatggaagaactgaagctgcccgagcacatggactccagccactctcattctctgagcgcccctcacgagtccttctcttacggcctgggctacacaagccccttcagctctcagcagaggccccacagacacagcatgtatgtgacccgggacaaagtgcgcgccaaaggcctggacggctctctgtctatcggccaaggcatggccgccagagccaattctctgcagctgctgtctcctcagcctggggaacaactgcctcctgagatgaccgtggccagatccagcgtgaaagaaaccagcagagagggcacctccagcttccacaccagacagaaaagcgaaggcggagtgtaccacgatcctcacagcgacgacggcacagcccctaaagagaacagacacctgtacaacgaccccgtgcctagaagagtgggctccttctacagagtgccttctcctagacctgacaactccttccacgagaacaacgtgtccaccagagtgtccagcctgccttccgaaagcagctccggcaccaaccactccaagagacagcctgctttcgacccctggaagtcccctgagaacatcagccactccgagcagctgaaagagaaagagaagcagggctttttccggtccatgaagaagaagaaaaagaagtcccagacggtccccaacagcgactccccagatctgctgaccctgcagaagtccatccactccgccagcacacctagcagcagacccaaagagtggcggccagagaagatcagcgatctgcagacccagtctcagcctctgaagtccctgaggaagctgctgcatctgagcagcgcctctaatcaccctgccagcagcgaccctagatttcagccactgacagcccagcagaccaagaactccttcagcgagatccggattcacccactgtctcaggccagcggcggcagcagcaatatcagacaagagcccgctcctaagggcagacctgctctgcaactgcctggccagatggatccaggctggcacgtgtcctctgtgaccagatccgccacagagggacctagctacagcgaacagctcggcgccaagtctggccctaacggacacccctacaaccggaccaaccggtccagaatgcccaacctgaacgacctcaaagagactgccctttga

SEQ ID NO: 42 - DNA, 2883 bp, synthetic construct
  misc_feature 1..2883: note = engineered nucleic acid sequence CDKL5coB1
  source 1..2883: mol_type = other DNA
atgaagatccctaatatcggcaacgtgatgaataagttcgagatcctgggagtggtgggagagggagcatacggagtggtgctgaagtgccgccacaaggagacccacgagatcgtggccatcaagaagtttaaggatagcgaggagaatgaggaggtgaaggagaccacactgagagagctgaagatgctgaggacactgaagcaggagaacatcgtggagctgaaggaggccttccggagaaggggcaagctgtacctggtgtttgagtacgtggagaagaacatgctggagctgctggaggagatgcccaatggcgtgccccctgagaaggtgaagtcctacatctatcagctgatcaaggccatccactggtgccacaagaacgacatcgtgcacagagatatcaagcctgagaacctgctgatctcccacaatgacgtgctgaagctgtgcgatttcggctttgccaggaacctgagcgagggcaacaacgccaattacacagagtacgtggccacccgctggtacagaagcccagagctgctgctgggagcaccatacggcaagagcgtggacatgtggtccgtgggctgcatcctgggcgagctgtctgacggccagcctctgttcccaggcgagagcgagatcgatcagctgtttaccatccagaaggtgctgggccctctgccaagcgagcagatgaagctgttctactccaaccccagattccacggcctgaggtttcccgccgtgaatcaccctcagagcctggagcgccggtatctgggcatcctgaactccgtgctgctggatctgatgaagaatctgctgaagctggaccccgccgatcgctacctgaccgagcagtgtctgaatcaccctacatttcagacccagcgcctgctggaccggagcccttccagatctgccaagaggaagccatatcacgtggagagctccaccctgtccaatagaaaccaggccggcaagtctacagccctgcagagccaccacaggagcaactccaaggacatccagaatctgtccgtgggactgcctagggcagacgagggactgccagcaaacgagagcttcctgaacggaaacctggcaggagcatctctgagcccactgcacacaaagacctaccaggcctctagccagcccggctccacatctaaggacctgaccaacaataacatcccacacctgctgtctcccaaggaggccaagagcaagaccgagttcgactttaatatcgatcccaagcctagcgagggccctggcacaaagtatctgaagagcaactcccgctctcagcagaatcggcactccttcatggagtcctctcagtctaaggccggcaccctgcagccaaacgagaagcagagcaggcactcctacatcgataccatcccccagagctccagaagcccttcctatagaacaaaggccaagtctcacggcgccctgtctgacagcaagtccgtgtctaatctgagcgaggcaagggcacagatcgcagagcccagcacctcccggtactttccttcttcctgtctggacctgaactctcctacaagcccaacacccacccgccactccgatacacggaccctgctgtctccaagcggcagaaataacaggaacgagggcaccctggattctagaaggaccacaaccagacacagcaagacaatggaggagctgaagctgccagagcacatggactcctctcactcccactctctgagcgcccctcacgagtccttctcttacggcctgggctatacctcccccttctcctcccaacaaaggccacaccggcacagcatgtatgtgacaagggacaaggtccgggcaaagggactggacggaagcctgtccatcggacagggaatggcagcaagggccaactccctgcagctgctgtctcctcagccaggagagcagctgccaccagagatgaccgtggcacgctctagcgtgaaggagacaagccgggagggcacctcctctttccacacacgccagaagtccgagggcggcgtgtatcacgacccccactctgacgacggcacagcccctaaggagaaccggcacctgtacaacgatcccgtgcctaggagggtgggatccttctatagagtgccatctcccaggcctgacaatagctttcacgagaataacgtgtccacccgcgtgagctccctgccatctgagtctagctccggcacaaaccactctaagcggcagcccgcctttgatccttggaagagcccagagaatatctctcacagcgagcagctgaaggagaaggagaagcagggcttctttagaagcatgaagaagaagaagaagaagagccagaccgtgcctaactccgactctccagacctgctgaccctgcagaagtccatccacagcgcctccacaccatctagcaggcctaaggagtggcggcctgagaagatcagcgatctgcagacccagagccagccactgaagtccctgaggaagctgctgcacctgtcctctgccagcaaccacccagccagctccgacccaagattccagcccctgacagcccagcagaccaagaacagcttcagcgagatcaggatccacccactgtcccaggcatctggaggatctagcaacatcagacaggagccagcaccaaagggcaggcccgccctgcagctgcctggccagatggacccaggctggcacgtgtcctctgtgacacgctccgccaccgagggaccatcctactctgagcagctgggagcaaagagcggacctaacggacacccatataaccggaccaatagatccaggatgcccaatctgaacgacctgaaggagacagccctgtga

SEQ ID NO: 43 - DNA, 2883 bp, synthetic construct
  misc_feature 1..2883: note = engineered nucleic acid sequence CDKL5coC1
  source 1..2883: mol_type = other DNA
atgaagatccccaacatcggcaacgtgatgaacaagttcgagatcctgggcgtggtgggcgagggcgcctacggcgtggtgctgaagtgcaggcacaaggagacccacgagatcgtggccatcaagaagttcaaggacagcgaggagaacgaggaggtgaaggagaccaccctgagggagctgaagatgctgaggaccctgaagcaggagaacatcgtggagctgaaggaggccttcaggaggaggggcaagctgtacctggtgttcgagtacgtggagaagaacatgctggagctgctggaggagatgcccaacggcgtgccccccgagaaggtgaagagctacatctaccagctgatcaaggccatccactggtgccacaagaacgacatcgtgcacagggacatcaagcccgagaacctgctgatcagccacaacgacgtgctgaagctgtgcgacttcggcttcgccaggaacctgagcgagggcaacaacgccaactacaccgagtacgtggccaccaggtggtacaggagccccgagctgctgctgggcgccccctacggcaagagcgtggacatgtggagcgtgggctgcatcctgggcgagctgagcgacggccagcccctgttccccggcgagagcgagatcgaccagctgttcaccatccagaaggtgctgggccccctgcccagcgagcagatgaagctgttctacagcaaccccaggttccacggcctgaggttccccgccgtgaaccacccccagagcctggagaggaggtatctgggcatcctgaacagcgtgctgctggacctgatgaagaacctgctgaagctggaccccgccgacaggtatctgaccgagcagtgcctgaaccaccccaccttccagacccagaggctgctggacaggagccccagcaggagcgccaagaggaagccctaccacgtggagagcagcaccctgagcaacaggaaccaggccggcaagagcaccgccctgcagagccaccacaggagcaacagcaaggacatccagaacctgagcgtgggcctgcccagggccgacgagggcctgcccgccaacgagagcttcctgaacggcaacctggccggcgccagcctgagccccctgcacaccaagacctaccaggccagcagccagcccggcagcaccagcaaggacctgaccaacaacaacatcccccacctgctgagccccaaggaggccaagagcaagaccgagttcgacttcaacatcgaccccaagcccagcgagggccccggcaccaagtacctgaagagcaacagcaggagccagcagaacaggcacagcttcatggagagcagccagagcaaggccggcaccctgcagcccaacgagaagcagagcaggcacagctacatcgacaccatcccccagagcagcaggagccccagctacaggaccaaggccaagagccacggcgccctgagcgacagcaagagcgtgagcaacctgagcgaggccagggcccagatcgccgagcccagcaccagcagatacttccccagcagctgcctggacctgaacagccccaccagccccacccccacccgccacagcgacaccaggaccctgctgagccccagcggcaggaacaacaggaacgagggcaccctggacagcaggaggaccaccaccaggcacagcaagaccatggaggagctgaagctgcccgagcacatggacagcagccacagccacagcctgagcgccccccacgagagcttcagctacggcctgggctacaccagccccttcagcagccagcagaggccccacaggcacagcatgtacgtgaccagggacaaagtgagggccaagggcctggacggcagcctgagcatcggccagggcatggccgccagggccaacagcctgcagctgctgagcccccagcccggcgagcagctgccccccgagatgaccgtggccaggagcagcgtgaaggagaccagcagggagggcaccagcagcttccacaccaggcagaagagcgagggcggcgtgtaccacgacccccacagcgacgacggcaccgcccccaaggagaacaggcacctgtacaacgaccccgtgcccaggagggtgggcagcttctacagggtgcccagccccaggcccgacaacagcttccacgagaacaatgtgagcaccagagtcagcagcctgcccagcgagagcagcagcggcaccaaccacagcaagaggcagcccgccttcgacccctggaagagccccgagaacatcagccacagcgagcagctgaaggagaaggagaagcagggcttcttcaggagcatgaagaagaagaagaagaagagccagaccgtgcccaacagcgacagccccgacctgctgaccctgcagaagagcatccacagcgccagcacccccagcagcaggcccaaggagtggaggcccgagaagatcagcgacctgcagacccagagccagcccctgaagagcctgaggaagctgctgcacctgagcagcgccagcaaccaccccgccagcagcgaccccaggttccagcccctgaccgcccagcagaccaagaacagcttcagcgagatcaggatccaccccctgagccaggccagcggcggcagcagcaacatcaggcaggagcccgcccccaagggcaggcccgccctgcagctgcccggccagatggaccccggctggcacgtgagcagcgtgaccaggagcgccaccgagggccccagctacagcgagcagctgggcgccaagagcggccccaacggccacccctacaacaggaccaacaggagcaggatgcccaacctgaacgacctgaaggagaccgccctgtga

SEQ ID NO: 44 - DNA, 2883 bp, synthetic construct
  misc_feature 1..2883: note = engineered nucleic acid sequence CDKL5coD1
  source 1..2883: mol_type = other DNA
atgaagattcccaacattgggaatgtcatgaacaagtttgagattctgggcgtggtgggagagggagcctacggcgttgttctcaagtgccgccacaaggaaactcacgagatcgtggctatcaagaaattcaaggatagtgaggaaaatgaagaagtcaaggagacaactcttcgagaattaaagatgctcagaaccttaaaacaagaaaatattgtggaattgaaagaggccttcagaagacgaggaaaactctacctggtgtttgaatatgtggagaagaacatgctggaactgttggaagagatgcctaatggggtccctcctgaaaaagtgaagtcctacatctatcagctaatcaaggccatccactggtgccacaaaaatgatattgtccaccgggacatcaaaccagagaatttgctcatcagccacaatgatgtgctcaaactttgtgattttggatttgcccgaaacctctccgaaggcaataatgccaactacaccgagtacgtggccacccgctggtatcgctctccagagcttctgcttggggccccctatggcaagtctgtggacatgtggagcgttggctgcatcctgggcgagctgtcagatgggcagccactcttcccaggggagtccgaaatcgaccagctgttcaccatccagaaagtcctggggccacttcccagcgagcaaatgaaactgttttacagcaatccaagatttcatggattacgcttccctgctgtcaaccaccctcaatccctggagaggagatacttgggcatcctcaacagtgtgctgctggacctcatgaagaatctcctcaagttggaccctgccgacagatatcttactgagcagtgcctgaaccatcccacatttcagacccagagattgctagacaggagcccatcaaggagtgccaagagaaagccttatcatgtggagagcagcacactatcaaacagaaaccaggctggaaagagtacagctttacaatctcaccaccgctccaactcaaaggacattcagaacttgtcagttggactgcccagagcagatgagggactccccgccaatgaaagcttcctgaatggaaacctggcaggtgcctccctttctcctctgcacacaaagacctaccaggcgtccagccaacctggatccacatcaaaagatctgaccaacaacaatatcccccaccttttatcaccaaaggaagccaaaagtaaaacagagttcgacttcaacatcgaccccaaaccttcagaagggcctggcaccaaatatttaaaatcaaattcaagaagccagcagaatcggcactcattcatggaatcctcacagagcaaagctgggacactccagccaaatgagaagcagtccagacatagttatattgacaccattcctcagtcatccagatccccaagctaccggacaaaagccaagagccatggcgcgctgtccgattccaagtcagtatcaaatctttcagaagcaagagctcagatcgcagagccctccacttcccgctacttcccctcttcatgtttggatctcaattcccctacttcacctacacccacccgtcatagtgacacacggaccctgctcagtccaagcggcaggaataaccggaacgagggcacactggacagccgccgcaccaccacaaggcattctaaaaccatggaggagctgaaattgcccgagcacatggactcttcacattctcattcactatctgcacctcatgaaagttttagctatggattgggatataccagccccttctccagtcagcagaggcctcacagacacagcatgtatgtcaccagggacaaggtgcgggcaaagggcctggatggatctttaagtattggccagggcatggctgccagagcaaattctcttcagctgctttcaccccagcctggggaacaactgccaccagaaatgacagtggcaaggagcagtgtaaaagagacctccagagaaggaacatccagcttccatactcgccagaagtcggaaggtggtgtgtaccacgacccacattctgatgatgggaccgctcccaaggaaaacaggcacctgtacaacgatcctgtgccccgccgcgtgggctctttctacagagttccttctcctagaccagacaactccttccacgaaaacaatgtgtccaccagagtgagcagtttaccctctgagagttctagtggaaccaatcattccaaaagacagcctgcatttgacccctggaagtccccggagaatatctcccactccgagcagctcaaagaaaaggagaaacagggattcttcaggtccatgaaaaagaagaagaaaaaaagccagacagtaccaaacagcgattctccagatcttctcactctgcaaaaatccatccattctgccagtactccatcatctcgacccaaggagtggagacctgagaaaatttcagacctgcagactcagtctcagcccctgaaatctttgaggaagttattacacctctcatcagcttccaatcacccagccagctcagatcccaggttccagccgctcacagcccagcaaacaaaaaactctttttcagaaatacgtattcatcctttgtcccaggcatcaggaggcagcagcaacatcaggcaggagccagctccaaaaggaaggcccgctctgcagctgccaggacagatggatccaggctggcatgtttcctctgtcactagatcagccacagaaggaccatcctattctgaacagctgggtgcaaaaagtggccccaatggccacccctacaatagaacaaatcgcagcagaatgcccaacttaaatgacttgaaagaaacagcactgtga

SEQ ID NO: 45 - DNA, 2883 bp, synthetic construct
  misc_feature 1..2883: note = engineered nucleic acid sequence CDKL5coA2
  source 1..2883: mol_type = other DNA
atgaagatccccaacatcggcaacgtgatgaacaagttcgagatcctgggcgtcgtcggcgaaggcgcttatggcgttgtgctgaagtgcagacacaaagaaacccacgagatcgtggccatcaagaagttcaaggacagcgaagagaacgaggaagtgaaagagacaaccctgcgcgagctgaagatgctgcggaccctgaagcaagagaacatcgtggaactgaaagaggccttccggcggagaggcaagctgtacctggtgttcgagtacgtggaaaagaacatgctcgagctgctggaagagatgcccaacggcgtgccacctgagaaagtgaagtcctacatctaccagctgatcaaggccatccactggtgccacaagaacgacatcgtgcaccgggacatcaagcccgagaacctgctgatctcccacaacgacgtgctgaagctgtgcgacttcggcttcgccagaaatctgagcgagggcaacaacgccaactacaccgaatacgtggccaccagatggtacagaagccctgagctgctgctgggagccccttacggcaagtccgtggatatgtggagcgtgggctgtatcctgggagagctgtctgacggccagcctctgtttcctggcgagtccgagatcgaccagctgttcaccatccagaaggtgctgggccctctgcctagcgagcagatgaagctgttctacagcaaccccagattccacggcctgagattccccgccgtgaatcaccctcagagcctggaaagaagatacctgggcatcctgaacagcgtgctgctggacctgatgaagaatctgctgaaactggaccccgccgacagatacctgaccgagcagtgcctgaaccatcctacctttcaaacccagagactgctggatcggagccctagcagaagcgccaagagaaagccctaccacgtggaaagcagcaccctgagcaacagaaaccaggccggcaagtctacagccctgcagagccaccacagaagcaacagcaaggacatccagaacctgtccgtgggcctgcctagagccgacgaaggactgcctgccaacgagagcttcctgaacggaaatctggctggcgcctctctgagccctctgcacacaaagacataccaggccagcagccagcctggcagcacaagcaaggatctgaccaacaacaacatccctcatctgctgagccccaaagaggccaagagcaagaccgagttcgacttcaacatcgaccccaagcctagcgaaggccctggcaccaagtacctgaagtccaacagcagaagccagcagaaccggcactccttcatggaaagctcccagagcaaggccggcacactgcagcctaacgagaagcagagcagacacagctacatcgacacaatcccacagagcagcaggtccccaagctacaggaccaaggccaaatctcacggcgccctgagcgatagcaagagcgtgtccaatctgtccgaggctagagcccagatcgccgagcctagcaccagcagatacttccctagctcctgcctggatctgaacagcccaacaagccccacacctaccaggcacagcgacaccagaacactgctgtccccatccggcagaaacaaccggaacgagggcaccctggacagcagacggaccaccacaagacacagcaagaccatggaagaactgaagctgcccgagcacatggactccagccactctcattctctgagcgcccctcacgagtccttctcttacggcctgggctacacaagccccttcagctctcagcagaggccccacagacacagtatgtatgtgacccgggacaaagtgcgcgccaaaggcctggacggctctctgtctatcggccaaggcatggccgccagagccaattctctgcagctgctgtctcctcagcctggggaacaactgcctcctgagatgaccgtggccagatccagcgtgaaagaaaccagcagagagggcacctccagcttccacaccagacagaaaagcgaaggcggagtgtaccacgatcctcacagcgacgacggcacagcccctaaagagaacagacacctgtacaacgaccccgtgcctagaagagtgggctccttctacagagtgccttctcctagacctgacaactccttccacgagaacaacgtgtccaccagagtgtccagcctgccttccgaaagcagctccggcaccaaccactccaagagacagcctgctttcgacccctggaagtcccctgagaacatcagccactccgagcagctgaaagagaaagagaagcagggctttttccggtccatgaagaagaagaaaaagaagtcccagacggtccccaacagcgactccccagatctgctgaccctgcagaagtccatccactccgccagcacacctagcagcagacccaaagagtggcggccagagaagatcagcgatctgcagacccagtctcagcctctgaagtccctgaggaagctgctgcatctgagcagcgcctctaatcaccctgccagcagcgaccctagatttcagccactgacagcccagcagaccaagaactccttcagcgagatccggattcacccactgtctcaggccagcggcggcagcagcaatatcagacaagagcccgctcctaagggcagacctgctctgcaactgcctggccagatggatccaggctggcacgtgtcctctgtgaccagatccgccacagagggacctagctacagcgaacagctcggcgccaagtctggccctaacggacacccctacaaccggaccaaccggtccagaatgcccaatctgaacgacctcaaagaaacggccctgtga

SEQ ID NO: 46 - DNA, 2883 bp, synthetic construct
  misc_feature 1..2883: note = engineered nucleic acid sequence CDKL5coB2
  source 1..2883: mol_type = other DNA
atgaagatcccaaatatcggcaacgtgatgaataagttcgagatcctgggagtggtgggagagggagcatatggcgtggtgctgaagtgcaggcacaaggagacccacgagatcgtggccatcaagaagtttaaggacagcgaggagaatgaggaggtgaaggagaccacactgagagagctgaagatgctgaggacactgaagcaggagaacatcgtggagctgaaggaggccttccggagaaggggcaagctgtacctggtgtttgagtacgtggagaagaacatgctggagctgctggaggagatgccaaatggcgtgccccctgagaaggtgaagtcctacatctatcagctgatcaaggccatccactggtgccacaagaacgacatcgtgcacagagatatcaagcccgagaacctgctgatctcccacaatgacgtgctgaagctgtgcgatttcggctttgccaggaacctgagcgagggcaacaacgccaattacacagagtacgtggccaccaggtggtacaggagcccagagctgctgctgggagcaccatacggcaagagcgtggatatgtggtccgtgggctgcatcctgggcgagctgtctgacggccagcctctgttcccaggcgagagcgagatcgatcagctgtttaccatccagaaggtgctgggccctctgccaagcgagcagatgaagctgttctactccaaccctagattccacggcctgaggtttcccgccgtgaatcaccctcagagcctggagcgccggtatctgggcatcctgaactccgtgctgctggatctgatgaagaatctgctgaagctggaccccgccgatagatacctgaccgagcagtgtctgaatcaccctacatttcagacccagcgcctgctggaccggagcccttccagatctgccaagaggaagccatatcacgtggagagctccaccctgtccaatagaaaccaggccggcaagtctacagccctgcagagccaccacaggagcaactccaaggacatccagaatctgtccgtgggactgcctagggcagacgagggactgccagccaacgagagcttcctgaacggaaacctggcaggagcatctctgagcccactgcacacaaagacctaccaggcctctagccagcccggctccacatctaaggacctgaccaacaataacatcccacacctgctgtctcccaaggaggccaagagcaagaccgagttcgactttaatatcgatccaaagcctagcgagggaccaggcacaaagtatctgaagagcaactcccgctctcagcagaatcggcactccttcatggagtcctctcagtctaaggccggcaccctgcagcctaacgagaagcagagcagacactcctacatcgataccatcccccagagctccagaagcccttcctatagaacaaaggccaagtctcacggcgccctgtctgacagcaagtccgtgtctaatctgagcgaggcaagggcacagatcgcagagcccagcacctcccggtactttccttcttcctgtctggacctgaactctcctacaagcccaacacccacccgccacagcgatacacggaccctgctgtctccaagcggccgcaataaccggaacgagggcaccctggattctagaaggaccacaacccgccacagcaagacaatggaggagctgaagctgccagagcacatggactcctctcactcccactctctgagcgcccctcacgagtccttctcttacggcctgggctatacctcccccttctcctcccaacaaagaccccacaggcacagcatgtacgtgacaagggacaaggtccgggcaaagggactggacggaagcctgtccatcggacagggaatggcagcaagggccaactccctgcagctgctgtctcctcagccaggagagcagctgccaccagagatgaccgtggcacgctctagcgtgaaggagacaagccgggagggcacctcctctttccacacacgccagaagtccgagggaggcgtgtaccacgacccacactctgacgacggcacagcccctaaggagaaccggcacctgtacaacgatcccgtgcctaggagggtgggatccttctatagagtgccatctcccaggcctgacaatagctttcacgagaataacgtgtccacccgcgtgagctccctgccatctgagtctagctccggcacaaaccactctaagcggcagcccgcctttgacccttggaagagcccagagaatatctctcacagcgagcagctgaaggagaaggagaagcagggcttctttcgcagcatgaagaagaagaagaagaagagccagaccgtgcctaactccgactctccagacctgctgaccctgcagaagtccatccacagcgcctccacaccatctagcaggcctaaggagtggcggcctgagaagatcagcgatctgcagacccagagccagccactgaagtccctgcggaagctgctgcacctgtcctctgcctccaaccacccagcaagctccgacccaagattccagcccctgacagcccagcagaccaagaacagcttcagcgagatcaggatccaccctctgtcccaagcatctggaggatctagcaacatcagacaggagccagcaccaaagggcaggcccgccctgcagctgcctggccagatggatccaggctggcacgtgtcctctgtgacacgcagcgccaccgagggaccatcctactctgagcagctgggagcaaagagcggacctaacggacacccatataaccggaccaatagatccaggatgcccaatctgaacgacctgaaggagacagccctgtga

SEQ ID NO: 47 - DNA, 2883 bp, synthetic construct
  misc_feature 1..2883: note = engineered nucleic acid sequence CDKL5coC2
  source 1..2883: mol_type = other DNA
atgaagatccccaacatcggcaacgtgatgaacaagttcgagatcctgggcgtggtgggcgagggcgcctacggcgtggtgctgaagtgcaggcacaaggagacccacgagatcgtggccatcaagaagttcaaggacagcgaggagaacgaggaggtgaaggagaccaccctgagggagctgaagatgctgaggaccctgaagcaggagaacatcgtggagctgaaggaggccttcaggaggaggggcaagctgtacctggtgttcgagtacgtggagaagaacatgctggagctgctggaggagatgcccaacggcgtgccccctgagaaggtgaagagctacatctaccagctgatcaaggccatccactggtgccacaagaacgacatcgtgcacagggacatcaagcccgagaacctgctgatcagccacaacgacgtgctgaagctgtgcgacttcggcttcgccaggaacctgagcgagggcaacaacgccaactacaccgagtacgtggccaccaggtggtacaggagccccgagctgctgctgggcgccccctacggcaagagcgtggacatgtggagcgtgggctgcatcctgggcgagctgagcgacggccagcccctgttccccggcgagagcgagatcgaccagctgttcaccatccagaaggtgctgggccccctgcccagcgagcagatgaagctgttctacagcaaccccaggttccacggcctgaggttccccgccgtgaaccacccccagagcctggagaggagatacctgggcatcctgaacagcgtgctgctggacctgatgaagaacctgctgaagctggaccccgccgacagatacctgaccgagcagtgcctgaaccaccccaccttccagacccagaggctgctggacaggagccccagcaggagcgccaagaggaagccctaccacgtggagagcagcaccctgagcaacaggaaccaggccggcaagagcaccgccctgcagagccaccacaggagcaacagcaaggacatccagaacctgagcgtgggcctgcccagggccgacgagggcctgcccgccaacgagagcttcctgaacggcaacctggccggcgccagcctgagccccctgcacaccaagacctaccaggccagcagccagcccggcagcaccagcaaggacctgaccaacaacaacatcccccacctgctgagccccaaggaggccaagagcaagaccgagttcgacttcaacatcgaccccaagcccagcgagggccccggcaccaagtacctgaagagcaacagcaggagccagcagaacaggcacagcttcatggagagcagccagagcaaggccggcaccctgcagcccaacgagaagcagagcaggcacagctacatcgacaccatcccccagagcagcaggagccccagctacaggaccaaggccaagagccacggcgccctgagcgacagcaagagcgtgagcaacctgagcgaggccagggcccagatcgccgagcccagcaccagcagatacttccccagcagctgcctggacctgaacagccccaccagccccacccccaccaggcacagcgacaccaggaccctgctgagccccagcggcaggaacaacaggaacgagggcaccctggacagcaggaggaccaccaccaggcacagcaagaccatggaggagctgaagctgcccgagcacatggacagcagccacagccacagcctgagcgcccctcacgagagcttcagctacggcctgggctacaccagccccttcagcagccagcagaggccccacaggcacagcatgtacgtgaccagggacaaagtgagggccaagggcctggacggcagcctgagcatcggccagggcatggccgccagggccaacagcctgcagctgctgagcccccagcccggcgagcagctgcctcccgagatgaccgtggccaggagcagcgtgaaggagaccagcagggagggcaccagcagcttccacaccaggcagaagagcgagggcggcgtgtaccacgacccccacagcgacgacggcaccgcccccaaggagaacaggcacctgtacaacgaccccgtgcccaggagggtgggcagcttctacagggtgcccagccccaggcccgacaacagcttccacgagaacaatgtgagcaccagagtgagcagcctgcccagcgagagcagcagcggcaccaaccacagcaagaggcagcccgccttcgacccctggaagagccccgagaacatcagccacagcgagcagctgaaggagaaggagaagcagggcttcttcaggagcatgaagaagaagaagaagaagagccagaccgtgcccaacagcgacagccccgacctgctgaccctgcagaagagcatccacagcgccagcacccccagcagcaggcccaaggagtggaggcccgagaagatcagcgacctgcagacccagagccagcccctgaagagcctgaggaagctgctgcacctgagcagcgccagcaaccaccccgccagcagcgaccccaggttccagcccctgaccgcccagcagaccaagaacagcttcagcgagatcaggatccaccccctgagccaggccagcggcggcagcagcaacatcaggcaggagcccgcccccaagggcaggcccgccctgcagctgcccggccagatggaccccggctggcacgtgagcagcgtgaccaggagcgccaccgagggccccagctacagcgagcagctgggcgccaagagcggccccaacggccacccctacaacaggaccaacaggagcaggatgcccaacctgaacgacctgaaggagaccgccctgtga

SEQ ID NO: 48 - DNA, 2883 bp, synthetic construct
  misc_feature 1..2883: note = engineered nucleic acid sequence CDKL5coD2
  source 1..2883: mol_type = other DNA
atgaagatccctaacatcgggaatgtcatgaacaagtttgaaatcctgggggtggtgggggagggagcttatggagttgttttgaaatgtcgtcataaggaaactcatgaaatcgtggccattaagaagttcaaggacagtgaggagaatgaagaggtgaaggagaccactctccgcgagctgaagatgctgaggacactgaagcaggaaaacattgtggagctgaaggaggccttccgacgacggggtaagttgtacctggtgtttgagtacgtggagaaaaacatgttagaacttctggaggaaatgccaaacggtgtacctccagaaaaagtcaagtcatatatctaccagttgatcaaagccatccactggtgtcataaaaatgacatcgtccacagagatataaaaccagagaatcttctgatctctcacaatgatgtattaaaactttgtgattttggttttgcccgtaatctgtctgaaggtaataatgctaactatactgagtatgtagcaactcgctggtacagatcgcctgaactgctactgggtgctccttacggaaaaagtgtggacatgtggagcgttggttgtatcctaggagagctgagtgatgggcagccattgttcccgggggagagtgaaattgaccaactattcacaatccagaaagtgctggggccactaccaagtgaacaaatgaaacttttttattcaaatcctagatttcacggtctacgtttccccgctgtgaaccatcctcagtccttagaacggcgatatttaggcattctgaactctgttctgttagatctgatgaagaatttactaaaattagacccggctgaccgctatttgacagagcagtgtttaaatcatccaaccttccagacacagagactcctagaccgttcaccatctagaagtgcgaaaagaaaaccctaccacgttgaatcaagtaccctctccaatcgcaaccaggctgggaagagtactgccctacaatcgcaccacaggagtaatagtaaagatattcagaatctatctgtagggcttcctcgagcggacgagggtctacctgccaatgaaagtttcctcaatggtaacttagctggggcatcactgtcgccccttcatactaagacctaccaagcctcatctcaaccggggagtactagtaaagacctcaccaacaataacatcccccatttattatctcccaaggaggcgaaatccaagactgagttcgatttcaacattgacccaaagccctctgaagggcctggcaccaaatacctgaaatctaatagcagaagtcaacaaaataggcacagcttcatggagtcctcgcagagcaaagctggcacactgcaacctaatgaaaagcaaagccgacattcatatattgataccatcccccaatctagtagaagcccttcataccgcacgaaagcaaagtctcatggcgcactgtcagattctaaatccgtcagcaatctcagtgaagccagggctcagatcgccgagccctctacatctcgctactttccctcatcctgcctggacctaaactccccgacctctcccacccccacgcgtcacagcgacacccgaacattactttcaccttcgggcaggaataatcgaaatgagggcaccctagattcgcgccgtacgactacacggcattcaaaaaccatggaagaattgaaattgcctgagcacatggattcgtcacactctcacagtctatcggcccctcacgagagtttcagttacgggcttggctacaccagtcccttctccagccagcaacgcccacaccgccactccatgtatgtcacccgcgacaaggtccgtgccaaaggactcgatggcagcctaagcataggacagggcatggccgccagggccaacagtctccagttactaagtccccagcccggagaacagctgccacccgagatgactgtggctagatcgtccgtgaaagagacaagtagggaagggacctctagcttccacaccagacagaaatccgaggggggcgtgtatcatgaccctcatagtgatgatggtacagccccgaaggaaaatagacacttgtacaatgaccctgtgccccgaagagttggctccttctaccgggtgccctctccacggccagacaacagcttccatgagaacaatgtttcaacccgggtaagctccctgcctagtgaaagttcatccggaacaaatcactcgaagcgtcagccagccttcgacccctggaagtctccggagaacataagtcacagcgagcagctcaaagaaaaagaaaaacaaggcttcttcagaagcatgaagaagaaaaaaaaaaagtctcaaacagtgccaaattccgattcccccgacttattaacactacaaaagagcatccattcagcttctacgccaagcagtaggcccaaagagtggagacccgagaagatttctgatctacagacccagtctcagcctttaaaatcgctcaggaagctgctgcacctgtcttccgcttcaaaccacccggcctccagtgaccctcggtttcagccactgactgcccagcagactaaaaatagtttttcagagatcaggattcatcccttatcacaggcctctggcggcagcagcaatatccggcaggagcctgcccctaaaggccggcccgcgttgcagctgcccggccagatggacccaggctggcatgtgtcatcagttacacgcagtgccacagaaggccccagctactcagaacaattgggtgctaagtcaggacccaatggccacccatacaaccgaaccaacagatcaaggatgcccaacctcaacgacctcaaagaaacagctctctga

SEQ ID NO: 49 - DNA, 4827 bp, synthetic construct
  source 1..4827: mol_type = other DNA
  repeat_region 1..130: note = 5' ITR
  regulatory 220..1448: note = UbC promoter; regulatory_class = promoter
  misc_feature 1458..1466: note = Kozak
  misc_feature 1464..4346: note = nucleic acid sequence encoding hCDKL5
  misc_feature 4364..4385: note = miRNA183
  misc_feature 4390..4411: note = miRNA183
  misc_feature 4418..4439: note = miRNA183
  misc_feature 4446..4467: note = miRNA183
  regulatory 4483..4609: note = rabbit beta globin polyA; regulatory_class = polyA_signal_sequence
  repeat_region 4698..4827: note = 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagcatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccggtagtgaattctaccagtgccataggatagtgaattctaccagtgccatacacgtgagtgaattctaccagtgccatagcatgcagtgaattctaccagtgccatagcggcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 50 - DNA, 4390 bp, synthetic construct
  source 1..4390: mol_type = other DNA
  regulatory 1..1229: note = UbC promoter; regulatory_class = promoter
  misc_feature 1239..1247: note = Kozak
  misc_feature 1245..4127: note = nucleic acid sequence encoding hCDKL5
  misc_feature 4145..4166: note = miRNA183
  misc_feature 4171..4192: note = miRNA183
  misc_feature 4199..4220: note = miRNA183
  misc_feature 4227..4248: note = miRNA183
  regulatory 4264..4390: note = rabbit beta globin polyA; regulatory_class = polyA_signal_sequence
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccggtagtgaattctaccagtgccataggatagtgaattctaccagtgccatacacgtgagtgaattctaccagtgccatagcatgcagtgaattctaccagtgccatagcggcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 51 - DNA, 130 bp, synthetic construct
  source 1..130: mol_type = other DNA; note = 5' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 52 - DNA, 1229 bp, synthetic construct
  source 1..1229: mol_type = other DNA; note = UbC promoter
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggta

SEQ ID NO: 53 - DNA, 127 bp, synthetic construct
  source 1..127: mol_type = other DNA; note = rabbit beta globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 54 - DNA, 130 bp, synthetic construct
  source 1..130: mol_type = other DNA; note = 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 55 - DNA, 4692 bp, synthetic construct
  source 1..4692: mol_type = other DNA
  regulatory 1..36: note = truncated p5 promoter; regulatory_class = promoter
  misc_feature 37..1899: note = AAV2 Rep
  misc_feature 1919..4129: note = AAVhu68 cap
  regulatory 4220..4350: note = P5 promoter; regulatory_class = promoter
  regulatory 4398..4692: note = LacZ promoter; regulatory_class = promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 56 - DNA, 1863 bp, adeno-associated virus 2
  source 1..1863: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 57 - DNA, 2211 bp, adeno-associated virus hu68
  source 1..2211: mol_type = other DNA
  misc_feature 1..2211: note = VP1
  misc_feature 412..2211: note = VP2
  misc_feature 607..2211: note = VP3
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 58 - DNA, 4809 bp, synthetic construct
  source 1..4809: mol_type = other DNA
  repeat_region 1..130: note = 5'ITR
  regulatory 226..1454: note = UbC promoter; regulatory_class = promoter
  misc_feature 1470..4352: note = nucleic acid sequence encoding hCDKL5
  regulatory 4376..4614: note = SV4- polyA; regulatory_class = polyA_signal_sequence
  repeat_region 4680..4809: note = 3' ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccgtcgatcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 59 - DNA, 4383 bp, synthetic construct
  source 1..4383: mol_type = other DNA
  regulatory 1..1229: note = UbC Promoter; regulatory_class = promoter
  misc_feature 1245..4127: note = nucliec acid sequence encoding hCDKL5
  misc_feature 4145..4166: note = miRNA183
  misc_feature 4171..4192: note = miRNA183
  misc_feature 4199..4220: note = miRNA183
  misc_feature 4227..4248: note = miRNA183
  regulatory 4257..4383: note = Rabbit beta globin polyA; regulatory_class = polyA_signal_sequence
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccggtagtgaattctaccagtgccataggatagtgaattctaccagtgccatacacgtgagtgaattctaccagtgccatagcatgcagtgaattctaccagtgccatagcggccgcgatctttttccctctgccaaaaattatggggacatcatgaagccccttgagcatctgacttctggctaataaaggaaatttattttcattgcaatagtgtgttggaattttttgtgtctctcactcg

SEQ ID NO: 60 - DNA, 3968 bp, synthetic construct
  source 1..3968: mol_type = other DNA
  regulatory 1..798: note = CBh promoter; regulatory_class = promoter
  misc_feature 830..3712: note = nucleic acid sequence encoding hCDKL5
  misc_feature 3730..3751: note = miRNA183
  misc_feature 3756..3777: note = miRNA183
  misc_feature 3784..3805: note = miRNA183
  misc_feature 3812..3833: note = miRNA183
  regulatory 3842..3968: note = rabbit beta globin polyA; regulatory_class = polyA_signal_sequence
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcttaagctgcagaccggaattcgccgccaccatgaagatccccaacatcggcaacgtgatgaacaagttcgagatcctgggcgttgtcggagaaggcgcttatggcgtggtgctgaagtgcagacacaaagaaacccacgagatcgtggccatcaagaagttcaaggacagcgaagagaacgaggaagtgaaagagacaaccctgcgcgagctgaagatgctgagaaccctgaagcaagagaacatcgtggaactgaaagaggccttccggcggagaggcaagctgtacctggtgttcgagtacgtggaaaagaacatgctcgagctgctggaagagatgcccaatggcgtgccacctgagaaagtgaagtcctacatctaccagctgatcaaggccatccactggtgccacaagaacgacatcgtgcacagagacatcaagcccgagaacctgctgatctcccacaacgacgtgctgaagctgtgcgacttcggcttcgccagaaatctgagcgagggcaacaacgccaactacaccgagtatgtggccaccagatggtacagaagccctgaactgctgctgggagccccttacggcaagtctgtggatatgtggtccgtgggctgtatcctgggagagctgtctgatggccaacctctgtttcctggcgagtccgagatcgaccagctgttcacaatccagaaggtgctgggccctctgcctagcgagcagatgaagctgttctacagcaaccccagattccacggcctgagattccctgccgtgaatcaccctcagagcctggaaagaagatacctgggcatcctgaacagcgtgctgctggacctgatgaagaatctgctgaaactggaccccgccgacagatacctgaccgagcagtgtctgaaccatcctacctttcagacccagagactgctggatagaagccccagcagaagcgccaagagaaagccctaccacgtggaaagcagcaccctgagcaatagaaaccaggccggaaagagcactgccctgcagagccaccacagaagcaacagcaaggacatccagaacctgagcgtgggcctgcctagagctgatgaaggactgcctgccaacgagagcttcctgaatggaaatctggctggcgcctctctgagccctctgcacacaaagacataccaggccagcagccagcctggcagcacaagcaaggacctgaccaacaacaacatccctcatctgctgagccccaaagaggccaagagcaagaccgagttcgacttcaacatcgaccccaagcctagcgaaggccctggcaccaagtacctgaagtccaactccagaagccagcagaaccggcactccttcatggaaagctctcagagcaaggccggcacactgcagcctaacgagaagcagagcagacacagctacatcgacacaatccctcagtcctctcggagccccagctacagaacaaaggccaaatctcacggcgccctgagcgatagcaagagcgtgtccaatctgtccgaggctagagcccagatcgccgagcctagcaccagcagatacttccctagcagctgcctggatctgaacagccctacaagccccacacctaccaggcacagcgataccagaacactgctgtccccaagcggccggaacaacagaaatgagggcaccctggacagcagacggaccaccacaagacacagcaagaccatggaagaactgaagctgcccgagcacatggactctagccactctcattctctgagcgcccctcacgagtcctttagctacggcctgggctacacaagcccattttccagtcagcagcggccccacagacactctatgtacgtgaccagagacaaagtccgcgccaaaggcctggatggctctctgtctattggacaaggcatggccgccagagccaatagcctgcaacttctgtctcctcagcctggcgaacagctgcctcctgaaatgacagtggcccggtccagcgtgaaagaaaccagcagagagggcacctccagcttccacaccagacagaaaagcgaaggcggcgtgtaccacgatcctcactctgatgatggcacagcccctaaagagaacagacacctgtacaacgaccccgtgcctagaagagtgggctccttctacagagtgccttctcctagacctgacaactccttccacgagaacaacgtgtcaacccgggtgtccagcctgccttctgagagcagctctggcacaaaccactccaagaggcagcccgccttcgatccttggaagtcccctgagaatatcagccactccgagcagctgaaagagaaagagaagcagggctttttccggtccatgaagaagaagaaaaagaagtcccagacagtccccaacagcgacagccctgatctcctgacactgcagaagtccatccacagcgccagcacacctagcagcagacctaaagagtggcggccagagaagatcagcgacctgcaaacacagagccagcctctgaagtccctgaggaagctgctgcatctgtcctccgcctctaatcaccctgccagcagcgatcccagatttcagccactgacagcccagcagaccaagaactccttcagcgagatccggattcacccactgtctcaggccagcggcggcagctctaatatcagacaagagcccgctcctaagggcagacctgctctccaactgcctggacagatggatcctggctggcacgtgtcctctgtgacaagatctgccaccgagggacctagctattctgagcagctgggcgccaagtctggccccaatggacacccctacaaccggaccaatcggagcagaatgcccaacctgaacgacctcaaagaaacagccctctgaagcttatcgataccggtagtgaattctaccagtgccataggatagtgaattctaccagtgccatacacgtgagtgaattctaccagtgccatagcatgcagtgaattctaccagtgccatagcggccgcgatctttttccctctgccaaaaattatggggacatcatgaagccccttgagcatctgacttctggctaataaaggaaatttattttcattgcaatagtgtgttggaattttttgtgtctctcactcg

SEQ ID NO: 61 - protein, 736 aa, adeno-associated virus hu68
  CHAIN 1..736: note = VP1
  CHAIN 138..736: note = VP2
  CHAIN 203..736: note = VP3
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2024/0425882 A1
(43) Pub. Date: Dec. 26, 2024

(54) COMPOSITIONS USEFUL IN TREATMENT OF CDKL5 DEFICIENCY DISORDER (CDD)

(71) Applicant: The Trustees of the University of Pennsylvania, Philadelphia, PA (US)

(72) Inventors: James M. Wilson, Philadelphia, PA (US); Ralf Schmid, Havertown, PA (US)

(73) Assignee: The Trustees of the University of Pennsylvania, Philadelphia, PA (US)

(21) Appl. No.: 18/702,258

(22) PCT Filed: Oct. 18, 2022
(86) PCT No.: PCT/US22/78327

§ 371 Date: Apr. 17, 2024

Related U.S. Application Data
(60) Provisional application No. 63/256,827, filed on Oct. 18, 2021.

(51) Int. Cl. C12N 15/864; A61K 48/00; A61P 25/28; C12N 9/12; C12N 15/52

(52) U.S. Cl. CPC C12N 15/8645; A61K 48/0066; A61K 48/0075; A61P 25/28; C12N 9/12; C12N 15/52; C12N 2750/14143; C12N 2800/60

(57) ABSTRACT

Provided is a recombinant adeno-associated virus (rAAV) having an AAV capsid and a vector genome which comprises a nucleic acid sequence encoding a functional CDKL5 (hCDKLK5). Also provided are a production system useful for producing the rAAV, a pharmaceutical composition comprising the rAAV, and a method of treating a subject having CDD, or ameliorating symptoms of CDD, or delaying progression of CDD via administrating an effective amount of the rAAV to a subject in need thereof.

### BACKGROUND OF THE INVENTION

CDKL5 Deficiency Disorder (CDD) is a serious neurodevelopmental disorder affecting young children. The underlying cause is lack of CDKL5 protein expression due to mutations in the X-linked Cyclin-Dependent Kinase-Like 5 gene, CDKL5 (Mendelian Inheritance in Man, MIM: 300203; previously known as STK9), resulting in a range of phenotypes, including EIEE2 (MIM: 300672), a form of early infantile epileptic encephalopathy [Bahi-Buisson, N. et al. Key clinical features to identify girls with CDKL5 mutations. Brain 131, 2647-2661, doi: 10.1093/brain/awn197 (2008)], and infantile spasms [Fehr, S. et al. Eur J Hum Genet 21, 266-273, doi:10.1038/ejhg.2012.156 (2013); Kalscheuer, V. M. et al., American Journal of Human Genetics, 72, 1401-1411, doi:10.1086/375538 (2003); Tao, J. et al. American Journal of Human Genetics 75, 1149-1154, doi:10.1086/426460 (2004). Weaving, L. S. et al. American Journal of Human Genetics 75, 1079-1093, doi:10.1086/426462 (2004)]. In addition to the characteristic early-onset seizures, the phenotype may also include a number of other features, such as stereotypic hand movements, severe psychomotor retardation and general hypotonia. The early postnatal onset of symptoms indicates that CDKL5 plays a crucial role in brain development. CDKL5 is also expressed within the mature adult nervous system. CDKL5 is expressed throughout the cell, including the nucleus and the cytoplasm of the cell soma and dendrites.

CDKL5 gene mutations are the cause of most cases of CDD, a progressive neurologic developmental disorder and one of the most common causes of cognitive disability in females. Males who have the genetic mutation that causes CDD are affected in devastating ways. Most of them die before birth or in early infancy. See, e.g., ninds.nih.gov/Disorders/Patient-Caregiver-Education/Fact-Sheets/Rett-Syndrome-Fact-Sheet and omim.org/entry/312750.

Currently, there is no cure for CDD, and treatment focuses on alleviating disease symptoms. In many cases seizures are poorly controlled, thus there is an urgent medical need to find novel therapeutic approaches.

### SUMMARY OF THE INVENTION

Provided herein is a recombinant adeno-associated virus (rAAV) which is useful for treating CDKL5 Deficiency Disorder (CDD) in a subject in need thereof. The rAAV carries a vector genome comprising inverted terminal repeats (ITR) and a novel nucleic acid sequence encoding a functional human CDKL5 protein under the control of regulatory sequences which direct the hCDKL5 expression in a target cell.

In certain embodiments, a recombinant adeno-associated virus (rAAV) useful for treating CDD is provided. The rAAV comprises:(a) an AAVhu68 or AAVrh91 capsid; and (b) a vector genome in the AAV capsid of (a), wherein the vector genome comprises a 5′ AAV inverted terminal repeat (ITR), an expression cassette comprising a human CDKL5 sequence of nucleotides 1 to 2883 of SEQ ID NO: 22 operably linked to regulatory sequences which direct expression thereof and which further comprise four tandem miR183 targeting sequences, and a 3′ AAV ITR. In certain embodiments, the regulatory sequences further comprise a UbC promoter or a hSyn promoter. In certain embodiments, the UbC promoter has the sequence of SEQ ID NO: 52. In certain embodiments, the expression cassette comprises nucleic acid sequence of nucleotides 220 to 4609 of SEQ ID NO: 49 (or SEQ ID NO: 50), nucleic acid sequence of nucleotides 226 to 4608 of SEQ ID NO: 29 (or SEQ ID NO: 59) or the nucleic acid sequence of nt 224 to 4191 of SEQ ID NO: 31 (or SEQ ID NO: 60). In certain embodiments, the AAV capsid is an AAVhu68 capsid. In certain embodiments, the vector genome comprises an AAV 5′ ITR, a UbC promoter, a Kozak sequence, the hCDKL5 coding sequence, four miR183 targeting sequences in the 3′ UTR of the hCDKL5 coding sequence, a rabbit globin polyA signal, and an AAV 3′ ITR. In certain embodiments, at least one the miR183 targeting sequences has the sequence of AGTGAATTCTACCAGTGCCATA (miR183, SEQ ID NO: 11). In certain embodiments, two, three or four of the miR183 targeting sequences has SEQ ID NO: 11. In certain embodiments, the four miR183 targeting sequences are located in tandem in the 3′ UTR and are separated by spacer sequences.

In certain embodiment, a composition comprising a stock of rAAV vector as described herein and an aqueous suspension media is provided.

In certain embodiments, a method of treating CDD is provided which comprises administrating an effective amount of the rAAV described herein to a subject in need thereof.

In certain embodiments, an rAAV production system useful for producing a vector as described herein is provided.

In a further aspect, provided herein is a composition comprising a rAAV or a vector as described herein and an aqueous suspension media.

In another aspect, a method of treating a subject having CDD, or ameliorating symptoms of CDD, or delaying progression of CDD is provided. The method comprises administrating an effective amount of a rAAV or a vector as described herein to a subject in need thereof. In certain embodiments, the vector or rAAV is administrable to a patient via an intra-cisterna magna injection (ICM).

These and other aspects of the invention are apparent from the following detailed description of the invention.

### BRIEF DESCRIPTION OF THE DRAWINGS

FIG. 1A shows an AAV vector design for an AAV CDKL5 vector genome comprising a 5′ AAV inverted terminal repeat (ITR), an expression cassette comprising: the human synapsin neuronal promoter, an engineered human CDKL5 DNA coding sequence, an Woodchuck Hepatitis Virus Posttranscriptional Regulatory Element (WPRE), and poly A, and an AAV 3′ ITR. FIG. 1B shows an AAV vector design for an AAV CDKL5 vector genome comprising a 5′AAV ITR, and expression cassette comprising: human ubiquitin C (UbC) promoter, an engineered human CDKL5 DNA coding sequence, a drg-miRNA to reduce drg expression, polyA, and an AAV 3′ITR. FIG. 1C shows an AAV vector design for an AAV CDKL5 vector genome comprising a 5′AAV ITR, and expression cassette comprising: a chicken beta-actin hybrid promoter (CBh), an engineered human CDKL5 DNA coding sequence, a miRNA sequence, polyA, and an AAV 3′ITR.

FIGS. 2A and 2B show analysis of mouse hippocampus, as assessed with anti-CDKL5 antibodies (S957D, University of Dundee, UK). Mice were treated with 5×10¹⁰ GC AAV-hSyn-CDKL5-1co.WPRE via neonatal intracerebroventricular injection. FIG. 2A shows CDKL5 expression by Western blotting, charting CDKL5/tubulin levels in wild-type mice with a PBS injection, KO mice with PBS injection, and treated KO mouse. FIG. 2B shows CDKL5 activity as determined using the pS222EB2 (Baltussen et al, 2018 Chemical genetic identification of CDKL5 substrates reveals its role in neuronal microtubule dynamics, EMBO J, 37:e99763) levels in wild-type mice with a PBS injection, KO mice with PBS injection, and treated KO mice.

FIG. 3 provides a graph of mouse maturation and survival following injection of AAV-hSyn-hCDKL5-1co.WPRE in a CDD mouse model. All injected mice survived treatment, gained weight. They mice did not display any overt signs of adverse outcomes.

FIG. 4 provides results from a behavioral assessment in a CDD mice receiving AAV-hSyn-CDKL5-1co.WPRE. This figure charts results from the Elevated Zero Maze, which assesses the balance between risk taking, curiosity and anxiety. The first bar represents wt mice receiving PBS. Wt mice are curious but cautious and spend a limited time out in the open. The middle bar shows Cdkl5-ko mice receiving PBS only, which showed decreased anxiety and spend more time out in the open, they entered the open zone more frequently. After AAV-CDKL5 treatment, behavior of Cdkl5-ko mice reverts to wt behavior (for time in open zone and for entries from closed into open zone).

FIGS. 5A and 5B provide results from a behavioral assessment in a CDD mice receiving AAV-hSyn-CDKL5-1co.WPRE. FIG. 5A shows exploratory activity of mice in an Open Field Arena, plotted as beam breaks/bin versus time (min). The dotted line shows wt mice, which are curious but within 10 min have explored the arena and calm down. The line with long dashes shows that Cdkl5-ko spend a longer time exploring but eventually calm down. The line with dashes and dots show that after AAV-CDKL5 treatment, activity of Cdkl5-ko mice is reduced, and their overall activity levels resemble wt mice. FIG. 5B shows cumulative activity data of total beam breaks taken by mice, which confirms results in FIG. 5A.

FIG. 6 shows a graph of measured latency to fall (sec) in a motor activity and agility assessment (rotarod) in mice over three consecutive days. The wild-type mice are observed to increase performance while learning over time. The Cdkl5-ko mice show improved performance, compared to wt mice, likely due to the previously observed initial hyperactivity. After AAV-hSyn-CDKL5-1co.WPRE treatment, behavior of Cdkl5-ko mice is close to WT mice, and matches WT mice performance after 2 days of learning.

FIGS. 7A and 7B show results of hippocampal learning and memory (Y-maze). FIG. 7A shows the percentage of spontaneous alterations for the test group and the two control groups. FIG. 7B shows the distance moved (m) for the test group and the two control groups. The wt mice show a strong tendency to explore the maze arm that they have not visited recently (spontaneous alteration behavior), whereas Cdkl5-ko mice have a lower propensity for this memory-dependent behavior. After gene therapy, performance shows a trend for improvement.

FIGS. 8A to 8D illustrate CDKL5 expression levels or activity levels for AAV.CDLK5 vector constructs for the expression isoform 1, isoform 2, isoform 3 or isoform 4. FIG. 8A shows quantified expression levels of CDKL5 isoforms 1, 2, 3, and 4 in knock-out mice injected with AAV vectors (5×10¹⁰ GC, neonatal ICV), as compared to a wild-type mouse injected with vehicle and a knock-out mouse injected with vehicle. FIG. 8B shows CDKL5 activity as determined from quantified signal of pS222EB2 from western blot analysis of tissue from treated a wild-type mouse (injected with vehicle (PBS)), a knock-out mouse (injected with vehicle, or the AAV.CDKL5-1co). FIG. 8C shows CDKL5 activity as determined from quantified signal of pS222EB2 from western blot analysis of tissue from treated a wild-type mouse (injected with vehicle (PBS)), a knock-out mouse (injected with vehicle, or the AAV.CDKL5-isoform 1, 2, 3 or 4 (from FIG. 8A)). FIG. 8D shows quantified CDKL5 expression levels of isoform 1 in KO mice (from FIG. 8B).

FIGS. 9A to 9F show therapeutic efficacy of the AAV.CDKL5 gene therapy in a mouse study comparing different vector doses (5×10¹⁰ GC, 2.5×10¹⁰ GC, 1×10¹⁰ GC, and 6×10⁹ GC) in knock out and wild type mice. FIG. 9A show body weight gain (g) over a period of 10 weeks for mice treated with AAV.CDKL5 at a dose of 5×10¹⁰ GC or PBS. FIG. 9B show body weight gain (g) over a period of 10 weeks for mice treated with AAV.CDKL5 at a dose of 2.5×10¹⁰ GC or PBS. FIG. 9C shows a dose-dependent results of the hindlimb clasping test for AAV.CDKL5-treated group at a dose of 5×10¹⁰ GC compared to untreated Cdkl5-ko mice. FIG. 9D shows a dose-dependent results of the hindlimb clasping test for AAV.CDKL5-treated group at a dose of a dose of 2.5×10¹⁰ GC compared to untreated Cdkl5-ko mice. FIG. 9E shows a dose-dependent results of the hindlimb clasping test for AAV.CDKL5-treated group at a dose of 1×10¹⁰ GC compared to untreated Cdkl5-ko mice.

FIG. 9F shows a dose-dependent results of the hindlimb clasping test for AAV.CDKL5-treated group at a dose of 6×10⁹ GC compared to untreated Cdkl5-ko mice. WT mice showed no clasping, while KO mice showed pronounced clasping. After treatment, KO mice displayed a significantly reduced among of clasping. Injected WT mice were not affected.

FIGS. 10A to 10E show therapeutic efficacy of the AAV.CDL5 gene therapy in the CDD ko mouse study. FIG. 10A shows results in nest building (nest quality/score) for the AAV.CDKL5-treated group at a dose of 5×10¹⁰ GC, compared to untreated Cdkl5-ko mice. FIG. 10B shows results from a marble burying tasks with a trend to normalization in the AAV.CDLK5-treated group at a dose of 5×10¹⁰ GC, compared to WT and Cdkl5-ko mice. FIG. 10C shows results in nest building (nest quality/score) for the AAV.CDKL5-treated group at a dose of 2.5×10¹⁰ GC, compared to untreated Cdkl5-ko mice. FIG. 10D shows results from a marble burying tasks with a trend to normalization in the AAV.CDLK5-treated group at a dose of 2.5×10¹⁰ GC, compared to WT and Cdkl5-ko mice. FIG. 10E shows results in nest building (nest quality/score) for the AAV.CDKL5-treated group at a dose of 1×10¹⁰ GC compared to untreated Cdkl5-ko mice. FIG. 10F shows results of the nest building test in male Cdkl5^{KO/Y} mice and female Cdkl5^{KO/x} mice following ICV administration of an AAV vector expressing human CDKL5, plotted as percent weight of original nestlet weight intact.

FIGS. 11A to 11F shows correction of hyperactivity in the ko mice receiving the AAV.CDKL5 treatment as assessed in the open field activity test. FIG. 11A shows ambulatory activity/bin vs. time (5-minute intervals to 30 min) in AAV.CDKL5-treated ko mice at a dose of 5×10¹⁰ GC. FIG. 11B shows total activity in AAV.CDKL5-treated ko mice at a dose of 5×10¹⁰ GC. FIG. 11C shows ambulatory activity/bin vs. time (5-minute intervals to 30 min) in AAV.CDKL5-treated ko mice at a dose of 2.5×10¹⁰ GC. FIG. 11D shows total activity in AAV.CDKL5-treated ko mice at a dose of 2.5×10¹⁰ GC. FIG. 11E shows ambulatory activity/bin vs. time (5-minute intervals to 30 min) in AAV.CDKL5-treated ko mice at a dose of 6×10⁹ GC. FIG. 11F shows total activity in AAV.CDKL5-treated ko mice at a dose of 6×10⁹ GC. Normalization of increase risk-taking was observed in the elevated zero maze and normalization of hippocampal learning deficits are seen in the Y-maze in the AAV.CDKL5-treated ko mice.

FIG. 12 illustrates that the expression of CDKL5 isoforms 2-4 provide significant correction of the hindlimb clasping phenotype when assessed at dose of 5×10¹⁰ GC in ko mouse.

FIGS. 13A to 13D illustrate a strong trend for correction in KO mice treated with AAV.CDKL5-isoform 1. FIG. 13A show activity in elevated in KO mice treated with AAV.CDKL5-isoform 1 at a dose of 5×10¹⁰ GC. FIG. 13B show activity in elevated in KO mice treated with AAV.CDKL5-isoform 1 at a dose of 2.5×10¹⁰ GC. FIG. 13C show activity in Y-Maze in KO mice treated with AAV.CDKL5-isoform 1 at a dose of 5×10¹⁰ GC. FIG. 13D show activity in Y-Maze in KO mice treated with AAV.CDKL5-isoform 1 at a dose of 2.5×10¹⁰ GC.

FIGS. 14A to 14C show gender specific results in hindlimb clasping following treating of knock-out mice with the AAV.CDKL5-isoform 1. FIG. 14A shows hindlimb clasping following treating of male knock-out mice with the AAV.CDKL5-isoform 1. FIG. 14B shows hindlimb clasping following treating of female knock-out mice with the AAV.CDKL5-isoform 1. Both hemizygous males and heterozygous females displayed hindlimb clasping in Cdkl5-ko mice, which was significantly reduced after treatment. None of the WT groups showed clasping. FIG. 14C shows an ambulatory activity at high dose (5×10¹⁰ GC, neonatal ICV) with significant improvement in female heterozygous mice.

FIGS. 15A to 15F show results of the open field test in Male Cdkl5^{KO/Y} Mice and Female Cdkl5^{KO/X} Mice following ICV administration of an AAV vector expressing human CDKL5. FIG. 15A shows results of the horizontal activity open field test in males, plotted as X/Y-Axis beam breaks. FIG. 15B shows results of the horizontal activity open field test in females, plotted as X/Y-Axis beam breaks. FIG. 15C shows results of the rearing open field test in males, plotted as Z-axis beam breaks. FIG. 15D shows results of the rearing open field test in females, plotted as Z-axis beam breaks. FIG. 15E shows results of the center activity open field test in males, plotted as percent center beam breaks. FIG. 15F shows results of the center activity open field test in females, plotted as percent center beam breaks.

FIGS. 16A and 16B show gender differences in ko mice treated with the AAV.CDKL5-isoform 1 vector. FIG. 16A shows results in open field—ambulatory activity in an elevated zero maze assessment in males (KO) mouse treated with AAV.CDKL5-isoform 1, plotted as time spent (s) in an open zone. FIG. 16B shows results in open field-ambulatory activity in an in an elevated zero maze assessment in females (ht) mouse treated with AAV.CDKL5-isoform 1, plotted as time spent (s) in an open zone. Risk-prone behavior is corrected, with a size effect being more pronounced in males.

FIG. 17 provides vector distribution in various tissue samples from the NHP study (representative for the 1×10¹⁴ GC dose). The graph provides rAAV.CDKL5 in gc/diploid genome for various non-neuronal tissue, spinal track, and brain tissue. Strong transduction of dorsal root ganglia (DRG) is observed. Moderate to low transduction of brain tissues is observed, with some leakage into non-neuronal tissues.

FIG. 18 provides results quantified hCDKL5 expression (measured by RT-qPCR) in the NHP study shown in cerebellum, frontal cortex, occipital cortex, parietal cortex, and temporal cortex.

FIGS. 19A and 19B show results of a dose escalation studies measuring behavioral changes following CDKL5 gene therapy administration to WT mice. FIG. 19A shows no significant hindlimb clasp severity score changes in WT mice injected at 7.5×10¹⁰ GC and 1×10¹¹ GC of AAV, as compared with control mice treated with PBS. FIG. 19B shows no significant ambulatory activity changes in WT mice injected at 7.5×10¹⁰ GC and 1×10¹¹ GC of AAV, as compared with control mice treated with PBS.

FIG. 20 shows CDKL5 expression as measured qualitatively by Western blotting, at 14 days following administration of either AAVrh91.UbC.CDKL5-1co.miR183 or AAVrh91.CBh.CDKL5-1co.miR183 via neonatal ICV at a dose of 3×10¹⁰ GC.

FIG. 21A shows CDKL5 expression as measured qualitatively by Western blotting, at 4 months of age, wherein mice which were administered AAVrh91.UbC.CDKL5-1co.miR183 at doses 3×10¹⁰, 6×10¹⁰ GC via neonatal ICV.

FIG. 21B shows CDKL5 expression as measured qualitatively by Western blotting, at 4 months of age, wherein mice which were administered AAVrh91.CBh.CDKL5-1co.miR183 at doses 3×10¹⁰, 1×10¹⁰ GC via neonatal ICV.

FIG. 21C shows CDKL5 expression quantified from Western blotting analysis, charted as CDKL5/tubulin levels in wild type and knockout mice which were administered AAVrh91.UbC.CDKL5-1co.miR183 at doses 3×10¹⁰, 6×10¹⁰ GC via neonatal ICV, and compared to AAVhu68.hSyn-CDKL5 at a dose of 5×10¹⁰ GC.

FIG. 22A shows a representative image from immunofluorescent microscopy analysis of CDKL5 expression following administration with AAVrh91.UbC.CDKL5-1co.miR183 at a dose of 3×10¹⁰ GC via neonatal ICV.

FIG. 22B shows a representative image from immunofluorescent microscopy analysis of CDKL5 expression following administration with AAVrh91.CBh.CDKL5-1co.miR183 at a dose of 3×10¹⁰ GC via neonatal ICV.

FIG. 23A shows a representative image (zoomed in view) from immunofluorescent microscopy analysis of CDKL5 expression (samples were also probed for NeuN, neuronal marker) following administration with AAVrh91.UbC.CDKL5-1co.miR183 at a dose of 3×1010 GC via neonatal ICV.

FIG. 23B shows a representative image (zoomed in view) from immunofluorescent microscopy analysis of CDKL5 expression (samples were also probed for NeuN, neuronal marker) following administration with AAVrh91.CBh.CDKL5-1co.miR183 at a dose of 3×10¹⁰ GC via neonatal ICV.

FIG. 24 shows quantification of CDKL5-expressing neurons (above background levels; plotted at percent CDKL5 in positively identified neurons) as compared to previous results following administration with AAVhu68.hSyn.CDKL5.

FIGS. 25A to 25C show results of the survival study with tallied survival at post-natal day 16 (PND16) of mice which were administered with AAVrh91.CBh.CDKL5-1co.miR183 at doses of 1×10¹⁰, 3×10¹⁰, 6×10¹⁰ GC via neonatal ICV. FIG. 25A shows results of the survival study with tallied survival at post-natal day 16 (PND16) of mice which were administered with AAVrh91.CBh.CDKL5-1co.miR183 at doses of 1×10¹⁰ GC via neonatal ICV. FIG. 25B shows results of the survival study with tallied survival at post-natal day 16 (PND16) of mice which were administered with AAVrh91.CBh.CDKL5-1co.miR183 at doses of 3×10¹⁰ GC via neonatal ICV. FIG. 25C shows results of the survival study with tallied survival at post-natal day 16 (PND16) of mice which were administered with AAVrh91.CBh.CDKL5-1co.miR183 at doses of 6×10¹⁰ GC via neonatal ICV.

FIG. 26A shows severity score observed in DRG neurons from cervical, thoracic, and lumbar collected tissue from NHPs treated with AAVrh91.UbC.CDKL5-1co.miR183 or AAVrh91.CBh.CDKL5-1co.miR183 vectors at a dose of 3×10¹⁰ GC via ICM route. Score 0 signifies no signs of toxicity, whereas score 5 signifies severe toxicity, as scored by a board-certified veterinary pathologist. A score of 0.5 or lower is considered background, based on similar evaluations of naïve tissue.

FIG. 26B shows severity score observed in spinal cord neurons from cervical, thoracic, and lumbar collected tissue from NHPs treated with AAVrh91.UbC.CDKL5-1co.miR183 or AAVrh91.CBh.CDKL5-1co.miR183 vectors at a dose of 3×10¹⁰ GC via ICM route.

FIG. 26C shows severity score observed in sural nerve from proximal and distal collected tissue from NHPs treated with AAVrh91.UbC.CDKL5-1co.miR183 or AAVrh91.CBh.CDKL5-1co.miR183 vectors at a dose of 3×10¹⁰ GC via ICM route.

FIG. 27 shows results of the vector copy number plotted as GC/diploid genome in various tissues oh NHP following administration with AAVrh91.UbC.CDKL5-1co.miR183 or AAVrh91.CBh.CDKL5-1co.miR183 vectors.

FIG. 28 shows relative expression of CDKL5, plotted as per 100 ng of cDNA, in various CNS tissue (motor cortex, somatosensory (som. Sens). Cortex, parietal cortex, hippocampus, thalamus) of NHP, as compared to results observed in mouse brain.

FIG. 29A shows CDKL5 expression quantified from Western blotting analysis, charted as CDKL5/tubulin levels in knockout mice which were administered AAVrh91.UbC.CDKL5-1co.miR183 at doses 3×10¹⁰ GC, as compared to WT and knockout mice treated with PBS (control groups).

FIG. 29B shows kinase activity quantified from Western blotting analysis, charted as pEB2pS222/total EB2 levels in knockout mice which were administered AAVrh91.UbC.CDKL5-1co.miR183 at doses 3×10¹⁰ GC, as compared to WT and knockout mice treated with PBS (control groups)

FIG. 30 shows kinase activity as measured qualitatively by Western blotting (using pEB-S222 antibody; Baltussen at al., Chemical genetic identification of CDKL5 substrates reveals its role in neuronal microtubule dynamics, 2018, EMBO J, 37:e99763), in knockout mice which were administered AAVrh91.UbC.CDKL5-1co.miR183 at doses 3×10¹⁰ GC, as compared to WT and knockout mice treated with PBS (control groups).

FIG. 31A shows results of percent neurons with CDKL5 protein expression in mouse cortex and hippocampus tissue following neonatal ICV administration with AAVrh91.UbC.CDKL5-1co.miR183 at doses of 1×10¹⁰, 3×10¹⁰, 6×10¹⁰ GC, as compared to WT mice treated with PBS.

FIG. 31B shows a representative microscopy image from immunofluorescent analysis staining with DAPI (nuclei), CDKL5, and NeuN (neuronal marker) of cortical section tissue following neonatal ICV administration with AAVrh91.UbC.CDKL5-1co.miR183 at doses of 3×10¹⁰ GC.

FIG. 32 shows analysis of measured body weights of wild type and CDKL5-ko when administered with PBS, or AAV.UbC.CDKL5-1co.miR183 at doses of 1×10¹⁰, 3×10¹⁰, 6×10¹⁰ GC.

FIG. 33A shows a results of the hindlimb clasping test for AAV.UbC.CDKL5-1co.miR183-treated group at a dose of 3×10¹⁰ GC compared to untreated group in Cdkl5-ko mice and WT mice. Statistically significant improvement from ko is indicated (*p<0.05, **p<0.01).

FIG. 33B shows dose-dependent effect on hyperactivity, as measured in the open field activity test and plotted as ambulatory activity (beam breaks) in Cdkl5-ko mice and WT mice following administration of AAV.UbC.CDKL5-1co.miR183 at doses of 1×10¹⁰, 3×10¹⁰, 6×10¹⁰ GC. Statistically significant improvement from ko is indicated (*p<0.05, **p<0.01)

FIG. 34A shows results of binned ambulatory activity for groups of WT and Cdkl5-ko mice administered with AAV.UbC.CDKL5-1co.miR183 at a low dose of 1×10¹⁰ GC. Statistically significant improvement from ko is indicated (*p<0.05, **p<0.01, ***p<0.001, ****p<0.0001).

FIG. 34B shows results of binned ambulatory activity for groups of WT and Cdkl5-ko mice administered with AAV.UbC.CDKL5-1co.miR183 at a medium dose of 3×10¹⁰ GC.

FIG. 34C shows results of binned ambulatory activity for groups of WT and Cdkl5-ko mice administered with AAV.UbC.CDKL5-1co.miR183 at a high dose of 6×10¹⁰ GC.

FIG. 35 shows results in nest building (nest quality/score) for the AAV.UbC.CDKL5-1co.miR183-treated WT and Cdkl5-ko mice at doses of 1×10¹⁰, 3×10¹⁰, 6×10¹⁰ GC.

FIG. 36A shows a schematic overview of the intra-cisterna magna (ICM) administration procedure.

FIG. 36B shows a more detailed overview of the ICM administration as a fluoroscopy-guided procedure.

FIG. 37A shows analysis of the brain transduction as measured by vector genome copies via qPCR of extracted DNA/RNA from different brain regions of NHP following administration with AAVrh91.UbC.CDKL5-1co.miR183.

FIG. 37B shows relative CDKL5 transgene expression (mRNA) as measured via qPCR of RNA extracted from different NHP brain regions following administration with AAVrh91.UbC.CDKL5-1co.miR183 (relative to expression in mouse brain when administered at a dose of 3×10¹⁰ GC).

FIG. 38A shows results of molecular analysis of CDKL5 gene therapy outcome on the single-neuron basis, plotted as percent of transduced neurons measured by vector genome copies.

FIG. 38B shows CDKL5 transgene expression levels as measured from detectable CDKL5 transgene mRNA in single neurons, plotted as percent transgene expressing neurons.

FIG. 39A shows results of the elevated zero maze test in male Cdkl5^{KO/Y} mice following ICV administration of an AAV vector expressing human CDKL5, plotted as time (sec) in open zone. FIG. 39B shows results of the elevated zero maze test in female Cdkl5^{KO/X} mice following ICV administration of an AAV vector expressing human CDKL5, plotted as time (sec) in open zone.

FIG. 40A shows results of the elevated zero maze test in male Cdkl5^{KO/Y} mice following ICV administration of an AAV vector expressing human CDKL5, plotted as open zone entries. FIG. 40B shows results of the elevated zero maze test in female Cdkl5^{KO/X} mice following ICV administration of an AAV vector expressing human CDKL5, plotted as open zone entries.

FIG. 41A shows results of the elevated zero maze test in male Cdkl5^{KO/Y} mice following ICV administration of an AAV vector expressing human CDKL5, plotted as total distance moved. FIG. 41B shows results of the elevated zero maze test in female Cdkl5^{KO/X} mice following ICV administration of an AAV vector expressing human CDKL5, plotted as total distance moved.

FIG. 42A shows results of Y maze test in male Cdkl5^{KO/Y} mice following ICV administration of an AAV vector expressing human CDKL5, plotted as percent spontaneous alterations. FIG. 42B shows results of Y maze test in female Cdkl5^{KO/X} mice following ICV administration of an AAV vector expressing human CDKL5, plotted as percent spontaneous alterations.

FIG. 43A shows results of contextual fear conditioning test in male Cdkl5^{KO/Y} mice following ICV administration of an AAV vector expressing human CDKL5, plotted as percent freezing. FIG. 43B shows results of contextual fear conditioning test in female Cdkl5^{KO/X} mice following ICV administration of an AAV vector expressing human CDKL5, plotted as percent freezing.

FIG. 44A shows results of a transgene product expression in male Cdkl5^{KO/Y} mice and female Cdkl5^{KO/X} mice following ICV administration of an AAV vector expressing human CDKL5 (CDKL5/tubulin). FIG. 44B shows results of an activity in male Cdkl5^{KO/Y} mice and female Cdkl5^{KO/X} mice following ICV administration of an AAV vector expressing human CDKL5 (pS222/Total EB2).

FIG. 45A shows results of the vector biodistribution in adult rhesus macaques following ICM administration of an AAVhu68.hSyn.hCDKL5-1co.WPRE.SV40 at a low dose. FIG. 45B shows results of the vector biodistribution in adult rhesus macaques following ICM administration of an AAVhu68.UbC.hCDKL5-1co.SV40 at a low dose. FIG. 45C shows results of the vector biodistribution in adult rhesus macaques following ICM administration of an AAVhu68.hSyn.hCDKL5-1co.WPRE.SV40 at a mid-dose. FIG. 45D shows results of the vector biodistribution in adult rhesus macaques following ICM administration of an AAVhu68.UbC.hCDKL5-1co.SV40 at a mid-dose. FIG. 45E shows results of the vector biodistribution in adult rhesus macaques following ICM administration of an AAVhu68.hSyn.hCDKL5-1co.WPRE.SV40 at a high dose. FIG. 45F shows results of the vector biodistribution in adult rhesus macaques following ICM administration of an AAVhu68.UbC.hCDKL5-1co.SV40 at a high dose.

FIG. 46 shows results of a transgene product expression in the brain of adult Rhesus Macaques following ICM administration of an AAV vector expressing human CDKL5-Adult (3-10 years old) male and female rhesus macaques received a single ICM administration of either AAVhu68.hSyn.hCDKL5-1co.WPRE.SV40 or AAVhu68.UbC.hCDKL5-1co.SV40 at a low dose (3.0×10¹² GC), mid-dose (1.0×10¹³ GC), or high dose (3.0×10¹³ GC) (N=1 animal per vector per dose).

FIG. 47A shows upstream manufacturing process flow diagram for drug substance.

FIG. 47B shows downstream manufacturing process flow diagram for drug substance.

FIG. 48 shows overview of manufacturing process flow diagram for drug substance.

### DETAILED DESCRIPTION OF THE INVENTION

Compositions and methods for treating CDD are provided herein. An effective amount of a recombinant adeno-associated virus (rAAV) having an AAV capsid (e.g., AAVhu68) and packaged therein a vector genome encoding a functional human cyclin dependent kinase like 5 (hCDKL5) is delivered to a subject in need.

### I. HUMAN CDKL5

Cyclin dependent kinase like 5 (CDKL5, also known as CFAP247, serine/threonine kinase 9, STK9; Uniprot #076039) gene is natively located on the short (p) arm of the X chromosome at position 22.13. The N-terminus of the CDLK5 protein acts as a kinase, which is an enzyme that changes the activity of other proteins. Several direct substrates for CDKL5 have been identified (Baltussen et al, 2018; Munoz et al, 2018). The CDKL5 C-terminus is of unknown function.

As used herein, a functional hCDKL5 protein refers to an isoform, a natural variant, a variant, a polymorph, or a truncation of a CKDL5 protein which is not associated with CDD and/or delivery or expression of which may ameliorate symptoms or delay progression of CDD in an animal model or a patient. See, OMIM #300203, each of the webpages is incorporated herein by reference in its entirety. In certain embodiments, the functional hCDKL5 has an amino acid sequence of SEQ ID NO: 2 (isoform 1) or an amino acid sequence at least about 90% (e.g., at least about 90%, 91%, 92%, 93%, 94%, 95%, 96%, 97%, 98%, 99%, or 99.9%) identical thereto. In certain embodiment, the functional hCDKL5 protein has an amino acid sequence of SEQ ID NO: 19 (isoform 2) or an amino acid sequence at least about 90% (e.g., at least about 90%, 91%, 92%, 93%, 94%, 95%, 96%, 97%, 98%, 99%, or 99.9%) identical thereto. In certain embodiment, the functional hCDKL5 protein has an amino acid sequence of SEQ ID NO: 20 (isoform 3) or an amino acid sequence at least about 90% (e.g., at least about 90%, 91%, 92%, 93%, 94%, 95%, 96%, 97%, 98%, 99%, or 99.9%) identical thereto. In certain embodiment, the functional hCDKL5 protein has an amino acid sequence of SEQ ID NO: 21 (isoform 4) or an amino acid sequence at least about 90% (e.g., at least about 90%, 91%, 92%, 93%, 94%, 95%, 96%, 97%, 98%, 99%, or 99.9%) identical thereto. In certain embodiment, the functional hCDKL5 is a truncated hCDKL5 which comprises a methyl-CpG binding domain (MBD) having the sequence and a NCoR/SMRT Interaction Domain (NID). See, WO2018172795A1, which is incorporated herein by reference in its entirety.

In certain embodiments, a functional hCDKL5 protein ameliorates symptoms or delays progression of CDD in an animal model. One exemplified animal model is a CDKL5-ko mouse. Other suitable models are described herein.

The CDD symptoms or progression may be evaluated using various assays/methods, including but not limited to, a survival plot (e.g., Kaplan-Meier survival plot), monitoring body weights, and observing behavior changes (for example, by hind limb clasping, Open Field Assay (motor function), Elevated Zone Maze (anxiety/risk vs. exploration), Y Maze (learning and memory/hippocampus), Marble Burying Assay (inborn behavior and locomotion), Nesting (inborn social behavior), and rotarod assay (motor function, coordination)). In certain embodiment, administration or expression of a functional hCDKL5 protein in an animal model leads to amelioration of CDD symptoms or delay in CDD progression shown by an assay result which is at least about 10%, 20%, 30%, 40%, 50%, 60%, 70%, 80%, 90%, 95%, 100%, or more than 100% of that obtained in a corresponding wildtype animal. In certain embodiment, administration or expression of a functional hCDKL5 protein in a CDD animal model leads to amelioration of CDD symptoms or delay in CDD progression shown by an improved assay result which is at least about 10%, 20%, 30%, 40%, 50%, 60%, 70%, 80%, 90%, 95%, 100%, or more than 100% of that obtained from a corresponding non-treated CDD animal.

Provided herein is a nucleic acid sequence encoding a functional hCDKL5 protein, termed as hCDKL5 coding sequence or CDKL5 coding sequence. In certain embodiments, the hCDKL5-coding sequence is SEQ ID NO: 3 or a sequence at least about 95% identical to SEQ ID NO: 3. In certain embodiments, the hCDKL5 coding sequence is selected from SEQ ID NO: 2 (referred to as CDKL5 or CDKL5co or CDKL5-1 or CDKL5-1co) or NCBI Reference Sequences NM_001037343.1 (referred to as CDKL5 or CDKL5e1; SEQ ID NO: 16) encoding amino acid sequence NP_001032420.1 (SEQ ID NO: 19), NM_001323289.2 (SEQ ID NO: 17) encoding amino acid sequence NP_001310218.1 (SEQ ID NO: 20), and NM_003159.2 (SEQ ID NO: 18) encoding amino acid sequence NP_003150.1 (SEQ ID NO: 21), or a nucleic acid sequence at least about 70% (e.g., at least about 75%, 80%, 85%, 90%, 91%, 92%, 93%, 94%, 95%, 96%, 97%, 98%, 99%, or 99.9%) identical thereto. Each of the NCBI Reference Sequences is incorporated herein by reference in its entirety. In certain embodiments, the hCDKL5 coding sequence is a modified or engineered (hCDKL5 or hCDKL5co or CDKL5-1 or CDKL5-1co). The modified or engineered shares less than about 70% (e.g., about 75%, 80%, 85%, 90%, 91%, 92%, 93%, 94%, 95%, 96%, 97%, 98%, 99%, or 99.9%) identity to the NCBI Reference Sequences.

In certain embodiments, the hCDKL5 coding sequence is SEQ ID NO: 22 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto. In certain embodiments, the hCDKL5 coding sequence is SEQ ID NO: 24 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto. In certain embodiments, the hCDKL5 coding sequence is SEQ ID NO: 25 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto.

In certain embodiments, the hCDKL5 coding sequence is SEQ ID NO: 26 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto.

In certain embodiments, the hCDKL5-coding sequence is an engineered sequence of SEQ ID NO: 37 or a sequence at least about 95% identical to SEQ ID NO: 37. In certain embodiments, the hCDKL5-coding sequence is an engineered sequence of SEQ ID NO: 38 or a sequence at least about 95% identical to SEQ ID NO: 38. In certain embodiments, the hCDKL5-coding sequence is an engineered sequence of SEQ ID NO: 39 or a sequence at least about 95% identical to SEQ ID NO: 39. In certain embodiments, the hCDKL5-coding sequence is an engineered sequence of SEQ ID NO: 40 or a sequence at least about 95% identical to SEQ ID NO: 40. In certain embodiments, the hCDKL5-coding sequence is an engineered sequence of SEQ ID NO: 41 or a sequence at least about 95% identical to SEQ ID NO: 41. In certain embodiments, the hCDKL5-coding sequence is an engineered sequence of SEQ ID NO: 42 or a sequence at least about 95% identical to SEQ ID NO: 42. In certain embodiments, the hCDKL5-coding sequence is an engineered sequence of SEQ ID NO: 43 or a sequence at least about 95% identical to SEQ ID NO: 43. In certain embodiments, the hCDKL5-coding sequence is an engineered sequence of SEQ ID NO: 44 or a sequence at least about 95% identical to SEQ ID NO: 44. In certain embodiments, the hCDKL5-coding sequence is an engineered sequence of SEQ ID NO: 45 or a sequence at least about 95% identical to SEQ ID NO: 45. In certain embodiments, the hCDKL5-coding sequence is an engineered sequence of SEQ ID NO: 46 or a sequence at least about 95% identical to SEQ ID NO: 46. In certain embodiments, the hCDKL5-coding sequence is an engineered sequence of SEQ ID NO: 47 or a sequence at least about 95% identical to SEQ ID NO: 47. In certain embodiments, the hCDKL5-coding sequence is an engineered sequence of SEQ ID NO: 48 or a sequence at least about 95% identical to SEQ ID NO: 48.

A “nucleic acid”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

### II. EXPRESSION CASSETTE

Provided herein is a nucleic acid sequence comprising the hCDKL5 coding sequence under control of regulatory sequences which direct the hCDKL5 expression in a target cell, also termed as an expression cassette. As used herein, an “expression cassette” refers to a nucleic acid molecule which comprises a coding sequence (e.g., a CDKL5 coding sequence), and regulatory sequences operably linked thereto. In certain embodiments, a vector genome contains two or more expression cassettes. The term “transgene” refers to a DNA sequence from an exogenous source which is inserted into a target cell; typically, the transgene encodes a product (e.g., CDKL5). Typically, such an expression cassette to be packed into a viral vector contains the coding sequence for the gene product described herein flanked by packaging signals of the viral genome and other expression control sequences such as those described herein. The regulatory sequences necessary are operably linked to the hCDKL5 coding sequence in a manner which permits its transcription, translation and/or expression in target cell. As used herein, “operably linked” sequences include sequences which modulate transcription, translation, and/or expression that are contiguous with the hCDKL5 coding sequence and regulatory sequences that act in trans or at a distance to control the hCDKL5 coding sequence. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. Such regulatory sequences typically include, e.g., one or more of a promoter, an enhancer, an intron, a Kozak sequence, a polyadenylation sequence, and a TATA signal. In certain embodiment, the promoter is a tissue-specific promoter, e.g., a CNS-specific or neuron-specific promoter. In certain embodiments, the promote is a human synapsin promoter (SEQ ID NO: 23). In certain embodiments, an additional or alternative neuron-specific promoter sequence may be selected from neuron-specific enolase (NSE) promoter (Andersen et al., (1993) Cell. Mol. Neurobiol., 13:503 15), neurofilament light chain gene promoter (Piccioli et al., (1991) Proc. Natl. Acad. Sci. USA, 88:5611 5), neuron-specific vgf gene promoter (Piccioli et al., (1995) Neuron, 15:373 84), and/or others.

In certain embodiments, the human synapsin promoter has a sequence (e.g., nt 213 to nt 678 of SEQ ID NOs: 1, 3, 5, 7, 9 or SEQ ID NO: 23, also termed as hSyn or Syn herein.

In other embodiments, the promoter is a constitutive promoter, e.g., a chicken beta actin promoter with a cytomegalovirus enhancer (CB7) promoter, a human elongation initiation factor 1 alpha promoter (EF 1a) promoter, a human ubiquitin C (UbC) promoter. In certain embodiments, the regulatory sequences direct hCDKL5 expression in central nervous system (CNS) cells. In certain embodiments, the UbC promoter comprises nucleic acid sequence of SEQ ID NO: 52.

In certain embodiment, the target cell may be a central nervous system cell. In certain embodiments, the target cell is one or more of an excitatory neuron, an inhibitory neuron, a glial cell, a cortex cell, a frontal cortex cell, a cerebral cortex cell, a spinal cord cell. In certain embodiments, the target cell is a peripheral nervous system (PNS) cell, for example a retina cell. Other cells other than those from nervous system may also be chosen as a target cell, such as a monocyte, a B lymphocyte, a T lymphocyte, a NK cell, a lymph node cell, a tonsil cell, a bone marrow mesenchymal cell, a stem cell, a bone marrow stem cell, a heart cell, an epithelium cell, a esophagus cell, a stomach cell, a fetal cut cell, a colon cell, a rectum cell, a liver cell, a kidney cell, a lung cell, a salivary gland cell, a thyroid cell, an adrenal cell, a breast cell, a pancreas cell, an islet of Langerhans cell, a gallbladder cell, a prostate cell, a urinary bladder cell, a skin cell, a uterus cell, a cervix cell, a testis cell, or any other cell which expresses a functional CDKL5 protein in a subject without CDD.

In certain embodiments, an additional or alternative promoter sequence may be included as part of the expression control sequences (regulatory sequences), e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

In addition to a promoter, a vector may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the regulatory sequences comprise one or more expression enhancers. In one embodiment, the regulatory sequences contain two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. In certain embodiments, the intron is a chimeric intron (CI)—a hybrid intron consisting of a human beta-globin splice donor and immunoglobulin G (IgG) splice acceptor elements. Other suitable introns include those known in the art, e.g., such as are described in WO 2011/126808. Examples of suitable polyA sequences include, e.g., Rabbit globin poly A, SV40, SV50, bovine growth hormone (bGH), human growth hormone, and synthetic polyAs. In certain embodiments, the polyA sequence is an SV40 polyA sequence. In certain embodiments, the polyA sequence is a rabbit beta globin (RBG or rbg or rBG) polyA sequence. In certain embodiments, the polyA is a rabbit beta globin polyA comprising nucleic acid sequence of SEQ ID NO: 53. Optionally, one or more sequences may be selected to stabilize mRNA. The expression cassettes provided may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expressions cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene (See, Zanta-Boussif et al., Gene Ther. 2009 May; 16(5):605-19, which is incorporated by reference). In other embodiments, enhancers are selected from a non-viral source. In certain embodiments, no WPRE sequence is present.

In certain embodiments, an expression cassette refers to nucleic acid molecule with sequence of nt 213-4439 of SEQ ID NO: 1, encoding for amino acid sequence of hCDKL5 (isoform 1; SEQ ID NO: 2). In certain embodiments, an expression cassette refers to nucleic acid molecule with sequence of nt 213-4562 of SEQ ID NO: 5 encoding for amino acid sequence of hCDKL5 (isoform 2 or 2GS SEQ ID NO: 6). In certain embodiments, an expression cassette refers to nucleic acid molecule with sequence of nt 213-4388 of SEQ ID NO: 7 encoding for amino acid sequence of hCDKL5 (isoform 3 or 3GS; SEQ ID NO: 8). In certain embodiments, an expression cassette refers to nucleic acid molecule with sequence of nt 213-4511 of SEQ ID NO: 9 encoding for amino acid sequence of hCDKL5 (isoform 4 or 4GS; SEQ ID NO: 10). In certain embodiments, an expression cassette comprises an engineered nucleic acid sequence selected from SEQ ID NOs: 37, 38, 39, 40, 41, 42, 43, 44, 45, 46, 47, or 48 and encoding for an amino acid sequence of hCDKL5 (isoform 1; SEQ ID NO: 2). In certain embodiments, an expression cassette refers to nucleic acid molecule with sequence of nt 213-4555 of SEQ ID NO: 3, encoding for amino acid sequence of hCDKL5 (isoform 1; SEQ ID NO: 4) and comprising of miRNA183 (SEQ ID NO: 11). In certain embodiments, these expression cassettes further comprise one, two, three, four or more miRNA sequences for reducing drg expression. In certain embodiments, an expression cassette refers to nucleic acid molecule with sequence of nt 226-4608 of SEQ ID NO: 29 (or SEQ ID NO: 59), encoding for amino acid sequence of hCDKL5 (isoform 1; SEQ ID NO: 30) and comprising 4 tandem repeats of miRNA183 (SEQ ID NO: 11). In certain embodiments, an expression cassette refers to nucleic acid molecule with sequence of nt 220-4609 of SEQ ID NO: 49 (or SEQ ID NO: 50), encoding for amino acid sequence of hCDKL5 (isoform 1) and comprising 4 tandem repeats of miRNA 183 (SEQ ID NO: 11). In certain embodiments, an expression cassette refers to nucleic acid molecule with sequence of nt 224-4191 of SEQ ID NO: 31 (or SEQ ID NO: 60), encoding for amino acid sequence of hCDKL5 (isoform 1; SEQ ID NO: 32) and comprising 4 tandem repeats of miRNA 183 (SEQ ID NO: 11). See, e.g., PCT/US19/67872, filed Dec. 20, 2019 and now published as WO 2020/132455.

In certain embodiments, the expression cassette comprises four copies of the miR183 expression cassette. In certain embodiments, the expression cassette contains a miR-183 target sequence that includes AGTGAATTCTACCAGTGCCATA (SEQ ID NO: 11), where the sequence complementary to the miR-183 seed sequence is underlined. In certain embodiments, the expression cassette contains more than one copy (e.g., two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 11 and, thus, when aligned to SEQ ID NO: 11, there are one or more mismatches. In certain embodiments, a miR-183 target sequence comprises a sequence having at least 1, 2, 3, 4, 5, 6, 7, 8, 9, or 10 mismatches when aligned to SEQ ID NO: 11, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette includes a miR-183 target sequence that comprises a truncated SEQ ID NO: 11, i.e., a sequence that lacks at least 1, 2, 3, 4, 5, 6, 7, 8, 9, or 10 nucleotides at either or both the 5′ or 3′ ends of SEQ ID NO: 11. In certain embodiments, the expression cassette comprises a transgene and one miR-183 target sequence. In yet other embodiments, the expression cassette comprises at least two, three or four miR-183 target sequences. In certain embodiments, the inclusion of at two, three or four miR-183 target sequences in the expression cassette results in increased levels of transgene expression in a target tissue, such as the heart.

In one embodiment, the expression cassette comprises a UbC promoter, the hCDKL5-1 coding sequence, 4 copies of a miR183 target sequence, and a polyA sequence. In another embodiment, the expression cassette comprises a hSyn promoter, the hCDKL5-1 coding sequence, 4 copies of a miR183 target sequence, and a polyA sequence. In another embodiment, the expression cassette comprises a CBh promoter, the hCDKL5-1 coding sequence, 4 copies of a miR183 target sequence, and a polyA sequence. In certain embodiments, the expression cassette further comprises at least one intron and/or at least one enhancer sequence. In certain embodiments, the enhancer is a mutant WPRE element lacking the ability to express the woodchuck hepatitis B virus X (WHX) protein.

In certain embodiments, a vector genome comprises a 5′-AAV ITR sequence, a spacer sequence, an expression cassette as described herein, a spacer sequence, and a 3′-AAV ITR. Suitably, there may be non-coding spacer sequences between the 5′ ITR sequences and the 5′ end of the expression cassette and non-coding spacer sequences between the 3′ end of the ITR sequences and the 3′ ITRs.

In certain embodiments, the expression cassette comprises a nucleic acid sequence of nt 220 to 4609 of SEQ ID NO: 49 (or SEQ ID NO: 50). In certain embodiments, the expression cassette comprises a nucleic acid sequence of nt 226 to 4608 of SEQ ID NO: 29 (or SEQ ID NO: 59). In certain embodiments, the expression cassette comprises nucleic acid sequence of nt 224 to 4191 of SEQ ID NO: 31 (or SEQ ID NO: 60).

### III. rAAV

Provided herein is a recombinant adeno-associated virus (rAAV) useful for treating CDD. The rAAV comprises (a) an AAV capsid; and (b) a vector genome packaged in the AAV capsid of (a). Suitably, the AAV capsid selected targets the cells to be treated. In certain embodiments, the capsid is from Clade F. However, in certain embodiments, another AAV capsid source may be selected. The vector genome comprises inverted terminal repeats (ITR) and a nucleic acid sequence encoding a functional human cyclin dependent kinase like 5 (hCDKL5) under control of regulatory sequences which direct the hCDKL5 expression. In certain embodiments CDKL5 may be referring to CDKL5 or hCDKL5, CDKL5-2GS or hCDKL5-2GS, CDKL5-3GS or hCDKL5-3GS, and CDKL5-4GS or hCDKL5-4GS. In certain embodiments, the hCDKL5-coding sequence is at least about 95% identical to SEQ ID NO: 22 (encoding amino acid sequence of CDKL5-1 or hCDKL5-1; SEQ ID NO: 2). In certain embodiments, the hCDKL5-coding sequence is at least about 95% identical to SEQ ID NO: 24 (encoding amino acid sequence of CDKL5-2GS or hCDKL5-2GS; SEQ ID NO: 6). In certain embodiments, the hCDKL5-coding sequence is at least about 95% identical to SEQ ID NO: 25 (encoding amino acid sequence of CDKL5-3GS or hCDKL5-3GS; SEQ ID NO: 8). In certain embodiments, the hCDKL5-coding sequence is at least about 95% identical to SEQ ID NO: 26 (encoding amino acid sequence of CDKL5-4GS or hCDKL5-4GS; SEQ ID NO: 10). In certain embodiments, the hCDKL5-coding sequence is less than 80% identical to any one of hCDKL5 transcript variants 1 to 3 (NM_001037343.1 with SEQ ID NO: 16 and encoding amino acid sequence NP_001032420.1 with SEQ ID NO: 19; NM_001323289.2 with SEQ ID NO: 17 and encoding amino acid sequence NP_001310218.1 with SEQ ID NO: 20; NM_003159.2 with SEQ ID NO: 18 encoding amino acid sequence NP_003150.1 with SEQ ID NO: 21). In certain embodiments, the hCDKL5-coding sequence is SEQ ID NOs: 37, 38, 39, 40, 41, 42, 43, 44, 45, 46 and 47 or at least about 95% identical thereto (encoding amino acid sequence of CDKL5-1 or hCDKL5-1; SEQ ID NO: 2). In certain embodiments, the functional hCDKL5 has an amino acid sequence of SEQ ID NO: 2 (CDKL5-1 or hCDKL5-1). In certain embodiments, the functional hCDKL5 has an amino acid sequence of SEQ ID NO: 6 (CDKL5-2GS or hCDKL5-2GS). In certain embodiments, the functional hCDKL5 has an amino acid sequence of SEQ ID NO: 8 (CDKL5-3GS or hCDKL5-3GS). In certain embodiments, the functional hCDKL5 has an amino acid sequence of SEQ ID NO: 10 (CDKL5-4GS or hCDKL5-4GS). In certain embodiments, the regulatory sequences direct hCDKL5 expression in central nervous system cells. In certain embodiments, the regulatory sequences comprise a human Synapsin promoter (hSyn) or a CB7 promoter. In certain embodiments, the regulatory sequences comprise a human Ubiquitin C (hUbC or UbC) promoter. In certain embodiments, the regulatory elements comprise one or more of a Kozak sequence, a polyadenylation sequence, an intron, an enhancer, and a TATA signal. In certain embodiments, the vector genome further comprises at least two tandem repeats of dorsal root ganglion (drg)-specific miRNA target sequences, wherein the at least two tandem repeats comprise at least a first miRNA target sequence and at least a second miRNA target sequence which may be the same or different. In certain embodiments, the vector genome is nt 1 to nt 4634 of SEQ ID NO: 1, or nt 1 to nt 4750 of SEQ ID NO: 3, or nt 1 to nt 4757 of SEQ ID NO: 5, or nt 1 to nt 4583 of SEQ ID NO: 7, or nt 1 to nt 4706 of SEQ ID NO: 9 or a nucleic acid sequence at least about 70% (e.g., at least about 75%, 80%, 85%, 90%, 91%, 92%, 93%, 94%, 95%, 96%, 97%, 98%, 99%, or 99.9%) identical thereto.

In certain embodiments, a vector genome refers to nucleic acid molecule comprising SEQ ID NO: 1, encoding for amino acid sequence of hCDKL5 (isoform 1; SEQ ID NO: 2). In certain embodiments, a vector genome refers to nucleic acid molecule comprising SEQ ID NO: 5 encoding for amino acid sequence of hCDKL5 (isoform 2 or 2GS SEQ ID NO: 6). In certain embodiments, a vector genome refers to nucleic acid molecule comprising SEQ ID NO: 7 encoding for amino acid sequence of hCDKL5 (isoform 3 or 3GS; SEQ ID NO: 8). In certain embodiments, a vector genome refers to nucleic acid molecule comprising SEQ ID NO: 9 encoding for amino acid sequence of hCDKL5 (isoform 4 or 4GS; SEQ ID NO: 10). In certain embodiments, a vector genome refers to nucleic acid molecule comprising SEQ ID NO: 3, encoding for amino acid sequence of hCDKL5 (isoform 1; SEQ ID NO: 4) and comprising of miRNA 183 (SEQ ID NO: 11). In certain embodiments, a vector genome refers to a nucleic acid molecule comprising SEQ ID NO: 29, encoding an amino acid sequence of hCDKL5 (isoform 1; SEQ ID NO: 30) and comprising tandem repeats of miRNA183 (SEQ ID NO: 11). In certain embodiments, a vector genome refers to nucleic acid molecule comprising SEQ ID NO: 31, encoding for amino acid sequence of hCDKL5 (isoform 1; SEQ ID NO: 32) and comprising tandem repeats of miRNA183 (SEQ ID NO: 11).

In certain embodiments, in addition to the hCDKL5 coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the Y untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

As used herein, an “miRNA target sequence” is a sequence located on the DNA positive strand (5′ to 3′) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. The term “miR183 cluster target sequence” refers to a target sequence that responds to one or members of the miR183 cluster (alternatively termed family), including miRs-183, -96 and -182 (as described by Dambal, S. et al. Nucleic Acids Res 43:7173-7188, 2015, which is incorporated herein by reference).

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides, and contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA.

As used herein, an “miRNA target sequence” is a sequence located on the DNA positive strand (5′ to 3′) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. The term “miR183 cluster target sequence” refers to a target sequence that responds to one or members of the miR183 cluster (alternatively termed family), including miRs-183, -96 and -182 (as described by Dambal, S. et al. Nucleic Acids Res 43:7173-7188, 2015, which is incorporated herein by reference).Without wishing to be bound by theory, the messenger RNA (mRNA) for the transgene (encoding the gene product) is present in a cell type to which the expression cassette containing the miRNA is delivered, such that specific binding of the miRNA to the 3′ UTR miRNA target sequences results in mRNA silencing and cleavage, thereby reducing or eliminating transgene expression only in the cells that express the miRNA.

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides, and contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA.

In certain embodiments, the miRNA target sequence for the at least first and/or at least second miRNA target sequence for the expression cassette mRNA or DNA positive strand is selected from (i) AGTGAATTCTACCAGTGCCATA (miR183, SEQ ID NO: 11); or (ii) AGTGTGAGTTCTACCATTGCCAAA (miR182, SEQ ID NO: 13). In other embodiments, AGGGATTCCTGGGAAAACTGGAC (SEQ ID NO: 14) is selected.

In certain embodiments, the vector genome or expression cassette contains at least one miRNA target sequence that is a miR-183 target sequence. In certain embodiments, the vector genome or expression cassette contains an miR-183 target sequence that includes AGTGAATTCTACCAGTGCCATA (SEQ ID NO: 11), where the sequence complementary to the miR-183 seed sequence is GTGCCAT. In certain embodiments, the vector genome or expression cassette contains more than one copy (e.g., two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 11 and, thus, when aligned to SEQ ID NO: 11, there are one or more mismatches. In certain embodiments, a miR-183 target sequence comprises a sequence having at least 1, 2, 3, 4, 5, 6, 7, 8, 9, or 10 mismatches when aligned to SEQ ID NO: 11, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 11, i.e., a sequence that lacks at least 1, 2, 3, 4, 5, 6, 7, 8, 9, or 10 nucleotides at either or both the 5′ or 3′ ends of SEQ ID NO: 11. In certain embodiments, the expression cassette or vector genome comprises a transgene and one miR-183 target sequence. In yet other embodiments, the expression cassette or vector genome comprises at least two, three or four miR-183 target sequences. (i) AGTGAATTCTACCAGTGCCATA (miR183, SEQ ID NO: 11).

In certain embodiments, the vector genome or expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 13). In certain embodiments, the vector genome or expression cassette contains more than one copy (e.g., two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 13 and, thus, when aligned to SEQ ID NO: 13, there are one or more mismatches. In certain embodiments, a miR-183 target sequence comprises a sequence having at least 1, 2, 3, 4, 5, 6, 7, 8, 9, or 10 mismatches when aligned to SEQ ID NO: 13, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 13, i.e., a sequence that lacks at least 1, 2, 3, 4, 5, 6, 7, 8, 9, or 10 nucleotides at either or both the 5′ or 3′ ends of SEQ ID NO: 13. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

In certain embodiments, the spacers between the miRNA target sequences are the same. As used herein, CDKL5 or hCDKL5 refers to isoform 1, unless otherwise specified. Isoforms 2-4 may be specified as: CDKL5-2GS or hCDKL5-2GS, CDKL5-3GS or hCDKL5-3GS, and CDKL5-4GS or hCDKL5-4GS. Expression cassettes and vector genomes with these isoforms may be constructed as described for isoform 1.

See, PCT/US19/67872, filed Dec. 20, 2019, now WO 2020/132455, which is incorporated by reference herein and U.S. Provisional Patent Applications No. 63/023,593, filed May 12, 2020; U.S. Provisional Patent Applications No. 63/038,488, filed Jun. 12, 2020; U.S. Provisional Patent Applications No. 63/043,562, filed Jun. 24, 2020; and U.S. Provisional Patent Applications No. 63/079,299, filed Sep. 16, 2020, U.S. Provisional Patent Application No. 63/152,042, filed Feb. 22, 2011, which are hereby incorporated by reference.

In certain embodiments, the Clade F AAV capsid is selected from an AAVhu68 capsid, an AAV9 capsid, an AAVhu31 capsid, an AAVhu32 capsid, or an engineered variant of one of these capsids. Nucleic acid sequences encoding AAVhu68 capsid protein, are utilized in the examples below for production of an AAV.hCDKL5 recombinant AAV (rAAV) carrying the vector genome. Additional details relating to AAVhu68 re provided in WO 2018/160582, and US 2015/0079038, each of which is incorporated herein by reference in its entirety. The Clade F vectors described herein are well suited for delivery of the vector genome comprising the hCDKL5 coding sequence to cells within the central nervous system, including brain, hippocampus, motor cortex, cerebellum, and motor neurons. These vectors may be used for targeting other cells within the central nervous system (CNS) and certain other tissues and cells outside the CNS.

In certain embodiments, the AAV capsid for the compositions and methods described herein is chosen based on the target cell. In certain embodiment, the AAV capsid transduces a CNS cell and/or a PNS cell. In certain embodiments, the AAV capsid is selected from a cy02 capsid, a rh43 capsid, an AAV8 capsid, a rh01 capsid, an AAV9 capsid, an rh8 capsid, a rhl0 capsid, a bb01 capsid, a hu37 capsid, a rh02 capsid, a rh20 capsid, a rh39 capsid, a rh64 capsid, an AAV6 capsid, an AAV1 capsid, a hu44 capsid, a hu48 capsid, a cy05 capsid a hu11 capsid, a hu32 capsid, a pi2 capsid, or a variation thereof. In certain embodiments, the AAV capsid is a Clade F capsid, such as AAV9 capsid, AAVhu68 capsid, hu31 capsid, hu32 capsid, or a variation thereof. See, e.g., WO 2005/033321 published Apr. 14, 2015, WO 2018/160582, and US 2015/0079038, each of which is incorporated herein by reference in its entirety. In certain embodiments, the AAV capsid is a non-clade F capsid, for example a Clade A, B, C, D, or E capsid. In certain embodiment, the non-Clade F capsid is an AAV1 or a variation thereof. In certain embodiment, the AAV capsid transduces a target cell other than the nervous system cells. In certain embodiments, the AAV capsid is a Clade A capsid (e.g., AAV1, AAV6, AAVrh91), a Clade B capsid (e.g., AAV 2), a Clade C capsid (e.g., hu53), a Clade D capsid (e.g., AAV7), or a Clade E capsid (e.g., rh10). In certain embodiments, the AAV capsid is a Clade A capsid, such as AAVrh91 capsid (nucleic acid sequence of SEQ ID NOs: 33 and 35). See, PCT/US20/030266, filed Apr. 29, 2020, now published WO2020/223231, which is incorporated by reference herein and US Provisional U.S. Patent Application No. 63/065,616, filed Apr. 29, 2019, which is hereby incorporated by reference. See also, U.S. Provisional Application No. 63/065,616, filed Aug. 14, 2020, and U.S. Provisional Patent Application No. 63/109,734, filed Nov. 4, 2020, and International Application No. PCT/US21/45945, filed Aug. 13, 2021 which are incorporated herein by reference. Still, other AAV capsid may be chosen.

In certain embodiments, the AAV capsid is a AAVhu68 capsid, or an AAVrh91 capsid. In certain embodiments, the AAVhu68 capsid is produced from a nucleic acid sequence encoding amino acid sequence of SEQ ID NO: 61. In certain embodiments, the AAVhu68 capsid comprises: (i) AAVhu68 vp1 proteins, AAVhu68 vp2 proteins, and AAVhu68 vp3 proteins produced from a nucleic acid sequence encoding SEQ ID NO: 61; or (ii) heterogenous populations of AAVhu68 vp1, AAVhu68 vp2 and AAVhu68 vp3 proteins, wherein the AAVhu68 vp1, AAVhu68 vp2 and AAV hu68 vp3 proteins comprise at least 50% to 100% deamidated asparagines (N) in asparagine-glycine pairs at each of positions 57, 329, 452, 512, relative to the amino acids in SEQ ID NO: 61, wherein the deamidated asparagines are deamidated to aspartic acid, isoaspartic acid, an interconverting aspartic acid/isoaspartic acid pair, or combinations thereof, as determined using mass spectrometry. In certain embodiments, the nucleic acid sequence encoding AAVhu68 vp1 protein is SEQ ID NO: 57, or a sequence at least 80% to at least 99% identical to SEQ ID NO: 57 which encodes the amino acid sequence of SEQ ID NO: 61; optionally wherein the nucleic acid sequence is at least 80% to 97% identical to SEQ ID NO: 57. See, e.g., WO 2018/160582 and WO2019/169004 which are incorporated by reference herein in its entirety.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp 1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321.

A rAAV is composed of an AAV capsid and a vector genome. An AAV capsid is an assembly of a heterogeneous population of vp 1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein when used to refer to vp capsid proteins, the term “heterogeneous” or any grammatical variation thereof, refers to a population consisting of elements that are not the same, for example, having vp 1, vp2 or vp3 monomers (proteins) with different modified amino acid sequences. The term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp 1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart.

As used herein, the term “vector genome” refers to a nucleic acid molecule which is packaged in a viral capsid, for example, an AAV capsid, and is capable of being delivered to a host cell or a cell in a patient. In certain embodiments, the vector genome is an expression cassette having inverted terminal repeat (ITR) sequences necessary for packaging the vector genome into the AAV capsid at the extreme 5′ and 3′ end and containing therebetween a CDLK5 gene as described herein operably linked to sequences which direct expression thereof. In certain embodiments, a vector genome may comprise at a minimum from 5′ to 3′, an AAV 5′ ITR, coding sequence(s), and an AAV 3′ ITR. In certain embodiments, the ITRs are from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. The vector genome is sometimes referred to herein as the “minigene”.

As used herein, the term “host cell” may refer to the packaging cell line in which the rAAV is produced from the plasmid. In the alternative, the term “host cell” may refer to the target cell in which expression of the transgene is desired.

As indicated above, a rAAV is provided which has an AAV capsid which targets the desired cells and a vector genome which comprises, at a minimum, AAV ITRs required to package the vector genome into the capsid, a hCDKL5 coding sequence and regulatory sequences which direct expression therefor. In certain embodiments, the vector genome is a single-stranded AAV vector genome. In certain embodiments, a rAAV vector may be utilized in the invention which contains self-complementary (sc) AAV vector genome.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed AITR, has been described in which the D-sequence and terminal resolution site (trs) are deleted. In other embodiments, the full-length AAV 5′ and 3′ ITRs are used. In certain embodiments, the vector genome includes a shortened AAV2 ITR of 130 base pairs, wherein the external A elements is deleted. The shortened ITR is reverted back to the wild-type length of 145 base pairs during vector DNA amplification using the internal A element as a template. In certain embodiments, the 5′ ITR comprises nucleic acid sequence of SEQ ID NO: 51. In certain embodiments, the 3′ ITR comprises nucleic acid sequence of SEQ ID NO: 54. However, ITRs from other AAV sources may be selected. Where the source of the ITRs is from AAV2 and the AAV capsid is from another AAV source, the resulting vector may be termed pseudotyped. However, other configurations of these elements may be suitable.

In certain embodiments, vector genomes are constructed which comprise a 5′ AAV ITR-promoter-optional enhancer-optional intron-hCDKL5 coding sequence-polyA-3′ ITR. In certain embodiments, vector genomes are constructed which comprise a 5′ AAV ITR-promoter-optional enhancer-optional intron-hCDKL5 coding sequence-optionally repeating miR (de)targeting sequences-polyA-3′ ITR. In certain embodiments, vector genomes are constructed which comprise a 5′ AAV ITR-promoter-optional intron hCDKL5 coding sequence-optional enhancer-polyA-3′ ITR. In certain embodiments, vector genomes are constructed which comprise a 5′ AAV ITR-promoter-optional enhancer-optional intron-hCDKL5 coding sequence-optional enhancer-optionally repeating miR (de)targeting sequences-polyA-3′ ITR. In certain embodiments, the ITRs are from AAV2. In certain embodiments, more than one promoter is present. In certain embodiments, the enhancer is present in the vector genome. In certain embodiments, more than one enhancer is present. In certain embodiments, an intron is present in the vector genome. In certain embodiments, the enhancer and intron are present. In certain embodiments, the polyA is an SV40 poly A (i.e., a polyadenylation (PolyA) signal derived from Simian Virus 40 (SV40) late genes). In certain embodiments, the polyA is a rabbit beta-globin (RBG) poly A. In certain embodiments, the vector genome comprises, at a minimum: a 5′ AAV ITR-hSyn promoter-hCDKL5 coding sequence-poly A-3′ ITR. In certain embodiments, the vector genome comprises, at a minimum, a 5′ AAV ITR-CB7 promoter-hCDKL5 coding sequence-RBG poly A-3′ ITR. In certain embodiments, the drg detargeting sequences are one, two, three, four or more miR183 sequences as described herein and are included in the expression cassette. In certain embodiments the hCDKL5 coding sequence is for CDKL5. In certain embodiments the hCDKL5 coding sequence is for CDKL5-2GS. In certain embodiments the hCDKL5 coding sequence is for CDKL5-3GS. In certain embodiments the hCDKL5 coding sequence is for CDKL5-4GS. Optionally, one or more of these vector genomes includes a WPRE element.

As used herein, a vector genome or a rAAV comprising the vector genome is illustrated herein as AAV.promoter (optional).Kozak (optional).intron (optional).CDKL5 coding sequence (e.g., hCDKL5, hCDKL5co, CDKL5, CDKL5co). miRNA (optional).polyA(optional).Stuffer (optional). In certain embodiments, a rAAV is illustrated herein as AAV capsid.promoter (optional).Kozak (optional).intron (optional).CDKL5 coding sequence. miRNA (optional).polyA (optional).Stuffer (optional). Optionally, one or more of these vector genomes includes a WPRE element.

In certain embodiments, the vector genome comprises at a minimum a 5′ AAV ITR-Ubiquitin C promoter-hCDKL5 coding sequence-RBG poly A-3′ ITR. In certain embodiments, the vector genome comprises nucleic acid sequence of SEQ ID NO: 58. In certain embodiments, the vector genome comprises at a minimum a 5′ AAV ITR-Ubiquitin C promoter-hCDKL5 coding sequence-one, two, three, four or more miR183 sequences-RBG poly A-3′ ITR. In certain embodiments, the vector genome comprises nucleic acid sequence of SEQ ID NO: 29. In certain embodiments, the vector genome comprises nucleic acid sequence of SEQ ID NO: 49. In certain embodiments, the vector genome comprises at a minimum a 5′ AAV ITR-Chicken-beta actin hybrid promoter-hCDKL5 coding sequence-one, two, three, four or more miR183 sequences-RBG poly A-3′ ITR. In certain embodiments, the vector genome comprises nucleic acid sequence of SEQ ID NO: 31. Optionally, one or more of these vector genomes includes a WPRE element.

Additionally, provided herein, is an rAAV production system useful for producing a rAAV as described herein. The production system comprises a cell culture comprising (a) a nucleic acid sequence encoding an AAV capsid protein; (b) the vector genome; and (c) sufficient AAV rep functions and helper functions to permit packaging of the vector genome into the AAV capsid. In certain embodiments, the vector genome is SEQ ID NOs: 1, 3, 5, 7, 9, 29 or 31. In certain embodiments, the cell culture is a human embryonic kidney 293 cell culture. In certain embodiments, the AAV rep is from a different AAV. In certain embodiments, wherein the AAV rep is from AAV2. In certain embodiments, the AAV2 rep is encoded by the nucleic acid sequence of SEQ ID NO: 56. In certain embodiments, the AAV rep coding sequence and cap genes are on the same nucleic acid molecule, wherein there is optionally a spacer between the rep sequence and cap gene. In certain embodiments, the spacer is atgacttaaaccaggt (SEQ ID NO: 15).

For use in producing an AAV viral vector (e.g., a recombinant (r) AAV), the vector genomes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99: 119-145; Buning et al., 2008, Recent developments in adeno-associated virus vector technology, J. Gene Med. 10:717-733; and the references cited below, each of which is incorporated herein by reference in its entirety. As used herein, a gene therapy vector refers to a rAAV as described herein, which is suitable for use in treating a patient. For packaging a gene into virions, the ITRs are the only AAV components required in cis in the same construct as the nucleic acid molecule containing the gene. The cap and rep genes can be supplied in trans.

In certain embodiments, the manufacturing process for rAAV involves method as described in U.S. Provisional Patent Application No. 63/371,597, filed Aug. 16, 2022, and U.S. Provisional Patent Application No. 63/371,592, filed Aug. 16, 2022, which are incorporated herein by reference in its entirety.

In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAVhu68 or AAVrh91 is provided. Such a cell culture contains a nucleic acid which expresses the AAVhu68 capsid protein in the host cell; a nucleic acid molecule suitable for packaging into the AAVhu68 capsid, e.g., a vector genome which contains AAV ITRs and a non-AAV nucleic acid sequence encoding a gene operably linked to regulatory sequences which direct expression of the gene in a host cell; and sufficient AAV rep functions and adenovirus helper functions to permit packaging of the vector genome into the recombinant AAVhu68, or AAVrh91 capsid. In one embodiment, the cell culture is composed of mammalian cells (e.g., human embryonic kidney 293 cells, among others) or insect cells (e.g., Spodoptera frugiperda (Sf9) cells). In certain embodiments, baculovirus provides the helper functions necessary for packaging the vector genome into the recombinant AAVhu68, or AAVrh91 capsid.

Optionally the rep functions are provided by an AAV other than hu68. In certain embodiments, at least parts of the rep functions are from AAVhu68, or AAVrh91. In another embodiment, the rep protein is a heterologous rep protein other than AAVhu68rep, for example but not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Any of these AAVhu68 or mutant AAV capsid sequences may be under the control of exogenous regulatory control sequences which direct expression thereof in a host cell.

In one embodiment, cells are manufactured in a suitable cell culture (e.g., HEK 293 or Sf9) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV vector genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following US patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in WO 2017/160360 filed Dec. 9, 2016 and its priority documents, US Patent Application Nos. 62/322,071, filed Apr. 13, 2016 and 62/226,357, filed Dec. 11, 2015 and entitled “Scalable Purification Method for AAV9”, which is incorporated by reference herein. In certain embodiments, the purification of vector drug product (e.g., AAVrh91) include those described in more detail in WO2017/100674, filed Dec. 9, 2016, and its priority documents, US Provisional Patent Application Nos. 62/266,351. Filed Dec. 9, 2015, and 62/322,083, filed Apr. 13, 2016 and titled “Scalable Purification Method for AAV1”, which is incorporated herein by reference.

To calculate empty and full particle content, VP3 band volumes for a selected sample (e.g., in examples herein an iodixanol gradient-purified preparation where # of genome copies (GC)=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAVhu68 (or AAVrh91) particles having packaged genomic sequences from genome-deficient AAVhu68 (or AAVrh91) intermediates involves subjecting a suspension comprising recombinant AAVhu68 (or rh91) viral particles and AAVhu68 (or AAVrh91) capsid intermediates to fast performance liquid chromatography, wherein the AAVhu68 (or AAVrh91) viral particles and AAVhu68 intermediates are bound to a strong anion exchange resin equilibrated at a pH of about 10.2 (or about 9.8 for AAVrh91), and subjected to a salt gradient while monitoring eluate for ultraviolet absorbance at about 260 nanometers (nm) and about 280 nm. Although less optimal for rAAVhu68 and AAVrh91, the pH may be in the range of about 10 to 10.4. In this method, the AAV full capsids are collected from a fraction which is eluted when the ratio of A260/A280 reaches an inflection point. In one example, for the Affinity Chromatography step, the diafiltered product may be applied to an affinity resin (Life Technologies) that efficiently captures the AAVhu68 or AAVrh91 serotype. Under these ionic conditions, a significant percentage of residual cellular DNA and proteins flow through the column, while AAV particles are efficiently captured.

The rAAV.hCDKL5 is suspended in a suitable physiologically compatible composition (e.g., a buffered saline). This composition may be frozen for storage, later thawed and optionally diluted with a suitable diluent. Alternatively, the vector may be prepared as a composition which is suitable for delivery to a patient without proceeding through the freezing and thawing steps.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

### IV. OTHER VECTOR

Provided herein is a vector comprising an expression cassette as described herein. In certain embodiments, the expression cassette comprises a nucleic acid sequence encoding a functional human cyclin dependent kinase like 5 (hCDKL5) under control of regulatory sequences which direct the hCDKL5 expression. In certain embodiments, the hCDKL5-coding sequence encodes a hCDKL5 protein comprising an amino acid sequence of

(SEQ ID NO: 2)
[MKIPNIGNVMNKFEILGVVGEGAYGVVLKCRHKETHEIVAIKKFKDSE
ENEEVKETTLRELKMLRTLKQENIVELKEAFRRRGKLYLVFEYVEKNML
ELLEEMPNGVPPEKVKSYIYQLIKAIHWCHKNDIVHRDIKPENLLISHN
DVLKLCDFGFARNLSEGNNANYTEYVATRWYRSPELLLGAPYGKSVDMW
SVGCILGELSDGQPLFPGESEIDQLFTIQKVLGPLPSEQMKLFYSNPRF
HGLRFPAVNHPQSLERRYLGILNSVLLDLMKNLLKLDPADRYLTEQCLN
HPTFQTQRLLDRSPSRSAKRKPYHVESSTLSNRNQAGKSTALQSHHRSN
SKDIQNLSVGLPRADEGLPANESFLNGNLAGASLSPLHTKTYQASSQPG
STSKDLTNNNIPHLLSPKEAKSKTEFDFNIDPKPSEGPGTKYLKSNSRS
QQNRHSFMESSQSKAGTLQPNEKQSRHSYIDTIPQSSRSPSYRTKAKSH
GALSDSKSVSNLSEARAQIAEPSTSRYFPSSCLDLNSPTSPTPTRHSDT
RTLLSPSGRNNRNEGTLDSRRTTTRHSKTMEELKLPEHMDSSHSHSLSA
PHESFSYGLGYTSPFSSQQRPHRHSMYVTRDKVRAKGLDGSLSIGQGMA
ARANSLQLLSPQPGEQLPPEMTVARSSVKETSREGTSSFHTRQKSEGGV
YHDPHSDDGTAPKENRHLYNDPVPRRVGSFYRVPSPRPDNSFHENNVST
RVSSLPSESSSGTNHSKRQPAFDPWKSPENISHSEQLKEKEKQGFFRSM
KKKKKKSQTVPNSDSPDLLTLQKSIHSASTPSSRPKEWRPEKISDLQTQ
SQPLKSLRKLLHLSSASNHPASSDPRFQPLTAQQTKNSFSEIRIHPLSQ
ASGGSSNIRQEPAPKGRPALQLPGQMDPGWHVSSVTRSATEGPSYSEQL
GAKSGPNGHPYNRTNRSRMPNLNDLKETAL],

wherein the hCDKL5-coding sequence is a nucleic acid sequence of SEQ ID NO: 22 or which has at least about 95% identical to SEQ ID NO: 22 and encodes the protein of SEQ ID NO:2. In certain embodiments, the hCDKL5-coding sequence encodes a hCDKL5-2GS protein comprising an amino acid sequence of [MKIPNIGNVMNKFEILGVVGEGAYGVVLKCRHKETHEIVAIKKFKDSEENEEVKET TLRELKMLRTLKQENIVELKEAFRRRGKLYLVFEYVEKNMLELLEEMPNGVPPEKV KSYIYQLIKAIHWCHKNDIVHRDIKPENLLISHNDVLKLCDFGFARNLSEGNNANYT EYVATRWYRSPELLLGAPYGKSVDMWSVGCILGELSDGQPLFPGESEIDQLFTIQKV LGPLPSEQMKLFYSNPRFHGLRFPAVNHPQSLERRYLGILNSVLLDLMKNLLKLDPA DRYLTEQCLNHPTFQTQRLLDRSPSRSAKRKPYHVESSTLSNRNQAGKSTALQSHHR SNSKDIQNLSVGLPRADEGLPANESFLNGNLAGASLSPLHTKTYQASSQPGSTSKDL TNNNIPHLLSPKEAKSKTEFDFNIDPKPSEGPGTKYLKSNSRSQQNRHSFMESSQSKA GTLQPNEKQSRHSYIDTIPQSSRSPSYRTKAKSHGALSDSKSVSNLSEARAQIAEPSTS RYFPSSCLDLNSPTSPTPTRHSDTRTLLSPSGRNNRNEGTLDSRRTTTRHSKTMEELK LPEHMDSSHSHSLSAPHESFSYGLGYTSPFSSQQRPHRHSMYVTRDKVRAKGLDGSL SIGQGMAARANSLQLLSPQPGEQLPPEMTVARSSVKETSREGTSSFHTRQKSEGGVY HDPHSDDGTAPKENRHLYNDPVPRRVGSFYRVPSPRPDNSFHENNVSTRVSSLPSES SSGTNHSKRQPAFDPWKSPENISHSEQLKEKEKQGFFRSMKKKKKKSQTTDSTNGE NPSIKKSLFPLFNSKNHLKHSSSLKKLPVVTPPMVPNSDSPDLLTLQKSIHSASTPSSR PKEWRPEKISDLQTQSQPLKSLRKLLHLSSASNHPASSDPRFQPLTAQQTKNSFSEIRI HPLSQASGGSSNIRQEPAPKGRPALQLPGQMDPGWHVSSVTRSATEGPSYSEQLGAK SGPNGHPYNRTNRSRMPNLNDLKETAL] (SEQ ID NO: 6), wherein the hCDKL5-coding sequence comprises a nucleic acid sequence of SEQ ID NO: 24 or a sequence which is at least about 95% identical to SEQ ID NO: 24 and encodes the protein of SEQ ID NO: 6. In certain embodiments, the hCDKL5-coding sequence encodes a hCDKL5-3GS protein comprising an amino acid sequence of [MKIPNIGNVMNKFEILGVVGEGAYGVVLKCRHKETHEIVAIKKFKDSEENEEVKET TLRELKMLRTLKQENIVELKEAFRRRGKLYLVFEYVEKNMLELLEEMPNGVPPEKV KSYIYQLIKAIHWCHKNDIVHRDIKPENLLISHNDVLKLCDFGFARNLSEGNNANYT EYVATRWYRSPELLLGAPYGKSVDMWSVGCILGELSDGQPLFPGESEIDQLFTIQKV LGPLPSEQMKLFYSNPRFHGLRFPAVNHPQSLERRYLGILNSVLLDLMKNLLKLDPA DRYLTEQCLNHPTFQTQRLLDRSPSRNQAGKSTALQSHHRSNSKDIQNLSVGLPRAD EGLPANESFLNGNLAGASLSPLHTKTYQASSQPGSTSKDLTNNNIPHLLSPKEAKSKT EFDFNIDPKPSEGPGTKYLKSNSRSQQNRHSFMESSQSKAGTLQPNEKQSRHSYIDTI PQSSRSPSYRTKAKSHGALSDSKSVSNLSEARAQIAEPSTSRYFPSSCLDLNSPTSPTP TRHSDTRTLLSPSGRNNRNEGTLDSRRTTTRHSKTMEELKLPEHMDSSHSHSLSAPH ESFSYGLGYTSPFSSQQRPHRHSMYVTRDKVRAKGLDGSLSIGQGMAARANSLQLL SPQPGEQLPPEMTVARSSVKETSREGTSSFHTRQKSEGGVYHDPHSDDGTAPKENRH LYNDPVPRRVGSFYRVPSPRPDNSFHENNVSTRVSSLPSESSSGTNHSKRQPAFDPW KSPENISHSEQLKEKEKQGFFRSMKKKKKKSQTVPNSDSPDLLTLQKSIHSASTPSSR PKEWRPEKISDLQTQSQPLKSLRKLLHLSSASNHPASSDPRFQPLTAQQTKNSFSEIRI HPLSQASGGSSNIRQEPAPKGRPALQLPGQMDPGWHVSSVTRSATEGPSYSEQLGAK SGPNGHPYNRTNRSRMPNLNDLKETAL] (SEQ ID NO: 8), wherein the hCDKL5-coding sequence comprises a nucleic acid sequence of SEQ ID NO: 25 or which is at least about 95% identical to SEQ ID NO: 25 and encodes the protein of SEQ ID NO: 8. In certain embodiments, the hCDKL5-coding sequence encodes a hCDKL5-4GS protein comprising an amino acid sequence of [MKIPNIGNVMNKFEILGVVGEGAYGVVLKCRHKETHEIVAIKKFKDSEENEEVKET TLRELKMLRTLKQENIVELKEAFRRRGKLYLVFEYVEKNMLELLEEMPNGVPPEKV KSYIYQLIKAIHWCHKNDIVHRDIKPENLLISHNDVLKLCDFGFARNLSEGNNANYT EYVATRWYRSPELLLGAPYGKSVDMWSVGCILGELSDGQPLFPGESEIDQLFTIQKV LGPLPSEQMKLFYSNPRFHGLRFPAVNHPQSLERRYLGILNSVLLDLMKNLLKLDPA DRYLTEQCLNHPTFQTQRLLDRSPSRNQAGKSTALQSHHRSNSKDIQNLSVGLPRAD EGLPANESFLNGNLAGASLSPLHTKTYQASSQPGSTSKDLTNNNIPHLLSPKEAKSKT EFDFNIDPKPSEGPGTKYLKSNSRSQQNRHSFMESSQSKAGTLQPNEKQSRHSYIDTI PQSSRSPSYRTKAKSHGALSDSKSVSNLSEARAQIAEPSTSRYFPSSCLDLNSPTSPTP TRHSDTRTLLSPSGRNNRNEGTLDSRRTTTRHSKTMEELKLPEHMDSSHSHSLSAPH ESFSYGLGYTSPFSSQQRPHRHSMYVTRDKVRAKGLDGSLSIGQGMAARANSLQLL SPQPGEQLPPEMTVARSSVKETSREGTSSFHTRQKSEGGVYHDPHSDDGTAPKENRH LYNDPVPRRVGSFYRVPSPRPDNSFHENNVSTRVSSLPSESSSGTNHSKRQPAFDPW KSPENISHSEQLKEKEKQGFFRSMKKKKKKSQTTDSTNGENPSIKKSLFPLFNSKNHL KHSSSLKKLPVVTPPMVPNSDSPDLLTLQKSIHSASTPSSRPKEWRPEKISDLQTQSQP LKSLRKLLHLSSASNHPASSDPRFQPLTAQQTKNSFSEIRIHPLSQASGGSSNIRQEPA PKGRPALQLPGQMDPGWHVSSVTRSATEGPSYSEQLGAKSGPNGHPYNRTNRSRMP NLNDLKETAL] (SEQ ID NO: 10), wherein the hCDKL5-coding sequence comprises a nucleic acid sequence of SEQ ID NO: 26 or which is at least about 95% identical to SEQ ID NO: 26 and encodes the protein of SEQ ID NO: 10.

In certain embodiments, the vector is a viral vector selected from a recombinant parvovirus, a recombinant lentivirus, a recombinant retrovirus, or a recombinant adenovirus; or a non-viral vector selected from naked DNA, naked RNA, an inorganic particle, a lipid particle, a polymer-based vector, or a chitosan-based formulation. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

### V. COMPOSITIONS

Provided herein is a composition comprising an rAAV or a vector as described herein and an aqueous suspension media. In certain embodiments, the suspension is formulated for intravenous delivery, intrathecal administration, or intracerebroventricular administration.

Provided herein are compositions containing at least one rAAV stock and an optional carrier, excipient and/or preservative. As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In certain embodiments, the composition containing the rAAV.hCDKL5 is delivered at a pH in the range of 6.8 to 8, or 7.2 to 7.8, or 7.5 to 8. For intrathecal delivery, a pH above 7.5 may be desired, e.g., 7.5 to 8, or 7.8.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2.

In another embodiment, the formulation may contain a buffered saline aqueous solution comprising 1 mM Sodium Phosphate (Na₃PO₄), 150 mM sodium chloride (NaCl), 3 mM potassium chloride (KCl), 1.4 mM calcium chloride (CaCl₂)), 0.8 mM magnesium chloride (MgCl₂), and 0.001% poloxamer (e.g., Kolliphor®) 188, pH 7.2. See, e.g., harvardapparatus.com/harvard-apparatus-perfusion-fluid.html. In certain embodiments, Harvard's buffer is preferred due to better pH stability observed with Harvard's buffer.

In certain embodiments, the formulation buffer is artificial CSF with Pluronic F68. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In certain embodiments, the ommaya reservoir is used for delivery. In one example, the composition is formulated for intrathecal delivery. In one example, the composition is formulated for intravenous (iv) delivery.

### VI. USES

Provided herein is a method of treating CDD, comprising administrating an effective amount of an rAAV or a vector as described herein to a subject in need thereof.

In certain embodiments, an “effective amount” herein is the amount which achieves amelioration of CDD symptoms and/or delayed CDD progression.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., brain, CSF, the liver (optionally via the hepatic artery), lung, heart, eye, kidney), oral, inhalation, intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired.

Dosages of the viral vector (for example, rAAV) depend primarily on factors such as the condition being treated, the age, weight and health of the patient, and can thus vary among patients. For example, a therapeutically effective human dosage of the viral vector is generally in the range of from about 25 to about 1000 microliters to about 100 mL of solution containing concentrations of from about 1×10⁹ to 1×10¹⁶ vector genome copies. In certain embodiments, a volume of about 1 mL to about 15 mL, or about 2.5 mL to about 10 mL, or about 5 mL suspension is delivered. In certain embodiments, a volume of about 1, about 2, about 3, about 4, about 5, about 6, about 7, about 8, about 9, about 10, about 11, about 12, about 13, about 14, or about 15 mL suspension is delivered. In certain embodiments, a dose of about 8.9×10¹² to 2.7×10¹¹ GC total is administered in this volume. In certain embodiments, a dose of about 1.1×10¹⁰ GC/g brain mass to about 3.3×10¹¹ GC/g brain mass is administered in this volume. In certain embodiments, a dose of about 3.0×10⁹, about 4.0×10⁹, about 5.0×10⁹, about 6.0×10⁹, about 7.0×10⁹, about 8.0×10⁹, about 9.0×10⁹, about 1.0×10¹⁰, about 1.1×10¹⁰, about 1.5×10¹⁰, about 2.0×10¹⁰, about 2.5×10¹⁰, about 3.0×10¹⁰, about 3.3×10¹⁰, about 3.5×10¹⁰, about 4.0×10¹⁰, about 4.5×10¹⁰, about 5.0×10¹⁰, about 5.5×10¹⁰, about 6.0×10¹⁰, about 6.5×10¹⁰, about 7.0×10¹⁰, about 7.5×10¹⁰, about 8.0×10¹⁰, about 8.5×10¹⁰, about 9.0×10¹⁰, about 9.5×10¹⁰, about 1.0×10¹¹ about 1.1×10¹¹, about 1.5×10¹¹, about 2.0×10¹¹, about 2.5×10¹¹, about 3.0×10¹¹, about 3.3×10¹¹, about 3.5×10¹¹, about 4.0×10¹¹, about 4.5×10¹¹, about 5.0×10¹¹, about 5.5×10¹¹ about 6.0×10¹¹, about 6.5×10¹¹, about 7.0×10¹¹, about 7.5×10¹, about 8.0×10¹¹, about 8.5×10¹¹, about 9.0×10¹¹ GC per gram brain mass is administered in this volume.

The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an subject) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10′¹⁵, 5×10¹⁵, 6×10¹⁵, 7×10¹⁵, 8×10¹⁵, or 9×10¹⁵ GC per dose including all integers or fractional amounts within the range.

In one embodiment, for human application the dose can range from 1×10¹⁰ to about 1×10¹⁵ GC per kg body weight including all integers or fractional amounts within the range. In one embodiment, the effective amount of the vector is about 1×10⁹, 2×10⁹, 3×10⁹, 4×10⁹, 5×10⁹, 6×10⁹, 7×10⁹, 8×10⁹, or 9×10⁹ GC per kg body weight including all integers or fractional amounts within the range. In another embodiment, the effective amount of the vector is about 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per kg body weight including all integers or fractional amounts within the range. In another embodiment, the effective amount of the vector is about 1×10¹¹, 2×10¹¹, 3×10¹¹, 4×10¹¹, 5×10¹¹, 6×10¹¹, 7×10¹¹, 8×10¹¹, or 9×10¹¹ GC per kg body weight including all integers or fractional amounts within the range. In another embodiment, the effective amount of the vector is about 1×10¹², 2×10¹², 3×10¹², 4×10¹², 5×10¹², 6×10¹², 7×10¹², 8×10¹², or 9×10¹² GC per kg body weight including all integers or fractional amounts within the range. In another embodiment, the effective amount of the vector is about 1×10¹³, 2×10¹³, 3×10¹³, 4×10¹³, 5×10¹³, 6×10¹³, 7×10¹³, 8×10¹³, or 9×10¹³ GC per kg body weight including all integers or fractional amounts within the range. In another embodiment, the effective amount of the vector is about 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per kg body weight including all integers or fractional amounts within the range. In another embodiment, the effective amount of the vector is about 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per kg body weight including all integers or fractional amounts within the range.

In certain embodiments, the dose is scaled by brain mass, which provides an approximation of the size of the CSF compartment. Without wishing to be bound by theory, dose conversions are based on a brain mass of 0.15 g for a neonatal mouse (Gu et al., 2012), 90 g for a juvenile NHP (Herndon et al., 1998), 610 g from a 6-8-month infant, 780 g for an 8-12-month infant, and 960 g for a >12-month infant (Dekaban, 1978). Estimated brain weights for each age range for human infants were derived from the male and female brain weights presented in (Dekaban, 1978) by assuming an approximately linear increase in brain weight between that of newborns (370 g) and infants aged 4-8 months, resulting in a mean estimated brain weight of 488 g for ≥1-≤4 month old infants. The value of 610 g corresponds to the average brain weight for males and females aged 4-8 months (Dekaban, 1978). An example of dose scaling from neonatal mice, juvenile NHPs, and equivalent human doses are presented in table immediately below. The administration volume may also be scaled from NHPs to humans based on the estimated volumes for cerebral CSF (Matsumae et al., 1996) and spinal CSF (Rochette et al., 2016).

| | | Total Dose (GC) | | | | |
|---|---|---|---|---|---|---|
| | Scaled Dose | | | Human Infant | | |
| | (GC/g Brain) | Neonatal | Juvenile | ≥6 Months-<8 | ≥8 Months-<12 | |
| | All Species | Mouse | NHP^{a} | Months | Months | ≥12 Months |
| Low | 1.1 × 10¹⁰ | 2.2 × 10⁹ | — | 6.8 × 10¹² | 8.7 × 10¹² | 1.1 × 10¹³ |
| Dose | | | | | | |
| Mid- | 3.3 × 10¹⁰ | 6.7 × 10⁹ | 3.0 × 10¹² | 2.1 × 10¹³ | 2.6 × 10¹³ | 3.2 × 10¹³ |
| Dose #1 | | | | | | |
| Mid- | 1.1 × 10¹¹ | 2.2 × 10¹⁰ | 1.0 × 10¹³ | 6.8 × 10¹³ | 8.7 × 10¹³ | 1.0 × 10¹⁴ |
| Dose #2 | | | | | | |
| High | 3.3 × 10¹¹ | 6.7 × 10¹⁰ | 3.0 × 10¹³ | 2.0 × 10¹⁴ | 2.6 × 10¹⁴ | 3.2 × 10¹⁴ |
| Dose | | | | | | |

In one embodiment, for human application the dose can range from 1×10¹⁰ to about 1×10¹⁵ GC per gram (g) brain mass including all integers or fractional amounts within the range. In one embodiment, the effective amount of the vector is about 1×10⁹, 2×10⁹, 3×10⁹, 4×10⁹, 5×10⁹, 6×10⁹, 7×10⁹, 8×10⁹, or 9×10⁹ GC per gram (g) brain mass including all integers or fractional amounts within the range. In another embodiment, the effective amount of the vector is about 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per gram (g) brain mass including all integers or fractional amounts within the range. In another embodiment, the effective amount of the vector is about 1×10¹¹, 2×10¹¹, 3×10¹¹, 4×10¹¹, 5×10¹¹, 6×10¹¹, 7×10¹¹, 8×10¹¹, or 9×10¹¹ GC per gram (g) brain mass including all integers or fractional amounts within the range. In another embodiment, the effective amount of the vector is about 1×10¹², 2×10¹², 3×10¹², 4×10¹², 5×10¹², 6×10¹², 7×10¹², 8×10¹², or 9×10¹² GC per gram (g) brain mass including all integers or fractional amounts within the range. In another embodiment, the effective amount of the vector is about 1×10¹³, 2×10¹³, 3×10¹³, 4×10¹³, 5×10¹³, 6×10¹³, 7×10¹³, 8×10¹³, or 9×10¹³ GC per gram (g) brain mass including all integers or fractional amounts within the range. In another embodiment, the effective amount of the vector is about 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per gram (g) brain mass including all integers or fractional amounts within the range. In another embodiment, the effective amount of the vector is about 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per gram (g) brain mass including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 1×10¹⁰ GC/g brain mass to about 3×10¹¹ GC/g brain mass. In certain embodiments, the dose may be in the range of about 1×10¹⁰ GC/g brain mass to about 2.5×10¹¹ GC/g brain mass. In certain embodiments, the dose may be in the range of about 5×10¹⁰ GC/g brain mass.

In one embodiment, the viral constructs may be delivered in doses of from at least about least 1×10⁹ GC to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage may be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other route of delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna. In certain embodiment, a rAAV, vector, or composition as described herein is administrated to a subject in need via the intrathecal administration. In certain embodiments, the intrathecal administration is performed as described in US Patent Publication No. 2018-0339065 A1, published Nov. 29, 2019, which is incorporated herein by reference in its entirety.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In certain embodiments, treatment of the composition described herein has minimal to mild asymptomatic degeneration of DRG sensory neurons in animals and/or in human patients, well-tolerated with respect to sensory nerve toxicity and subclinical sensory neuron lesions.

### VII. APPARATUS AND METHOD FOR DELIVERY OF A PHARMACEUTICAL COMPOSITION INTO CEREBROSPINAL FLUID

In one aspect, the vectors provided herein may be administered intrathecally via the method and/or the device provided in this section and described in WO 2018/160582, which is incorporated by reference herein. Alternatively, other devices and methods may be selected. In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis. In certain embodiments, vectors and/or compositions thereof as described herein are administered via computed tomography- (CT-) guided sub-occipital injection into the cisterna magna (intra-cisterna magna [ICM]). In certain embodiments, the Ommaya Reservoir is used for delivery of a pharmaceutical composition. In certain embodiments, the apparatus is described in US Patent Publication No. 2018-0339065 A1, published Nov. 29, 2019, which is incorporated herein by reference in its entirety.

In certain embodiments, the AAVhu68.UbC.hCDKL5-1co.miR183.rBG is administered as a single dose to hospitalized participants on Day 1 via CT-guided sub-occipital injection into the cisterna magna. On Day 1, a syringe containing AAVhu68.UbC.hCDKL5-1co.miR183.rBG (final volume≤5 ml) at the appropriate titer is prepared by the Investigational Pharmacy associated with the study and delivered to the procedure room. Prior to study drug administration, the participant is anesthetized, intubated, and the injection site is prepped and draped using sterile technique. A lumbar puncture is performed to remove a predetermined volume of CSF, after which iodinated contrast is IT injected to aid in visualization of relevant anatomy of the cisterna magna. IV contrast may be administered prior to or during needle insertion as an alternative to the IT contrast. The decision to use IV or IT contrast is at the discretion of the interventionalist performing the procedure. A spinal needle (22-25 G) is advanced into the cisterna magna under fluoroscopic guidance. A larger introducer needle may be used to assist with needle placement. After confirmation of needle placement, the extension set is attached to the spinal needle and allowed to fill with CSF. At the discretion of the interventionalist, a syringe containing contrast material may be connected to the extension set and a small amount injected to confirm needle placement in the cisterna magna. After the needle placement is confirmed, the syringe containing rAAV is connected to the extension set. The syringe contents are slowly injected over 1-2 minutes, delivering a volume of ≤5.0 ml.

### VIII. CDD

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human. In certain embodiment, the subject of these methods and compositions is diagnosed with CDD and/or with symptoms of CDD.

The methods and compositions may be used for treatment of any of the stages of CDD. In certain embodiments, the patient is about 1, 2, 3, 4, 5, 6, 7, 8, 9, 10, 11 month(s) old, or about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old. In certain embodiments, the patient is a toddler, e.g., 18 months to 3 years of age. In certain embodiments, the patient is from 3 years to 6 years of age, from 3 years to 12 years of age, from 3 years to 18 years of age, from 3 years to 30 years of age. In certain embodiments, patients are older than 18 years of age.

Symptoms in CDD include seizures that usually begin within the first 3 months of life, and can appear as early as the first week after birth. The types of seizures change with age, and may follow a predictable pattern. The most common types are generalized tonic-clonic seizures, which involve a loss of consciousness, muscle rigidity, and convulsions; tonic seizures, which are characterized by abnormal muscle contractions; and epileptic spasms, which involve short episodes of muscle jerks. Seizures occur daily in most people with CDKL5 deficiency disorder, although they can have periods when they are seizure-free. Seizures in CDKL5 deficiency disorder are typically resistant to treatment.

Development is impaired in children with CDKL5 deficiency disorder. Most have severe intellectual disability and little or no speech. The development of gross motor skills, such as sitting, standing, and walking, is delayed or not achieved. About one-third of affected individuals are able to walk independently. Fine motor skills, such as picking up small objects with the fingers, are also impaired; about half of affected individuals have purposeful use of their hands. Most people with this condition have vision problems (cortical visual impairment).

Other common features of CDKL5 deficiency disorder include repetitive hand movements (stereotypies), such as clapping, hand licking, and hand sucking; teeth grinding (bruxism); disrupted sleep; feeding difficulties; and gastrointestinal problems including constipation and backflow of acidic stomach contents into the esophagus (gastroesophageal reflux). Some affected individuals have episodes of irregular breathing. Distinctive facial features in some people with CDKL5 deficiency disorder include a high and broad forehead, large and deep-set eyes, a well-defined space between the nose and upper lip (philtrum), full lips, widely spaced teeth, and a high roof of the mouth (palate). Other physical differences can also occur, such as an unusually small head size (microcephaly), side-to-side curvature of the spine (scoliosis), and tapered fingers.

As described above, the terms “increase” “decrease” “reduce” “ameliorate” “improve” “delay” or any grammatical variation thereof, or any similar terms indication a change, means a variation of about 5 fold, about 2 fold, about 1 fold, about 90%, about 80%, about 70%, about 60%, about 50%, about 40%, about 30%, about 20%, about 10%, about 5% compared to the corresponding reference (e.g., untreated control or a subject in normal condition without CDD), unless otherwise specified.

In certain embodiments, the patient receives medications controlling some signs and symptoms associated with the CDD, such as seizures, muscle stiffness, or problems with breathing, sleep, the gastrointestinal tract or the heart.

In certain embodiments, a diuretic agent may be used in co-therapy in a subject in need thereof. Diuretic agent used may be acetazolamine (Diamox) or other suitable diuretics. In some embodiments, the diuretic agent is administered at the time of gene therapy administration. In some embodiments, the diuretic agent is administered prior to gene therapy administration. In some, embodiments the diuretic agent is administered where the volume of injection is 3 mL.

In certain embodiments, co-therapies may be utilized, which comprise co-administration of Cdkl5-isoform 1, isoform 2, isoform 3, and/or isoform 4-expressing vectors, or various two- or three-way combinations thereof. Optionally, co-therapy may further comprise administration of another active agent. In certain embodiments, co-therapy may comprise enzyme replacement therapy.

Optionally, an immunosuppressive co-therapy may be used in a subject in need. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 3, 4, 5, 6, 7, or more days prior to or after the gene therapy administration. Such immunosuppressive therapy may involve administration of one, two or more drugs (e.g., glucocorticoids, prednelisone, micophenolate mofetil (MMF) and/or sirolimus (i.e., rapamycin)). Such immunosuppressive drugs may be administrated to a subject in need once, twice or for more times at the same dose or an adjusted dose. Such therapy may involve co-administration of two or more drugs, e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “expression” is used herein in its broadest meaning and comprises the production of RNA or of RNA and protein. With respect to RNA, the term “expression” or “translation” relates in particular to the production of peptides or proteins. Expression may be transient or may be stable.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

Throughout the specification, exponents are referred to using the term “e” followed by a numerical value (n). It will be understood with this refers to “×10ⁿ). For example, “3e9” is the same as 3×10 and “1e13” is the same as 1×10¹³.

As described above, the term “about” when used to modify a numerical value means a variation of ±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### IX. EXAMPLES

The following examples are illustrative only and are not intended to limit the present invention. The following table provides a list of certain abbreviations used in the following examples. Other abbreviations or meanings may be readily apparent to one of skill in the art.

| Abbreviations | Description |
|---|---|
| 6MWT | 6 Minute Walk Test |
| A | Absorbance |
| Ad | Adenovirus |
| Ad5 | Adenovirus Serotype 5 |
| AE | Adverse Events |
| AEX | Anion Exchange |
| AmpR | Ampicillin Resistance (gene) |
| ANOVA | Analysis of Variance |
| AUC | Analytical Ultracentrifugation |
| BA | Chicken β-Actin Promoter |
| BCA | Bicinchoninic Acid |
| BDS | Bulk Drug Substance |
| BMCB | Bacterial Master Cell Bank |
| bp | Base Pairs |
| BRF | Batch Record Form |
| BSC | Biological Safety Cabinet |
| BWCB | Bacterial Working Cell Bank |
| cap | Capsid (gene) |
| CB7 | Chicken β-Actin |
| CBC | Complete Blood Count |
| CBER | Center for Biologics Evaluation and Research |
| CE | Capillary Electrophoresis |
| CFU | Colony Forming Units |
| cGMP | Current Good Manufacturing Practice |
| CI | Chimeric Intron |
| CMC | Chemistry Manufacturing and Controls |
| CMO | Contract Manufacturing Organization |
| CMV | Cytomegalovirus |
| CMV IE | Cytomegalovirus Immediate-Early Enhancer |
| CNS | Central Nervous System |
| COA | Certificate of Analysis |
| CPE | Cytopathic Effects |
| CRO | Contract Research Organization |
| CSF | Cerebrospinal Fluid |
| CT | Computed Tomography |
| CTCAE | Common Terminology Criteria |
| ddPCR | Droplet Digital Polymerase Chain Reaction |
| DLS | Dynamic Light Scattering |
| DMEM | Dulbecco's Modified Eagle Medium |
| DMF | Drug Master File |
| DNA | Deoxyribonucleic Acid |
| DO | Dissolved Oxygen |
| DP | Drug Product |
| DRG | Dorsal Root Ganglia |
| DS | Drug Substance |
| DSMB | Data and Safety Monitoring Board |
| E1A | Early Region 1A (gene) |
| EC | Ethics Committee |
| ECG | Electrocardiogram |
| EDTA | Ethylenediaminetetraacetic Acid |
| ELISA | Enzyme-Linked Immunosorbent Assay |
| ELISpot | Enzyme-Linked Immunospot |
| EU | Endotoxin Units |
| F | Female |
| F/U | Follow-Up |
| FBS | Fetal Bovine Serum |
| FDA | Food and Drug Administration |
| FDP | Final Drug Product |
| FFB | Final Formulation Buffer |
| FIH | First-in-Human |
| FSS | Fatigue Severity Scale |
| FVC | Forced Vital Capacity |
| GC | Genome Copies |
| GFR | Glomerular Filtration Rate |
| GLP | Good Laboratory Practice |
| GMP | Good Manufacturing Practice |
| HCDNA | Host Cell Deoxyribonucleic Acid |
| HCP | Host Cell Protein |
| HCV | Hepatitis C Virus |
| HEK293 | Human Embryonic Kidney 293 |
| HHD | Handheld Dynamometry |
| HPLC | High-Performance Liquid Chromatography |
| hSyn | Human Synapsin |
| ICH | International Conference on Harmonization |
| ICF | Informed Consent Form |
| ICM | Intra-Cisterna Magna |
| ICP | Intracranial Pressure |
| ICV | Intracerebroventricular |
| ID | Identification |
| IFN-γ | Interferon Gamma |
| IHC | Immunohistochemistry |
| IND | Investigational New Drug |
| INR | International Normalized Ratio |
| IOPI | Iowa Oral Performance Index |
| IP | Investigational Product |
| IRB | Institutional Review Board |
| IT | Intrathecally |
| ITFFB | Intrathecal Final Formulation Buffer |
| ITR | Inverted Terminal Repeat |
| IU | Infectious Unit |
| IV | Intravenous |
| KanR | Kanamycin Resistance (gene) |
| KO | Knockout |
| L | Left |
| LFTs | Liver Function Tests |
| LLOQ | Lower Limit of Quantification |
| LOD | Limit of Detection |
| LOQ | Limit of Quantification |
| LP | Lumbar Puncture |
| LTFU | Long-Term Follow-Up |
| M | Male |
| MBR | Master Batch Record |
| MCB | Master Cell Bank |
| MED | Minimum Effective Dose |
| miRNA | MicroRNA |
| MOI | Multiplicity of Infection |
| MRC-5 | Medical Research Council Cell Strain 5 |
| MRI | Magnetic Resonance Imaging |
| mRNA | Messenger Ribonucleic Acid |
| MS | Mass Spectrometry |
| MTD | Maximum Tolerated Dose |
| N | Number of Subjects or Animals |
| N/A | Not Applicable |
| NAbs | Neutralizing Antibodies |
| NGS | Next-Generation Sequencing |
| NHP | Non-Human Primate |
| NSAID | Non-Steroidal Anti-Inflammatory Drug |
| NT | Not Tested |
| OL | Open-Label |
| PBMCs | Peripheral Blood Mononuclear Cells |
| PBS | Phosphate-Buffered Saline |
| PDMA | Pharmaceuticals and Medical Devices Agency |
| PEI | Polyethylenimine |
| PES | Polyethersulfone |
| Ph. Eur. | European Pharmacopoeia |
| PI | Principal Investigator |
| PND | Postnatal Day |
| PNS | Peripheral Nervous System |
| POC | Proof-of-Concept |
| PolyA | Polyadenylation |
| QC | Quality Control |
| qPCR | Quantitative Polymerase Chain Reaction |
| R | Right |
| rBG | Rabbit β-Globin |
| rDNA | Ribosomal Deoxyribonucleic Acid |
| rep | Replicase (gene) |
| RNA | Ribonucleic Acid |
| ROA | Route of Administration |
| SAE | Serious Adverse Event |
| SDS | Sodium Dodecyl Sulfate |
| SDS-PAGE | Sodium Dodecyl Sulfate Polyacrylamide |
| | Gel Electrophoresis |
| SF-36 | Short Form 36 |
| SOC | Standard of Care |
| SOP | Standard Operating Procedure |
| SRT | Safety Review Trigger |
| ssDNA | Single-Stranded Deoxyribonucleic Acid |
| SV40 | Simian Virus 40 |
| TBD | To Be Determined |
| TCID₅₀ | 50% Tissue Culture Infective Dose |
| TE | Tris-EDTA |
| TFF | Tangential Flow Filtration |
| TMA | Thrombotic Microangiopathy |
| TMV | Thigh Muscle Volume |
| TUG | Timed Up and Go |
| ULN | Upper Limit of Normal |
| UPLC | Ultra-Performance Liquid Chromatography |
| US | United States |
| USP | United States Pharmacopeia |
| UV | Ultraviolet |
| WBC | White Blood Cell |
| WCB | Working Cell Bank |
| WPRE | Woodchuck Hepatitis Virus Post-Transcriptional |
| | Regulatory Element |
| WT | Wild Type |

### Example 1. Mouse Model and Preliminary Study

CDD is caused by a splice site mutation of the CDKL5 gene, which encodes a phosphorylated serine/threonine protein kinase that is highly expressed in the brain. This mutation results in the disruption of CDKL5 kinase activity, leading to a reduction in signal transduction of AKT-mTOR and other related pathways, along with deficits in neural circuit communication.

Several models for CDD have been developed and may selected for use in evaluating therapeutic effect. These following models are null for CDKL5 expression: g., a Cdkl5-ko mouse having a deletion in exon 6 (Δ exon 6), Wang et al, Proceedings of the National Academy of Sciences December 2012, 109 (52) 21516-21521; DOI: 10.1073/pnas.1216988110; a Cdkl5-ko mouse having a deletion in exon 4 (Δexon 4) (see, Amendola et al. (2014) Mapping Pathological Phenotypes in a Mouse Model of CDKL5 Disorder. PLoS ONE 9(5): e91613. doi.org/10.1371/joumal.pone.0091613 (2014); a Cdkl5 (R59X knock-in) model (available form Jackson Laboratory; see, Tang et al (2019), Tang S, et al. Altered NMDAR signaling underlies autistic-like features in mouse models of CDKL5 deficiency disorder. Nat Commun. 2019; 10(1):2655. Published 2019 Jun. 14. doi:10.1038/s41467-019-10689-w, and a Cdkl5 (D471fs) model (Rodney Samaco, Baylor College of Medicine, Houston, TX).

CDD mice, harboring a truncated CDKL5 gene lacking exon 6 that corresponds to exon 7 of human CDKL5, exhibits a phenotype from 10 to 11 weeks with clinically relevant symptoms observed in humans with CDD, including motor coordination impairment, poor cognition function and social behavior as well as AKT and mTOR signaling). Of note, hindlimb clasping and autistic-like and non-social behavior was observed in the CDD mice, indicating the development of neurodegeneration mice within 2 or 2.5 months.

The current CDD mouse model, however, does not exhibit any forms of spontaneous or refractory epilepsy, as opposed to the disease phenotype attributed to the pathogenic variants of CDKL5 in humans. The absence of this phenotype in CDD mice may be attributed to the age of animals, study duration, and increased seizure resistance conferred by genetic background of C57BL/6 mice (Wang et al., 2012 and Amendola et al., 2014). Based on this observation, it is not possible to observe the seizure phenotype in neonatal CDD mice. In addition, although CDD mainly affects heterozygous female individuals and, consequently, the proposed clinical trial patient population includes heterozygous females, signs of CDD development are evident in heterozygous male Cdkl5^{KO/Y} mice but not in heterozygous female Cdkl5^{KO/+} mice. Therefore, heterozygous male Cdkl5^{KO/Y} mice were used in the current nonclinical program as they best represent the disease phenotype of CDD in humans, which also tends to be more severe in male patients. Several other mouse models for CDKL5 deficiency have been reported as described herein. These mice all lack CDKL5 protein expression, exhibit a normal life span and display a broad spectrum of mild behavior abnormalities. CDKL5 expression is developmentally regulated in mouse brain. Accordingly, CDKL5 is found highly expressed throughout the mouse brain, including the cortex and hippocampus, suggesting that Cdkl5-deficiency in these areas may relate to the observed phenotypes in Cdkl5 deficient mice. The most pronounced phenotypes have been observed in male Cdkl5-ko mice, manifesting at about 11 weeks of age, and most studies involving neurobehavior phenotypes have been conducted with male Cdkl5-ko mice.

In this study we show that restoring CDKL5 expression in the CNS of three CDD mouse models (Cdkl5-ko (exon 6), Cdkl5(R59X). Cdkl5(D471fs)) significantly improves disease symptoms. We developed an AAV gene therapy vector comprised of the AAVhu68 capsid, and an expression cassette with the human synapsin promoter and a engineered human CDKL5 transgene. When the AAV-CDKL5 vector was administered to Cdkl5 knock-out mice via neonatal injection into the lateral brain ventricle we detected robust expression in up to 50% of neurons throughout the brain. AAV administration and human CDKL5 expression were tolerated well. The human CDKL5 was mostly localized to the cytoplasm; protein expression and kinase activity persisted for over 4 months. We subjected cohorts of treated Cdkl5-ko mice to a battery of neurobehavior tests and found significant improvement from untreated Cdkl5-ko phenotype towards behavior outcomes observed in wildtype mice. We then repeated the same study in 2 different CDD mouse models that carry patient-derived frameshift mutations (Cdkl5(R59X) and CDKL5(D471fs) models) instead of the gene knock-out. We obtained very similar results, thus reiterating the curative benefit of our CDKL5 gene therapy vector.

CDKL5 is expressed as at least four different isoforms in the human brain. We generated similar AAV gene therapy vectors for the three alternatively spliced isoforms which were all found to express and exhibit kinase activity in transduced mouse brains.

To test for expression of our AAV-CDKL5 gene therapy vector in a larger animal we conducted a study with rhesus macaques. Through infusion into the cerebrospinal fluid via the cisterna magna, we have been able to achieve vector distribution throughout the entire brain at 0.1 to 1 vector copies per diploid genome. Much higher vector transduction was observed in dorsal root ganglia (DRG). Accordingly, in situ hybridization with probes specific for the human CDKL5 sequence showed abundant expression in DRG neurons but much sparser expression in cortical grey matter neurons. CDKL5 administration and expression was generally tolerated well in all six rhesus macaques for 60 days, however, we noticed mild axonopathy of spinal white matter tracts.

In summary, we show promising evidence that CDKL5 gene therapy provides a lasting curative benefit to model mice and is tolerated well in non-human primates. Further optimization of this approach may eventually offer an option for clinical intervention in children affected by CDD.

### Materials and Methods

Plasmids. The amino acid sequences for four CDKL5 (cyclin-dependent kinase-like 5, Uniprot ID 076039) that express in human brain¹ were reverse translated into a DNA sequence. The coding sequence was further engineered, e.g., by considering codon frequencies found in the human genome, cryptic RNA splice sites and alternative reading frames. The engineered sequence was cloned into an AAV expression plasmid under the control of the human synapsin promoter². The coding sequence is preceded by a Kozak sequence, followed by an WPRE enhancer cassette (Woodchuck Hepatitis Virus Posttranscriptional Regulatory Element), the SV40 poly A sequence, and framed by AAV2 inverted terminal repeats (ITR). To suppress expression in dorsal root ganglia (DRG), in some experiments the above plasmid was modified to contain four repeats of the miR183 binding site (agtgaattctaccagtgccata) (SEQ ID NO: 11) directly after the CDKL5 coding sequence and before the WPRE sequence. AAV CDKL5 vector was produced using either as capsid PHP.B for mouse studies or AAVhu68⁴ or AAVrh91 for mouse and non-human primate studies.

Mouse Studies. All studies involving mice were approved by the University of Pennsylvania IACUC. We obtained Cdkl5-ko mice from Jackson Laboratories (B6.129(FVB)-Cdkl5tm1.1Joez/J, strain #021967) and crossed heterozygous female ko mice with wt C57B16 males to obtain the following genotypes of littermates: male hemizygous Cdkl5-ko (also referenced as KO mice or mice), male wt, female heterozygous Cdkl5-ko and female wt. Mice received AAV Cdkl5 vector (dose range 1×10¹¹ GC to 5×10¹¹ GC) or vehicle control (sterile phospho-buffered saline) at 18-21 days of age by retro-orbital injections in a total volume of 100 μl. Alternatively, mice were injected at day of birth intracerebroventricular with a dose of 1×10¹⁰ GC to 5×10¹⁰ GC in a total volume of 2 μl. Mice were housed mixed in regard of genotype and injection product, weighed and observed at least twice a week, and aged to 11 weeks for males or 14 week for females when they were subjected to behavior testing. We did not observer treatment-related morbidity.

Western Blotting and Tissue Staining. After euthanasia, one cortical hemisphere was flash frozen and subsequently protein lysates were generated using RIPA buffer. Western blotting was carried out with antibodies against human Cdkl5 (S957D, University of Dundee, UK), EB2 (ab45767, Abcam) or EB2 phospo222 (pab01032-P, Covalab, UK). The other brain half was fixed overnight in formalin, embedded in paraffin and thin sections were processed for immunofluorescence staining with the same CDKL5 antibodies.

Behavior Testing. Mice underwent one test per day. Testing time of day, operator and environment was kept the same (60 dB white noise background and 1000 lumens incandescent indirect lighting). Mice were habituated in their home cages before each test for 30 minutes. For the Open Field Assay, a new cage with minimal amount of bedding was placed into an infrared beam array (MedAssociates, Inc.). A single mouse was added in the middle of the cage and the number of beam brakes over the next 30 minutes was automatically recorded, separated into beam brakes close to the ground (general activity) and 3 inches above ground (rearing activity). For the Elevated Zero Maze (EZM, Stoelting Co.), an elevated circular platform with two opposite enclosed quadrants and two open was used to allow uninterrupted exploration. A single mouse was place in the middle of an open quadrant and movement was video-recorded for 15 minutes. For the Y-Maze (Stoelting Co.), an enclosed platform containing three identical arms in the shape of a Y was used. A single mouse was placed in the arm closest to the operator and its movements were video-recorded for 5 minutes. For the Marble Burying Assay, a fresh cage was filled with 3 inches of AlphaDri bedding (Shepherd Specialty Papers) and gently compacted down. 12 solid-blue marbles were spaced equally onto the bedding and a single mouse placed in the middle of the cage. The number of marbles that were at least half covered by bedding was recorded after 30 minutes.

Data analysis. Data was graphed and analyzed using GraphPad Prims software. Video files were recorded in mp4 format at 20 fps and analyzed using EthoVision XT software (version 14, Noldus Information Technology). For the Nest Building Assay, mice were single-house overnight in a new cage, supplied with a standardized 2×2 inch nestlet squares (cotton based). After 24 hours, the quality of the nest was scored on a scale of 1-5 and any remaining untouched nestlet material was weighed.

Non-human primate experiments. All studies involving non-human primates were approved by the University of Pennsylvania IACUC and carried out according to USDA regulations. Non-human primates (NHPs) for the species Macaca mulatta (rhesus macaques) were obtained from Covance Research Products, Inc. Quarantine and animal husbandry was performed according to Gene Therapy Program SOPs. In the month before AAV vector administration and throughout the study body weight, temperature, respiratory rate and heart rate was periodically monitored, and blood and CSF samples obtained. Whole blood was used for cell counts and differentials, and a clinical blood chemistry panel. CSF samples were used for blood cell counts and differentials, and total protein quantification. For AAV vector delivery into the CSF via puncture of the cisterna magna, anesthetized macaques were placed on a procedure table in the lateral decubitus position with the head flexed forward. Using aseptic technique, a 21-27 gauge, 1-1.5 inch Quincke spinal needle (Becton Dickinson) was advanced into the sub-occipital space until the flow of CSF is observed. The needle will be directed at the wider superior gap of the cisterna magna to avoid blood contamination and potential brainstem injury. Correct placement of needle puncture will be verified via myelography, using a fluoroscope. 1 mL of CSF was collected for baseline analysis, prior to dosing. After CSF collection, a leur access extension catheter was connected to the spinal needle to facilitate dosing of 1 ml Iohexol (Trade Name: Omnipaque 180 mg/mL, General Electric Healthcare) contrast media. After verifying needle placement, a syringe containing the test article (volume equivalent to 1 mL plus the volume of syringe and linker dead space) was connected to the flexible linker and injected over 30±5 seconds. The needle was removed, and direct pressure applied to the puncture site.

AAVhu68.hSyn.Cdkl5-1co and AAVhu68.hSyn.Cdkl5-1co vector has injected at a dose of up to 3×10¹³ GC/NHP. At study days 0, 14, 18, 41 and the last study day, a neurological assessment was given to all macaques for detailed evaluation of neurological function. Briefly, evaluation included posture and gait assessment, cranial nerve assessment, proprioceptive assessment and spinal/nerve reflexes. At study day 56, macaques were euthanized, and gross postmortem examination and necropsy was performed. 25 major tissues were harvested from each macaque in duplicate for either snap freezing or fixation in formalin. DNA or RNA was purified from snap-frozen tissues and used for vector biodistribution or transgene expression analysis, respectively. For vector biodistribution, genome copies (GC) per total DNA weight were determined using a TaqMan qPCR assay with probes directed again the polyA region of the transgene cassette and an internal standard. To quantify transgene expression, total RNA was used to generate cDNA via first strand synthesis with polyT oligonucleotides, followed by TaqMan qPCR with probes specific for the transgene that did not cross-react with the endogenous rhesus CDKL5 sequence. For histopathological analysis, macaque tissue was embedded into paraffin and these sections were stained with H&E solution or CDKL5 antibodies, respectively. Same spinal cord sections were incubated with Luxol Fast Blue to stain myelin. All stained tissues sections were reviewed by a board-certified veterinary pathologist, and abnormal findings verified by peer review.

### REFERENCES

1. Hector, R. D. et al. Characterization of CDKL5 Transcript Isoforms in Human and Mouse. PloS one 11, e0157758, (2016).
2. Thiel, G., Greengard, P. & Sudhof, T. C. Characterization of tissue-specific transcription by the human synapsin I gene promoter. Proc Natl Acad Sci USA 88, 3431-3435, (1991).
3. Deverman, B. E. et al. Cre-dependent selection yields AAV variants for widespread gene transfer to the adult brain. Nat Biotechnol 34, 204-209, (2016).
4. Hinderer, C. et al. Severe Toxicity in Nonhuman Primates and Piglets Following High-Dose Intravenous Administration of an Adeno-Associated Virus Vector Expressing Human SMN. Human Gene Therapy 29, 285-298, (2018).

### Example 2: Gene Therapy AAV Vector

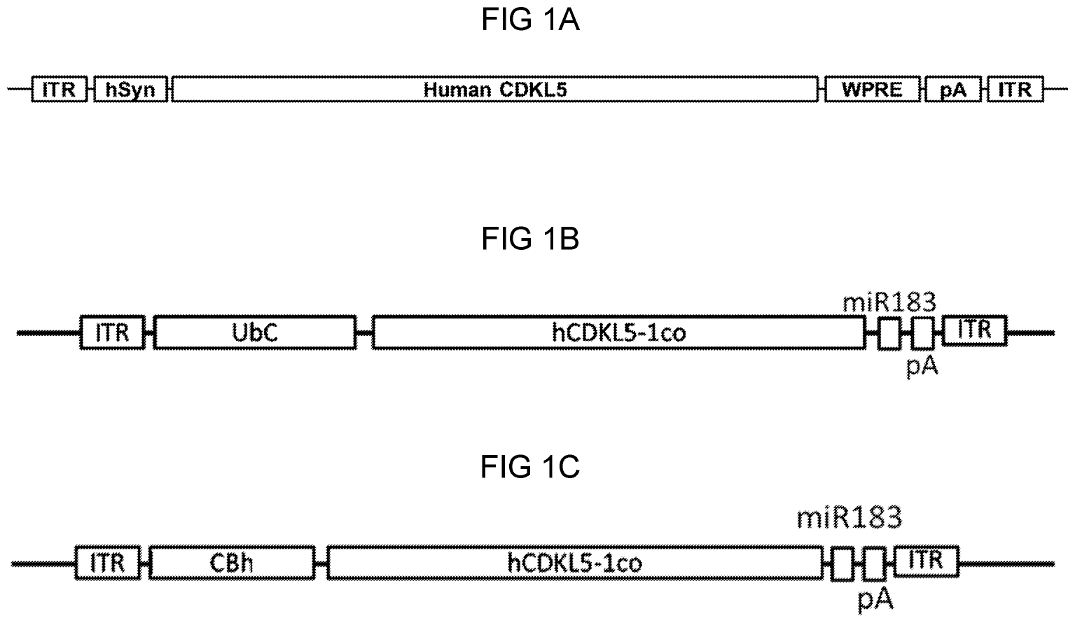

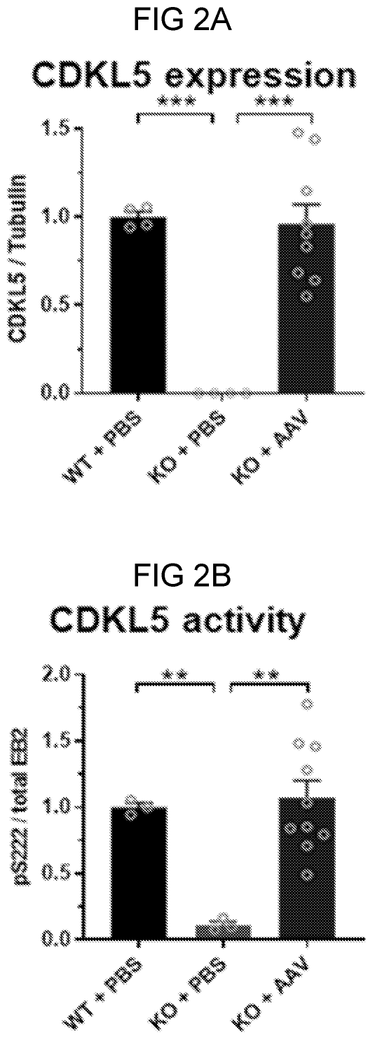

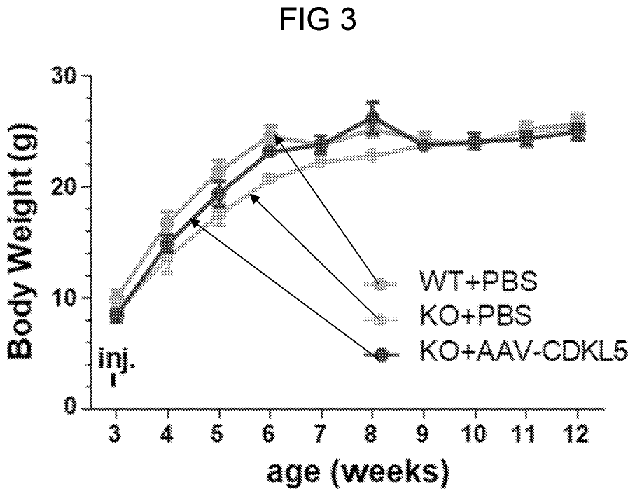

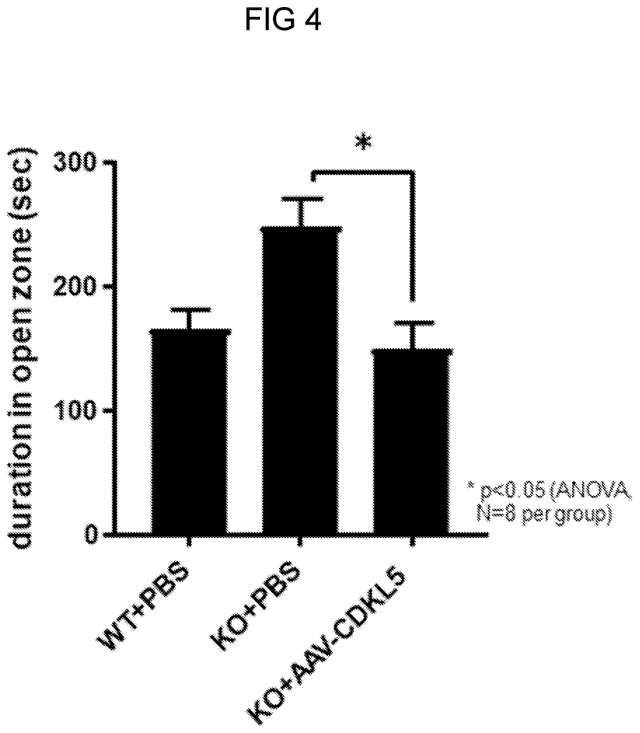

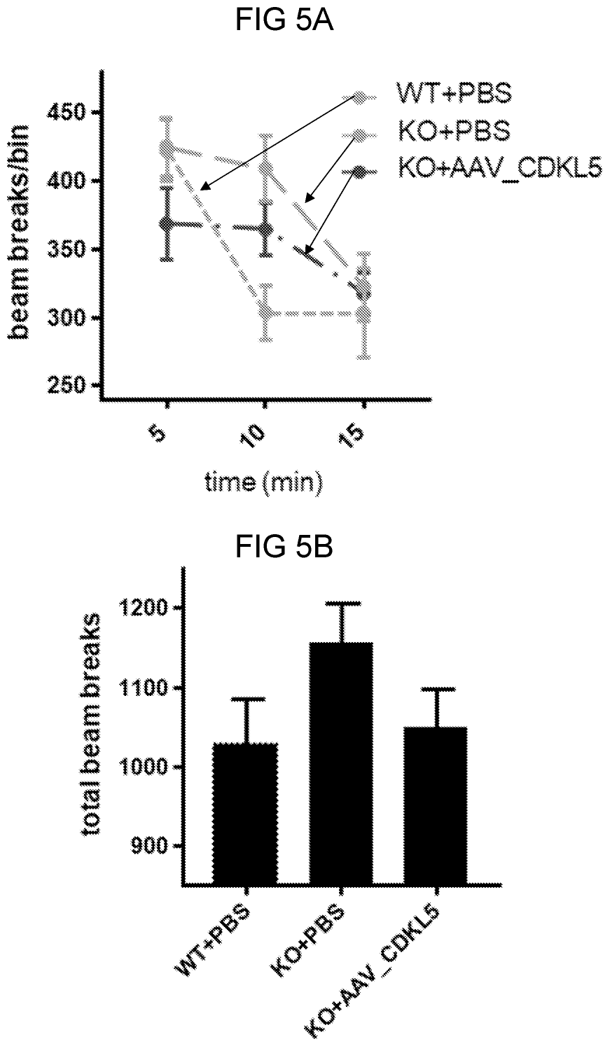

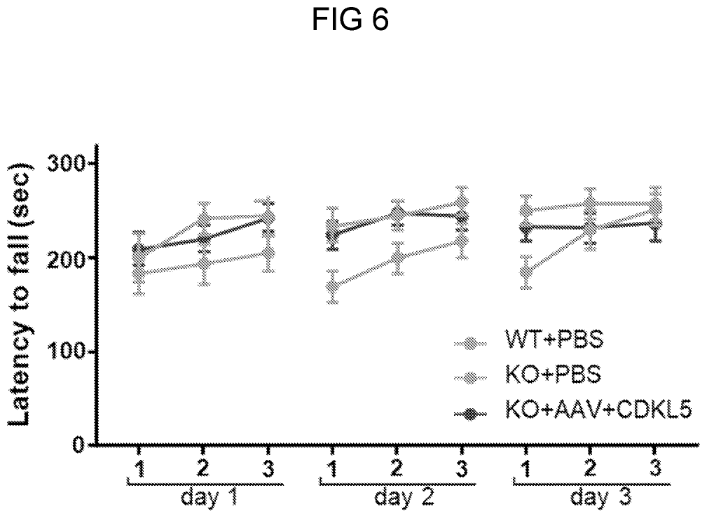

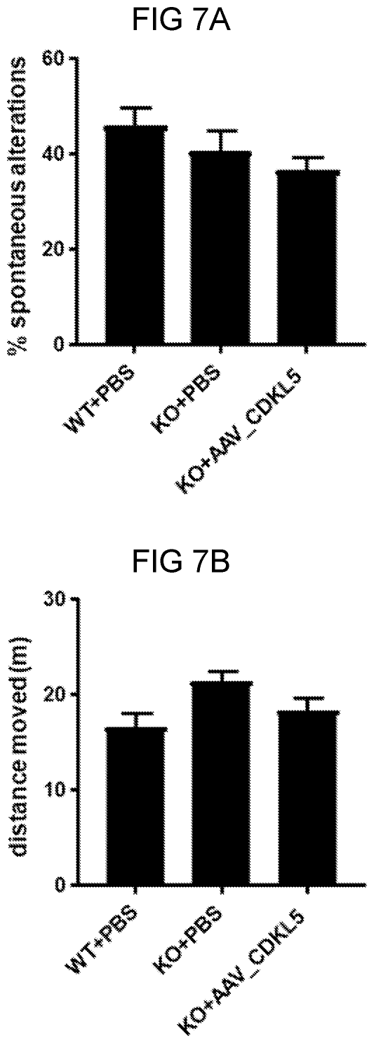

The expression construct between the ITRs is comprised of the human synapsin promoter (SEQ ID NO: 23), the engineered coding sequence for human CDKL5, isoform 1 (SEQ ID NO: 22), the WPRE expression enhancer (SEQ ID NO: 27) and SV40 poly A sequence (SEQ ID NO: 28) (FIG. 1A). Similarly, the alternative expression constructs contain engineered coding sequences for human CDKL5, isoform 2 (CDKL5-2GS or hCDKL5-2GS; SEQ ID NO: 24), 3 (CDKL5-3GS or hCDKL5-3GS; SEQ ID NO: 25) or 4 (CDKL5-4GS or hCDKL5-4GS; SEQ ID NO: 26), respectively, instead of isoform 1. All tested plasmids expressed well in mouse brain, showing minor differences in ability to phosphorylate EB2 (measurement of CDKL5 kinase activity). We found that the WPRE enhancer is required to obtain human CDKL5 expression levels in the mouse brain that resemble wildtype mouse Cdkl5 expression (FIG. 2). CDKL5 was fully active, as determined by its ability to phosphorylate its endogenous target EB2 protein (FIG. 2). The expression and localization of CDKL5 was confirmed via IHC.

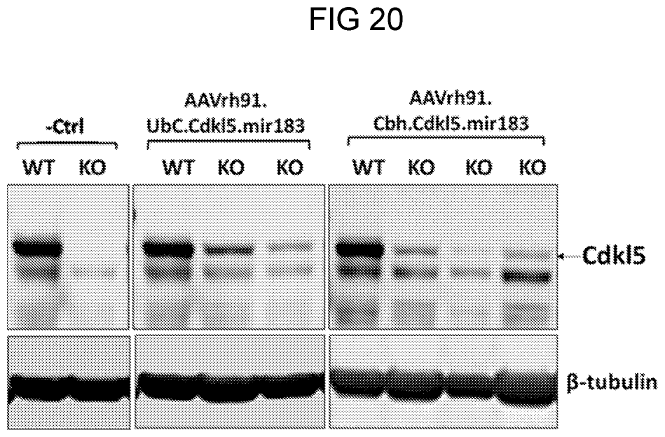

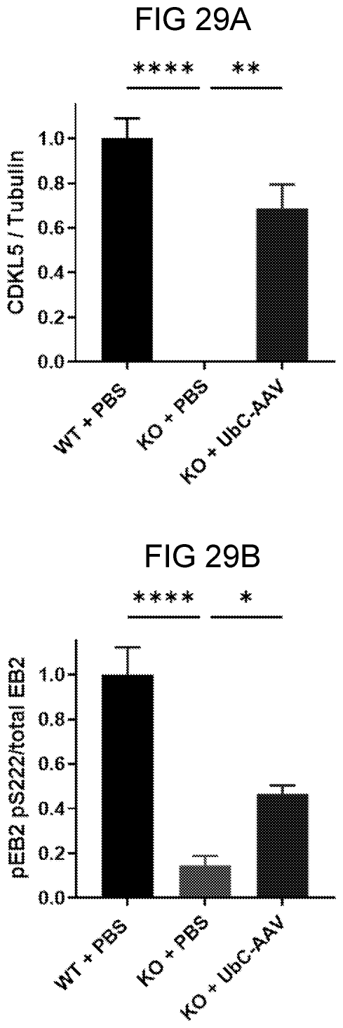

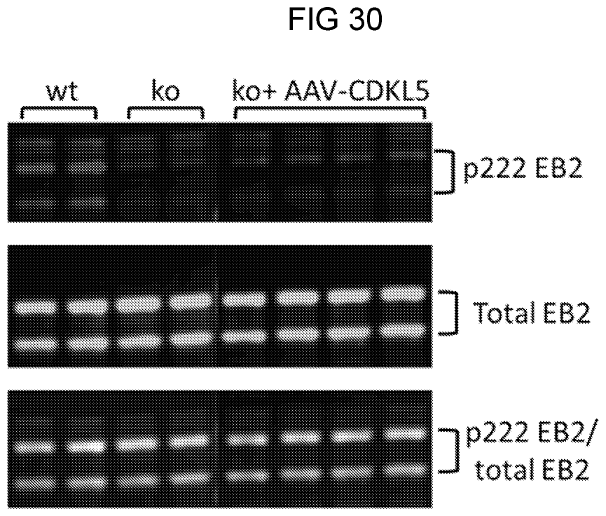

We also generated an alternative expression construct by swapping the human synapsin with the human Ubiquitin C promoter (Ubc) (FIG. 1B) or with the chicken-beta actin hybrid promoter (FIG. 1C). The AAV vectors AAVrh91.UbC.CDKL5-1co.miR183 and AAVrh91.CBh.CDKL5-1co.miR183, in AAVrh91 capsid, were administered at a dose of 3×10¹⁰ GC via neonatal ICV in Cdkl5-ko mice and necropsy was performed at P14. Western blot analysis of mouse brain tissue confirmed the expression of CDKL5 following transduction with AAV vector genomes comprising UbC or CBh promoters (FIG. 20). FIG. 20 shows CDKL5 expression as measured qualitatively by Western blotting, at 14 days following administration of either AAVrh91.UbC.CDKL5-1co.miR183 or AAVrh91.CBh.CDKL5-1co.miR183. Overall, we observed robust restoration of CDKL5 expression and kinase function in CDKL5-ko mouse brain (analyzed cortex tissue) when treated with AAVrh91.UbC.CDKL5-1co.miR183 at a dose of 3×10¹⁰ GC (FIGS. 29A, 29B and 30). FIG. 29A shows CDKL5 expression quantified from Western blot analysis, charted as CDKL5/tubulin levels in knockout mice which were administered AAVrh91.UbC.CDKL5-1co.miR183 at doses 3×10¹⁰ GC, as compared to WT and knockout mice treated with PBS (control groups). FIG. 29B shows kinase activity quantified from Western blot analysis, charted as pEB2pS222/total EB2 levels in knockout mice which were administered AAVrh91.UbC.CDKL5-1co.miR183 at doses 3×10¹⁰ GC, as compared to WT and knockout mice treated with PBS (control groups). FIG. 30 shows kinase activity as measured qualitatively by Western blotting (using pEB-S222 antibody; Baltussen et al., (2018)), in knockout mice which were administered AAVrh91.UbC.CDKL5-1co.miR183 at doses 3×10¹⁰ GC, as compared to WT and knockout mice treated with PBS (control groups).

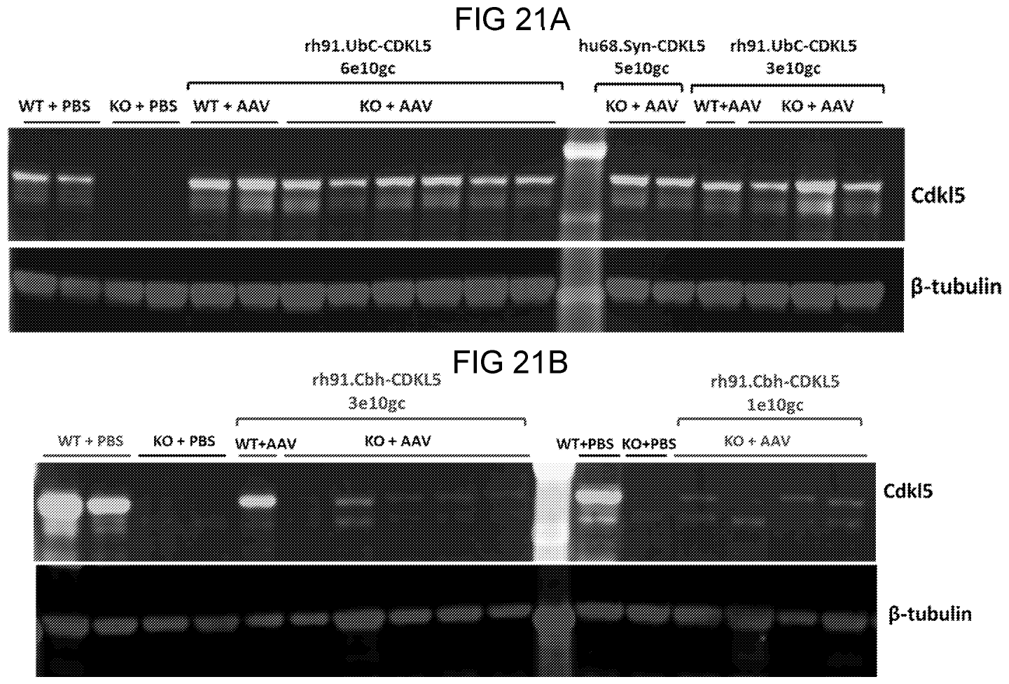

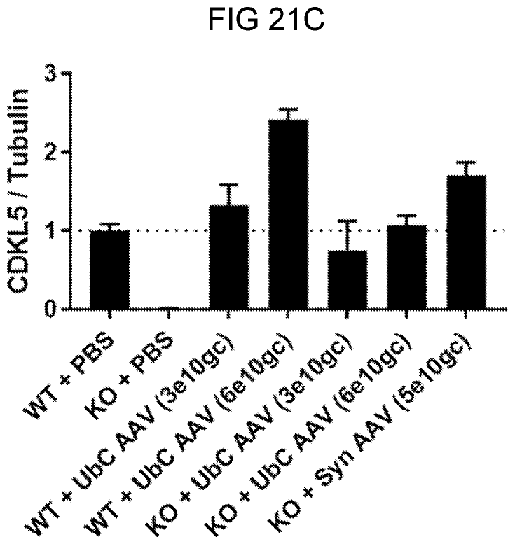

Next, we examined CDKL5 expression levels following administration of the AAVrh91.UbC.CDKL5-1co.miR183 and AAVrh91.CBh.CDKL5-1co.miR183 at varied doses. In this study, mice were administered with AAVrh91.UbC.CDKL5-1co.miR183 or AAVrh91.CBh.CDKL5-1co.miR183 via neonatal ICV at doses of 1×10¹⁰, 3×10¹⁰, 6×10¹⁰ GC. Tissue samples of cortex were harvested form mice at 4 months age and examined for CDKL5 expression via Western blotting (FIGS. 21A, 21B and 21C). FIG. 21A shows CDKL5 expression as measured qualitatively by Western blotting, at 4 months of age, wherein mice which were administered AAVrh91.UbC.CDKL5-1co.miR183 at a dose of 3×10¹⁰ or 6×10¹⁰ GC via neonatal ICV. FIG. 21C shows CDKL5 expression quantified from Western blotting analysis, charted as CDKL5/tubulin levels in wild type and knockout mice which were administered AAVrh91.UbC.CDKL5-1co.miR183 at a dose of 3×10¹⁰ or 6×10¹⁰ GC via neonatal ICV, and compared to AAVhu68.hSyn-CDKL5 at a dose of 5×10¹⁰ GC. Western blot analysis of cortex showed robust hCDKL5 transgene expression. From these results, we observed a a dose-dependent expression, a saturation at a dose of 6×10¹⁰ GC. When compared result to those observed from CDKL5 expression driven by hSyn promoter, UbC promoter allowed for CDKL5 expression to reach levels closer to Wild Type (WT) CDKL5 expression levels in a healthy subject. Western blot analysis CDKL5 expression following administration of AAVrh91.CBh.CDKL5-1co.miR183 at various doses, showed a less robust hCDKL5 transgene expression (FIG. 21B). FIG. 21B shows CDKL5 expression as measured qualitatively by Western blotting, at 4 months of age, wherein mice which were administered AAVrh91.CBh.CDKL5-1co.miR183 at a dose of 3×10¹⁰ or 1×10¹⁰ GC via neonatal ICV.

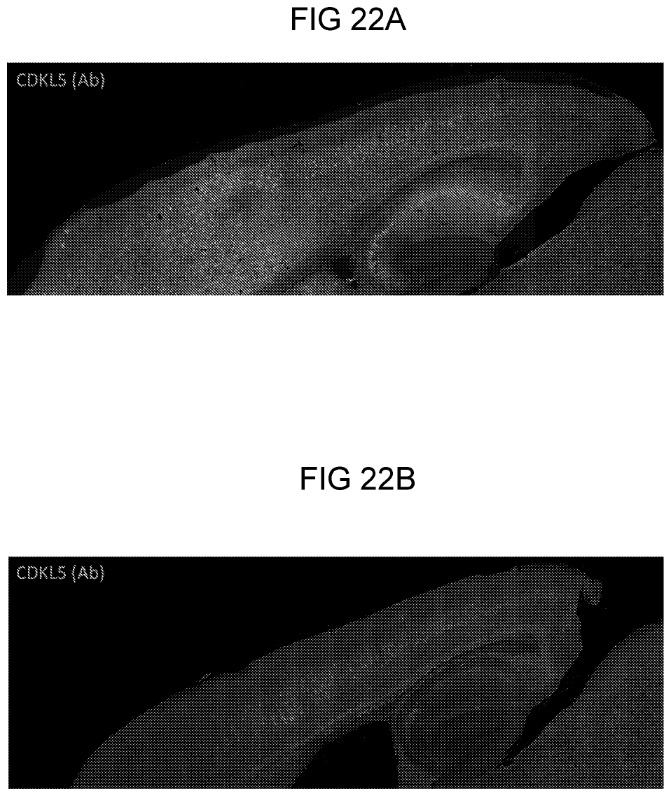

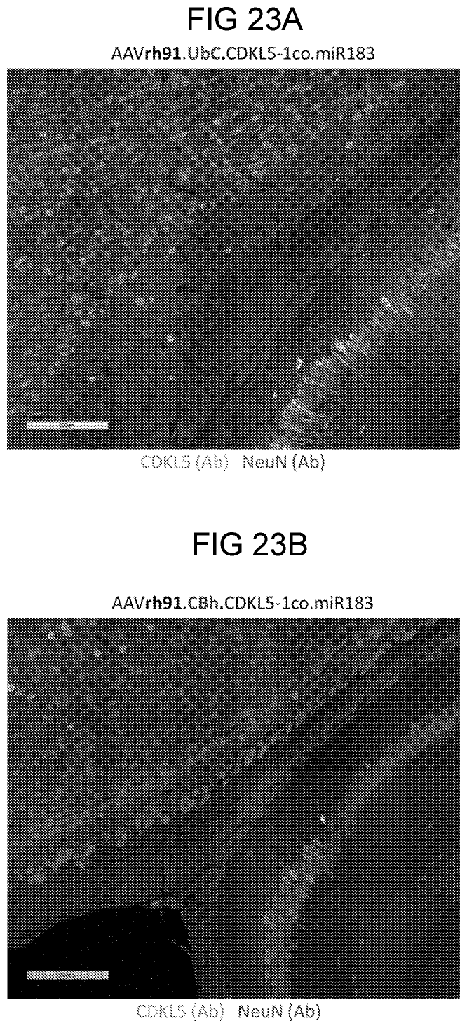

CDKL5 varied expression following administration AAVrh91.UbC.CDKL5-1co.miR183 or AAVrh91.CBh.CDKL5-1co.miR183, as observed by western blot analysis, was further confirmed by fluorescent microscopy. Representative immunofluorescent microscopy images showed significant expression of CDKL5 throughout brain post administration of AAVrh91.UbC.CDKL5-1co.miR183, and a dim expression was observed post administration with AAVrh91.CBh.CDKL5-1co.miR183, while mostly being visible in cortex (FIGS. 22A and 22B). FIG. 22A shows a representative image from immunofluorescent microscopy analysis of CDKL5 expression following administration with AAVrh91.UbC.CDKL5-1co.miR183 at a dose of 3×10¹⁰ GC via neonatal ICV. FIG. 22B shows a representative image from immunofluorescent microscopy analysis of CDKL5 expression following administration with AAVrh91.CBh.CDKL5-1co.miR183 at a dose of 3×10¹⁰ GC via neonatal ICV. Upon further analysis, we observed that the UbC promoter drives CDKL5 expression in more cells than the CBh promoter, which localizes mostly in neurons (FIGS. 23A and 23B). FIG. 23A shows a representative image (zoomed in view) from immunofluorescent microscopy analysis of CDKL5 expression (samples were also probed for NeuN, neuronal marker) following administration with AAVrh91.UbC.CDKL5-1co.miR183 at a dose of 3×10¹⁰ GC via neonatal ICV. FIG. 23B shows a representative image (zoomed in view) from immunofluorescent microscopy analysis of CDKL5 expression (samples were also probed for NeuN, neuronal marker) following administration with AAVrh91.CBh.CDKL5-1co.miR183 at a dose of 3×10¹⁰ GC via neonatal ICV.

In summary, we observed expression of CDKL5 transgene in mouse brain following the transduction with AAVrh91.UbC.CDKL5-1co.miR183 and AAVrh91.CBh.CDKL5-1co.miR183. Upon further analysis, a higher CDKL5 expression per cell was observed in hippocampus of mouse brain after transduction with AAVrh91.UbC.CDKL5-1co.miR183. While, lower expression of CDKL5 per cell was observed in hippocampus of mouse brain after transduction with AAVrh91.CBh.CDKL5-1co.miR183, wherein. A higher expression of CDKL5 per cell was observed in cortex of mouse brain after transduction with AAVrh91.UbC.CDKL5-1co.miR183. A lower expression of CDKL5 per cell was observed in cortex of mouse brain after transduction with AAVrh91.CBh.CDKL5-1co.miR183. Furthermore, we compared CDKL5 expression in Cdkl5-ko mouse brain following administration of AAVrh91.UbC.CDKL5-1co.miR183 (3×10¹⁰ GC, neonatal ICV) and AAVhu68.hSyn.CDKL5-1co.miR183 (2.5×10¹⁰ GC). Very similar expression patterns were observed in mouse brain following administration of the AAV as specified. A CDKL5 expression was observed in mouse brain after transduction either with AAVrh91.UbC.CDKL5-1co.miR183 (AAVrh91 capsid; Ubiquitin C promoter) or with AAVhu68.hSyn.CDKL5-1co.miR183 (AAVhu68 capsid; Synapsin promoter).

### Example 3: Pre-Clinical Therapeutic Benefit of CDKL5 Gene Therapy in CDD Mouse Model

To test of the therapeutic benefit CDKL5 gene therapy on Cdkl5 deficient mice, we treated cohort of juvenile Cdkl5-ko (also referenced as KO mice or mice) and wild type (wt) littermates with 5×10¹¹ GC (5e11 gc) of AAV9-PHP.B-hSyn-hCDKL5-1co.WPRE vector by retro-orbital IV injection. All treatment groups continued to grow at the same rate and no treatment-related deaths were observed. At 10 weeks of age, mice were subjected to the battery of behavior testing. We observed a robust and statistically significant normalization for the treated group in the Elevated Zero Maze and Open Field Activity tests. There was a marginal improvement in the same group in the rotarod and Y-Maze test, and no improvement in thermal sensitivity.

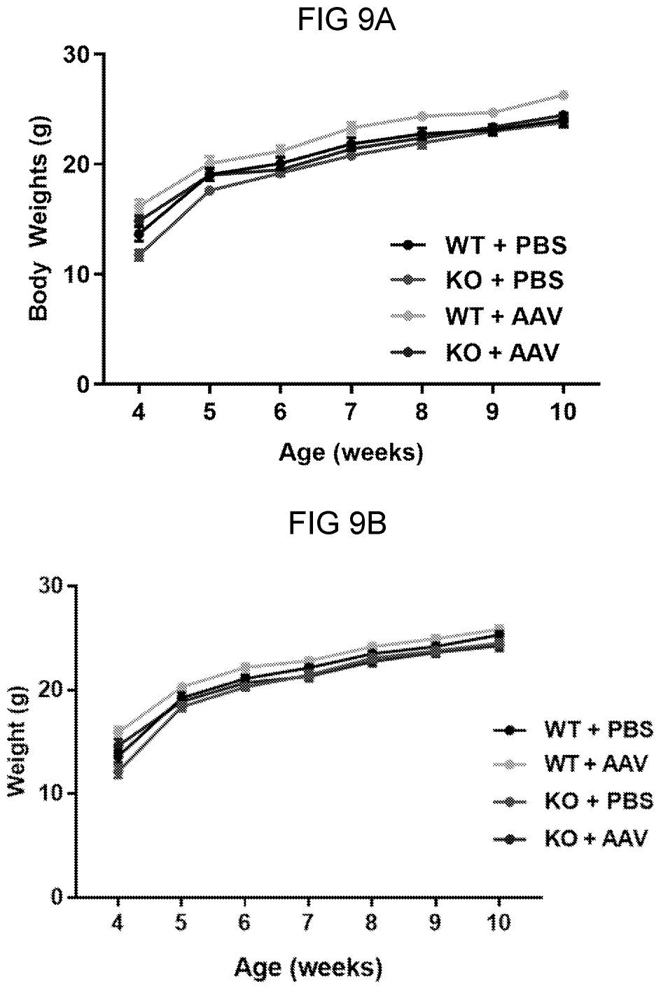

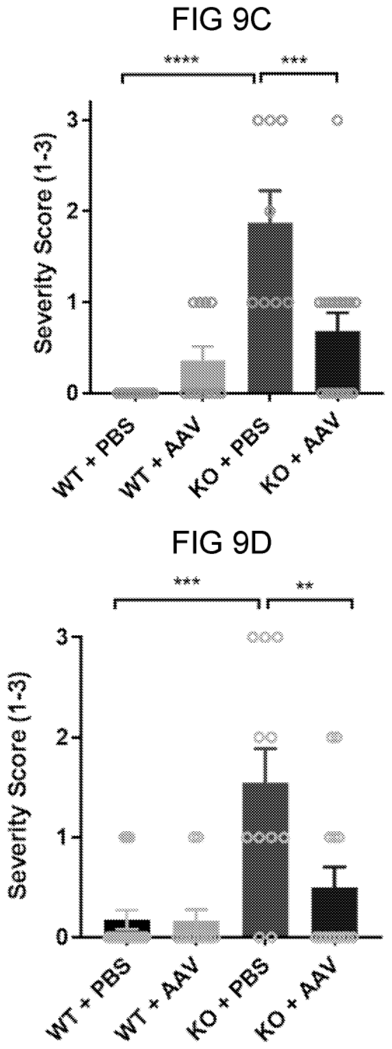

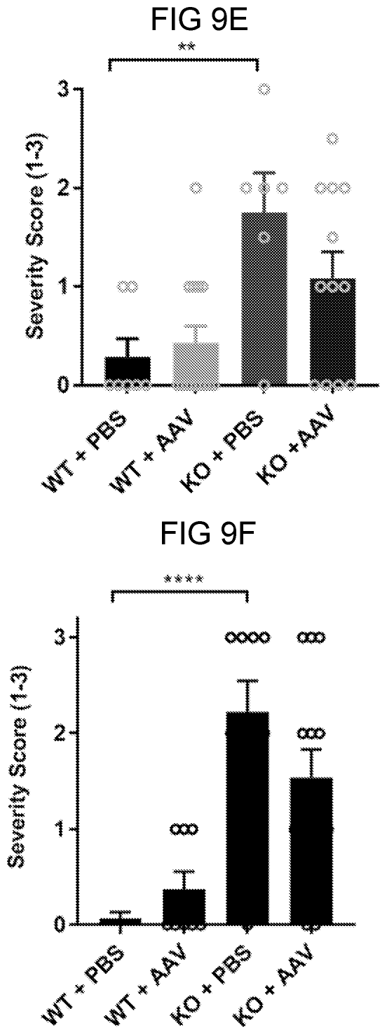

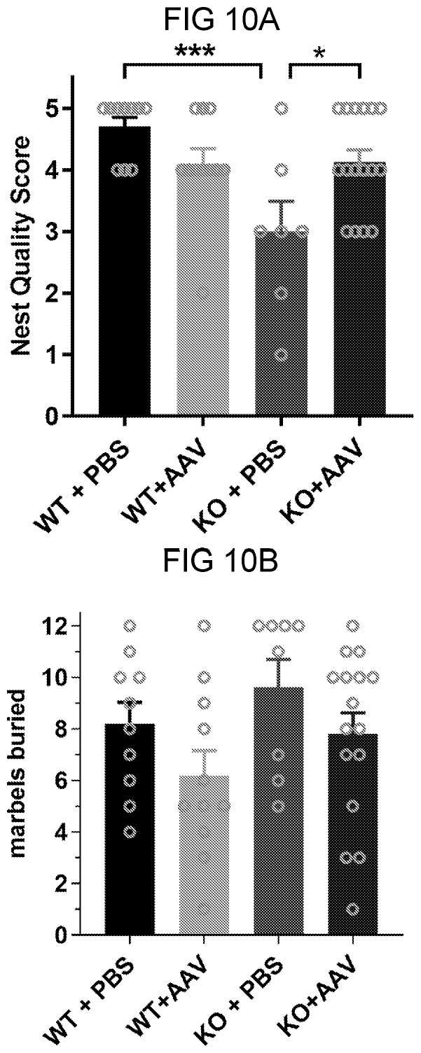

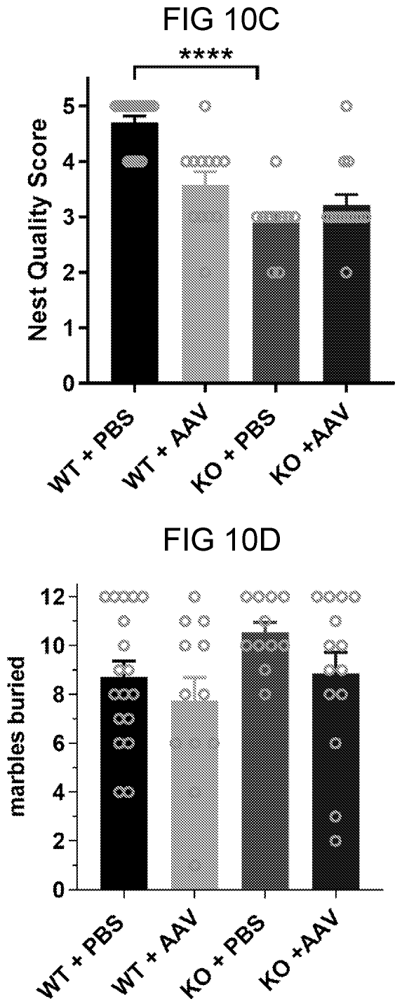

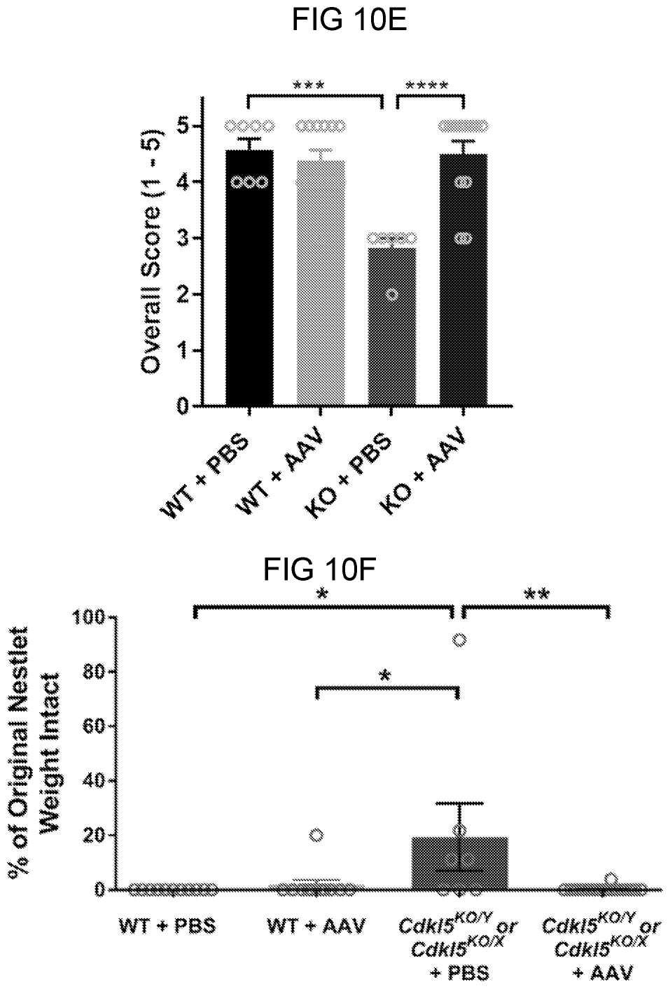

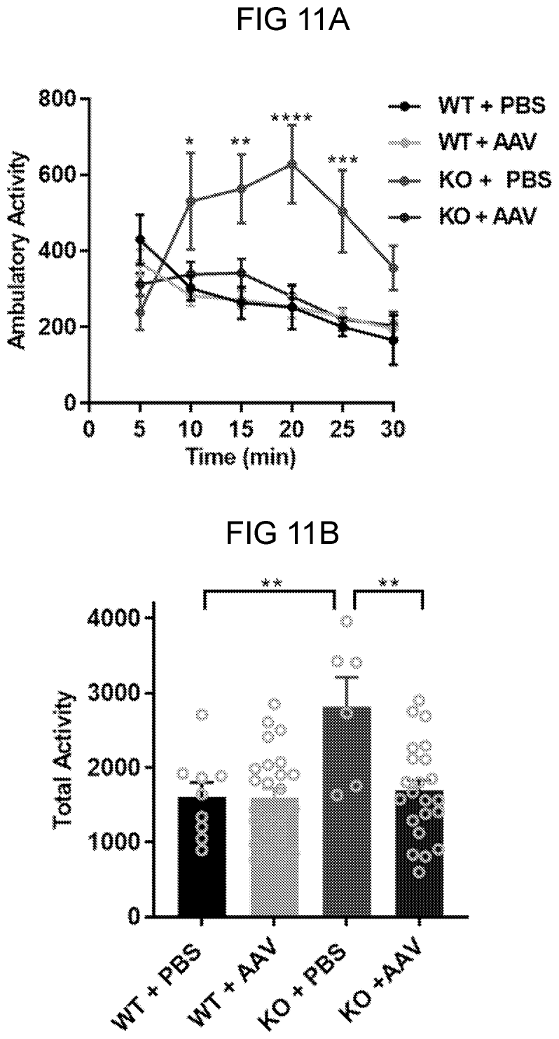

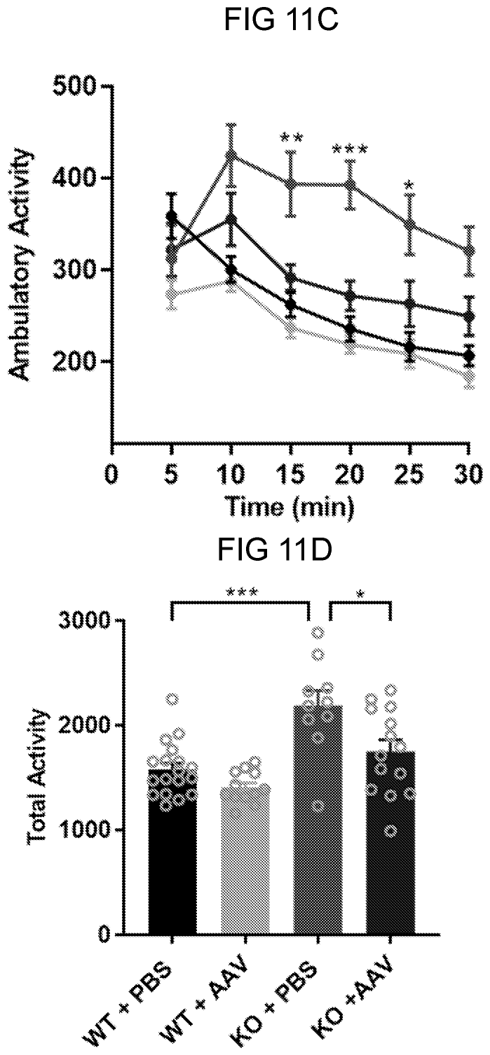

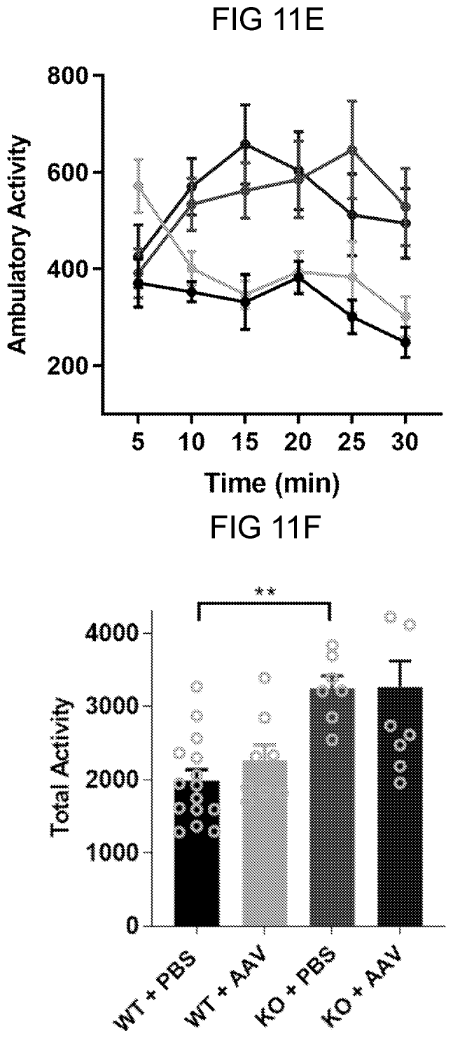

Given that Cdkl5 is important for neurodevelopment, we wondered whether earlier administration of gene therapy might improve therapeutic outcome in mice. We next treated cohorts of neonatal Cdkl5-ko or wt littermates with doses ranging 6×10⁹ GC to 5×10¹⁰ GC of AAVhu68-hSyn-hCDKL5-1co.WPRE vector per mouse by intraventricular (ICV) injection. Behavioral testing stated at 10 weeks of age or at 11 to 14 weeks, where indicated. Overall observation across all groups was that the treatment was well tolerated, there was no treatment related deaths, and normal weight gain (FIGS. 9A and 9B) and overall development was observed. A dose-dependent expression of CDKL5 transgene in Cdkl5-ko mouse brain at a dose of 5×10¹⁰ GC, a dose of 2.5×10¹⁰ GC, and a dose of 1×10¹⁰ GC was observed. At 10 weeks of age, KO mice show the characteristic hind limb clasping phenotype, which was substantially ameliorated in treated ko mice. Therapeutic efficacy of CDKL5 gene therapy was measured by hind limb clasping test. A dose dependent improvement was observed of the severity score in treated CDKL5-ko mice (FIGS. 9C-9F). Likewise, the persistent hyperactivity and rearing phenotype found in ko mice was normalized towards wildtype activity. Hippocampal learning and memory were highly improved in treated ko mice in the Y-Maze test (spontaneous alteration index). Therapeutic efficacy of CDKL5 gene therapy was further measured by open filed activity test, which measures for correction of hyperactivity in KO and AAV-treated mice. A dose-dependent resolution of hyperactivity in treated Cdkl5-ko mice was observed (FIGS. 11A-11F). KO mice are poor nest-builders when overnight single-housed in a fresh cage and do not tear up all nesting material. Treated KO mice drastically improved their nest building capabilities in a dose-dependent manner (FIGS. 10A, 10C, 10E). Additional two other neurobehavior assays (marble burying and Y-Maze) showed a strong trend to improvement of phenotype in treated Cdkl5-ko mice (FIGS. 10B and 10D). EEG phenotype was rescued in a preliminary collaborative study and may become useful as translational biomarker. Validation of outcomes of key assays (hindlimb clasping, ambulatory activity) are additionally performed with an independent experimental cohort. Larger cohort size (N=18/group) led to more robust statistical significance. Validation of outcomes of key assays (hindlimb clasping, ambulatory activity) with alternative CDD mouse models (R59X, D471fs) is also performed. In summary, CDKL5 gene therapy delivers a functional CDKL5 protein to mouse brain and has dose-dependent therapeutic effect for several neurobehavior outcomes in CDD male mouse models.

After finishing behavior testing, brains were harvested when the mice were about 3 months old. Western blotting revealed robust expression of human CDKL5 in Cdkl5-ko brains, even 3 months after AAV administration. Lastly, human CDKL5 showed robust kinase activity when blotting for phosphorylated EB2 protein.

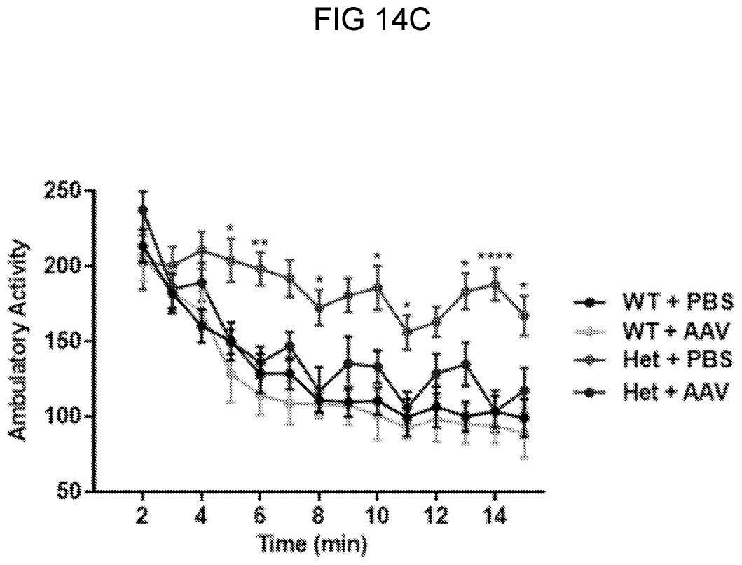

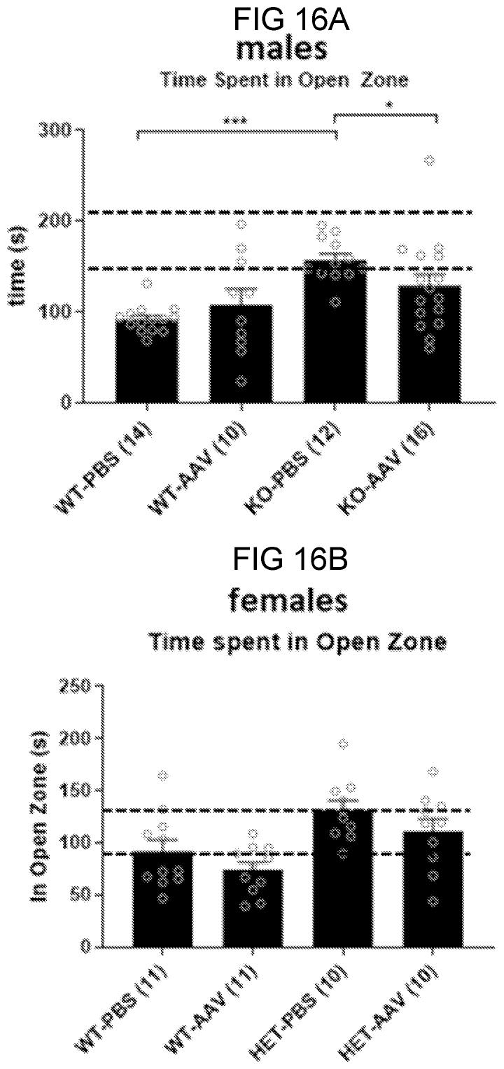

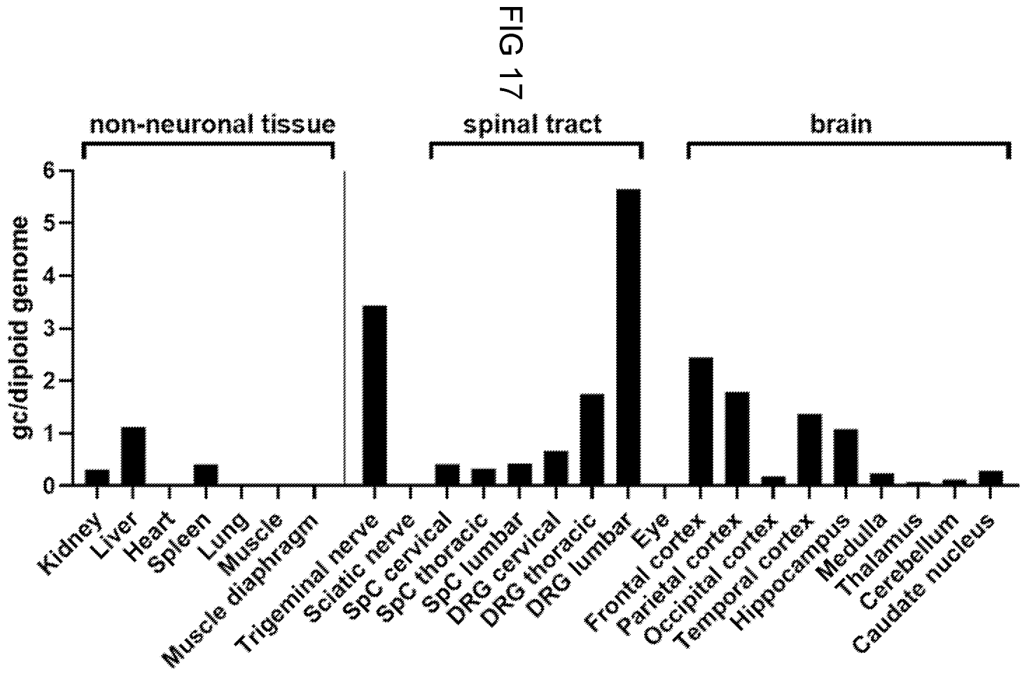

We also examined the outcome of CDKL5 gene therapy with an alternative CDD mouse model. Cdkl5(D471fs) carry a patient point mutation that leads to a premature stop codon. As in patients, no CDKL5 protein and highly reduced EB phosphorylation levels were found in the brains of these mice. Thus, Cdkl5(D471fs) share the absence of Cdkl5 protein with the previously used Cdkl5-ko mice, however, the genetic background is slightly different due the method of generation for the mouse models. Neonatal pups were injected at the same concentration as before (5×10¹⁰ GC, neoICV) and showed robust hCDKL5 protein expression and EB2 phosphorylation 3 months later. A small pilot cohort was subjected to behavior testing. AAV treatment was well tolerated and no morbidity was observed. Hind limb clasping was significantly corrected in treated mutant mice; likewise, the mutant phenotype in the Elevated Zero Maze test was corrected to wildtype behavior in treated mutant mice (In Open Zone, Open Zone Entries, FIGS. 14, 15, 16). Mutant mice show poor interaction and burying behavior with glass marbles arrange in a grid in a new cage, whereas wildtype mice typically burry almost all. Treated mutant mice show a strong correction of their behavior to wildtype mice. See also, Examples 8 and 9, described below. Significant improvement was observed in Cdkl5-ko mice in treated group at higher dose of 5×10¹⁰ GC/mouse.

We also examined the outcome of CDKL5 gene therapy with an alternative CDD mouse model. Cdkl5(R59X) carry a patient point mutation that introduces a premature stop codon. As in patients, no CDKL5 protein and highly reduced EB phosphorylation levels were found in the brains of these mice. Thus, Cdkl5(R59X) share the absence of Cdkl5 protein with the previously used Cdkl5-ko mice, however, the genetic background is slightly different due the method of generation for the mouse models. Neonatal pups were injected at the same concentration as before (5×10¹⁰ GC, neoICV) and showed robust hCDKL5 protein expression and EB2 phosphorylation 3 months later. A small pilot cohort was subjected to behavior testing. AAV treatment was well tolerated, and no morbidity was observed. Hind limb clasping was significantly corrected in treated mutant mice.

We also examined the outcome of CDKL5 gene therapy with alternative CDD mouse model in heterozygous female Cdkl5-ko mice. Such model reflects most CDD patient (CDD females). Overall assessment showed that heterozygous female Cdkl5-ko mouse neurobehavior phenotype is much milder, with later onset and thus makes robust assessment of a therapeutic outcome more difficult. However, a representative data for hindlimb clasping and ambulatory activity at high dose (5×10¹⁰ GC, neonatal ICV) shows significant improvement (FIGS. 14B and 14C). See also, Examples 8 and 9.

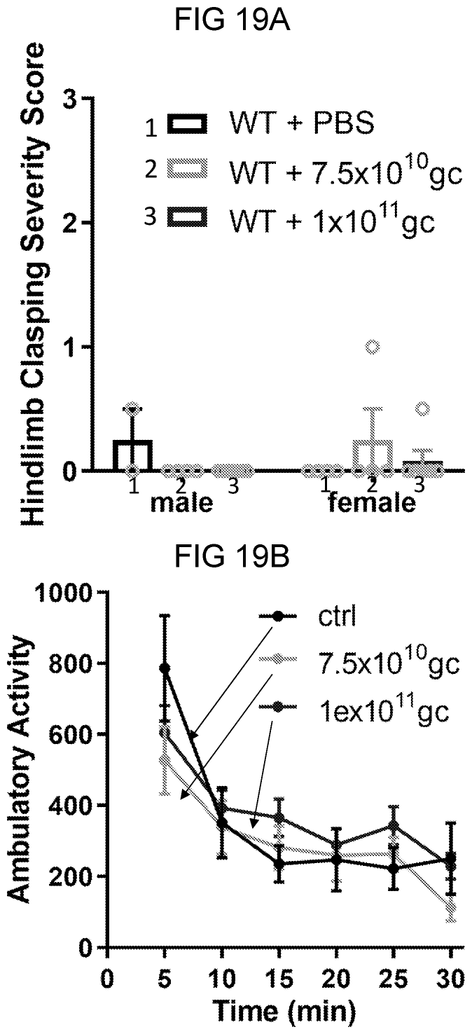

Additionally, we examined CDKL5 Gene therapy dose escalation in WT mice. WT (C57B16/J) mice were injected at 7.5×10¹⁰ GC and 1×10¹¹ GC (i.e., 1.5× or 2× of previously highest used dose) via neonatal ICV. There was no overt effect observed on weight, development, and survival. Mice appeared normal and did not show hindlimb clasping or activity changes. There was no observed effects in pathologist review of CNS tissues (FIGS. 19A and 19B).

### Example 4. Pre-Clinical Therapeutic Benefit of CDKL5 Gene Therapy (AAVrh91.UbC.CDKL5-1Co.miR183) in CDD Mouse Model

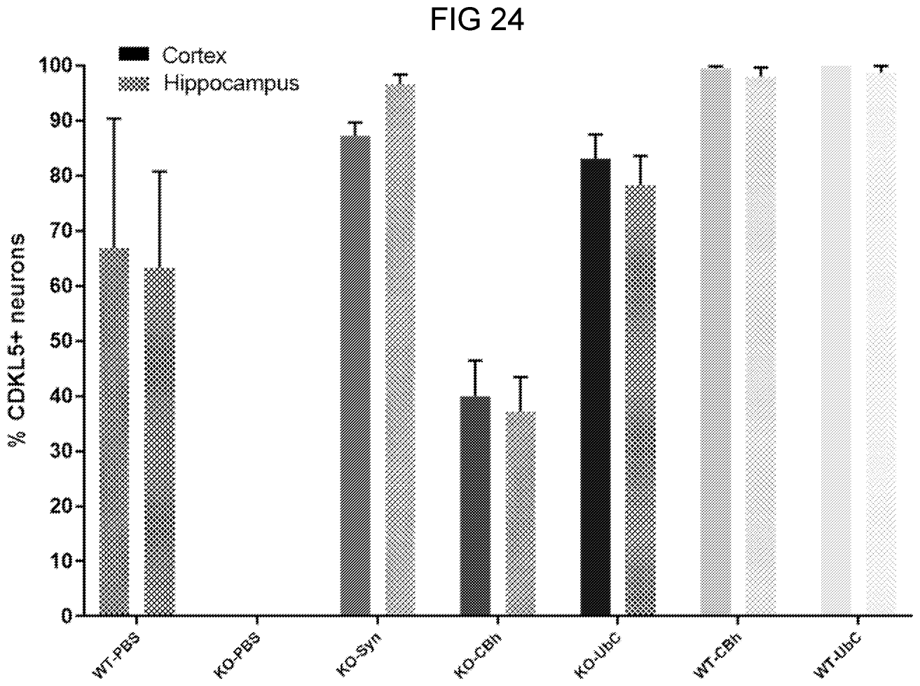

Additionally, varying doses of AAVrh91.UbC.CDKL5-1co.miR183 and AAVrh91.CBh.CDKL5-1co.miR183 at 1×10¹⁰ GC, 3×10¹⁰ GC, or 6×10¹⁰ GC were examined in Cdkl5-ko and wild type mice for behavior effects (i.e., therapeutic effects). In this study, Cdkl5-ko (also referenced as KO mice or mice) and wild type (WT) mice were administered either AAVrh91.UbC.CDKL5-1co.miR183 or AAVrh91.CBh.CDKL5-1co.miR183 via neonatal ICV at a dose of 3×10¹⁰ GC. FIG. 24 shows quantification of CDKL5-expressing neurons (above background levels) as compared to previous results following administration with AAVhu68.hSyn.CDKL5. When compared, AAVrh91.UbC.CDKL5-1co.miR183 administration was observed to achieve expression within the range of WT expression in normal subjects. While AAVrh91.CBh.CDKL5-1co.miR183 administration was observed to achieve suboptimal expression levels in mouse brain at a dose of 3×10¹⁰ GC. As expected, in WT brains the number of neurons and intensity was elevated.

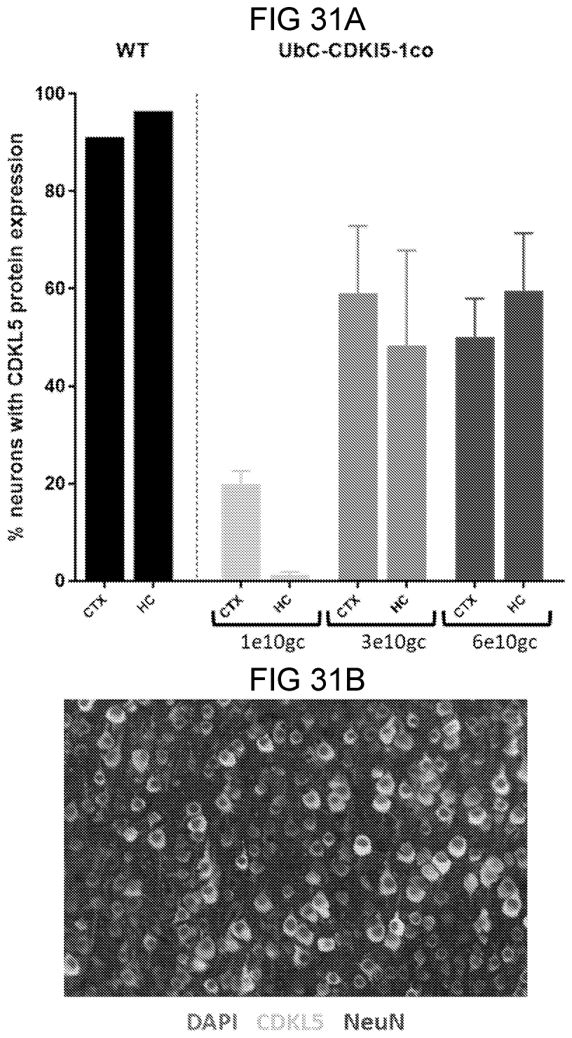

We further confirmed expression of CDKL5 in mice which were administered AAVrh91.UbC.CDKL5-1co.miR183 at a dose of 1×10¹⁰, 3×10¹⁰, or 6×10¹⁰ GC via neonatal ICV. FIG. 31A shows results as the percent neurons with CDKL5 protein expression in mouse cortex and hippocampus tissue following neonatal ICV administration with AAVrh91.UbC.CDKL5-1co.miR183 at a dose of 1×10¹⁰, 3×10¹⁰, or 6×10¹⁰ GC, as compared to WT mice treated with PBS. FIG. 31B shows a representative microscopy image from immunofluorescent analysis staining with DAPI (nuclei), CDKL5, and NeuN (neuronal marker) of cortical section tissue following neonatal ICV administration with AAVrh91.UbC.CDKL5-1co.miR183 at doses of 3×10¹⁰ GC. We observed abundant, dose-dependent CDKL5 expression in cortex and hippocampus.

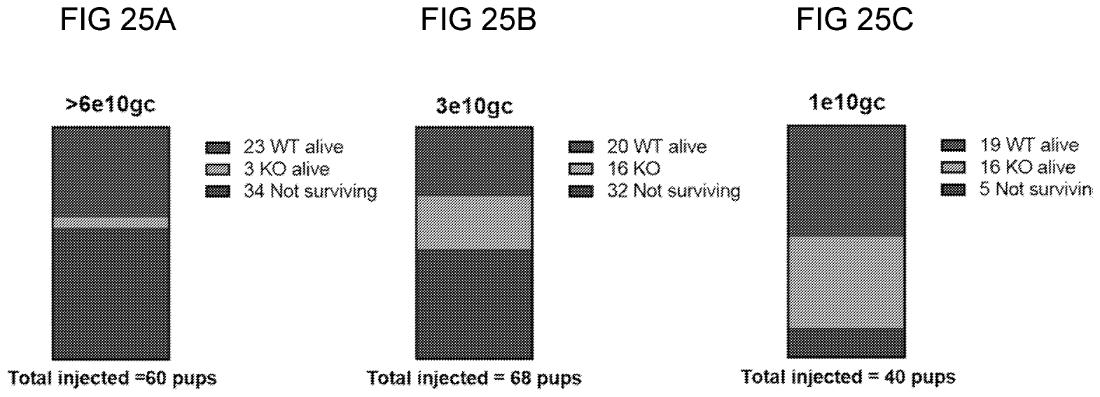

A survival and viability study results showed a trend of a dose-limiting viability after neonatal ICV injection (FIG. 25). FIG. 25 shows results of the survival study with tallied survival at post-natal day 16 (PND16) of mice which were administered with AAVrh91.CBh.CDKL5-1co.miR183 at doses of 1×10¹⁰, 3×10¹⁰, or 6×10¹⁰ GC via neonatal ICV.

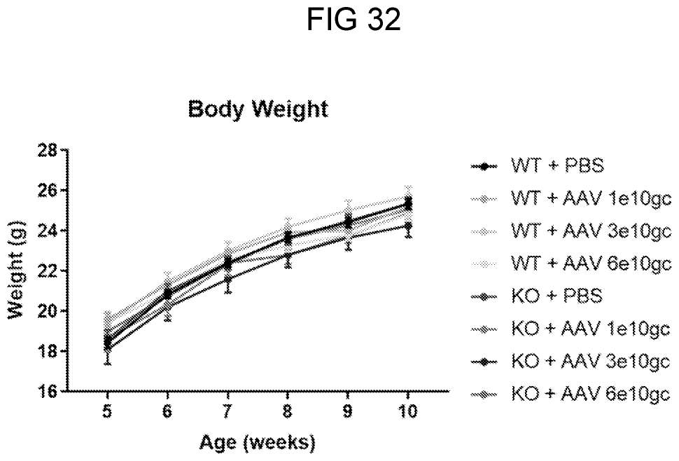

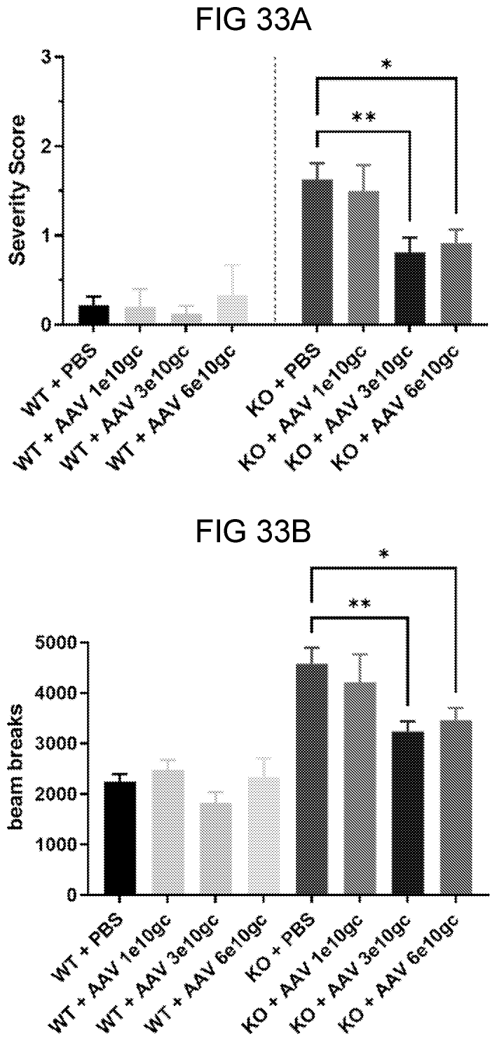

In mice administered with AAVrh91.UbC.CDKL5-1co.miR183, normal development was observed at all doses (i.e., 1×10¹⁰, 3×10¹⁰, 6×10¹⁰ GC). Normal weight gain was observed for all cohorts. Additionally, there was treatment related morbidity observed. The control group of WT mice tolerated CDKL5 expression well. FIG. 32 shows analysis of measured body weights of wild type and CDKL5-ko when administered with PBS, or AAV.UbC.CDKL5-1co.miR183 at doses of 1×10¹⁰, 3×10¹⁰, 6×10¹⁰ GC. Furthermore, an improvement of hindlimb clasping was observed (FIG. 33A). FIG. 33A shows a results of the hindlimb clasping test for AAV.UbC.CDKL5-1co.miR183-treated group at a dose of 3×10¹⁰ GC compared to untreated group in Cdkl5-ko mice and WT mice.

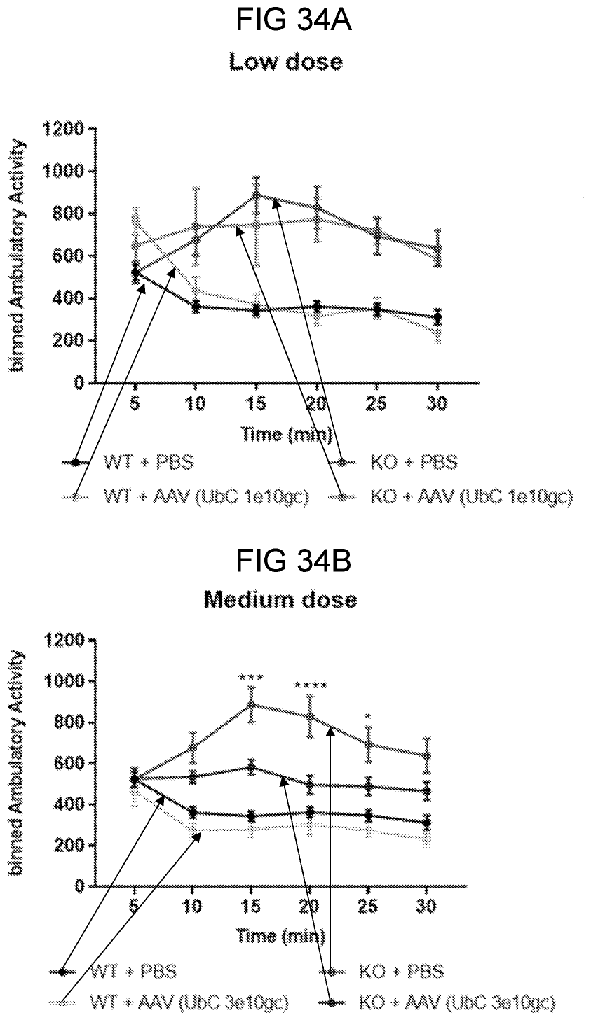

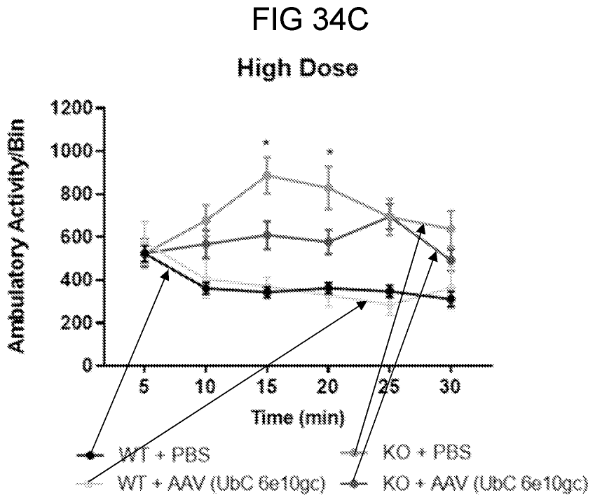

FIG. 33B shows a dose-dependent effect on hyperactivity, as measured in the open field activity test and plotted as ambulatory activity (beam breaks) in Cdkl5-ko mice and WT mice following administration of AAV.UbC.CDKL5-1co.miR183 at a dose of 1×10¹⁰, 3×10¹⁰, or 6×10¹⁰ GC. FIG. 34A shows results of binned ambulatory activity for groups of WT and Cdkl5-ko mice administered with AAV.UbC.CDKL5-1co.miR183 at a low dose of 1×10¹⁰ GC. FIG. 34B shows results of binned ambulatory activity for groups of WT and Cdkl5-ko mice administered with AAV.UbC.CDKL5-1co.miR183 at a medium dose of 3×10¹⁰ GC. FIG. 34C shows results of binned ambulatory activity for groups of WT and Cdkl5-ko mice administered with AAV.UbC.CDKL5-1co.miR183 at a high dose of 6×10¹⁰ GC. These result show a dose-dependent improvement of hyperactivity in Cdkl5-ko mice administered with AAV.UbC.CDKL5-1co.miR183.

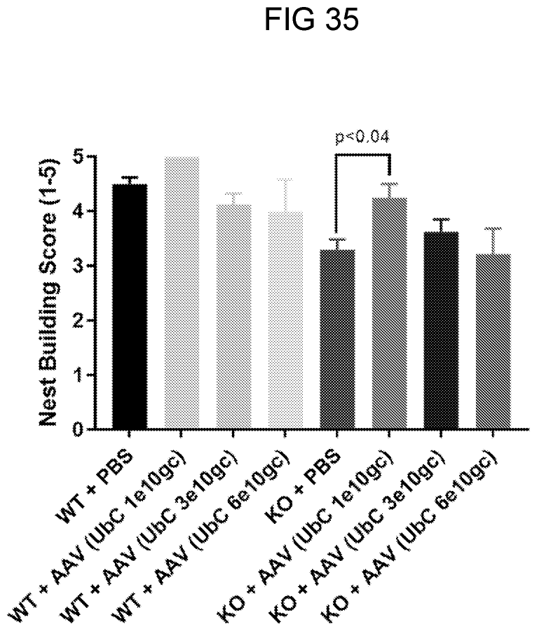

Furthermore, we observed improvement in nest building scores at lowest dose of AAV.UbC.CDKL5-1co.miR183 administered in mice (FIG. 35). FIG. 35 shows results in nest building (nest quality/score) for the AAV.UbC.CDKL5-1co.miR183-treated WT and Cdkl5-ko mice at a dose of 1×10¹⁰, 3×10¹⁰, or 6×10¹⁰ GC.

In summary, we observed a significant improvement in Cdkl5-ko mice when treated with AAV.CDKL5 (under hSyn, UbC) promoter in comparison to the Cdkl5-ko mice treated with PBS.

| | AAVhu68.hSyn.CDKL5-1co | AAVrh91.UbC.CDKL5-1co | | | AAVrh91.CBh.CDKL5-1co | | |
|---|---|---|---|---|---|---|---|
| | 5 × 10¹⁰ | 1 × 10¹⁰ | 3 × 10¹⁰ | 6 × 10¹⁰ | 1 × 10¹⁰ | 3 × 10¹⁰ | 6 × 10¹⁰ |
| Test | GC | GC | GC | GC | GC | GC | GC |
| Hindlimb | Yes | No | Yes | No | No | No | (N too |
| Clasping | | | | | | | due to |
| Open | Yes | No | Yes | Yes | No | No | early |
| Filed | | | | | | | death) |
| Activity | | | | | | | |
| Nest | Yes | Yes | No | No | No | No | |
| Building | | | | | | | |

The AAV.UbC.CDKL5-1co.miR183 vector showed therapeutic utility in mice similar to when CDKL5 expression was driven by hSyn. The rAAV.CDLK5 vector having an AAVrh91 capsid used with an AAV vector genome comprising an engineered nucleic acid sequence achieved CDKL5 expression levels similar to that of the rAAVhu68.CDKL5. CDKL5 protein levels in mouse brain were higher with the Ube promoter compared with the hSyn promoter.

### Example 5: Utility Human CDKL5 Isoforms 2-4

It has been shown that there are at last 4 detectable Cdkl5 mRNA splice variants found in human and mouse brain, but it has not been established of isoforms 2-4 exist as stable proteins. Isoform 1 accounts for >85% of brain CDKL5.

We codon engineered coding sequences for human CDKL4 isoforms 2-4 and cloned the into the same AAV vector as before. AAV9-PHP.B vector coding for isoforms 1-4 was tail IV injected in adult Cdkl5-ko mice and brains harvested after 2 weeks for Western Blot analysis. We found all four isoforms robustly expressing and displaying the expected gel migration patterns. The engineered sequence which yielded expression for isoforms 2-4 that closely resembled the amount of expression of isoform 1, were chosen for follow up. EB2 phosphorylation levels generated by isoform 2 were slightly higher, and by isoforms three or 4 slightly lower compared to isoform 1.

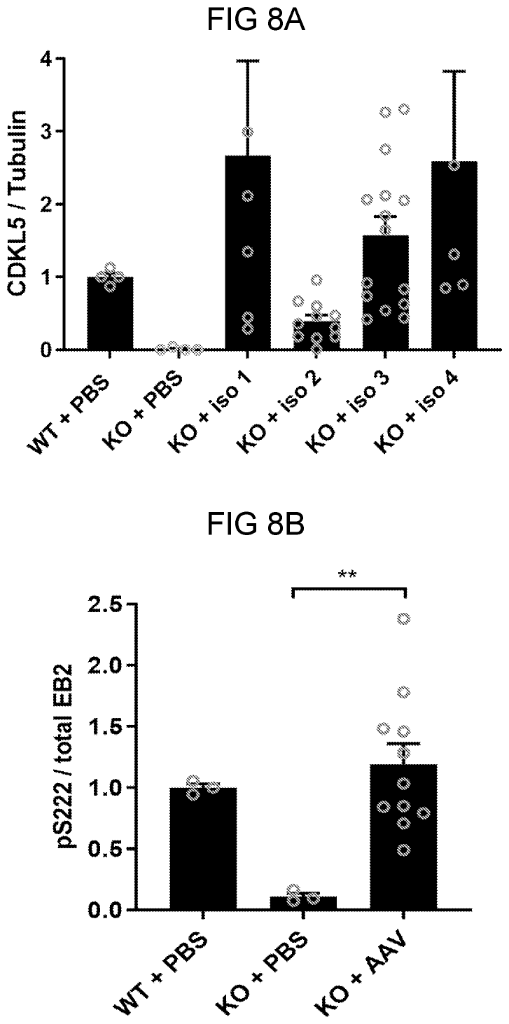

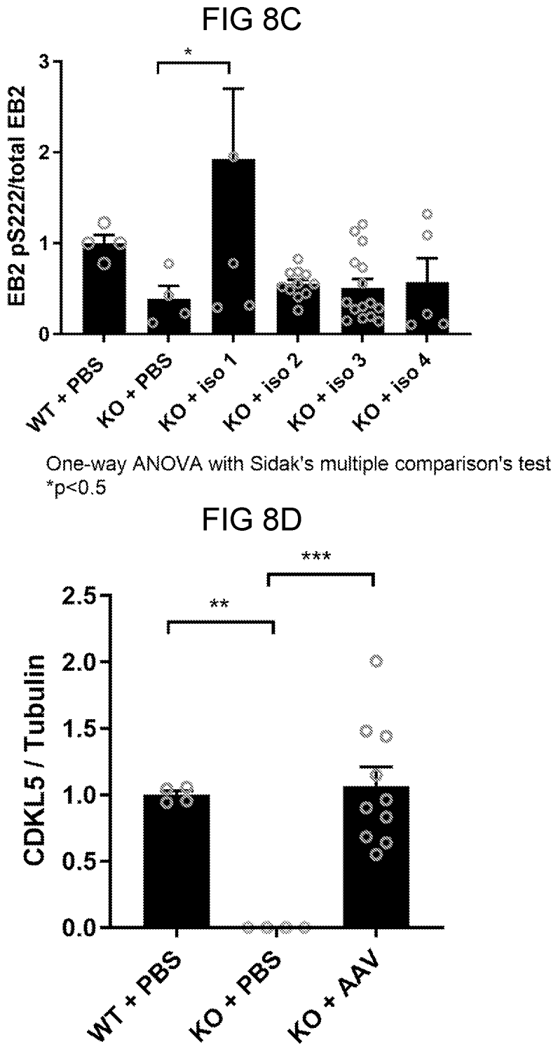

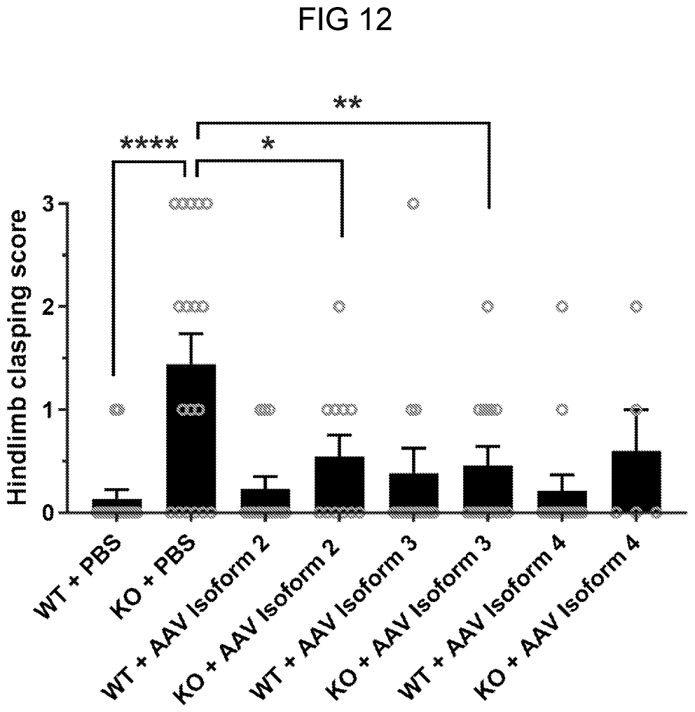

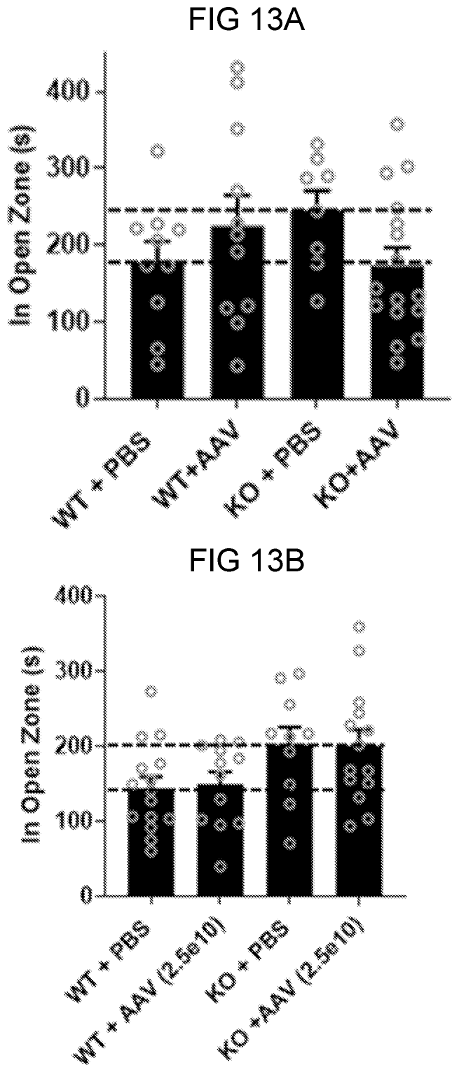

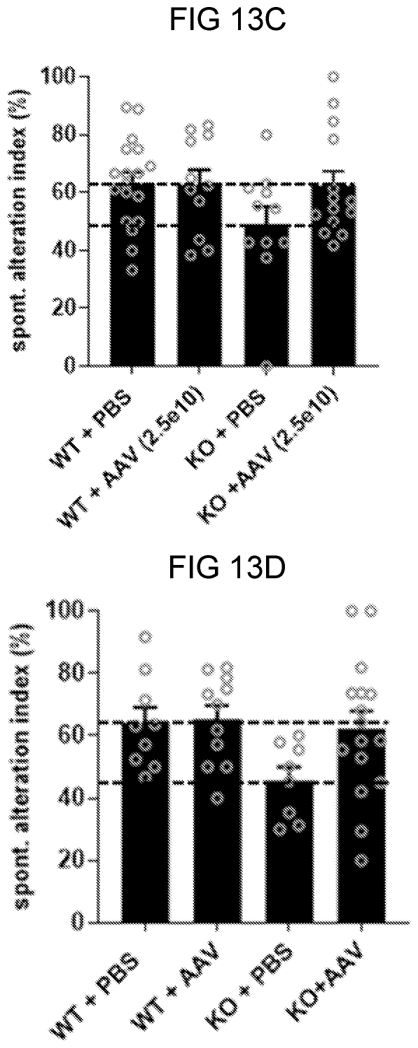

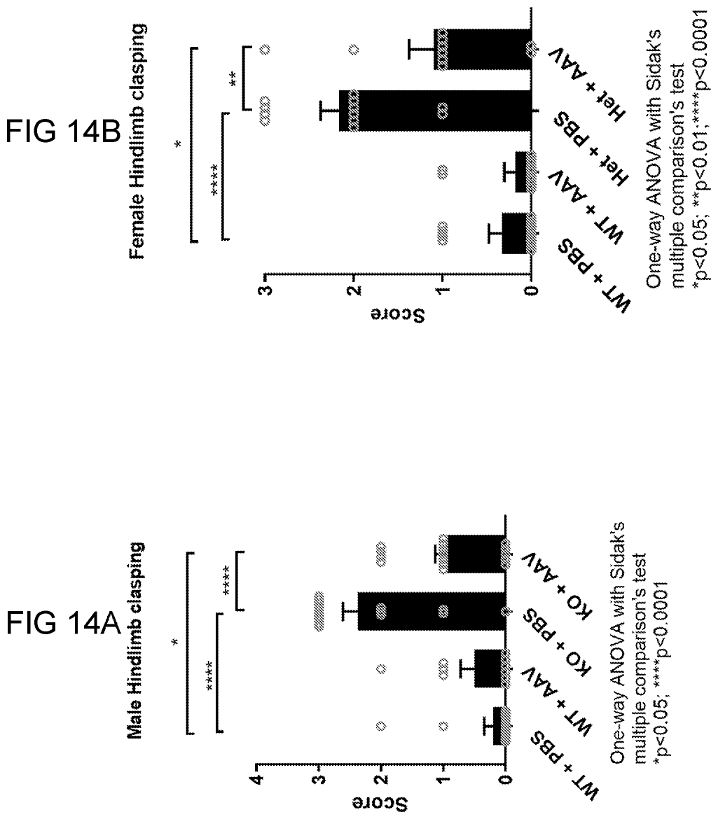

The three alternative CDKL5 isoforms have been injected in neonatal Cdkl5-ko mice to test how a potential therapeutic benefit compares to that of isoform 1. FIG. 12 shows significant correction of clasping phenotype with treatment by alternative CDKL5 isoforms (2, 3, and 4). FIGS. 8A to 8D provide CDKL5 expression levels or activity for AAV.CDLK5 vector constructs for the expression isoform 1, isoform 2, isoform 3 or isoform 4. FIG. 8A shows expression levels in knock-out mice injected with AAV vectors (5×10¹⁰ GC, neonatal ICV) expressing each one of these isoforms, as compared to a wild-type mouse injected with vehicle and a knock-out mouse injected with vehicle. FIG. 8B shows CDKL5 activity as determined using the pS222EB2 in a wild-type mouse injected with vehicle (PBS), a knock-out mouse injected with vehicle, or the AAV.CDKL5-1co. FIG. 8C shows CDKL5 activity as determined using the pS222EB2 for the groups in FIG. 8A. FIG. 8D shows CDKL5 expression levels for the groups in FIG. 8B.

In summary, all isoforms expressed as proteins and have comparable catalytic activity. Therapeutic outcome with isoforms 2, 3 or 4 in CDD mouse model are comparable to isoforms 1 when tested head-to-head (5×10¹⁰ GC, neonatal ICV). Overall, CDKL5 gene therapy with CDKL5 isoform 1 is a promising and safe approach in male CDD model mice with a significant therapeutic benefit.

### Example 6: Pilot NHP Study for Toxicity and Safety Testing of hCDKL5 Gene Therapy

We wanted to investigate the expression pattern and safety profile of the AAV-hSyn-CDKL5-1co.WPRE construct packaged in an AAVhu68 capsid in NHPs. Vector was generated using the vector genome described herein and production methods which have been previously described. See, e.g., WO 2018/160582, which is incorporated herein by reference. A group of six rhesus macaques (age 4-6 years old) was injected via the cisterna magna (ICM). We tested different conditions:
A. dose 1×10¹⁴ GC, injected in 1 ml buffer
B. dose 1×10¹⁴ GC, injected in 3 or 5 ml buffer
C. dose 3×10¹⁴ GC, injected in 3 ml buffer
D. dose 1×10¹⁴ GC, injected in 1 ml buffer, after 2 days of pre-treatment with the diuretic acetazolamine (Diamox®) to reduce CSF production.

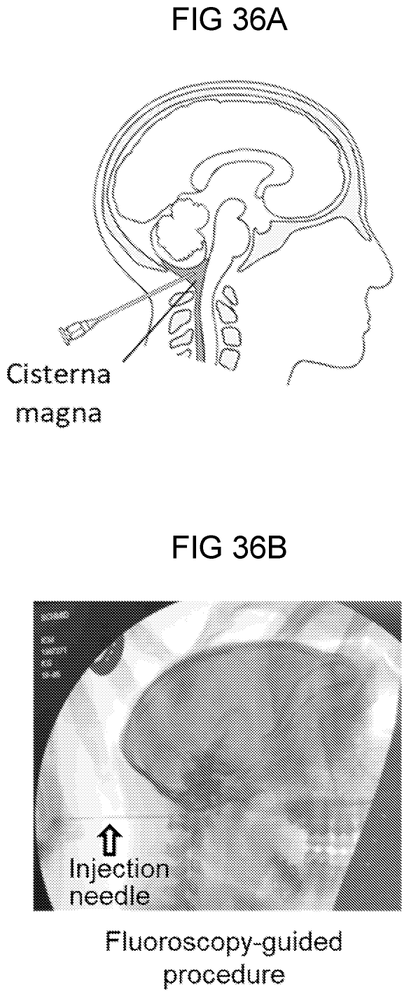

FIG. 36A shows a schematic overview of the intra-cisterna magna (ICM) administration procedure. FIG. 36B shows a more detailed overview of the ICM administration as a fluoroscopy-guided procedure. Injection into CSF via the cisterna magna (ICM) provides best access to brain.

Briefly, for non-human primate (NHP) study, the toxicity and safety testing of hCDKL5 gene therapy was performed using an AAVhu68-hSyn-Cdkl5-1co-WPRE vector. In a preliminary study, three different doses were assessed, 3×10¹² GC/animal, 1×10¹³ GC/animal and 3×10¹³ GC/animal. For the pilot study, a dose of 1×10¹⁴ GC/animal was selected, and two different volumes (3 mL and 5 mL) were assessed for delivery of the AAV vector to the cerebrospinal fluid (CSF) via cisternal magna. Other study arms used 1×10¹⁴ GC/animal with a diuretic (e.g., a Diamox brand acetazolamide) or 3×10¹⁴ GC/animal (subject).

Additionally, we tested a dose of 2×10¹², 1×10¹³, and 3×10¹³ GC/subject. Treatment with CDKL5 vector was well tolerated, no signs of changes in clinical blood chemistry was observed. Observations from cage-side neurological exams found no changes from baseline.

Necropsy was carried out 28 days after injection, followed by molecular analysis, histology and pathology review. Overall, no major transduction differences in main organs outside CNS were found (e.g., transduction of liver has likely already reach a maximum). No major transduction differences in spinal cord and DRGs (remain at very high transduction rates) were observed, however significant changes in brain tissues depending on the injection parameters became evident. The highest transduction increase of transduction was seen in the cortex. Diamox lead to a slight increase in transduction efficiency through the brain.

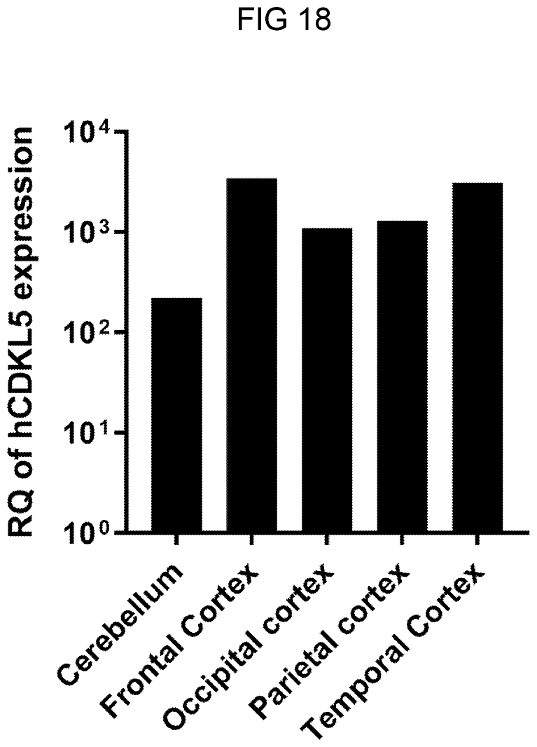

FIG. 17 shows vector biodistribution for each NHP across non-neuronal tissue, spinal tract tissues and brain tissue. In FIG. 18, only the vector biodistribution data for brain is shown. The results show that there is an observed strong transduction of dorsal root ganglion tissue (DRGs), moderate to low transduction of brain tissues, and present transduction leakage into neuronal tissues. The pathology result indicated mild axonopathy in dorsal white matter tracts. Slight differences in hCDKL5 mRNA levels were observed. There is a trend for higher mRNA expression when a 3 ml injection volume was used. We also visualized hCDKL5 mRNA distribution by in-situ hybridization (ISH). Dorsal root ganglia (DRG) showed very strong mRNA expression across all six NHP. In the motor cortex small numbers of transduced neurons were observed (<10%). Occasionally, small clusters of transduced neurons were found. There was no significant difference in hCDKL5 mRNA positive neurons in the motor cortex across NHPs in this study.

A pathology review did not find any gross lesions across tissues and NHP. The NHP that had received the highest dose showed mild signs of inflammatory cell infiltration in the liver. Additionally, mild to moderate spinal cord axonopathy and DRG satellitosis in all NHPs.

Additionally, we examined vector expression and safety testing in NHPs of AAVrh91.UbC.CDKL5-1co.miR183 and AAVrh91.CBh.CDKL5-1co.miR183 vectors. Vectors are administered to C. macaques at a dose of 3×10¹⁰ GC via ICM route. Pathology analysis and neuro-examination was performed to evaluate the effects of CDKL5 expression following AAV vector administration.

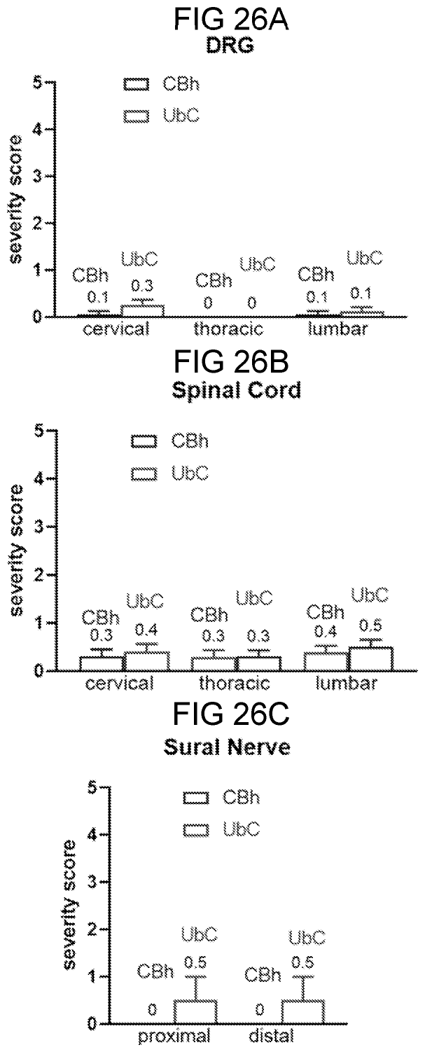

Toxicity and safety were evaluated following administration AAVrh91.UbC.CDKL5-1co.miR183 and AAVrh91.CBh.CDKL5-1co.miR183 vectors to NHPs, at a dose of 3×10¹⁰ GC via ICM route. In examining clinical blood chemistry, the result appeared normal throughout study, no elevation of ALT or AST observed. We observed no signs of safety or toxicity concerns. In analysis of cerebrospinal fluid (CSF), we observed 2 NHP with persistent mild pleocytosis, but there was no evidence of inflammation at ICM injection site during necropsy. In cage-side neurological examination, no overt changes or defects were observed. In a pathology tissue slide review we examined DRG/SpC, peripheral nerves and other organs. FIG. 26A shows severity score observed in DRG neurons from cervical, thoracic, and lumbar collected tissue from NHPs treated with AAVrh91.UbC.CDKL5-1co.miR183 or AAVrh91.CBh.CDKL5-1co.miR183 vectors at a dose of 3×10¹⁰ GC administered via the ICM route. FIG. 26B shows severity score observed in spinal cord neurons from cervical, thoracic, and lumbar collected tissue from NHPs treated with AAVrh91.UbC.CDKL5-1co.miR183 or AAVrh91.CBh.CDKL5-1co.miR183 vectors at a dose of 3×10¹⁰ GC via ICM route. FIG. 26C shows severity score observed in sural nerve from proximal and distal collected tissue from NHPs treated with AAVrh91.UbC.CDKL5-1co.miR183 or AAVrh91.CBh.CDKL5-1co.miR183 vectors at a dose of 3×10¹⁰ GC via ICM route.

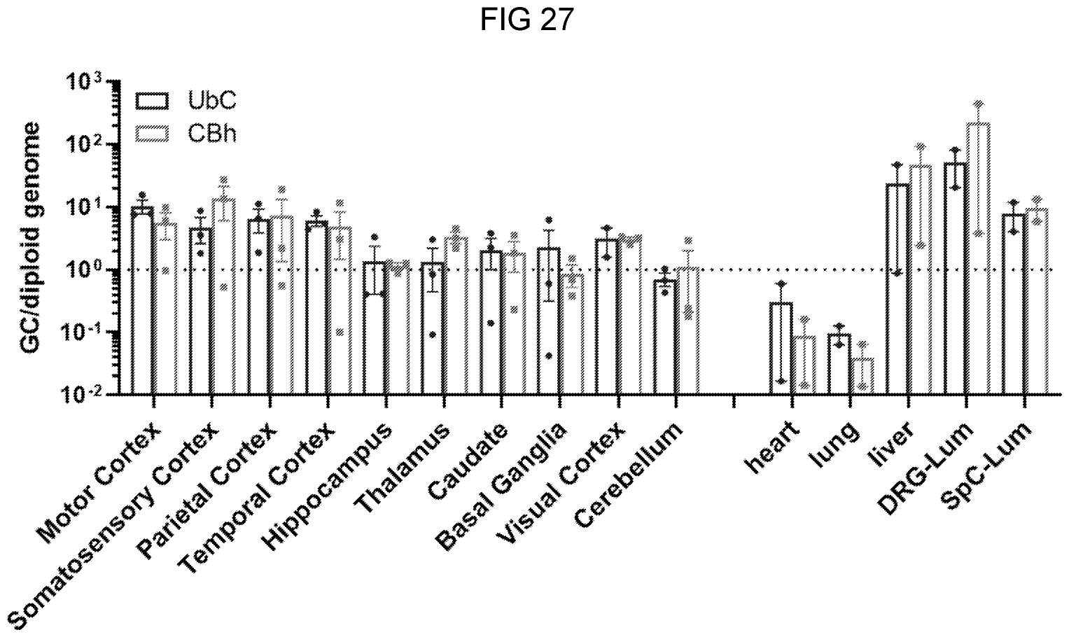

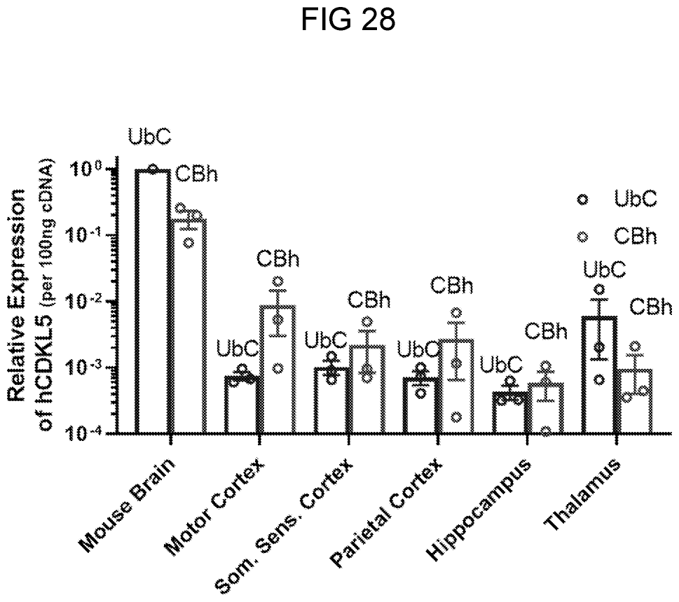

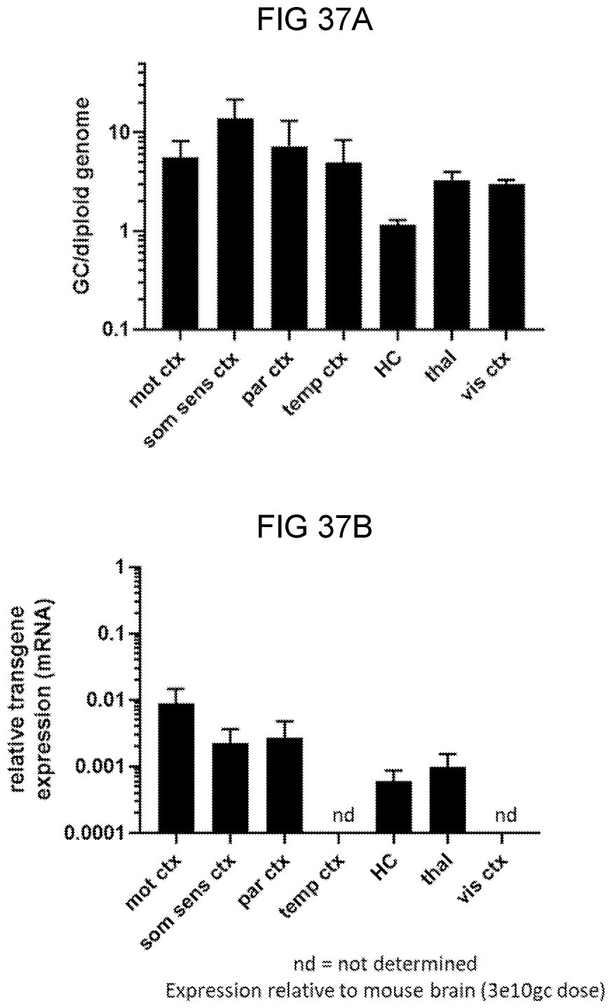

Next, we examined vector copy numbers in different tissue (FIGS. 27 and 37). FIG. 27 shows results of the vector copy number plotted as GC/diploid genome in various tissues oh NHP following administration with AAVrh91.UbC.CDKL5-1co.miR183 or AAVrh91.CBh.CDKL5-1co.miR183 vectors. FIG. 37A shows analysis of the brain transduction as measured by vector genome copies via qPCR of extracted DNA/RNA from different brain regions of NHP following administration with AAVrh91.UbC.CDKL5-1co.miR183. We observed good transduction in cortex (˜10 GC/cell), and high transduction in DRG neurons and liver cells (˜100 gc/cell). Furthermore, we analyzed transgene expression in different tissues (FIGS. 28 and 37B). FIG. 28 shows relative expression of CDKL5, plotted as per 100 ng of cDNA, in various CNS tissue (motor cortex, som. sens. Cortex, parietal cortex, hippocampus, thalamus) of NHP, as compared to results observed in mouse brain. FIG. 37B shows relative CDKL5 transgene expression (mRNA) as measured via qPCR of RNA extracted from different NHP brain regions following administration with AAVrh91.UbC.CDKL5-1co.miR183 (relative to expression in mouse brain when administered at a dose of 3×10¹⁰ GC).

Next, we performed In Situ Hybridization (ISH) microscopy to examine transgene expression in DRG tissue. We observed that CDKL5 transgene mRNA was sparsely detected throughout the NHP brain. Although we observed sporadic transgene expression, it should be noted that some of the DRG neurons were overexposed during analysis (data not shown). Also, the sensitivity of detection technology may underestimate number neurons expressing CDKL5 transgene.

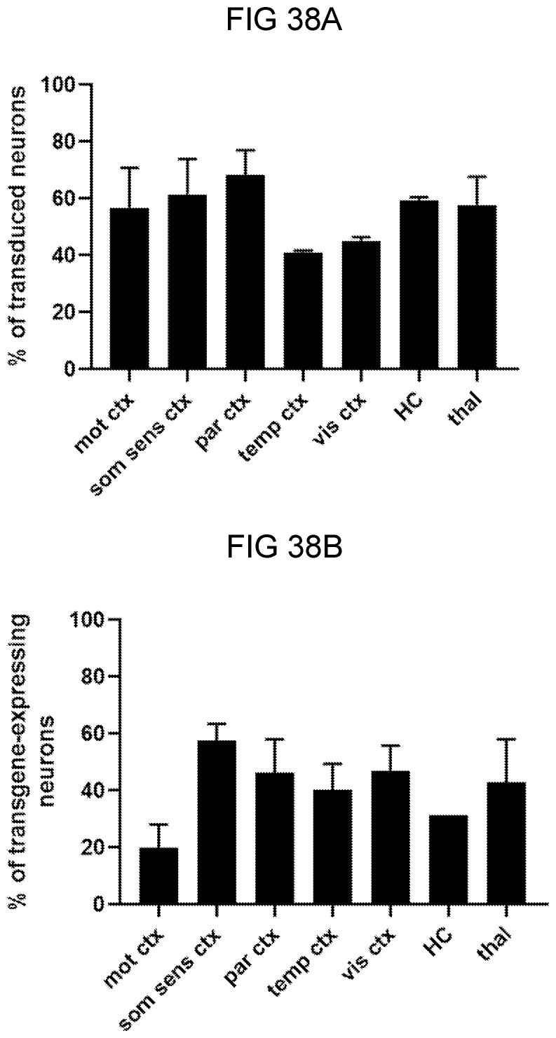

Additionally, we performed a single neuron analysis of brain tissue following administration with AAVrh91.UbC.CDKL5-1co.miR183 via a 2-step PCR detection of presence of vector genome and presence of CDK15 expression, and confirmed by PCR detection of vector genome copies or CDKL5 mRNA from bulk tissue (FIGS. 38A and 38B). FIG. 38A shows results of molecular analysis of CDKL5 gene therapy outcome on the single-neuron basis, plotted as percent of transduced neurons measured by vector genome copies. FIG. 38B confirms results obtained from single neuron analysis. FIG. 38B shows CDKL5 transgene expression levels as measured from bulk mRNA, plotted as percent transgene expressing neurons. From these results we observed many neurons expressing CDKL5 transgene, but at modest levels.

In conclusion, AAV-CDKL5 vector (SEQ ID NO: 1) examined in our studies can be used to achieve stable CDKL5 protein expression in neurons. AAV-CDKL5 gene therapy significantly improved the phenotype of a CDD mouse model. Additionally, AAV-CDKL5 vector can be efficiently delivered via the cisterna magna to non-human primates and expresses in throughout the CNS.

### Example 7: hCDKL5 Gene Therapy AAVhu68.UbC.hCDKL5-1co.miR183.rBG

AAVhu68.UbC.hCDKL5-1co.miR183.rBG is an AAV serotype hu68 (AAVhu68) vector expressing a mutant coding sequence of the human CDKL5 isoform 1 gene. AAVhu68.UbC.hCDKL5-1co.miR183.rBG addresses the significant unmet need by providing functional CDKL5 protein in the CNS and thereby correcting the underlying cause of the disease as described below. The described first-in-human (FIH) trial is an open-label, multi-center, dose escalation study of AAVhu68.UbC.hCDKL5-1co.miR183.rBG administered via an intra-cisterna magna (ICM) injection to evaluate safety, tolerability, and exploratory efficacy endpoints in pediatric (≥30 days of age) and adult subjects with CDKL5 deficiency disorder (CDD).

### Route of Administration

Many animal models of monogenic central nervous system (CNS) diseases have been successfully treated using AAV-mediated gene transfer, and several early human studies using a first-generation AAV vector demonstrated the safety of vector delivery to the brain (Janson et al., 2002; Mandel and Burger, 2004; Kaplitt et al., 2007; Mittermeyer et al., 2012; Bartus et al., 2014). However, the low efficiency of these vectors prevented the translation of efficacy in animal models into clinical benefits. With the advent of second-generation AAV vectors, the potential for gene transfer to the brain has been greatly enhanced. In particular, some clade F isolates, such as AAV9, have demonstrated extremely efficient brain transduction (Gray et al., 2013; Haurigot et al., 2013; Hinderer et al., 2014b; Hinderer et al., 2015). Using these more efficient vectors, gene therapy has shown greatly enhanced potential to treat a variety of neurological disorders, and several programs utilizing second-generation vectors have progressed into the clinic (Haurigot et al., 2013; Hinderer et al., 2014b; Hinderer et al., 2015; Gurda et al., 2016).

Early studies of CNS gene transfer were challenged not only by the low gene transfer efficiency of first-generation AAV vectors, but also limitations in the available delivery methods. Most early nonclinical and clinical studies utilized direct vector injection into the parenchyma of the brain or spinal cord (Vite et al., 2005; Worgall et al., 2008; Colle et al., 2010; Ellinwood et al., 2011; Tardieu et al., 2014). While this method yields robust transduction near the injection site, translating this approach to diseases affecting cells throughout the CNS was difficult because large numbers of vector injections were required to achieve widespread transgene delivery. An additional obstacle to CNS gene transfer was the finding that intraparenchymal vector injection could trigger inflammation at the injection site, which could promote adaptive immune responses against the transgene product (Worgall et al., 2008; Colle et al., 2010; Ellinwood et al., 2011; Ciesielska et al., 2013). Two alternative vector delivery methods have been developed to more safely and effectively target large regions of the CNS.

The first was based on the discovery that some AAV vectors, including AAV9, can transduce cells within the CNS after IV delivery (Foust et al., 2009). However, IV vector delivery has two critical limitations. First, the low efficiency of vector penetration into the CNS necessitates extremely large vector doses to achieve therapeutic levels of transgene expression, increasing the risk of systemic toxicity and potentially requiring quantities of vector that may not be feasible to manufacture for many patient populations (Gray et al., 2011; Hinderer et al., 2014b; Gurda et al., 2016). Second, gene transfer to the CNS after IV vector delivery is profoundly limited by pre-existing NAbs to the vector capsid (Gray et al., 2011). Given the high prevalence of AAV NAbs in humans, this leaves a significant population of patients who would not be candidates for IV AAV treatment. In order to circumvent the limitations of IV AAV for targeting the CNS, intrathecal (IT) vector delivery has been developed as an alternative approach. Using the cerebrospinal fluid (CSF) as a vehicle for vector dispersal, the IT ROA has the potential to achieve transgene delivery throughout the CNS and peripheral nervous system (PNS) with a single minimally invasive injection. Animal studies have demonstrated that by obviating the need to cross the blood-brain barrier, IT delivery results in substantially more efficient CNS gene transfer with much lower vector doses than those required for the IV approach (Gray et al., 2011; Hinderer et al., 2014b). Since antibodies are present at very low levels in CSF, IT vector delivery is not affected by pre-existing NAbs to the AAV capsid, making this approach applicable to a broader patient population (Haurigot et al., 2013). IT AAV delivery can be performed using a variety of routes for CSF access. Lumbar puncture (LP) is the most common method for accessing CSF, and was therefore evaluated as a route for AAV administration in NHPs. Delivery of an AAV9 vector into the CSF via an LP was found to be at least 10-fold less efficient at transducing cells of the brain and spinal cord compared to injection of the vector more superiorly at the level of the cisterna magna (Hinderer et al., 2014b).

The superior brain transduction achieved with a single ICM injection in NHPs resulted in the selection of this ROA for the clinical studies of AAVhu68.CB7.CI.hARmiR3610.WPRE.rBG. Once a common procedure, ICM injection (also known as suboccipital puncture) was ultimately supplanted by LPs in the pre-imaging era due to rare cases of injury to the brainstem or nearby blood vessels (Saunders and Riordan, 1929). Today, the procedure can be performed under real-time computed tomography (CT) guidance, allowing for visualization of critical structures, such as the medulla, vertebral arteries, and posterior inferior cerebellar arteries during needle insertion (Pomerantz et al., 2005; Hinderer et al., 2014b).

The AAVhu68.UbC.hCDKL5-1co.miR183.rBG filled drug product (FDP) consists of a non-replicating recombinant adeno-associated viral (rAAV) vector active ingredient and a formulation buffer. The rAAV vector is produced at a contract manufacturing organization (CMO). AAVhu68.UbC.hCDKL5-1co.miR183.rBG is produced using procedures that ensure the safety, identity, quality, purity, and strength of the product with practices consistent with both the “U.S. Food and Drug Administration's (FDA's) Guidance for Industry cGMP for Phase 1 Investigational Drugs” (July 2008) and “FDA Guidance for Industry: Chemistry, Manufacturing, and Control (CMC) Information for Human Gene Therapy Investigational New Drug Applications (INDs)” (January 2020).

The manufacturing process for AAVhu68.UbC.hCDKL5-1co.miR183.rBG involves transient transfection of human embryonic kidney 293 (HEK293) cells with plasmid DNA. To support clinical development, a single-batch or multiple batches of the bulk drug substance (BDS) are produced by polyethylenimine- (PEI-) mediated triple transfection of HEK293 cells in bioreactors. Harvested AAV material are purified sequentially by clarification, tangential flow filtration (TFF), affinity chromatography, and anion exchange chromatography in disposable, closed bioprocessing systems where possible. The drug substance (DS) and drug product (DP) is formulated in intrathecal final formulation buffer (ITFFB; artificial CSF with 0.001% poloxamer 188). The BDS batch or batches is frozen, subsequently thawed, pooled if necessary, adjusted to the target concentration, sterile-filtered through a 0.2 μm filter, and filled into vials. Fill data is provided as part of the lot documentation package.

Manufacturing process independent of scale is monitored using droplet digital polymerase chain reaction (ddPCR) titer assay that is also used to define the clinical dose. The assay was developed and qualified for use by assessing linearity, accuracy and precision. The same assay is used throughout program development.

### Description of Biological Product

The biological product comprises hCDKL5-1co, human cyclin-dependent kinase-like 5, isoform 1 (engineered mutant); ITR, inverted terminal repeats; miR183, microRNA-183; PolyA, polyadenylation; rBG, rabbit β-globin; UbC, ubiquitin C), and its sequence elements are detailed below (See also, SEQ ID NO: 49 (vector genome), and SEQ ID NO: 50 (expression cassette)).

### Manufacturing: Components and Materials

AAVhu68.UbC.hCDKL5-1co.miR183.rBG is produced by triple plasmid transfection of HEK293 cells with the AAV cis plasmid (pAAV.UbC.hCDKL5-1co.miR183.rBG.KanR (vector genome of SEQ ID NO: 49)), the AAV trans plasmid encoding the AAV2 rep and AAVhu68 cap genes (pAAV2/hu68n.KanR (comprising SEQ ID NO: 55), and the helper adenovirus plasmid (pAdAF6.KanR). The size of the AAVhu8.UbC.hCDKL5-1co.mirR183.RBG packaged vector genome is 4857 bases (including wild-type length ITRs). The vector genome in the plasmid, as described in SEQ ID NO: 49, includes a shortened AAV2 ITR of 130 base pairs, wherein the external A elements is deleted. The shortened ITR is reverted back to the wild-type length of 145 base pairs during vector DNA amplification using the internal A element as a template.

The cis plasmid contains the following vector genome sequence elements:
Inverted Terminal Repeat (ITR): The ITRs are identical, reverse complementary sequences derived from AAV2 (130 base pairs [bp] (SEQ ID NO: 51), GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.
Human Ubiquitin C (UbC) Promoter: This ubiquitous promoter (1229 bp, GenBank: D63791.1) was selected to drive transgene product expression in a CNS cell type (SEQ ID NO: 52).
Coding Sequence: The coding sequence is an engineered version of the human CDKL5 isoform 1 gene (2883 bp, GenBank: NP_001310218.1 (SEQ ID NO: 20)). This isoform comprises >85% of the total brain CDKL5 expression and is considered the primary brain isoform of CDKL5. (Note: The original name for the predominant brain isoform of CDKL5 was isoform 2. However, CDKL5 isoform 2 was recently redesignated CDKL5 isoform 1 in Hector et al 2016. While the GenBank sequence listed above has not been updated to reflect this nomenclature change and is still designated CDKL5 isoform 2, we will refer to the coding sequence in AAVhu68.UbC.hCDKL5-1CO.miR183.RBG as CDKL5 isoform 1 to reflect the current nomenclature used in this field for the predominant brain isoform of CDKL5) (SEQ ID NO: 22).
MicroRNA-183 (miR183): Four 22-bp target sequences for miR183 (GenBank: NR_029615.1) are included in the 3′ untranslated region of the human CDKL5 sequence. microRNAs post-transcriptionally downregulate expression of target messenger ribonucleic acids (mRNAs) in multicellular organisms by affecting both mRNA stability and translation. Since miR183 expression is largely restricted to the DRG, the miR183 target sequences enable DRG-specific downregulation of the human CDKL5 transgene product. (SEQ ID NO: 11).
Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail (SEQ ID NO: 53).

A AAV2/hu68 trans plasmid pAAV2/hu68 (comprising SEQ ID NO: 55) is used. The AAVhu68 trans plasmid encodes the four WT AAV serotype 2 (AAV2) Rep proteins and the three WT AAV VP capsid proteins from AAVhu68. An adenovirus helper plasmid used which contains the regions of adenovirus genome that are important for AAV replication; namely, E2A, E4, and VA RNA (the adenovirus E1 functions are provided by the HEK293 cells). However, the plasmid does not contain other adenovirus replication or structural genes. The plasmid does not contain the cis elements critical for replication, such as the adenoviral ITRs. The E2, E4, and VA adenoviral genes that remain in this plasmid, along with E1, which is present in HEK293 cells, are necessary for AAV vector production.

### Overview of the Manufacturing Process

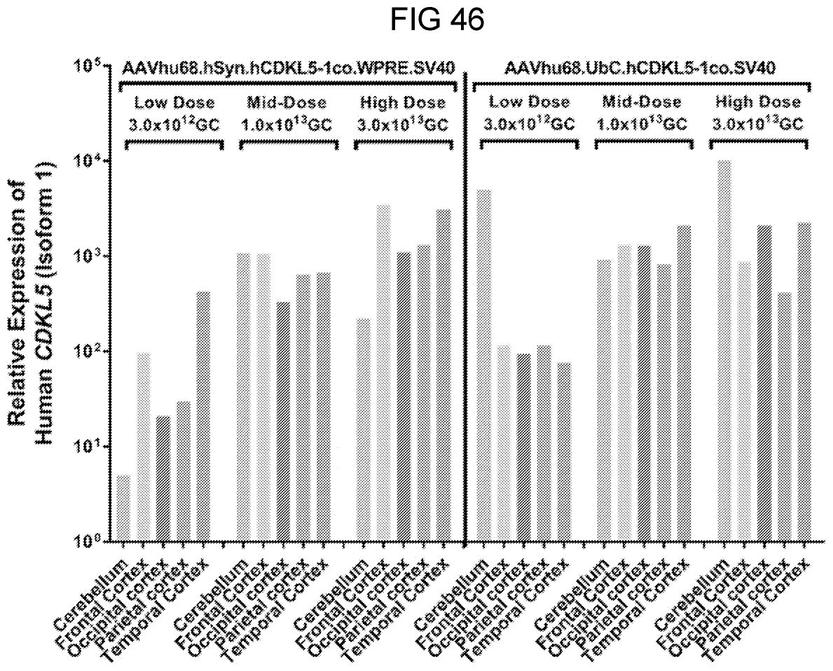

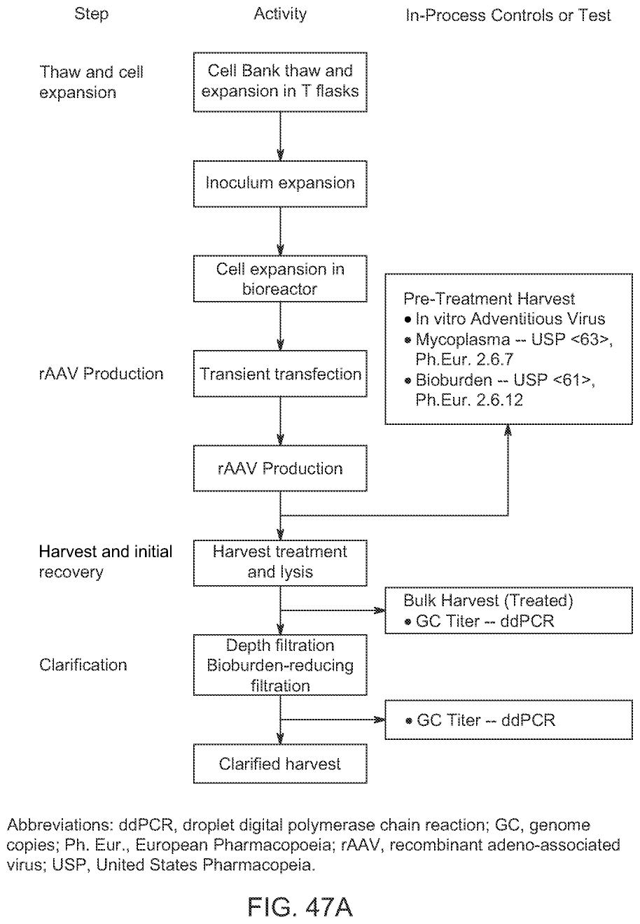

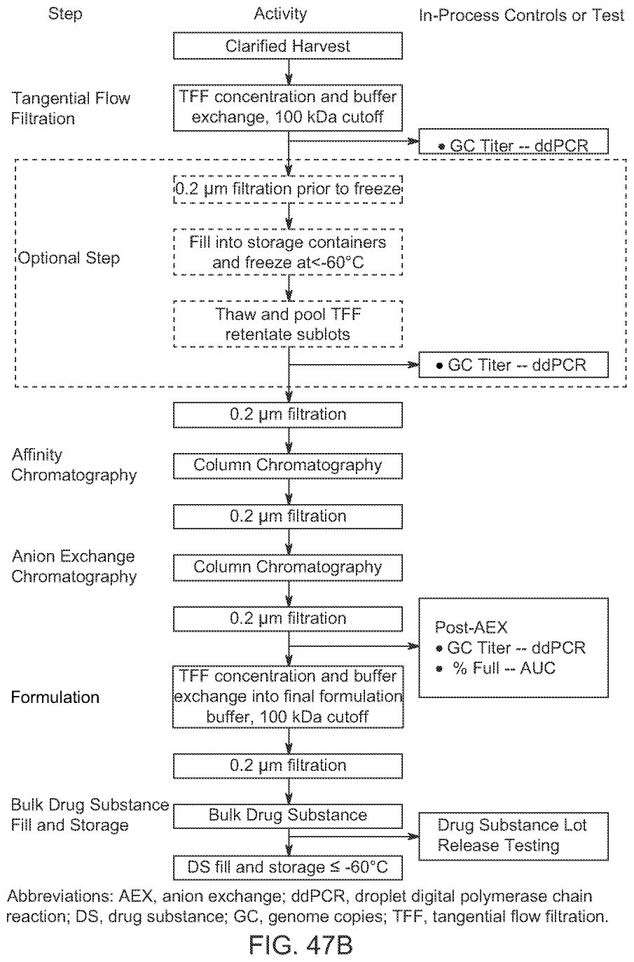

AAVhu68.UbC.hCDKL5-1co.miR183.rBG for the FIH clinical trial is manufactured by transient transfection of HEK293 cells using plasmid DNA followed by downstream purification. A manufacturing process flow diagram is shown in FIGS. 47A, 47B. FIG. 46A shows upstream manufacturing process flow diagram for drug substance. FIG. 46B shows downstream manufacturing process flow diagram for drug substance. The proposed in-process tests are depicted on the right side of the diagram. A description of each production and purification step is also provided.

### ITFFB Manufacturing

The intrathecal final formulation buffer (ITFFB) solution is used at the clinical site to dilute drug product prior to administration following the study protocol. ITFFB diluent is a sterile aqueous solution containing the same excipients as drug product without the active substance. The ITFFB solution will be stored frozen at ≤−60° C.

### Example 8—Further Evaluation of the Pharmacology, Safety, and Toxicology of AAVhu68.UbC.hCDKL5-1co.miR183.rBG

The pharmacology, safety, and toxicology of AAVhu68.UbC.hCDKL5-1co.miR183.rBG, an AAV serotype hu68 (AAVhu68) vector encoding an engineered, mutant, version of the human cyclin-dependent kinase-like 5 (CDKL5) isoform 1 gene has been assessed in various studies with other candidates. As described herein,

Improvements in behavioral phenotypes correlated with an increase in transgene product expression (CDKL5 protein) and activity (phosphorylation of the EB2 substrate) to wild type levels in the disease-relevant target tissue (brain) 14 weeks post treatment. Since the open field test, nest building test, and hindlimb clasping test were found to be the most sensitive assays for evaluating the efficacy of AAV administration in the mouse model of CDD, these assays were selected as readouts for future subsequent murine pharmacology studies.

A pharmacology study evaluates efficacy and determine the MED of AAVhu68.UbC.hCDKL5-1co.miR183.rBG in neonatal (PND 0-1) male Cdkl5^{KO/Y} mice. The MED is determined based on transgene product expression (human CDKL5) and the impact on neurological and behavioral phenotypes reminiscent of clinical features observed in CDD patients. Seizures, which are a clinical feature of CDD in humans, are not assessed in the MED study. Finally, a toxicology study assesses the safety, tolerability, pharmacology (transgene product expression), biodistribution, and excretion of AAVhu68.UbC.hCDKL5-1co.miR183.rBG following ICM administration to juvenile male and female rhesus macaques.

### Methods: Pharmacology Studies

In vivo pharmacology studies are performed in C57BL/6J (wild type) mice, a mouse model of CDD (male Cdkl5 KO mice and female Cdkl5 HET mice), and two species of NHP (Rhesus macaques and African green monkeys).

### A. Mouse Model of CDD

Nonclinical pharmacology studies described herein utilize a knockout mouse model of CDD in which exon 6 of the X-linked murine Cdkl5 has been deleted, resulting in significant reductions in Cdkl5 mRNA and no detectable CDKL5 protein (Wang et al. 2012). This knockout mutation in the CDD mouse model recapitulates a CDKL5 patient-associated splice site mutation that causes skipping of human exon 7 (homologous to murine exon 6) and results in a premature stop codon in human exon 8, leading to a lack of residual CDKL5 protein expression in humans with this mutation. Male mice are commonly utilized by groups studying the CDD mouse phenotype because male Cdkl5^{KO/Y} mice (which are hemizygous for the X-linked Cdkl5 knockout allele) typically exhibit a more severe and consistent phenotype than that of female Cdkl5^{KO/X} mice (which are heterozygous for the X linked Cdkl5 knockout allele and also demonstrate variable tissue mosaicism for expression of the wild type Cdkl5 gene due to random X chromosome inactivation). Many of the phenotypes observed in male Cdkl5^{KO/Y} mice are also reminiscent of those seen in CDD patients, including social behavior phenotypes, motor coordination abnormalities/hyperactivity, impaired cognition function, deficits in neuronal circuit communication, and biochemical defects resulting from impaired CDKL5 kinase activity. The phenotypes observed in male Cdkl5^{KO/Y} mice are discussed below.

### Social Behavior Phenotypes:

Non-social and autistic-like behaviors reminiscent of those observed in CDD patients have been observed in male Cdkl5^{KO/Y} mice by around 8 weeks of age. On the three-chamber social approach test, male Cdkl5^{KO/Y} mice spent more time occupying and sniffing a novel object in the nonsocial chamber compared to time spent in the social chamber with a novel stimulus mouse, and when exposed to a barrier-free chamber, the interaction time of male Cdkl5^{KO/Y} mice with other mice in the social chamber was significantly reduced compared to that of wild type mice, suggesting a reduction in social preference. Nesting behavior of Cdkl5^{KO/Y} mice was also impaired in a home cage environment by this age, which was suggestive of a defect in social behavior, and could not be attributed to olfactory system defects (Wang et al., 2012).

### Motor Coordination/Anxiety-Like Phenotypes:

Male Cdkl5^{KO/Y} mice exhibit various motor phenotypes reminiscent of symptoms in CDD patients, which manifest by around 10-11 weeks of age. For example, a decrease in fall latency on the RotaRod assay and abnormal hindlimb clasping were observed in male Cdkl5^{KO/Y} mice by this age, which was indicative of loss of motor coordination. Anxiety-like motor behaviors (e.g., compulsiveness, hyperactivity, and/or risk-prone behaviors) have also been observed in male Cdkl5^{KO/Y} mice on the elevated zero maze test and open field test (Wang et al., 2012).

### Cognitive Phenotypes and Neural Circuit Defects:

Male Cdkl5^{KO/Y} mice exhibit impaired cognitive function, which is reminiscent of the cognitive symptoms observed in patients with CDD. Cognitive phenotypes include deficits in motor activity, along with impaired learning and memory. For example, a significant deficit in the context- and cue-dependent behavioral responses of male Cdkl5^{KO/Y} mice was observed by 9-12 weeks of age based on the contextual fear conditioning assay. Moreover, Male Cdkl5^{KO/Y} mice also exhibit deficits in auditory-evoked event-related potentials (ERPs), which is indicative of impaired neural circuit activity reminiscent of that seen in patients with CDD. ERPs are stereotyped, electrophysiological responses to specific sensory, cognitive, or motor stimuli. As a measure of sensory information processing, ERPs have been leveraged as a readout of neural circuit communication and have been shown to be altered in disorders of cognition, such as schizophrenia and autism. Disturbances within the neuronal circuit network may contribute to the delay in behavioral responses observed in Cdkl5 KO mice. Circuit communication is dependent upon oscillations over low or high frequencies and low frequencies are associated with long-range neuronal circuit communication. Similar to the neuronal defects reported in autism spectrum disorder patients, oscillatory strength at low delta, theta, and alpha frequencies is attenuated in Cdkl5 KO mice (Wang et al., 2012).

### Biochemical Defects:

In contrast to patients with CDD, Cdkl5 KO mice do not exhibit spontaneous or refractory epilepsy. The absence of this phenotype in CDD mice may be attributed to the age of the animals, study duration, and increased seizure resistance conferred by the genetic background of the Cdkl5 KO mouse model (C57BL/6) (Wang et al., 2012 and Amendola et al., 2014). Abnormal EEG patterns were not observed in Cdkl5 KO mice before 12 weeks age. Several mouse models of CDD have been generated through heterozygous mutations in mouse strains. Recently, a high frequency of epileptic events was observed in aged female Cdkl5 KO mice at 42 weeks of age (Mulcahey et al., 2020). An age-dependent effect on seizure-like events was also noted in a specific strain of Cdkl5 KO mice (Cdkl5RS9X/+ females), which demonstrated an earlier onset of seizures at 16 weeks of age, myoclonic seizure-like events (Racine Stage 3 to 5) at 32 weeks of age, and severe seizure-like events (Racine Stages 3 to 5) at 57 weeks of age (Terzic et al. 2021). The seizure phenotype is age-dependent and requires assessments to be performed in older mice. Based on this observation, it is not possible to observe the seizure phenotype in the completed and planned murine pharmacology studies, which utilize neonatal Cdkl5 KO mice.

Given the similarities to human CDD, neonatal male Cdkl5^{KO/Y} mice treated at a presymptomic stage of disease represent the most relevant animal model of the intended patient population for assessment of the potential efficacy of AAVhu68.UbC.hCDKL5-1co.miR183.rBGin the MED study.

### B. Non-Human Primate

The NHP have been selected for POC large-animal pharmacology studies. These studies include both rhesus macaques and African green monkeys. The NHP was selected for pilot pharmacology studies because the toxicological and immune responses of the NHP closely represent that of a human. Furthermore, the dimensions of the NHP central nervous system (CNS) of both rhesus macaques and African green monkeys act as a representative model of the target clinical population and allow administration of AAVhu68.UbC.hCDKL5-1co.miR183.rBG via the intended clinical route (ICM administration).

### Genotypes

Since Cdkl5 is an X-linked gene, initial POC pharmacology studies utilize female Cdkl5^{KO/X} mice (heterozygous for the Cdkl5 KO allele) and male Cdkl5^{KO/Y} mice (hemizygous for the Cdkl5 KO allele) to model CDD in humans. Since the MED study is conducted in male mice, male Cdkl5^{KO/Y} mice are evaluated. However, because genotypes and sex are challenging to determine in neonatal mice (PND 0-1), full litters of neonatal mice is dosed in the MED study, including female Cdkl5^{KO/X}; however, only male Cdkl5^{KO/Y} mice are enrolled and analyzed to determine the MED. Wild type C57BL/6 mice have also been selected for initial POC and the MED study because they are a similar genetic background as the Cdkl5 KO mouse model and are therefore useful as a healthy control group.

### Sex

Male Cdkl5^{KO/Y} mice, female Cdkl5^{KO/X} mice, and sex-matched C57BL/6J wild type controls have been included in initial POC studies to characterize the severity and progression of the CDD phenotype. Since the data obtained and described herein demonstrated that male Cdkl5^{KO/Y} mice have a more severe phenotype than that of female Cdkl5^{KO/X} mice on certain assessments that are critical for determining the MED (e.g., the open field test), male Cdkl5^{KO/Y} mice have been selected for the MED study. Male Cdkl5^{KO/Y} mice are also preferred for the planned MED study because random X chromosome inactivation leads to mosaic Cdkl5 expression in females due to Cdkl5 being an X-linked gene. Mosaicism for Cdkl5 expression can cause phenotype variability due to inter-animal variability in the overall percentage of cells expressing the wild type Cdkl5 allele versus the Cdkl5 knockdown allele, making female mice suboptimal for use in an MED study.

Male and female rhesus macaques and African green monkeys have been used in POC pharmacology studies. Both sexes were selected to model the intended patient population in the planned clinical trial (male and female CDD patients).

### Age

All mouse pharmacology studies have evaluated neonatal mice administered vector on PND 0-1. This age has been selected because it is the earliest feasible treatment time point and represents a pre-symptomatic stage of disease prior to the onset of overt clinical symptoms, which normally begin to manifest around 8-10 weeks of age in male Cdkl5^{KO/Y} mice depending on the assay employed. The neonatal (PND 0-1) mouse model therefore mirrors the disease stage of the youngest intended patient population to the greatest extent feasible.

The POC studies conducted in NHPs have evaluated adult (3-10 years old) animals. This age range was considered sufficient for initial comparisons of the expression profile and safety/toxicity of lead candidate vectors and for modeling the size and anatomy of the cisterna magna of the youngest intended patient population to the greatest extent feasible.

### Dose Selection

The completed POC mouse pharmacology study evaluated test article at a dose of 5.0×10¹⁰ GC because this is close to the highest feasible dose for ICV administration in mice based on expected vector titers and volume constraints. Another POC mouse pharmacology study evaluates AAVhu68.UbC.hCDKL5-1co.miR183.rBG at a 2-fold lower dose (2.5×10¹⁰ GC) because studies conducted with other candidate AAV vectors expressing human CDKL5 have demonstrated efficacy at this dose comparable to that of the highest feasible dose (data not shown). The MED study subsequently includes a high dose, two mid-doses, and a low dose and are selected based upon the results of the aforementioned POC studies. The selected doses permit an assessment of dose-dependent efficacy while ensuring that the dose levels evaluated in the MED study are distinct.

The NHP POC pharmacology study conducted in rhesus macaques utilized a high dose of 3.0×10¹³ GC, which is close to the highest feasible dose for ICM administration in NHPs based on expected vector titers and volume constraints. The mid-dose and low dose were approximately 3-fold and 10-fold lower than the maximum feasible dose, respectively. This range was selected to ensure that doses were distinct and encompassed a dose range similar to that which may be evaluated in the mouse MED pharmacology study and the GLP-compliant NHP toxicology study. The NHP POC pharmacology study conducted in African green monkeys has utilized a dose of 5.0×10¹³ GC because this dose is close to the highest feasible dose for ICM administration in NHPs based on expected vector titers and volume constraints.

### Study Duration

All pharmacology studies conducted in neonatal mice are 13-14 weeks in length, which is a sufficient duration to evaluate the behavioral defects that begin to manifest by 8-11 weeks of age along with any biochemical phenotypes related to loss of Cdkl5 kinase activity.

Both POC pharmacology studies conducted in NHPs are 56 days in length, which is a sufficient duration to evaluate animals during the expected onset, peak, and plateau of transgene product expression and to assess for possible acute safety signals.

### Route of Administration

The ICV route (i.e., administration of vector directly into the cerebral ventricles) has been selected for pharmacology studies in mice because it enables efficient delivery of AAV vector to the disease relevant target tissue (brain). Use of the intended clinical route (ICM administration into the cisterna magna), which employs CSF as a vehicle for vector dispersal with the potential to achieve transgene product expression throughout the CNS via a single minimally invasive injection, is not feasible in mice due to the small size of the animals. However, the ICM route has been selected for the NHP POC pharmacology studies to mirror the intended clinical route and enable the use of a clinical administration system comparable to the one utilized in the planned clinical trial.

### C. Pharmacology Endpoints

### Open Field Test (Hyperactive Behavior Testing)

The open field test measures locomotor activity and can be used to measure anxiety-like behavior in rodents. It consists of a circular or square enclosure with an open, unobstructed field in the center of the apparatus. The open field arena sends out infrared beams from one side of the enclosure to the other. When a beam is broken by an animal moving through it, this is counted as a “beam break.” During the test, a mouse is placed in the enclosure, and study personnel exit the room. Mouse behavior is recorded using video tracking software for 30 minutes and beam breaks are quantified. Activity is and anxiety are evaluated based on movement occurring away from the walls of within the enclosure, including overall ambulatory activity in the center of the field (horizontal activity based on the number of x/y axis beam breaks) and along with the percent center beam breaks) and rearing behaviors (z-axis beam breaks). Cdkl5^{KO/Y} mice have been shown to exhibit hyperactivity on open field testing consisting of increased ambulatory activity (increased x/y axis beam breaks) in the center of the field and increased rearing (increased z-axis beam breaks). Movement within the center of the arena (determined as the percent of beam breaks occurring in the center of the maze [i.e., in contrast to the percent of beams breaks occurring at the periphery of the maze near the walls]) denotes reduced anxiety-like behaviors. A reduction in activity in the center of the field (x/y axis beam breaks and percent center beam breaks) and/or a reduction in rearing behavior (z axis beam breaks) would be expected to indicate an improvement in the hyperactivity phenotype of Cdkl5 KO mice following AAV administration.

### Marble Burying (Hyperactive Behavior Testing)

The marble burying assay evaluates compulsive and hyperactivity phenotypes in rodents. Marble burying was evaluated in Cdkl5 KO mice because these mice have demonstrated hyperactivity phenotypes in other assays (e.g., open field test). To perform the marble burying assay, mice are first acclimated in the home cage for 30 minutes. Mice are then placed in a test cage in which 12 marbles are pre-placed on top of a level mound of dry cage bedding (3 marbles by 4 marbles). The study personnel leaves the room, and the mouse is left for 30 minutes in the cage. After 30 minutes, the mouse is returned to its home cage, and the number of marbles that have been buried in the cage bedding by ≥50% are counted. A decrease the number of buried marbles would be expected to indicate an improvement in the hyperactivity phenotype of Cdkl5 KO mice following AAV administration.

### Elevated Zero Maze (Risk-Prone Behavior Testing)

The elevated zero maze measures behaviors in rodents based on the animal's ability to balance exploration/foraging behaviors (curiosity) and avoidance of potential dangers (risk-taking) (a test for anxiety-like behaviors). The elevated zero maze is a circularly shaped maze with alternating “open” and closed” quadrants. The elevated zero maze test is performed by first acclimating the mouse in the home cage for 30 minutes. The animal is then placed into an open area of the maze, which is illuminated by a lamp at one end of the apparatus to create brightly lit open areas and poorly lit enclosed areas. The study personnel leaves the room, and the animal is video recorded for approximately 15 minutes. The number of entries into the illuminated open zones, time spent in the open zones, and the total distance traveled are subsequently determined in EthoVisionXT video tracking software. Cdkl5 KO mice have been shown to be more risk-prone on elevated zero maze testing, spending an increased amount of time in the brighter open zones compared to normal control mice, which instead spend a greater amount of time in the darker enclosed areas of the apparatus (Wang et al., 2012). A reduction in the number of entries into the open zone, time spent in the open zone, and/or total distance traveled by Cdkl5 KO mice while in the maze would be expected to indicate an improvement in the disease phenotype following AAV administration.

### Nest Building (Social Behavior Testing)

Nest building is a home cage social behavior in rodents that is important for shelter, heat conservation, and reproduction. Nesting involves mice shredding materials, such as tightly packed cotton or twine that has been placed in the cage, and then arranging it into a nest (Deacon 2006). Cdkl5 KO mice demonstrate impaired nest-building behaviors characterized by either failed nest building (i.e., no shredding of the nestlet to create material for nest construction) or poor quality nest building, suggesting a defect in social behavior. Nest building is assessed by first acclimating the mouse for approximately 24 hours in the test room. Mice are then singly housed with a pre-weighed cotton square nestlet in the late afternoon. Approximately 20 hours later, the quality of the newly created nests are scored according to the 5-point scoring system presented in Table immediately below. Additionally, any un-shredded nestlet remaining is weighed and recorded (i.e., ≥˜0.1 g of a nestlet remains). An increase in shredded nestlets (i.e., a decrease in the percent of intact nestlets) and an increase in nest building quality scores would suggest an enhanced quality of nest building and would be expected to indicate an overall improvement in the social behavior phenotype of Cdkl5 KO mice following AAV administration.

| Score | Behavior |
|---|---|
| 1 | Nestlet not noticeably touched (more than 90% intact) |
| 2 | Nestlet partially torn (50-90% remaining intact) |
| 3 | Nestlet mostly shredded but often no identifiable |
| | nest site: less than 50% of the Nestlet remains |
| | intact, but less than 90% is within a quarter of |
| | the cage floor area; i.e., the cotton is not |
| | gathered into a nest but is spread around the cage. |
| | The material may sometimes be in a broadly defined |
| | nest area, but the critical definition here is |
| | that 50-90% has been shredded. |
| 4 | An identifiable but flat nest: more than 90% |
| | of the Nestlet is torn, and the material is gathered |
| | into a nest within a quarter of the cage floor area, |
| | but the nest is flat, with walls higher than mouse |
| | body height (of a mouse curled up on its side) for |
| | less than 50% of its circumference. |
| 5 | A near perfect nest: More than 90% of the Nestlet |
| | is torn and the nest is a crater, with walls higher |
| | than mouse body height for more than 50% of its |
| | circumference. |

Source: Deacon, 2006.

### Hindlimb Clasping (Motor Control Testing)

Hindlimb clasping is a motor control phenotype observed in Cdkl5 KO mice in which animals pull their hindlimbs in toward their body and clasp them together when held upside down (Wang, 2012). Hindlimb clasping is assessed by suspending a mouse over its cage by its tail for 20-30 seconds and observing the behavior of its hindlimbs according to the scoring system presented in Table immediately below. A reduction in cumulative hindlimb clasping scores would be expected to indicate an improvement in the motor phenotype of Cdkl5 KO mice following AAV administration.

| Score | Behavior |
|---|---|
| 0 | Both feet are outstretched laterally with no |
| | hindlimb clasping |
| 1 | Mild phenotype with both feet outstretched |
| | laterally but toes are curling |
| 2 | Animal pulls one limb in towards the body |
| | and toe curling is observed |
| 3 | Both limbs are pulled in towards the body |
| | and feet are clasped together |

### Y Maze Spontaneous Alternation Test (Spatial Working Memory Testing)

The Y maze spontaneous alteration test assesses the exploratory activity of mice and evaluates spatial working memory. The apparatus for the test is an opaque Y-shaped enclosed maze with three arms placed at 120° angles from each other. For this test, the animal is first placed in the center of the maze. The study personnel exit the room, and the mouse is allowed to freely explore the maze for approximately 5 minutes. The animal's movements are recorded on video to assess arm entries, which are defined as the animal moving all four limbs fully into an arm of the maze. Since normal mice usually prefer to investigate a new arm of the maze instead of returning to a portion of the maze that it previously explored, normal mice are expected to exhibit a tendency to explore an arm of the maze that it visited less recently. The tendency to visit an arm of the maze that was less recently explored is referred to as spontaneous alternation, and the percent spontaneous alternation is calculated by dividing the number of spontaneous alternations by the total number of entries minus 2 and multiplying the result by 100 Since Cdkl5 KO mice exhibit reduced spontaneous alternations on the Y maze test relative to WT, an increase in the percentage of spontaneous alternations would be expected to indicate an improvement in the exploratory/spatial working memory phenotype of Cdkl5 KO mice following AAV administration.

### Contextual Fear Conditioning Test (Learning and Memory Testing)

The contextual fear condition test assesses the learning and memory capacity of rodents. For this test, a training phase is performed in which mice are placed in conditioning chambers for 3 minutes, at the end of which time, the mice receive a 1.5 mA shock to the foot. Mice are then left in the chamber for 1 minute after the shock. The next day, mice are returned to the conditioning chamber again for the testing phase for 5 minutes. The animal is video recorded and the proportion of time that the animals spends “frozen” (i.e., no motion detected except for respiratory movements) during the test phase is determined. Cdkl5 KO mice typically spend a shorter proportion of the test phase frozen, which is indicative of a deficiency in learning and memory. An increase in the proportion of the test time spent frozen would therefore indicate an improvement in the learning and memory deficits of Cdkl5 KO mice following AAV administration (Yennawar, 2019).

Transgene Product Expression—CDKL5 Protein (Western Blot, Immunofluorescence), CDKL5 mRNA (In Situ Hybridization, qPCR, Single-Cell RNAseq)

For NHP pharmacology studies, expression of the transgene product in the disease-relevant target tissue (brain) has been evaluated at the level of mRNA by human CDKL5 in situ hybridization, human CDKL5 qPCR, and single-cell RNAseq. For mouse pharmacology studies, expression of the transgene product in the disease-relevant target tissue (brain) has been evaluated at the level of protein by CDKL5 Western blotting, which detects both human CDKL5 and endogenous mouse CDKL5, and human CDKL5 immunofluorescence using an anti-human CDKL5 antibody. AAV administration would be expected to increase CDKL5 expression in the brain where the protein is needed for normal neuronal function.

### Transgene Product Activity—CDKL5 Substrate Phosphorylation (Phospho-EB2 Western Blot)

The kinase activity of CDKL5 can be assessed by measuring phosphorylation of its substrates, including the microtubule-associated protein EB2. CDKL5 kinase activity is assessed in the disease-relevant target tissue (brain) of Cdkl5 KO mice by phospho-EB2 Western blotting using an antibody that recognizes phosphorylation of serine 222. AAV administration would be expected to increase the abnormally low level of EB2 phosphorylation that is typically observed in the brain of Cdkl5 KO mice.

### Example 9—Further Evaluation the Pharmacology, Safety, and Toxicology of AAVhu68.UbC.hCDKL5-1co.miR183.rBG

A. Proof-of-Concept Pharmacology and Assay Development Study Evaluating the Efficacy of a rAAV Vector in a Mouse Model of CDD

This POC pharmacology study evaluated the efficacy of AAVhu68.hSyn.hCDKL5-1co.WPRE.SV40 following ICV administration to neonatal male Cdkl5^{KO/Y} mice and female Cdkl5^{KO/X} mice. AAVhu68.hSyn.hCDKL5 1co.WPRE.SV40 utilizes the same capsid (AAVhu68) and expresses the same transgene product (human CDKL5 isoform 1). However, AAVhu68.hSyn.hCDKL5-1co.WPRE.SV40 includes a different promoter (hSyn versus UbC) and polyA (SV40 versus rBG), incorporates a WPRE sequence 3′ to the transgene sequence, and lacks the miR183 target sequences for DRG detargeting.

Neonatal (PND 0-1) male Cdkl5^{KO/Y} mice and female Cdkl5^{KOA} mice received a single ICV administration of AAVhu68.hSyn.hCDKL5-1co.WPRE.SV40 at a dose of 5.0×10¹⁰ GC. Age-matched male and female wild type C57BL/6 mice were also administered vehicle (PBS) as a control. In-life assessments included viability checks performed daily, weekly body weight measurements starting at 4 weeks post treatment, and behavioral assessments performed at 11-14 weeks post treatment (open field test, nest building test, marble burying test, hindlimb clasping test, Y maze test, elevated zero maze test, and a contextual fear conditioning test). At 14 weeks post treatment, mice were necropsied, and Western blot analysis was performed to evaluate CDKL5 knockdown and effects on substrate phosphorylation (phospho EB2) in the disease-relevant target tissue (brain).

AAV administration was well-tolerated. Male Cdkl5^{KO/Y} mice and female Cdkl5^{KO/X} mice administered either AAV or vehicle gained weight over the course of the study comparable to that of sex-matched wild type mice administered either AAV or vehicle (data not shown), confirming observations in the published literature that defects in weight gain post birth (i.e., failure to thrive) is not a feature of the Cdkl5 knockout mouse phenotype.

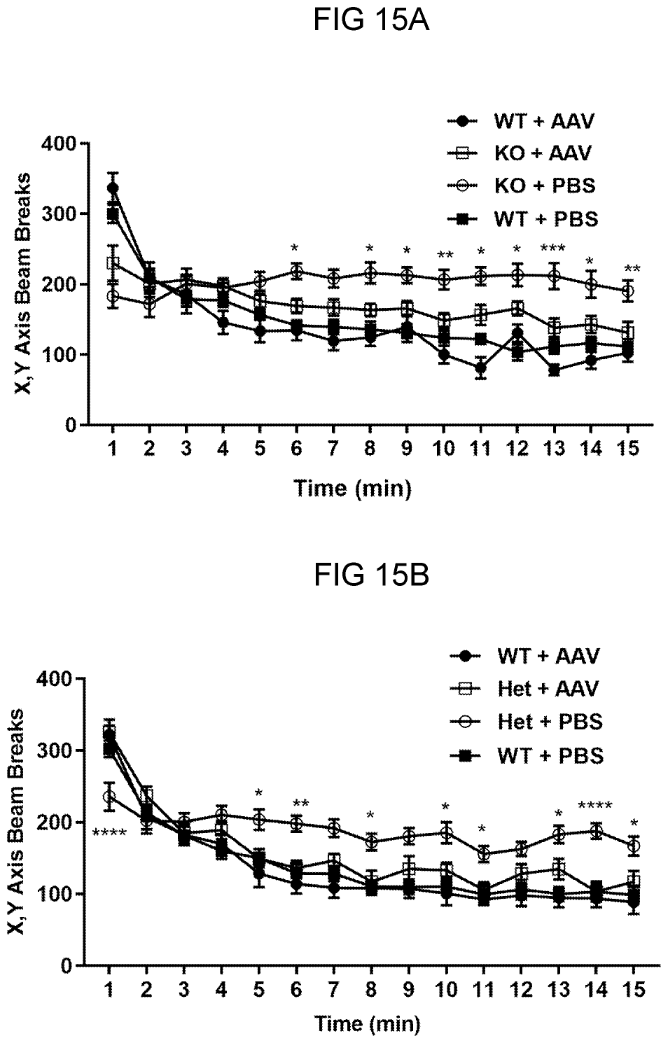

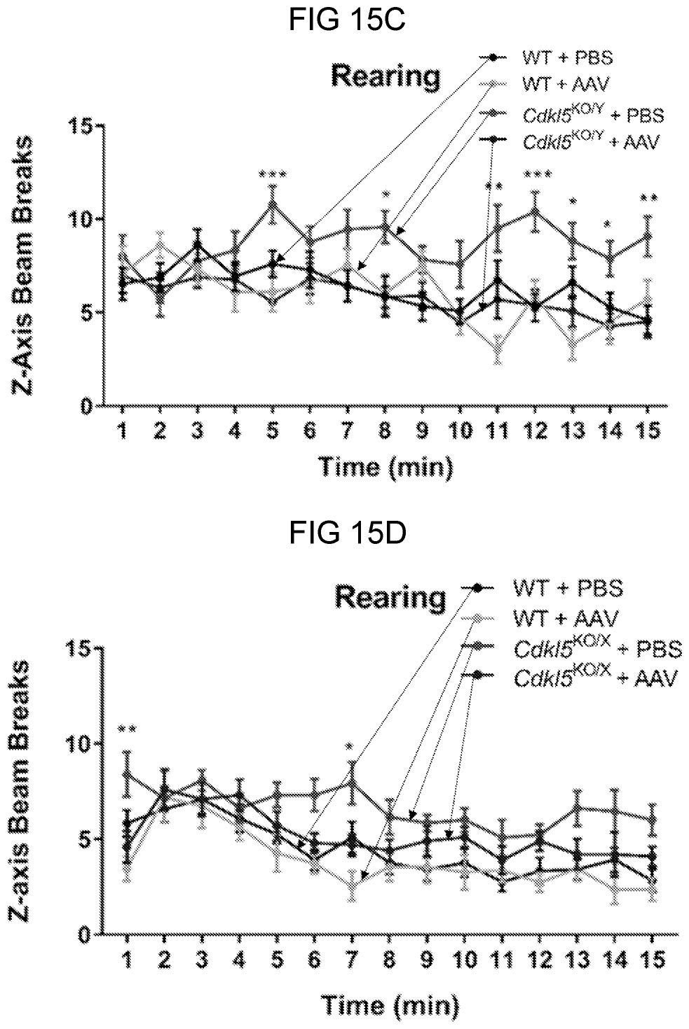

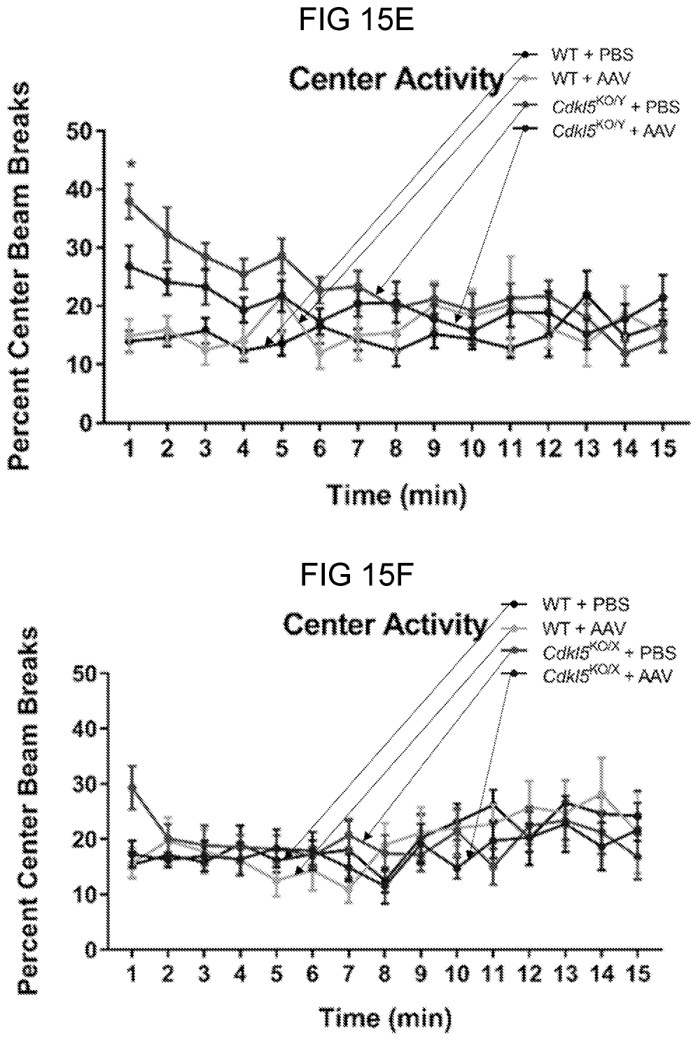

On the open field test, AAV-treated male Cdkl5^{KO/Y} mice and female Cdkl5^{KO/X} mice exhibited reduced hyperactivity (significantly reduced horizontal activity and rearing, and trending reduction in center activity) compared to vehicle-treated male Cdkl5^{KO/Y} mice and female Cdkl5^{KO/X} mice, respectively, with AAV treatment normalizing activity to near wild type levels for all three parameters evaluated. The correction in the hyperactivity phenotype was most evident in male Cdkl5^{KO/Y} mice, as males exhibited a generally more severe hyperactivity phenotype for all three parameters evaluated when compared to that of female Cdkl5^{KO/X} mice (FIGS. 15A-F, See also, Example 3).

FIGS. 15A to 15F show results of the open field test in Male Cdkl5^{KO/Y} Mice and Female Cdkl5^{KO/X} Mice following ICV administration of an AAV vector expressing human CDKL5. FIG. 15A shows results of the horizontal activity open field test in males, plotted as X/Y-Axis beam breaks. FIG. 15B shows results of the horizontal activity open field test in females, plotted as X/Y-Axis beam breaks. FIG. 15C shows results of the rearing open field test in males, plotted as Z-axis beam breaks. FIG. 15D shows results of the rearing open field test in females, plotted as Z-axis beam breaks. FIG. 15E shows results of the center activity open field test in males, plotted as percent center beam breaks. FIG. 15F shows results of the center activity open field test in females, plotted as percent center beam breaks. Briefly, neonatal (PND 0-1) male Cdkl5^{KO/Y} mice and female Cdkl5^{KO/X} mice received a single ICV administration of either AAVhu68.hSyn.hCDKL5 1co.WPRE.SV40 at a dose of 5.0×10¹⁰ GC (N=16 males, 10 females) or vehicle (PBS; N=16 males, 13 females). Additional age-matched C57BL/6 wild type mice were ICV-administered either AAVhu68.hSyn.hCDKL5-1co.WPRE.SV40 (5.0×10¹⁰ GC; N=10 males, 11 females) or vehicle (PBS; N=15 males, 12 females) as controls. At 11-14 weeks post treatment, the open field test was performed. *p<0.05; **p<0.01; ***p<0.001; ****p<0.0001 based on a two-way ANOVA followed by Tukey's multiple comparisons test comparing AAV-treated Cdkl5 KO mice (dark blue line) to vehicle-treated Cdkl5 KO (red line). Abbreviations: AAV, adeno-associated virus; ANOVA, analysis of variance; Cdkl5, cyclin-dependent kinase-like 5 (gene, mouse); GC, genome copies; ICV, intracerebroventricular; KO, knockout; N, number of animals; PBS, phosphate-buffered saline; PND, postnatal day; WT, wild type.

While vehicle-treated male Cdkl5^{KO/Y} mice and female Cdkl5^{KO/X} mice trended toward burying more marbles than that of healthy vehicle-treated sex-matched wild type controls in the marble burying assay, the difference in number of buried marbles was not statistically significant. Thus, this result precluded the use of this assay for assessment of AAV treatment efficacy (FIG. 10B. See also, Example 3).

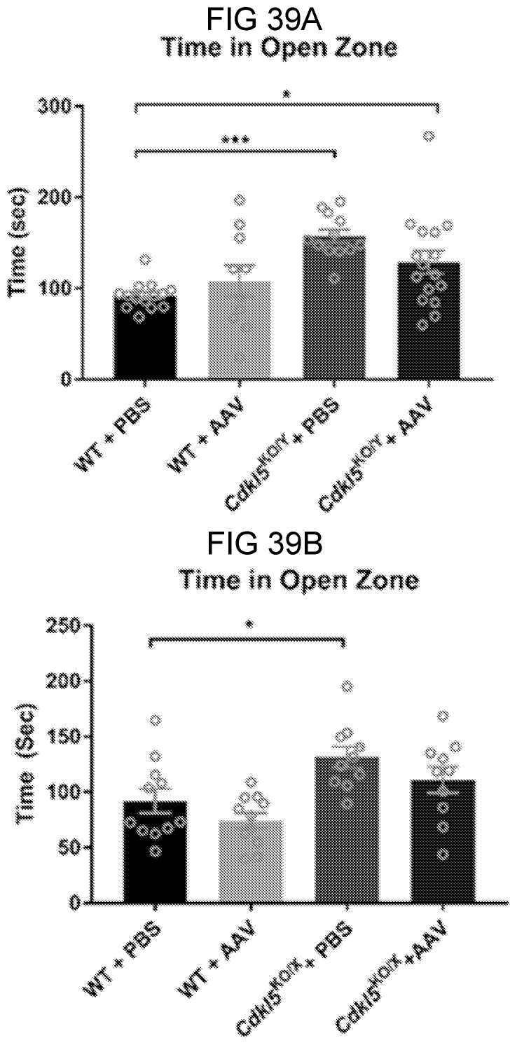

On the elevated zero maze test, AAV-treated male Cdkl5 KO mice exhibited a trending reduction in risk-prone behavior as evidenced by spending less time on average in the open zone of the maze when compared to vehicle-treated male Cdkl5^{KO/Y} mice; however, the reduction was not statistically significant. For female Cdkl5^{KO/X} mice, the increase in risk-prone behavior compared to that of healthy sex-matched vehicle-treated wild type controls was minor, and while a trending reduction in open zone time following AAV administration in female Cdkl5^{KO/X} mice was observed, the difference was not significant (FIGS. 39A, 39B, 40A, 40B, 41A, 41B).

With regard to open zone entries in the elevated zero maze test, AAV-treated female Cdkl5^{KO/X} mice exhibited a marked reduction in risk-prone behavior as evidenced by significantly fewer open zone entries when compared to that of vehicle-treated female Cdkl5^{KO/X} mice, with AAV treatment normalizing entries into the open zone to near-wild type levels. In contrast, the risk-prone phenotype assessed by this parameter was not evident in male Cdkl5^{KO/X} mice, with vehicle-treated male Cdkl5^{KO/Y} mice exhibiting a similar number of entries into the open zone as that of healthy sex-matched vehicle-treated wild type controls, therefore precluding the use of this parameter in males for evaluation of AAV treatment efficacy (FIGS. 39A, 39B, 40A, 40B, 41A, 41B).

With regard to the distance moved in the elevated zero maze, no significant differences were observed for vehicle-treated male Cdkl5^{KO/Y} mice or female Cdkl5^{KO/X} mice compared to that of healthy sex-matched vehicle-treated wild type controls, precluding the use of this parameter in both sexes for assessment of AAV treatment efficacy (FIGS. 39A, 39B, 40A, 40B, 41A, 41B).

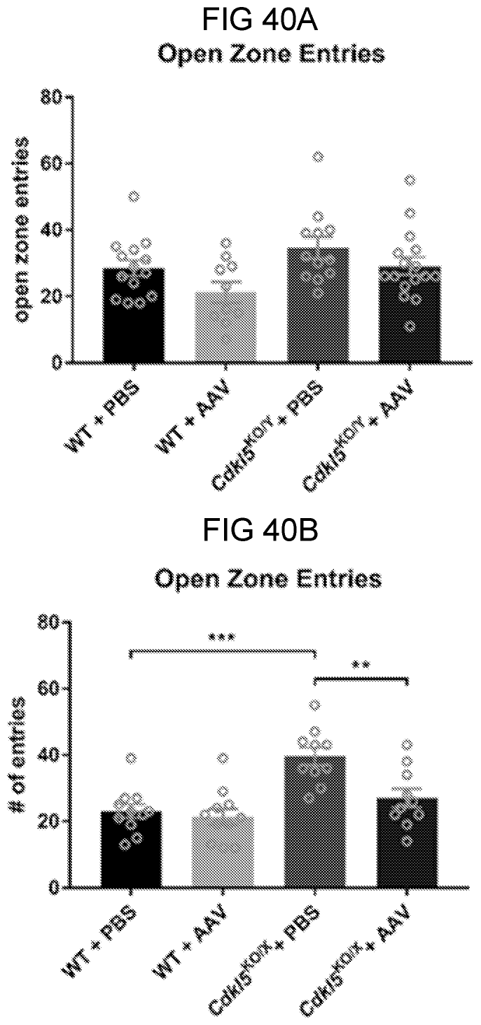

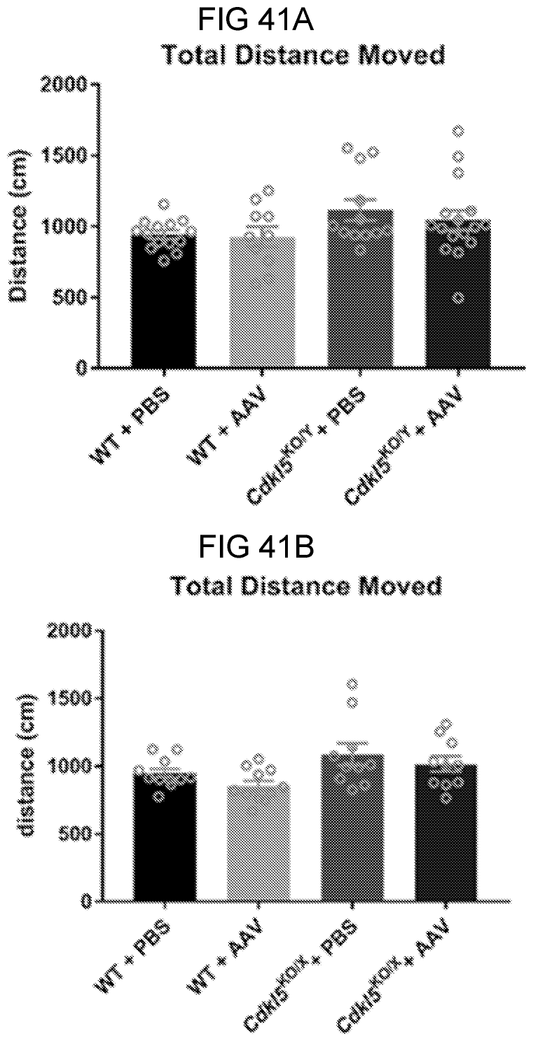

FIG. 39A shows results of the elevated zero maze test in male Cdkl5^{KO/Y} mice following ICV administration of an AAV vector expressing human CDKL5, plotted as time (sec) in open zone. FIG. 39B shows results of the elevated zero maze test in female Cdkl5^{KO/X} mice following ICV administration of an AAV vector expressing human CDKL5, plotted as time (sec) in open zone. FIG. 40A shows results of the elevated zero maze test in male Cdkl5^{KO/Y} mice following ICV administration of an AAV vector expressing human CDKL5, plotted as open zone entries. FIG. 40B shows results of the elevated zero maze test in female Cdkl5^{KO/X} mice following ICV administration of an AAV vector expressing human CDKL5, plotted as open zone entries. FIG. 41A shows results of the elevated zero maze test in male Cdkl5^{KO/Y} mice following ICV administration of an AAV vector expressing human CDKL5, plotted as total distance moved. FIG. 41B shows results of the elevated zero maze test in female Cdkl5^{KO/X} mice following ICV administration of an AAV vector expressing human CDKL5, plotted as total distance moved. Briefly, neonatal (PND 0-1) male Cdkl5^{KO/Y} mice and female Cdkl5^{KO/X} mice received a single ICV administration of either AAVhu68.hSyn.hCDKL5 1co.WPRE.SV40 at a dose of 5.0×10¹⁰ GC (N=16 males, 10 females) or vehicle (PBS; N=12 males, 10 females). Additional age-matched C57BL/6 wild type mice were ICV-administered either AAVhu68.hSyn.hCDKL5-1co.WPRE.SV40 (5.0×10¹⁰ GC; N=10 males, 11 females) or vehicle (PBS; N=14 males, 11 females) as controls. At 11-14 weeks post treatment, the elevated zero maze test was performed. *p<0.05; **p<0.01; ***p<0.001 based on a one-way ANOVA followed by Sidak's multiple comparisons test comparing all groups to each other except for AAV-treated wild type mice. Abbreviations: AAV, adeno-associated virus; ANOVA, analysis of variance; Cdkl5, cyclin-dependent kinase-like 5 (gene, mouse); GC, genome copies; ICV, intracerebroventricular; KO, knockout; N, number of animals; PBS, phosphate-buffered saline; PND, postnatal day; WT, wild type.

On the nest building test, AAV-treated male Cdkl5^{KO/Y} and female Cdkl5^{KO/X} mice displayed an improved quality of nests, as evidenced by a significant increase in nest building quality scores and a significant decrease in the percent of intact nestlets compared to that of vehicle-treated male Cdkl5^{KO/X} mice and female Cdkl5^{KO/X} mice, respectively. Of note, AAV treatment normalized both nest building quality scores and the size of intact nestlets to wild type levels (FIGS. 10A and 10F).

FIG. 10F shows results of the nest building test in male Cdkl5^{KO/Y} mice and female Cdkl5^{KO/X} mice following ICV administration of an AAV vector expressing human CDKL5, plotted as percent weight of original nestlet weight intact. Briefly, neonatal (PND 0-1) male Cdkl5^{KO/Y} mice and female Cdkl5^{KO/X} mice received a single ICV administration of either AAVhu68.hSyn.hCDKL5 1co.WPRE.SV40 at a dose of 5.0×10¹⁰ GC (N=15) or vehicle (PBS; N=8). Additional age-matched male and female C57BL/6 wild type mice were ICV-administered either AAVhu68.hSyn.hCDKL5-1co.WPRE.SV40 (5.0×10¹⁰ GC; N=11) or vehicle (PBS; N=11) as controls. At 11-14 weeks post treatment, the nest building test was performed. Nest quality scores were assigned and the percent of the original intact nestlet based on weight was measured. *p<0.5, **p<0.01, ***p<0.001 based on a one-way ANOVA followed by Sidak's multiple comparisons test comparing all groups to each other except for AAV-treated wild type mice (for nest quality score) and a one-way ANOVA followed by Tukey's multiple comparisons test comparing all groups to each other (for percent of original nestlet). Abbreviations: AAV, adeno-associated virus; ANOVA, analysis of variance; Cdkl5, cyclin-dependent kinase-like 5 (gene, mouse); GC, genome copies; ICV, intracerebroventricular; KO, knockout; N, number of animals; PBS, phosphate-buffered saline; PND, postnatal day; WT, wild type.

AAV-treated male Cdkl5^{KO/Y} mice and female Cdkl5^{KO/X} mice displayed significantly reduced hindlimb clasping scores compared to that of vehicle-treated male Cdkl5^{KO/Y} mice and female Cdkl5^{KO/X} mice, respectively, indicating a significant improvement in motor control. However, AAV treatment did not fully normalize this motor phenotype, as hindlimb clasping scores of AAV-treated male Cdkl5^{KO/Y} mice and female Cdkl5^{KO/X} mice still remained higher than that of healthy sex-matched wild type controls (FIGS. 14A and 14B; See also, Example 3).

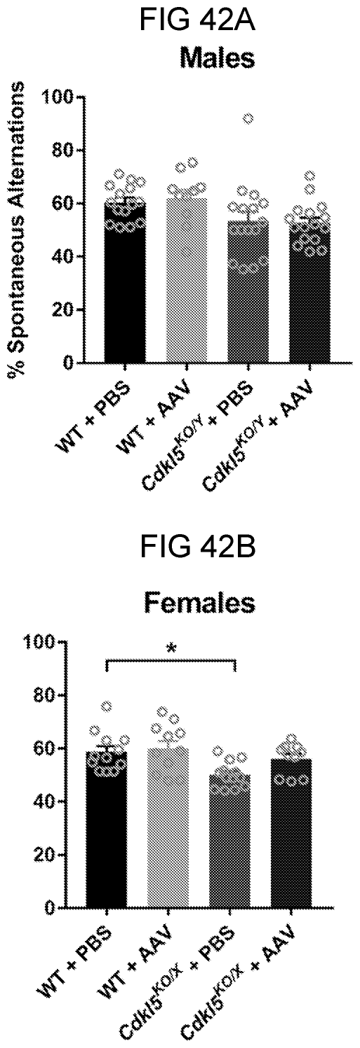

On the Y maze test, AAV-treated female Cdkl5^{KO/X} mice exhibited an increase in the percent of spontaneous alternations when compared to vehicle-treated female Cdkl5^{KO/X} mice, with AAV treatment normalizing the percent alternations to near-wild type levels. This result indicated that AAV treatment increased the tendency of female Cdkl5^{KO/X} mice to explore less recently visited arms of the maze, which is suggestive of an improvement in spatial learning/memory. In contrast, this phenotype was not evident in male Cdkl5^{KO/Y} mice, as vehicle-treated male Cdkl5^{KO/Y} mice displayed a similar percent spontaneous alternations as that of healthy sex-matched vehicle-treated wild type controls, thus precluding the use of this parameter in males for evaluation of AAV treatment efficacy (FIGS. 42A and 42B).

FIG. 42A shows results of Y maze test in male Cdkl5^{KO/Y} mice following ICV administration of an AAV vector expressing human CDKL5, plotted as percent spontaneous alterations. FIG. 42B shows results of Y maze test in female Cdkl5^{KO/X} mice following ICV administration of an AAV vector expressing human CDKL5, plotted as percent spontaneous alterations. Briefly, neonatal (PND 0-1) male Cdkl5^{KO/Y} mice and female Cdkl5^{KO/X} mice received a single ICV administration of either AAVhu68.hSyn.hCDKL5 1co.WPRE.SV40 at a dose of 5.0×10¹⁰ GC (N=16 males, 10 females) or vehicle (PBS; N=16 males, 13 females). Additional age-matched C57BL/6 wild type mice were ICV-administered either AAVhu68.hSyn.hCDKL5-1co.WPRE.SV40 (5.0×10¹⁰ GC; N=10 males, 11 females) or vehicle (PBS; N=15 males, 12 females) as controls. At 11-14 weeks post treatment, the Y maze test was performed. *p<0.05 based on a one-way ANOVA followed by Sidak's multiple comparisons test comparing all groups to each other except for AAV-treated wild type mice. Abbreviations: AAV, adeno-associated virus; ANOVA, analysis of variance; Cdkl5, cyclin-dependent kinase-like 5 (gene, mouse); GC, genome copies; ICV, intracerebroventricular; KO, knockout; N, number of animals; PBS, phosphate-buffered saline; PND, postnatal day; WT, wild type.

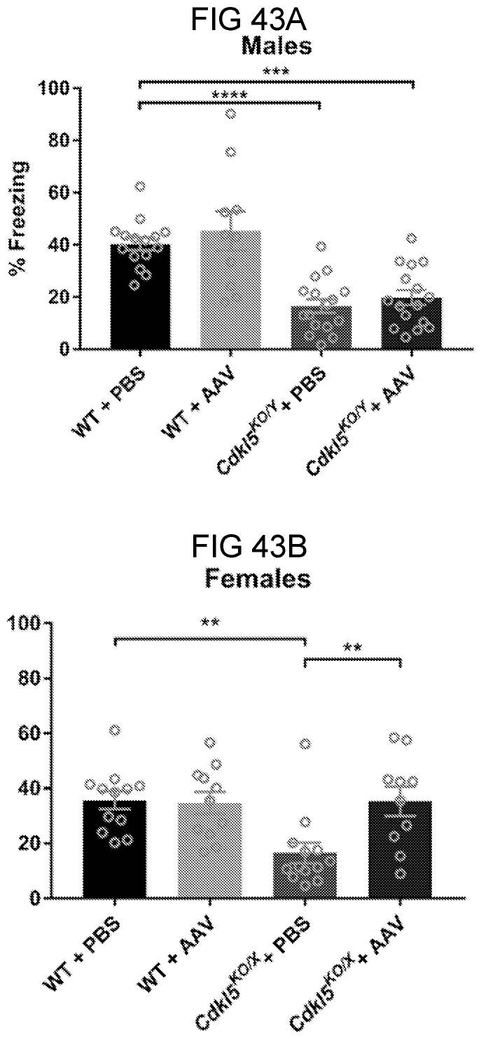

On the contextual fear conditioning test, AAV-treated female Cdkl5^{KO/X} mice displayed a significant increase in percent freezing behavior compared that of vehicle-treated female Cdkl5^{KO/X} mice, indicating a significant improvement in the phenotype following AAV administration. Of note, AAV treatment in female Cdkl5^{KO/X} mice increased percent freezing to the level of healthy sex-matched wild type controls, indicating a normalization of the phenotype. In contrast, AAV treatment in male Cdkl5^{KO/Y} mice did not significantly increase the percent freezing compared that of vehicle-treated male Cdkl5^{KO/Y} mice, indicating that AAV administration did not improve this phenotype in male Cdkl5^{KO/Y} mice despite significant efficacy in female Cdkl5^{KO/X} mice (FIGS. 43A and 43B).

FIG. 43A shows results of contextual fear conditioning test in male Cdkl5^{KO/Y} mice following ICV administration of an AAV vector expressing human CDKL5, plotted as percent freezing. FIG. 43B shows results of contextual fear conditioning test in female Cdkl5^{KO/X} mice following ICV administration of an AAV vector expressing human CDKL5, plotted as percent freezing. Briefly, neonatal (PND 0-1) male Cdkl5^{KO/Y} mice and female Cdkl5^{KO/X} mice received a single ICV administration of either AAVhu68.hSyn.hCDKL5 1co.WPRE.SV40 at a dose of 5.0×10¹⁰ GC (N=16 males, 10 females) or vehicle (PBS; N=16 males, 13 females). Additional age-matched C57BL/6 wild type mice were ICV-administered either AAVhu68.hSyn.hCDKL5-1co.WPRE.SV40 (5.0×10¹⁰ GC; N=10 males, 11 females) or vehicle (PBS; N=15 males, 12 females) as controls. At 11-14 weeks post treatment, the contextual fear conditioning test was performed. **p<0.01, ***p<0.001, ****p<0.0001 based on a one-way ANOVA followed by Sidak's multiple comparisons test comparing all groups to each other except for AAV-treated wild type mice. Abbreviations: AAV, adeno-associated virus; ANOVA, analysis of variance; Cdkl5, cyclin-dependent kinase-like 5 (gene, mouse); GC, genome copies; ICV, intracerebroventricular; KO, knockout; N, number of animals; PBS, phosphate-buffered saline; PND, postnatal day; WT, wild type.

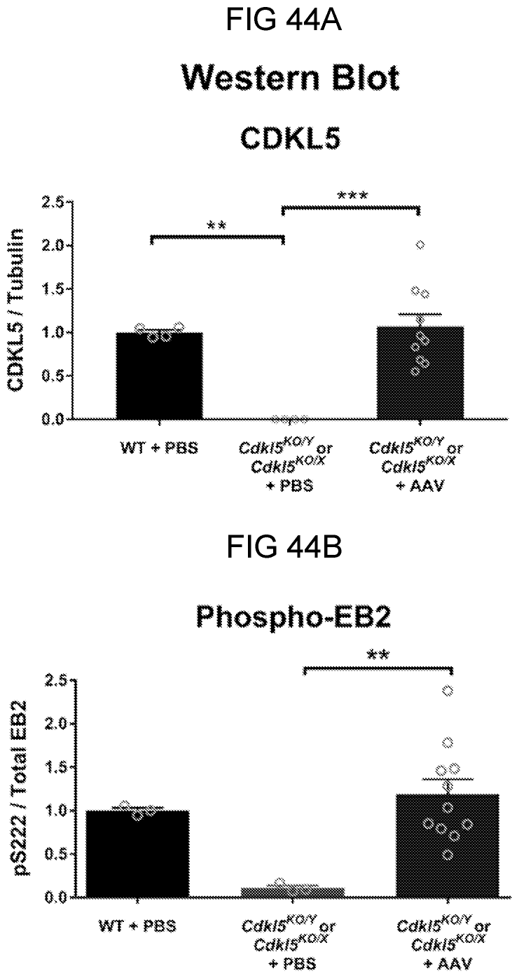

Improvements in behavioral phenotypes correlated with a normalization of transgene product expression and activity following AAV administration to male Cdkl5^{KO/Y} mice and female Cdkl5^{KO/X} mice. Specifically, the absence of detectable brain CDKL5 protein expression that was observed in vehicle-treated male Cdkl5^{KO/Y} mice and female Cdkl5^{KO/X} mice was restored to wild type levels in male Cdkl5^{KO/Y} mice and female Cdkl5^{KO/X} mice at 14 weeks following AAV administration. Similarly, the observed reduction in substrate phosphorylation by CDKL5 (as determined by phospho-EB2 levels) in the brain of vehicle-treated male Cdkl5^{KO/Y} mice and female Cdkl5^{KO/X} mice was restored to wild type levels in male Cdkl5^{KO/Y} mice and female Cdkl5^{KO/X} mice 14 weeks post AAV administration (FIGS. 44A and 44B).

FIG. 44A shows results of a transgene product expression in male Cdkl5^{KO/Y} mice and female Cdkl5^{KO/X} mice following ICV administration of an AAV vector expressing human CDKL5 (CDKL5/tubulin). FIG. 44B shows results of an activity in male Cdkl5^{KO/Y} mice and female Cdkl5^{KO/X} mice following ICV administration of an AAV vector expressing human CDKL5 (pS222/Total EB2). Briefly, neonatal (PND 0-1) male Cdkl5^{KO/Y} mice and female Cdkl5^{KO/X} mice received a single ICV administration of either AAVhu68.hSyn.hCDKL5 1co.WPRE.SV40 at a dose of 5.0×10¹⁰ GC or vehicle (PBS). Additional age-matched C57BL/6 wild type mice were ICV-administered either AAVhu68.hSyn.hCDKL5-1co.WPRE.SV40 (5.0×10¹⁰ GC) or vehicle (PBS) as controls. At necropsy 14 weeks post treatment, brain tissue was collected for evaluation of transgene product expression (CDKL5 protein expression) and transgene product activity (phosphorylation of EB2) by Western blot. **p<0.01; ***p<0.001 based on a one-way ANOVA followed by Sidak's multiple comparisons test comparing all groups to each other except for AAV-treated wild type mice. Abbreviations: AAV, adeno-associated virus; ANOVA, analysis of variance; Cdkl5, cyclin-dependent kinase-like 5 (gene, mouse); GC, genome copies; ICV, intracerebroventricular; KO, knockout; N, number of animals; PBS, phosphate-buffered saline; PND, postnatal day; pS222, phospho-serine 222; WT, wild type.

Cumulatively, this POC pharmacology and assay development study demonstrated that a single ICV administration of an AAV vector similar to AAVhu68.UbC.hCDKL5-1co.miR183.rBG utilizing the same capsid (AAVhu68) and transgene (human CDKL5 isoform 1) led to significant improvements in behavioral phenotypes of a neonatal mouse model of CDD. Improvements in behavioral phenotypes in this mouse model correlated with an increase in transgene product expression (CDKL5 protein) and activity (phosphorylation of the EB2 substrate) to wild type levels in the disease-relevant target tissue (brain) 14 weeks post treatment.

The most sensitive assays for evaluating the efficacy of AAV administration in the CDD mouse model were the open field test, nest building test, and hindlimb clasping test. Specifically, on the open field test, both male Cdkl5^{KO/Y} mice and female Cdkl5^{KO/X} mice exhibited normalization of hyperactivity following AAV treatment, as evidenced by a significant reduction in horizontal activity and rearing to wild type levels. The treatment effect on the open field test was most obvious in male Cdkl5^{KO/Y} mice, as males displayed a markedly more severe phenotype than that of female Cdkl5^{KO/X} mice on this assay, resulting in increased test sensitivity for male Cdkl5^{KO/Y} mice. On the nest building test, both male Cdkl5^{KO/Y} mice and female Cdkl5^{KO/X} mice exhibited a normalization in the nest building defects following AAV administration characterized by significantly increased nest building scores and significantly reduced nestlet weights, both of which were normalized to wild type levels. On the hindlimb clasping test, both male Cdkl5^{KO/X} mice and female Cdkl5^{KO/X} mice also exhibited an improvement in the motor coordination phenotype following AAV administration characterized by significantly reduced hindlimb clasping scores, although the phenotype was not fully normalized to wild type levels.

Some tests (marble burying test and Y maze test) were shown to be ineffective for evaluating the CDD mouse model phenotype because minimal to no phenotypic abnormalities were observed for vehicle-treated male Cdkl5^{KO/Y} mice and/or female Cdkl5^{KO/X} mice when compared to healthy wild type controls, making the future evaluation of a dose-dependent treatment effect challenging with these assessments. Furthermore, one additional test (contextual fear conditioning) demonstrated an AAV treatment effect in only one sex (female Cdkl5^{KO/X} KO mice but not male Cdkl5^{KO/Y} mice), which precludes the use of this assessment in future pharmacology studies.

Based on the results of this study, the open field test, nest building test, and hindlimb clasping test were found to be the most sensitive assays for evaluating the efficacy of AAV administration in both male Cdkl5^{KO/Y} mice and female Cdkl5^{KO/X} mice and were therefore selected for use in future pharmacology studies.

### B. Proof-of-Concept Vector Comparison Pharmacology Study of Clinical Candidate Leads Following ICM Administration to Adult Rhesus Macaques

This POC vector comparison study aimed to assess the safety, tolerability, and transgene product expression of two lead candidates (AAVhu68.hSyn.hCDKL5 1co.WPRE.SV40 [evaluated in Example 3 and Example 9A] and AAVhu68.UbC.hCDKL5-1co.SV40) following ICM administration to adult rhesus macaques. AAVhu68.hSyn.hCDKL5 1co.WPRE.SV40 and AAVhu68.UbC.hCDKL5 1co.SV40 utilize the same capsid (AAVhu68) and express the same transgene product (human CDKL5). AAVhu68.UbC.hCDKL5-1co.SV40 also includes the same promoter as AAVhu68.UbC.hCDKL5-1co.miR183.rBG (UbC). However, AAVhu68.hSyn.hCDKL5 1co.WPRE.SV40 includes a different promoter (hSyn versus UbC) and a WPRE sequence 3′ to the transgene sequence, while both vectors have a different polyA (SV40 versus rBG) and lack the miR183 target sequences for DRG detargeting found in AAVhu68.UbC.hCDKL5-1co.miR183.rBG.

Briefly, adult (3-10 years old) male and female rhesus macaques received a single ICM administration of either AAVhu68.hSyn.hCDKL5-1co.WPRE.SV40 or AAVhu68.UbC.hCDKL5-1co.SV40 at a low dose (3.0×10¹² GC), mid-dose (1.0×10¹³ GC), or high dose (3.0×10¹³ GC). In-life evaluations included observations performed daily, body weights, neurological monitoring, and clinical pathology of the blood (CBC, coagulation panel, serum chemistry) and CSF. All NHPs were necropsied on Day 56. At necropsy, the disease-relevant target tissue (brain) along with additional highly perfused CNS (spinal cord), PNS (DRG, TRG, and sciatic nerve), and peripheral tissues were collected for evaluation of vector biodistribution. Brain, spinal cord, and PNS tissues were evaluated for histopathology as these are tissues that are highly transduced by the ICM route. Pituitary gland tissues were also evaluated for histopathology. Additional brain tissue was collected to assess transgene product expression (human CDKL5 mRNA expression by ISH and qPCR) in this disease-relevant target tissue. Serum was collected and stored for possible future assessment of NAbs to the vector capsid. PBMCs and tissue-resident lymphocytes were also collected and stored for possible future evaluation of T cell responses to the vector capsid and/or transgene product (IFN-γ ELISpot).

AAVhu68.UbC.hCDKL5-1co.miR183.rBG was well-tolerated, and no test article-related findings were observed on cage-side observations, neurological monitoring, or blood clinical pathology. A transient mild CSF lymphocytic pleocytosis (≥6 white blood cells [WBCs]/μL) that was considered test article-related was observed in a single animal administered the mid-dose (1.0×10¹³ GC) of AAVhu68.UbC.hCDKL5 1co.SV40 on Day 28. The pleocytosis was asymptomatic and resolved without treatment by the following time point on Day 42. An additional animal administered the mid-dose (1.0×10¹³ GC) of AAVhu68.hSyn.hCDKL5-1co.WPRE.SV40 exhibited a marked lymphocytic pleocytosis on Day 8; however, this asymptomatic finding was likely attributable to hemodilution in the sample (1780 RBCs) and was not observed at any other time point evaluated.

On histopathology at Day 56, DRG sensory neuron degeneration was not observed for either vector at any dose (N=0/3 DRG segments per animal). However, axonopathy in both the dorsal white matter tracts of the spinal cord and in the peripheral nerves (sciatic nerve) was observed for most animals. The findings of axonopathy were suggestive of DRG sensory neuron pathology because axons from these DRG neurons project into this region of the spinal cord and into the peripheral nerves. In all cases, the spinal cord and peripheral nerve axonopathy was asymptomatic, with no clinical abnormalities noted on daily observations or neurological exams.

With regard to the axonopathy in the dorsal white matter tracts of the spinal cord, the incidence and severity appeared generally dose-dependent for both vectors, increasing from no axonopathy at the lowest dose (N=0/3 segments for both vectors) to a minimal severity (Grade 1) at the mid dose and high dose (N=1/3 segments and N=3/3 segments for AAVhu68.hSyn.hCDKL5 1co.WPRE.SV40, respectively; N=2/3 segments for AAVhu68.UbC.hCDKL5 1co.SV40 for both doses). Comparing across vectors, both vectors exhibited a similar severity of spinal cord axonopathy, with minimal (Grade 1) pathology observed in all cases (N=2/3 animals for each vector). Furthermore, no clear difference in incidence of spinal cord axonopathy between these vectors was observed. AAVhu68.hSyn.hCDKL5-1co.WPRE.SV40 resulted in a lower incidence of spinal cord axonopathy at the mid-dose (N=1/3 segments) compared to that of AAVhu68.UbC.hCDKL5 1co.SV40 at the same dose (N=2/3 segments). However, the opposite was observed at the high dose, with AAVhu68.hSyn.hCDKL5-1co.WPRE.SV40 exhibiting a higher incidence of spinal cord axonopathy (N=3/3 segments) compared to that of AAVhu68.UbC.hCDKL5 1co.SV40 (N=2/3 segments).

With regard to the axonopathy in the peripheral nerves (sciatic nerve), the severity and incidence did not appear dose-dependent for AAVhu68.UbC.hCDKL5 1co.SV40, with minimal (Grade 1) peripheral nerve axonopathy observed for each animal at all doses (N=3/3 sciatic nerves; N=3/3 animals). In contrast, AAVhu68.hSyn.hCDKL5-1co.WPRE.SV40 resulted in peripheral nerve axonopathy that was dose-dependent in terms of both severity and incidence, with no axonopathy observed at the low dose or mid-dose (N=2/2 sciatic nerves; N=2/2 animals) and mild axonopathy (Grade 2) observed at the high dose (N=1/1 sciatic nerves; N=1/1 animals). Comparing across vectors, AAVhu68.hSyn.hCDKL5-1co.WPRE.SV40 led to a lower overall incidence of peripheral axonopathy, while AAVhu68.UbC.hCDKL5 1co.SV40 led to a lower overall severity of axonopathy.

An evaluation of vector biodistribution at Day 56 revealed a high level of transduction throughout the brain for both vectors. Both vectors also demonstrated a relatively high level of transduction in the spinal cord, DRG, peripheral nerve (trigeminal), and spleen. Comparatively lower transduction was observed for both vectors in peripheral tissues, including the lung, muscle, heart, kidney, liver, and eye. A dose response was not obvious for either vector, possibly due to the low number of animals evaluated for this POC study. Comparing across vectors, transduction levels for each tissue, including each brain region evaluated, were generally similar for each respective dose when taking into account expected inter-animal variability. Thus, ICM administration of AAVhu68.hSyn.hCDKL5 1co.WPRE.SV40 or AAVhu68.UbC.hCDKL5 1co.SV40 to NHPs resulted in a comparable biodistribution profile, with both vectors efficiently transducing the disease-relevant target tissue (brain).

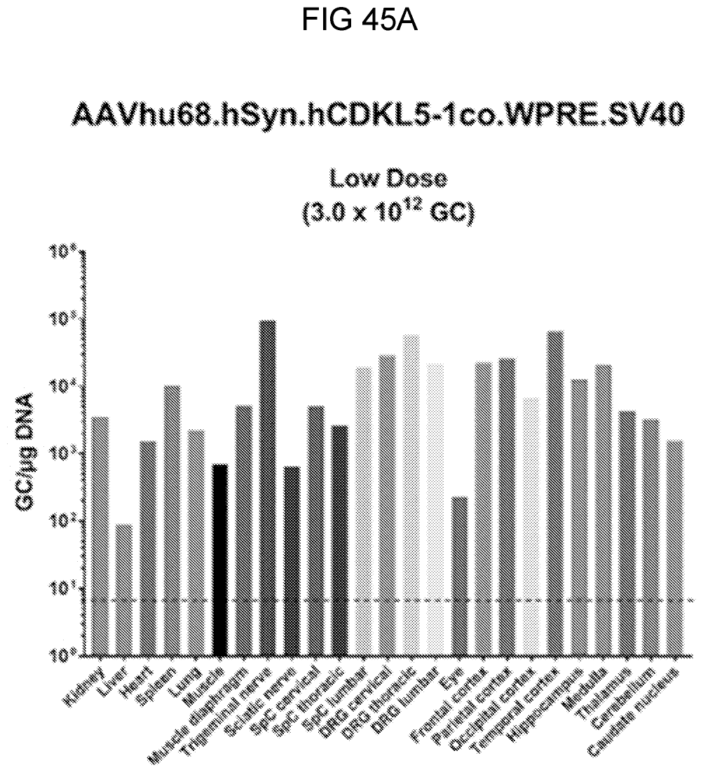

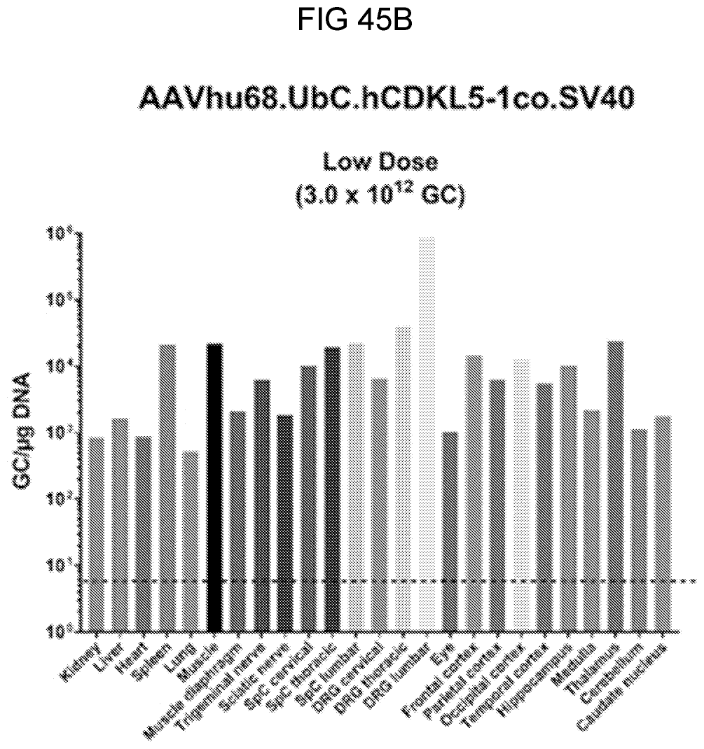

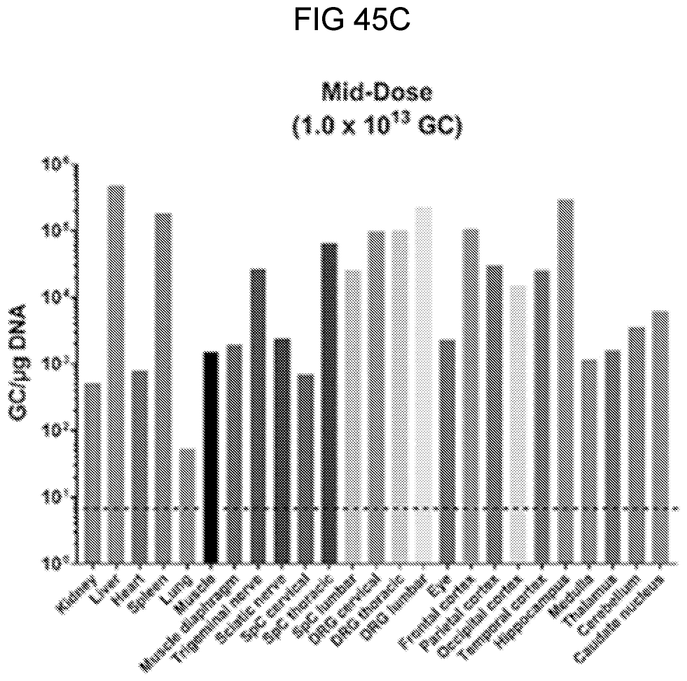

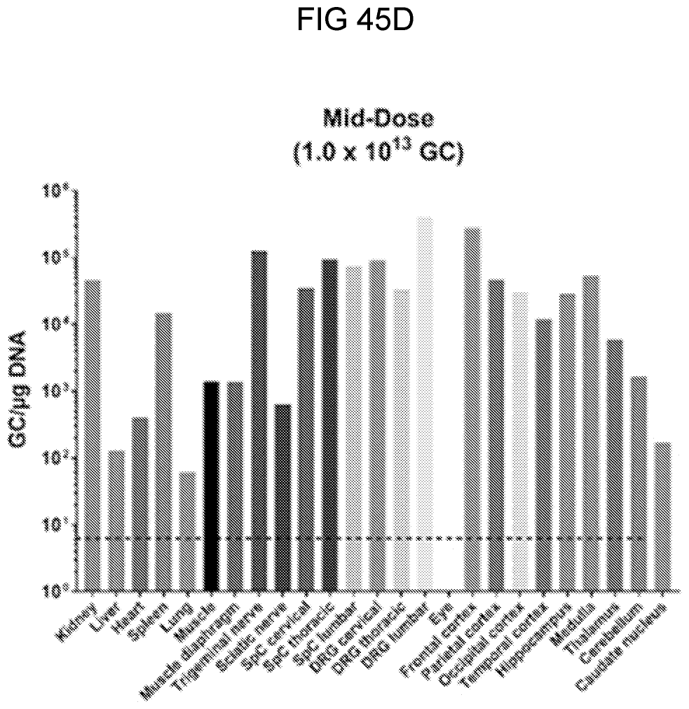

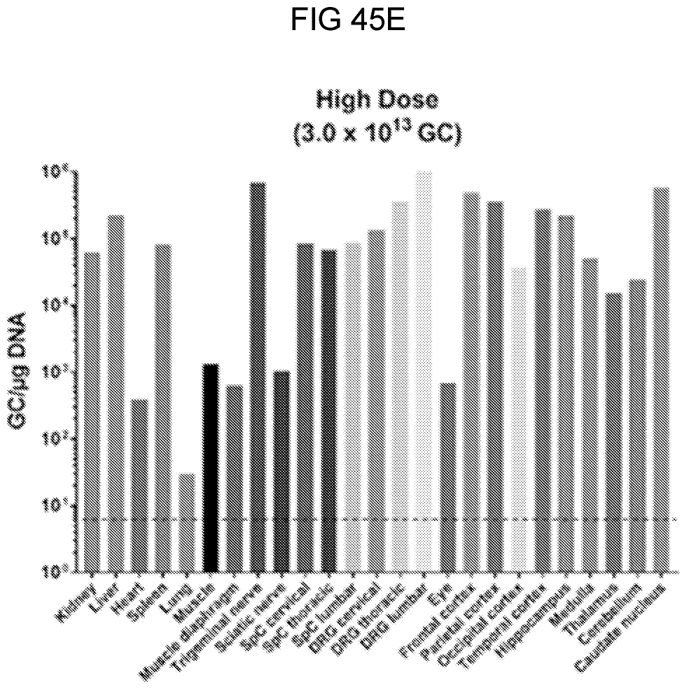

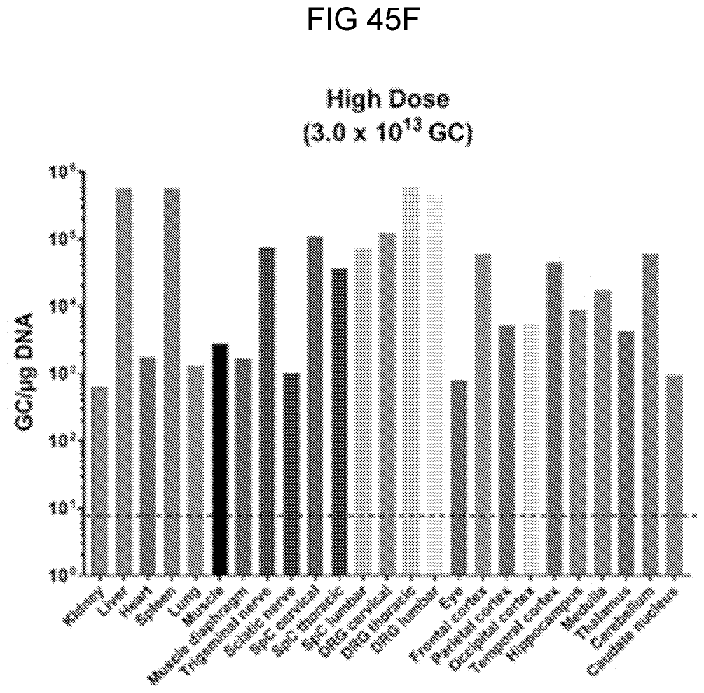

FIG. 45A shows results of the vector biodistribution in adult rhesus macaques following ICM administration of an AAVhu68.hSyn.hCDKL5-1co.WPRE.SV40 at a low dose. FIG. 45B shows results of the vector biodistribution in adult rhesus macaques following ICM administration of an AAVhu68.UbC.hCDKL5-1co.SV40 at a low dose. FIG. 45C shows results of the vector biodistribution in adult rhesus macaques following ICM administration of an AAVhu68.hSyn.hCDKL5-1co.WPRE.SV40 at a mid-dose. FIG. 45D shows results of the vector biodistribution in adult rhesus macaques following ICM administration of an AAVhu68.UbC.hCDKL5-1co.SV40 at a mid-dose. FIG. 45E shows results of the vector biodistribution in adult rhesus macaques following ICM administration of an AAVhu68.hSyn.hCDKL5-1co.WPRE.SV40 at a high dose. FIG. 45F shows results of the vector biodistribution in adult rhesus macaques following ICM administration of an AAVhu68.UbC.hCDKL5-1co.SV40 at a high dose. Briefly, adult (3-10 years old) male and female rhesus macaques received a single ICM administration of either AAVhu68.hSyn.hCDKL5-1co.WPRE.SV40 or AAVhu68.UbC.hCDKL5-1co.SV40 at a low dose (3.0×10¹² GC), mid-dose (1.0×10¹³ GC), or high dose (3.0×10¹³ GC) (N=1 animal per vector per dose). All NHPs were necropsied on Day 56±4, and the indicated tissues were collected for evaluation of AAVhu68 vector biodistribution (TaqMan qPCR). The dashed line represents the limit of detection (50 GC/g DNA). Abbreviations: AAVhu68, adeno-associated virus hu68; DNA, deoxyribonucleic acid; GC, genome copies; ICM, intra-cisterna magna; N, number of animals; NHP, non-human primate; qPCR, quantitative polymerase chain reaction.

Consistent with the observed biodistribution profile for each vector, transgene product expression (human CDKL5 isoform 1 mRNA) was detectable at all doses evaluated for both vectors in brain regions relevant to the treatment of CDD, including the cerebellum and throughout the cortex (FIG. 46). Transgene product expression was generally dose-dependent for both vectors, with a lower level of expression observed for most brain regions at the low dose compared to that of the mid-dose and high dose for each vector. Comparing across vectors, expression levels for each brain region were generally similar at each respective dose when taking into account expected inter-animal variability. Thus, ICM administration of AAVhu68.hSyn.hCDKL5 1co.WPRE.SV40 or AAVhu68.UbC.hCDKL5 1co.SV40 to NHPs led to a similar level of dose-dependent transgene product expression in the disease-relevant target tissue (brain).

FIG. 46 shows results of a transgene product expression in the brain of adult Rhesus Macaques following ICM administration of an AAV vector expressing human CDKL5-Adult (3-10 years old) male and female rhesus macaques received a single ICM administration of either AAVhu68.hSyn.hCDKL5-1co.WPRE.SV40 or AAVhu68.UbC.hCDKL5-1co.SV40 at a low dose (3.0×10¹² GC), mid-dose (1.0×10¹³ GC), or high dose (3.0×10¹³ GC) (N=1 animal per vector per dose). All NHPs were necropsied on Day 56+4, and brains were collected for evaluation of transgene product expression (human CDKL5 isoform 1 mRNA qPCR). Abbreviations: CDKL5-1, cyclin-dependent kinase-like 5 (isoform 1); GC, genome copies; ICM, intra-cisterna magna; mRNA, messenger ribonucleic acid; N, number of animals; NHP, non-human primate; qPCR, quantitative polymerase chain reaction.

Cumulatively, ICM administration of either AAVhu68.hSyn.hCDKL5 1co.WPRE.SV40 or AAVhu68.UbC.hCDKL5 1co.SV40 to adult male and female rhesus macaques was well-tolerated, with no test article-related findings observed on cage-side observations, neurological monitoring, or blood clinical pathology for either vector. A transient mild asymptomatic CSF lymphocytic pleocytosis that was likely test-article related was observed in a single animal administered the mid-dose (1.0×10¹³ GC) of AAVhu68.UbC.hCDKL5 1co.SV40 on Day 28, which resolved without treatment by Day 42. Histopathologic evaluation for both vectors on Day 56 revealed an asymptomatic axonopathy of the dorsal white matter tracts of the spinal cord and a peripheral nerve, which was considered secondary to DRG sensory neuron degeneration. While the incidence and severity of the axonopathy in the spinal cord was similar for both vectors, the severity in the peripheral nerve was slightly lower for AAVhu68.UbC.hCDKL5 1co.SV40 compared to that of AAVhu68.hSyn.hCDKL5 1co.WPRE.SV40 (Grade 1 versus Grade 2, respectively). Both vectors also demonstrated similarly robust vector transduction profiles and led to transgene product (human CDKL5 mRNA) in the disease-relevant target tissue (brain) on Day 56.

Ultimately, this study led to the selection of the AAVhu68 capsid, UbC promoter, and hCDKL5 1co transgene sequence lacking the 3′ WPRE element for further evaluation. These elements were selected based on the favorable vector biodistribution and transgene product expression profile in the disease-relevant target tissue (brain) following ICM administration of AAVhu68.UbC.hCDKL5 1co.SV40 to NHPs, along with the observation of that animals treated with AAVhu68.UbC.hCDKL5 1co.SV40 exhibited less severe peripheral nerve pathology than those administered the other vector.

C. Proof-of-Concept Pharmacology and Assay Development Study of AAVhu68.UbC.hCDKL5-1co.miR183.rBG in a Mouse Model of CDD

This POC pharmacology study evaluates the therapeutic efficacy of AAVhu68.UbC.hCDKL5-1co.miR183.rBG following ICV administration to neonatal male Cdkl5^{KO/Y} mice and female Cdkl5^{KO/X} mice in order to optimize the study design and assays to be used in the planned MED pharmacology study.

Briefly, neonatal (PND 0-1) male Cdkl5^{KO/Y} mice and female Cdkl5^{KO/X} mice received a single ICV administration of either AAVhu68.UbC.hCDKL5-1co.miR183.rBG at a dose of 2.5×10¹⁰ GC or vehicle (PBS) (N=12/group). Additional age-matched male and female C57BL/6J wild type mice received vehicle (PBS) as a control (N=12). Ongoing in-life assessments includes viability checks performed daily, body weight measurements performed weekly, and behavioral assessments performed at 10-11 weeks post treatment (open field, nest building, hindlimb clasping tests). Necropsies are performed 13-14 weeks post treatment. At necropsy, blood is collected for CBC/differentials and serum clinical chemistry analysis. A list of tissues are collected for histopathological evaluation. Transgene product expression (CDKL5 Western blot, CDKL5 immunofluorescence) and transgene product activity (phosphorylation of EB2 [phospho-EB2 Western blot]) are evaluated in the disease-relevant target tissue (brain) and highly transduced peripheral tissues.

D. Efficacy of AAVhu68.UbC.hCDKL5-1co.miR183.rBG Following ICV Administration to Neonatal Male Cdkl5^{KO/Y} Mice to Determine the MED

This pharmacology study evaluates the efficacy and determine the MED of ICV-administered AAVhu68.UbC.hCDKL5-1co.miR183.rBG in neonatal male Cdkl5^{KO/Y} mice. The vector used for this study is the toxicological vector lot that is manufactured for the planned GLP compliant NHP toxicology study.

Briefly, in this study evaluation is performed, N=60 neonatal (PND 0-1) AAVhu68.UbC.hCDKL5-1co.miR183.rBG-treated male Cdkl5^{KO/Y} mice and N=12 age-matched vehicle-treated male C57BL/6J wild type controls. The study includes one necropsy time point (13-14 weeks post treatment). Four dose levels of AAVhu68.UbC.hCDKL5-1co.miR183.rBG is evaluated using ICV administration. The dose levels are selected based on the results from the ongoing POC pharmacology study evaluating the efficacy of AAVhu68.UbC.hCDKL5-1co.miR183.rBG administration in this mouse model of CDD (as described above), in addition to the POC safety and pharmacology study of AAVhu68.UbC.hCDKL5-1co.miR183.rBG conducted in adult African green monkeys (also, as described above).

In-life assessments include viability checks performed daily, body weight measurements, and behavioral assessments (open field, nest building, hindlimb clasping tests). Necropsies are performed 13-14 weeks post treatment. At necropsy, blood is collected for CBC/differentials and serum clinical chemistry analysis. A list of tissues are collected for histopathological evaluation. Transgene product expression (CDKL5 Western blot, CDKL5 immunofluorescence) and activity (phosphorylation of EB2) are evaluated in the disease-relevant target tissue (brain) and highly transduced peripheral tissues.

E. Toxicology Study of ICM Administration of AAVhu68.UbC.hCDKL5-1co.miR183.rBG to Juvenile Rhesus Macaques

A 180 day GLP-compliant toxicology study assesses the safety, tolerability, pharmacology (CDKL5 mRNA expression), biodistribution, and excretion profile of GTP-213 following a single ICM administration at a low dose, mid-dose, or high dose (N=4/dose) to juvenile (1.5-2 years old) male and female rhesus macaques. Additional age-matched male and female NHPs are administered vehicle (intrathecal final formulation buffer [ITFFB]) as a control (N=2).

The NHP (rhesus macaque) was selected for the planned toxicology study (Genotoxicity, Carcinogenicity, Reproductive Toxicity, and Developmental Toxicity Assessments). The highest dose evaluated is the maximum feasible dose based on anticipated vector titers and the maximum administration volume. The mid-dose and low dose are approximately 3-fold and 10-fold lower than the maximum feasible dose, respectively. This range was selected to ensure that doses are distinct and encompass the dose range evaluated in the mouse MED pharmacology study. A 180 day study duration with an interim Day 90 necropsy time point for this toxicology study.

Using CSF as a vehicle for vector dispersal, the intrathecal (IT) ROA has the potential to achieve transgene delivery throughout the CNS. Studies in large animal models of lysosomal storage diseases (such as mucopolysaccharidosis [MPS] type I and MPS type VII) demonstrated that CSF delivery of AAV results in widespread transduction of neurons throughout the brain, which is the key target tissue for the treatment of CDD (Hinderer et al., 2014a; Hinderer et al., 2015; Gurda et al., 2016). A recent study examining different routes for CSF access demonstrated that delivery of an AAV vector via ICM administration was at least 10-fold more efficient at transducing cells of the brain, spinal cord, and spinal cord motor neurons when compared to injection of the vector via a lumbar puncture (Hinderer et al., 2014b). ICM administration was therefore selected for the planned clinical trial, and the ICM route will be utilized in the planned NHP toxicology study to replicate the intended clinical ROA.

Method for Scaling from Nonclinical Doses to Clinical Doses

ICM vector administration results in immediate vector distribution within the CSF compartment, and it is anticipated that both efficacy and toxicity is related to CNS vector exposure. Doses are therefore scaled by brain mass, which provides an approximation of the size of the CSF compartment. Dose conversions are based on a brain mass of 0.15 g for a neonatal mouse (Gu et al., 2012), 90 g for a juvenile NHP (Herndon et al., 1998), 610 g from a 6-8-month infant, 780 g for an 8-12-month infant, and 960 g for a >12-month infant (Dekaban, 1978). Estimated brain weights for each age range for human infants were derived from the male and female brain weights presented in (Dekaban, 1978) by assuming an approximately linear increase in brain weight between that of newborns (370 g) and infants aged 4-8 months, resulting in a mean estimated brain weight of 488 g for ≥1-<4 month old infants. The value of 610 g corresponds to the average brain weight for males and females aged 4-8 months (Dekaban, 1978).

An example of dose scaling from neonatal mice, juvenile NHPs, and equivalent human doses are presented in table immediately below. The administration volume will also be scaled from NHPs to humans based on the estimated volumes for cerebral CSF (Matsumae et al., 1996) and spinal CSF (Rochette et al., 2016).

| | | Total Dose | | | | |
|---|---|---|---|---|---|---|
| | | (GC) | | | | |
| | Scaled Dose | | | Human Infant | | |
| | (GC/g Brain)^{a} | Neonatal | | ≥6 Months-<8 | ≥8 Months-<12 | |
| | All Species | Mouse | Juvenile NHP^{a} | Months | Months | ≥12 Months |
| Low | 1.1 × 10¹⁰ | 2.2 × 10⁹ | — | 6.8 × 10¹² | 8.7 × 10¹² | 1.1 × 10¹³ |
| Dose | | | | | | |
| Mid- | 3.3 × 10¹⁰ | 6.7 × 10⁹ | 3.0 × 10¹² | 2.1 × 10¹³ | 2.6 × 10¹³ | 3.2 × 10¹³ |
| Dose #1 | | | | | | |
| Mid- | 1.1 × 10¹¹ | 2.2 × 10¹⁰ | 1.0 × 10¹³ | 6.8 × 10¹³ | 8.7 × 10¹³ | 1.0 × 10¹⁴ |
| Dose #2 | | | | | | |
| High | 3.3 × 10¹¹ | 6.7 × 10¹⁰ | 3.0 × 10¹³ | 2.0 × 10¹⁴ | 2.6 × 10¹⁴ | 3.2 × 10¹⁴ |
| Dose | | | | | | |

^{a}Doses are scaled based on a brain mass of 0.15 g for a neonatal mouse (Gu et al., 2012), 90 g for a juvenile NHP (Herndon et al., 1998), 610 g from a 6-8-month infant, 780 g for an 8-12-month infant, and 960 g for a >12-month infant (Dekaban, 1978).
Abbreviations: GC, genome copies; NHP, non-human primate.

### Example 10: First-in-Human Clinical Trial Protocol Synopsis

### Overview of First-In-Human Trial

The FIH trial is an open-label, multi-center, dose escalation study of AAVhu68.UbC.hCDKL5-1co.miR183.rBG administered via an intra-cisterna magna (ICM) injection to evaluate safety, tolerability, and exploratory efficacy endpoints in pediatric (≥30 days of age) and adult subjects with CDKL5 deficiency disorder (CDD). A maximum of 36 subjects with CDD may be enrolled in the study. This study is initially enrolling subjects who are ≥12 years of age in the first dose escalation cohort. Staggered enrollment and treatment of younger age groups (≥2 and <12 years, ≥30 days to <2 years) commences only after available safety, laboratory and clinical data from the next higher age group have been reviewed by the independent Data Safety Monitoring Board (DSMB). Each age group has a dose escalation where advancement to the next dose level requires agreement from the DSMB.

The dose escalation (Cohorts 1 and 2) assesses a single ICM administration of two dose levels of AAVhu68.UbC.hCDKL5-1co.miR183.rBG. The AAVhu68.UbC.hCDKL5-1co.miR183.rBG dose levels to be tested is determined based on data from the murine MED study and GLP NHP toxicology study, and consists of a low dose (administered to Cohort 1) and a high dose (administered Cohort 2). Both dose levels are anticipated to confer therapeutic benefit, with the understanding that, if tolerated, the higher dose would be expected to be advantageous and advanced. Our standard approach is that a safety margin is applied so that the high dose selected for human subjects is 30-50% of the equivalent MTD in NHPs. The low dose is typically 2-3-fold less than the selected high dose, provided it is a dose that exceeds the equivalent scaled MED in the animal studies.

The dose escalation portion of this study follows a 3+3 design. For each age group, three subjects are enrolled in a dose cohort. If safety data are deemed acceptable by the DSMB, the age cohort may proceed to the next dose level. Additionally, the next younger age cohort may begin enrolling at the same dose level tested in the older age cohort. If one of the first three subjects develops a safety review trigger (SRT) or based on DSMB guidance, additional up to three subjects will be enrolled into that same age and dose cohort.

Although 2 dose-level cohorts (up to 12 subjects) are planned for the dose escalation phase, the performance of the second dose-level cohort depends on the evolving safety, tolerability and efficacy data available. The dose level, size of the cohort, safety monitoring for subsequent cohorts are confirmed with the DSMB prior to enrollment.

As the proposed clinical trial is the first to assess AAVhu68.UbC.hCDKL5-1co.miR183.rBG in humans, Investigational Product (IP) dosing of subjects with AAVhu68.UbC.hCDKL5-1co.miR183.rBG is staggered by at least 6 weeks between each subject to monitor for liver enzyme increases and evaluation of Adverse Events (AEs) indicative of ICM administration complications, immune reactions, or other dose-limiting toxicities. Furthermore, this 6-week window captures the time when maximal gene expression is expected based on nonclinical data. This duration between IP dosing of subjects may be further refined based on emerging nonclinical data to either shorten or prolong the interval between subject IP dosing in the final protocol.

All treated subjects will be followed for 2 years to evaluate the safety profile and characterize the pharmacodynamic and efficacy properties of AAVhu68.UbC.hCDKL5-1co.miR183.rBG in the Phase 1 FIH study. Subjects are followed for an additional 3 years (for a total of 5 years post-dose) in a subsequent long-term follow up study to evaluate long-term clinical outcomes, which is in line with the draft “FDA Guidance for Industry: Long Term Follow-Up after Administration of Human Gene Therapy Products” (January 2020).

| | |
|---|---|
| Protocol(s) | A Phase 1/2 Open-Label, Multi-Center, Dose Escalation Study to Assess the Safety, |
| Title | Tolerability and Efficacy of Single Doses of AAVhu68.UbC.hCDKL5- |
| | 1co.miR183.rBG Delivered into the Cisterna Magna (ICM) of Pediatric Subjects |
| | with CDKL5 Deficiency Disorder (CDD) |
| Objectives | This is a Phase 1 first-in-human dose escalation study. The cumulative data collected |
| and | during this study is the basis for the design of a subsequent confirmatory study to |
| Endpoints | support marketing approval. Doses administered are expected to be safe and with the |
| | potential to confer therapeutic benefit to study participants as determined in the |
| | nonclinical development program. |
| | Primary: |
| | The primary objective of this study is to assess the safety and tolerability of an ICM |
| | injection of a single dose of AAVhu68.UbC.hCDKL5-1co.miR183.rBG through the |
| | evaluation of: |
| | Adverse Events (AEs) and serious adverse events (SAEs) |
| | Vital signs and physical examinations |
| | Neurological examinations |
| | Seizure frequency and severity |
| | Cardiac monitoring - ECG |
| | Laboratory assessments (serum chemistry, hematology, coagulation |
| | studies, liver function tests [LFTs], and urinalysis) |
| | Immunogenicity of the vector capsid and transgene product in blood |
| | and CSF |
| | Vector shedding |
| | Nerve conduction velocity (NCV) measurements of the deep |
| | peroneal, tibial, ulnar, sural, median nerves (to assess sensory and |
| | motor nerve peripheral neuropathy) |
| | Secondary/Exploratory (pharmacodynamic, efficacy): |
| | The secondary efficacy objectives of this study are to assess the pharmacodynamics |
| | and efficacy of an ICM injection of a single dose of AAVhu68.UbC.hCDKL5- |
| | 1co.miR183.rBG through evaluation of: |
| | Seizure frequency and severity |
| | 28-day primary seizure frequency |
| | Primary seizure types include bilateral tonic (sustained motor |
| | activity ≥3 seconds), generalized tonic-clonic, bilateral |
| | clonic, atonic/drop seizures and focal to bilateral tonic-clonic) |
| | Bayley Scales of Infant and Toddler Development, Third Edition |
| | (BSID-III) (up to 42 months of age) and/or the Kaufman Assessment |
| | Battery for Children, Second Edition (>42 months of age). Including |
| | focus on gross and fine motor scores. |
| | Vineland Adaptive Behavior Scales, Second Edition. Including focus |
| | on the sub-domain raw scores. |
| | Sleep Disturbance Scale for Children (SDSC) |
| | Raw scores from the Cortical Visual Impairment - Range |
| | Score from the Observer-Reported Communication Ability (ORCA) |
| | measure |
| | Quality of Life Inventory-Disability (QI-Disability) |
| | EEC-ERP (auditory) |
| | Exploratory: |
| | The exploratory objectives of this study are to assess the clinical efficacy of |
| | AAVhu68.UbC.hCDKL5-1co.miR183.rBG through evaluation of: |
| | Death |
| | Hospitalization and healthcare utilization |
| Study | This is a Phase 1/2 prospective, proof-of-concept, FIH, multi-center, dose escalation |
| Design | study to evaluate safety, tolerability, pharmacodynamics and efficacy of single ICM |
| | administration of AAVhu68.UbC.hCDKL5-1co.miR183.rBG in up to 36 subjects |
| | with CDD. The subjects are grouped into 3 age cohorts: Group A, ≥12 years; Group |
| | B, ≥2 and <12 years; and Group C, ≥30 days to <2 years of age. Each age cohort |
| | is enrolled in a staggered approach from older to younger, and each age cohort has a |
| | dose escalation portion consisting of Cohort 1 followed by Cohort 2, The DSMB |
| | reviews available data and is required to agree with beginning enrollment in a |
| | specific dose and age cohort. |
| | Patients are administered corticosteroids immediately prior to and for 30 days after |
| | ICM administration (with subsequent taper) to minimize any potential inflammation |
| | related to the study procedure. Furthermore, if at any time post-ICM administration |
| | transaminase elevations are observed, the steroid dose is increased or reinitiated. A |
| | prolonged regimen of corticosteroids may also be utilized to mitigate potential |
| | immune related injury, such as hepatotoxicity. Subjects are closely monitored for |
| | signs of inflammation and immune toxicity as well as issues related to steroid dosing. |
| | A full dosing regimen is provided in the eventual FIH clinical study protocol. |
| | Dosing in each stage and cohort consists of a one-time administration of a single |
| | dose of AAVhu68.UbC.hCDKL5-1co.miR183.rBG delivered via the ICM route-of- |
| | administration (ROA). Dose administration occurs in an inpatient setting. |
| | The dose escalation phase of the proposed study (Cohorts 1 and 2) uses a 3 + 3 design. |
| | 3 patients are enrolled in Cohort 1 prior to consideration of dose escalation. There is |
| | at least 6 weeks duration between AAVhu68.UbC.hCDKL5-1co.miR183.rBG |
| | treatment of each subject for Cohorts 1 and 2 to allow for monitoring of safety data |
| | and available pharmacodynamic data. The safety, tolerability, and all available |
| | clinical data collected from Cohorts 1 and 2 are used for determination of the MTD |
| | to be evaluated in Cohort 3. Development of a severe SRT in one subject or a |
| | moderate SRT in more than 1 subject for a specific dose cohort indicates that the |
| | MTD may have been exceeded. |
| Study | Enrollment occurs on a rolling basis. Each subject is enrolled in this Phase 1/2 |
| Duration | clinical trial for 2 years. Thereafter, subjects are enrolled into a separate long-term |
| | follow up study for an additional 3 years (for a total duration of 5 years post- |
| | treatment with AAVhu68.UbC.hCDKL5-1co.miR183.rBG). The 5-year duration of |
| | participation in this protocol is in accordance with “FDA Guidance for Industry: |
| | Long Term Follow-Up after Administration of Human Gene Therapy Products” |
| | (January 2020). |
| Number of | Up to 36 evaluable subjects (up to 12 evaluable subjects in each of the three age |
| Subjects | cohorts). |
| Study | Multiple centers in the US and outside of the US capable of performing |
| Center | administration of investigational gene therapy product serves as |
| | AAVhu68.UbC.hCDKL5-1co.miR183.rBG dosing centers for all enrolled study |
| | participants. Study follow-up visits and assessments may be performed at select |
| | regional medical centers. These sites may be dosing centers or non-dosing centers |
| | that will only perform study visits and assessments that do not involve the dosing |
| | visit. |
| Main | 1. Male or female ≥30 days of age. |
| Inclusion | 2. Documented diagnosis of CDKL5 deficiency disorder (CDD) confirmed by |
| Criteria | following: |
| | Pathogenic or likely pathogenic CDKL5 gene variant identified by |
| | DNA sequencing |
| | Epileptic encephalopathy |
| | Eligibility is confirmed by an independent external expert adjudication committee. |
| | 3. Subject or Parent/Legal Guardian must be able to comprehend and be willing |
| | to provide an institutional review board/ethics. committee (IRB/EC) |
| | approved Informed Consent Form (ICF). |
| | Note: If deemed cognitively able, the participant will provide assent as well. |
| | Investigators will determine individually whether patients are able to provide assent. |
| | 4. Subjects and Parent/Legal Guardian must be able to comply with all study- |
| | related procedures and be available for the duration of the study. |
| Main | 1. CDKL5 duplication |
| Exclusion | 2. Any clinically significant neurocognitive or physical deficit not attributable |
| Criteria | to CDD or a secondary cause that may, in the opinion of the investigator, |
| | confound interpretation of study results. |
| | 3. Patients with a positive test result for human immunodeficiency virus (HIV) |
| | or Hepatitis C (HepC) |
| | 4. Any condition (e.g., history of any disease, evidence of any current disease, |
| | any finding upon physical examination, or any laboratory abnormality) that, |
| | in the opinion of the investigator, would put the subject at undue risk or |
| | would interfere with evaluation of the investigational product or |
| | interpretation of subject safety or study results. |
| | 5. Any contraindication to ICM administration or MRI . . . This includes |
| | fluoroscopic imaging and/or anesthesia for these procedures. This includes |
| | but is not limited to: local infection, thrombocytopenia (platelet count <100,000 |
| | per μL), coagulopathy (INR >1.5 or activated partial thromboplastin |
| | time [aPTT] >40 seconds), elevated intracranial pressure ([ICP] due to a |
| | space-occupying lesion. |
| | 6. Liver function tests (LFTs) at screening (AST or ALT >3 × upper limit of |
| | normal (ULN) and/or total bilirubin of >1.5 × ULN unless subject has |
| | unconjugated hyperbilirubinemia due to Gilbert's syndrome). |
| | 7. History of cirrhosis or chronic liver disease based on documented |
| | histological evaluation or non-invasive imaging or testing. |
| | 8. Any contraindication to corticosteroids or immune suppressing therapies |
| | 9. Enrollment in any other clinical study with an investigational product within |
| | 4 weeks prior to Screening or within 5 half-lives of the investigational |
| | product used in that clinical study, whichever is longer |
| | 10. Prior treatment with hematopoietic stem cell transplantation, gene therapy, |
| | cell therapy, or enzyme replacement therapy |
| | 11. Receipt of a vaccine within 14 days of gene therapy administration |
| Investigational | AAVhu68.UbC.hCDKL5-1co.miR183.rBG |
| Product | |
| Route of | AAVhu68.UbC.hCDKL5-1co.miR183.rBG is administered as a single dose to |
| Administration | hospitalized participants on Day 1 via CT-guided sub-occipital injection into the |
| and | cisterna magna. On Day 1, a syringe containing AAVhu68.UbC.hCDKL5- |
| Procedure | 1co.miR183.rBG (final volume ≤5 ml) at the appropriate titer is prepared by the |
| | Investigational Pharmacy associated with the study and delivered to the procedure |
| | room. Prior to study drug administration, the participant is anesthetized, intubated, |
| | and the injection site is prepped and draped using sterile technique. A lumbar |
| | puncture is performed to remove a predetermined volume of CSF, after which |
| | iodinated contrast is IT injected to aid in visualization of relevant anatomy of the |
| | cisterna magna. IV contrast may be administered prior to or during needle insertion |
| | as an alternative to the IT contrast. The decision to use IV or IT contrast is at the |
| | discretion of the interventionalist performing the procedure. A spinal needle (22-25 |
| | G) is advanced into the cisterna magna under fluoroscopic guidance. A larger |
| | introducer needle may be used to assist with needle placement. After confirmation of |
| | needle placement, the extension set is attached to the spinal needle and allowed to fill |
| | with CSF. At the discretion of the interventionalist, a syringe containing contrast |
| | material may be connected to the extension set and a small amount injected to |
| | confirm needle placement in the cisterna magna. After the needle placement is |
| | confirmed, the syringe containing rAAV is connected to the extension set. The |
| | syringe contents are slowly injected over 1-2 minutes, delivering a volume of ≤5.0 |
| | ml. |
| Safety | Safety assessments include collection of AEs and SAEs, physical and neurologic |
| Assessments | examinations, vital signs, clinical laboratory tests (serum chemistry, hematology, |
| | coagulation, LFTs, urinalysis), ECGs, nerve conduction studies. These are performed |
| | at the times indicated in the study schedule. Neurocognitive testing and EEGs |
| | provide safety in addition to efficacy evaluation. Brain MRI, lumbar puncture for |
| | CSF analysis may be performed for safety assessment if clinically indicated. |

### REFERENCES

1. Bahi-Buisson, N. et al. Key clinical features to identify girls with CDKL5 mutations. Brain 131, 2647-2661, (2008).
2. Fehr, S. et al. The CDKL5 disorder is an independent clinical entity associated with early-onset encephalopathy. Eur J Hum Genet 21, 266-273, (2013).
3. Kalscheuer, V. M. et al. Disruption of the serine/threonine kinase 9 gene causes severe X-linked infantile spasms and mental retardation. American journal of human genetics 72, 1401-1411, (2003).
4. Tao, J. et al. Mutations in the X-linked cyclin-dependent kinase-like 5 (CDKL5/STK9) gene are associated with severe neurodevelopmental retardation. American journal of human genetics 75, 1149-1154, (2004).
5. Weaving, L. S. et al. Mutations of CDKL5 cause a severe neurodevelopmental disorder with infantile spasms and mental retardation. American journal of human genetics 75, 1079-1093, (2004).
6. Hector, R. D. et al. Characterization of CDKL5 Transcript Isoforms in Human and Mouse. PloS one 11, e0157758, (2016).
7. Baltussen, L. L., et al. (2018). “Chemical genetic identification of CDKL5 substrates reveals its role in neuronal microtubule dynamics.” EMBO Journal 37(24).
8. Munoz, I. M., et al. (2018). “Phosphoproteomic screening identifies physiological substrates of the CDKL5 kinase.” EMBO Journal.

All documents cited in this specification are incorporated herein by reference, as is U.S. Provisional Patent Application No. 63/016,036, filed Apr. 27, 2020, and U.S. Provisional Patent Application No. 63/091,032, filed Oct. 13, 2020, U.S. Provisional Application No. 63/109,608, filed Nov. 4, 2020, International Patent Application No. PCT/US21/29185, filed Apr. 26, 2021, and U.S. Provisional Patent Application No. 63/256,827, filed Oct. 18, 2021 are incorporated by reference. The electronic sequence listing filed herewith named “UPN-22-9863PCT_SequenceListing_20221018.xml” with size of 281,561 bytes, created on date of Oct. 18, 2022, and the contents of the electronic sequence listing (e.g., the sequences and text therein) are incorporated herein by reference in entirety. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) useful for treating CDKL5 deficiency disorder (CDD), wherein the rAAV comprises:
(a) an AAVhu68 or AAVrh91 capsid; and
(b) a vector genome in the AAV capsid of (a), wherein the vector genome comprises a 5′ AAV inverted terminal repeat (ITR), an expression cassette comprising a human CDKL5 sequence of nucleotides 1 to 2883 of SEQ ID NO: 22 operably linked to regulatory sequences which direct expression thereof and which further comprise four tandem miR183 targeting sequences, and a 3′ AAV ITR.

2. The rAAV according to claim 1, wherein the regulatory sequences further comprise a UbC promoter or a hSyn promoter.

3. The rAAV according to claim 1, wherein the expression cassette comprises nucleic acid sequence of nucleotides 220 to 4609 of SEQ ID NO: 49 (or SEQ ID NO: 50), nucleic acid sequence of nucleotides 226 to 4608 of SEQ ID NO: 29 (or SEQ ID NO: 59) or the nucleic acid sequence of nt 224 to 4191 of SEQ ID NO: 31 (or SEQ ID NO: 60).

4. The rAAV according to claim 1, wherein the AAV capsid is an AAVhu68 capsid.

5. The rAAV according to claim 1, wherein the vector genome comprises an AAV 5′ ITR, a UbC promoter, an hCDKL5 coding sequence, four miR183 targeting sequences, a rabbit globin polyA signal, and an AAV 3′ ITR.

6. The rAAV according to claim 5, wherein the vector genome further comprises a Kozak sequence.

7. The rAAV according to claim 2, wherein the UbC promoter has the sequence of SEQ ID NO: 52.

8. The rAAV according to claim 1, wherein at least one of the miR183 targeting sequences has the sequence of
(miR183, SEQ ID NO: 11)
AGTGAATTCTACCAGTGCCATA.

9. The rAAV according to claim 1, wherein the four miR183 targeting sequences are located in tandem and are separated by spacer sequences.

10. The rAAV according to claim 1, which has an AAVhu68 capsid comprising a nucleic acid molecule comprising the vector genome of SEQ ID NO: 49.

11. A pharmaceutical composition comprising rAAV of claim 1 and one or more of a carrier, preservative, excipient, or an aqueous diluent.

12. The pharmaceutical composition according to claim 11 which comprises an aqueous liquid suitable for intracerebroventricular or intracisternal magma injection.

13. A method for treating CDKL5 deficiency disorder comprising administering an rAAV according to claim 1.

14-15. (canceled)

16. A nucleic acid molecule useful for producing an rAAV vector comprising a vector genome comprising a 5′ AAV inverted terminal repeat (ITR), an expression cassette comprising a human CDKL5 sequence of nucleotides 1 to 2883 of SEQ ID NO: 22 operably linked to regulatory sequences which direct expression thereof and which further comprise four tandem miR183 targeting sequences, and a 3′ AAV ITR.

17. The nucleic acid molecule according to claim 16, wherein the vector genome comprises SEQ ID NO: 49.

18. The nucleic acid molecule according to claim 16 which is a plasmid.

19. An rAAV production host cell comprising:
(a) a nucleic acid molecule according to claim 16;
(b) a nucleic acid molecule comprising an AAV capsid coding sequence, and optionally further comprising an AAV rep coding sequence; and
(c) adenovirus helper genes.
